SEQUENCE LISTING (45 sequences)

SEQ ID NO: 1 - protein, 16 aa, synthetic construct
  SITE 3: note = G at position 3 is N-allylglycine
  SITE 16: note = C-terminal amidation
FEGIYRTDILRTEEGN

SEQ ID NO: 2 - protein, 16 aa, synthetic construct
  SITE 3: note = G at position 3 is N-allylglycine
  SITE 16: note = C-terminal amidation
FEGIYRTELLKAEEAN

SEQ ID NO: 3 - protein, 16 aa, synthetic construct
  SITE 3: note = G at position 3 is N-allylglycine
  SITE 16: note = C-terminal amidation
FEGIYRLELLKAEEAN

SEQ ID NO: 4 - protein, 11 aa, synthetic construct
  SITE 3: note = G at position 3 is N-allylglycine
  SITE 11: note = C-terminal amidation
FEGIYRLELLK

SEQ ID NO: 5 - protein, 16 aa, synthetic construct
  SITE 3: note = A at position 3 is N-allylalanine
  SITE 16: note = C-terminal amidation
FEAIYRLELLKAEEAN

SEQ ID NO: 6 - protein, 17 aa, synthetic construct
  SITE 3: note = X is N-allylglycine
  MOD_RES 14: note = X is 2-Aminoisobutyric acid
  MOD_RES 17: note = X is 2-Aminoisobutyric acid
  SITE 17: note = C-terminal amidation
FEXGIYRLELLKXEEXN

SEQ ID NO: 7 - protein, 16 aa, synthetic construct
  SITE 3: note = G at position 3 is N-allylglycine
  SITE 16: note = C-terminal amidation
AEGIYRLELLKAEAAA

SEQ ID NO: 8 - protein, 16 aa, synthetic construct
  SITE 16: note = C-terminal amidation
  SITE 3: note = G at position 3 is N-allylglycine
AEGIYRLELLKAEAAA

SEQ ID NO: 9 - protein, 16 aa, synthetic construct
  SITE 16: note = C-terminal amidation
  SITE 1: note = N-terminal acetylation
FIGRLCTEILKLREGN

SEQ ID NO: 10 - protein, 16 aa, synthetic construct
  SITE 16: note = C-terminal amidation
  SITE 1: note = N-terminal acetylation
LAWRLRELERELARLC

SEQ ID NO: 11 - protein, 16 aa, synthetic construct
  SITE 1: note = N-terminal acetylation
  SITE 16: note = C-terminal amidation
WIGRLCTEILRLRNGN

SEQ ID NO: 12 - protein, 16 aa, synthetic construct
  SITE 16: note = C-terminal amidation
  SITE 1: note = N-terminal acetylation
LAWRLRELERELARLC

SEQ ID NO: 13 - protein, 16 aa, synthetic construct
  SITE 1: note = N-terminal acetylation
  SITE 16: note = C-terminal amidation
AIGRLCTEILRLRNGA

SEQ ID NO: 14 - protein, 16 aa, synthetic construct
  SITE 16: note = C-terminal amidation
  SITE 1: note = N-terminal acetylation
LAWRLRELERELARLC

SEQ ID NO: 15 - protein, 16 aa, synthetic construct
  SITE 1: note = N-terminal acetylation
  SITE 16: note = C-terminal amidation
WIGRLCTEILRLRNGN

SEQ ID NO: 16 - protein, 16 aa, synthetic construct
  SITE 1: note = N-terminal acetylation
  SITE 16: note = C-terminal amidation
LAWALRELERELARLC

SEQ ID NO: 17 - protein, 16 aa, synthetic construct
  SITE 10: note = X is L-homoarginine
  SITE 16: note = C-terminal amidation
  SITE 1: note = N-terminal acetylation
WIGRLCTEIXRLRNGN

SEQ ID NO: 18 - protein, 16 aa, synthetic construct
  SITE 1: note = N-terminal acetylation
  SITE 16: note = C-terminal amidation
LAWRLRELERELARLC

SEQ ID NO: 19 - protein, 16 aa, synthetic construct
  SITE 1: note = N-terminal acetylation
  SITE 16: note = C-terminal amidation
WIGRLCTEIRRLRNGN

SEQ ID NO: 20 - protein, 16 aa, synthetic construct
  SITE 1: note = N-terminal acetylation
  SITE 16: note = C-terminal amidation
LAWRLRELERELARLC

SEQ ID NO: 21 - protein, 16 aa, synthetic construct
  SITE 1: note = N-terminal acetylation
  SITE 16: note = C-terminal amidation
WIGRLCTEILRLRNGN

SEQ ID NO: 22 - protein, 16 aa, synthetic construct
  SITE 1: note = N-terminal acetylation
  SITE 16: note = C-terminal amidation
LAWRLRELERELARLC

SEQ ID NO: 23 - protein, 16 aa, synthetic construct
  SITE 1: note = N-terminal acetylation
  SITE 16: note = C-terminal amidation
FIGRLCTEILKLREGN

SEQ ID NO: 24 - protein, 17 aa, synthetic construct
  SITE 1: note = L at position 1 is L-beta-alanine
  SITE 1: note = FITC is 5-fluorescein isothiocyanate linked via thiourea bond to N-terminal amine
  SITE 17: note = C-terminal amidation
ALAWRLRELERELARLC

SEQ ID NO: 25 - protein, 16 aa, synthetic construct
  SITE 1: note = N-terminal acetylation
  SITE 16: note = C-terminal amidation
WIGRLCTEILRLRNGN

SEQ ID NO: 26 - protein, 17 aa, synthetic construct
  SITE 1: note = X is L-beta-alanine
  SITE 1: note = FITC is 5-fluorescein isothiocyanate linked via thiourea bond to N-terminal amine
  SITE 17: note = C-terminal amidation
XLAWRLRELERELARLC

SEQ ID NO: 27 - protein, 16 aa, synthetic construct
  SITE 1: note = N-terminal acetylation
  SITE 16: note = C-terminal amidation
AIGRLCTEILRLRNGA

SEQ ID NO: 28 - protein, 17 aa, synthetic construct
  SITE 1: note = X is L-beta-alanine
  SITE 1: note = FITC is 5-fluorescein isothiocyanate linked via thiourea bond to N-terminal amine
  SITE 17: note = C-terminal amidation
XLAWRLRELERELARLC

SEQ ID NO: 29 - protein, 16 aa, synthetic construct
  SITE 1: note = N-terminal acetylation
  SITE 16: note = C-terminal amidation
WIGRLCTEILRLRNGN

SEQ ID NO: 30 - protein, 17 aa, synthetic construct
  SITE 1: note = X is L-beta-alanine
  SITE 1: note = FITC is 5-fluorescein isothiocyanate linked via thiourea bond to N-terminal amine
  SITE 17: note = C-terminal amidation
XLAWALRELERELARLC

SEQ ID NO: 31 - protein, 16 aa, synthetic construct
  SITE 10: note = X is L-homoarginine
  SITE 1: note = N-terminal acetylation
  SITE 16: note = C-terminal amidation
WIGRLCTEIXRLRNGN

SEQ ID NO: 32 - protein, 17 aa, synthetic construct
  SITE 1: note = X is L-beta-alanine
  SITE 1: note = FITC is 5-fluorescein isothiocyanate linked via thiourea bond to N-terminal amine
  SITE 17: note = C-terminal amidation
XLAWRLRELERELARLC

SEQ ID NO: 33 - protein, 16 aa, synthetic construct
  SITE 1: note = N-terminal acetylation
  SITE 16: note = C-terminal amidation
WIGRLCTEIRRLRNGN

SEQ ID NO: 34 - protein, 17 aa, synthetic construct
  SITE 1: note = X is L-beta-alanine
  SITE 1: note = FITC is 5-fluorescein isothiocyanate linked via thiourea bond to N-terminal amine
  SITE 17: note = C-terminal amidation
XLAWRLRELERELARLC

SEQ ID NO: 35 - protein, 16 aa, synthetic construct
  SITE 10: note = X is L-homoarginine
  SITE 1: note = N-terminal acetylation
  SITE 16: note = C-terminal amidation
WIGRLCTEIXRLRNGN

SEQ ID NO: 36 - protein, 17 aa, synthetic construct
  SITE 17: note = C-terminal amidation
  SITE 1: note = diazirine photocrosslinker
GLAWRLRELERELARLC

SEQ ID NO: 37 - protein, 16 aa, synthetic construct
  CROSSLNK 10..11: note = diazirine photocrosslinker
  SITE 1: note = N-terminal acetylation
  SITE 16: note = C-terminal amidation
WIGRLCTEIKRLRNGN

SEQ ID NO: 38 - protein, 16 aa, synthetic construct
  SITE 1: note = N-terminal acetylation
  SITE 16: note = C-terminal amidation
LAWRLRELERELARLC

SEQ ID NO: 39 - protein, 107 aa, synthetic construct
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 40 - protein, 9 aa, synthetic construct
IEVMYPPPY

SEQ ID NO: 41 - protein, 5 aa, synthetic construct
GGGGS

SEQ ID NO: 42 - protein, 15 aa, synthetic construct
GGGGSGGGGSGGGGS

SEQ ID NO: 43 - protein, 574 aa, synthetic construct
  REGION 316..320: note = The entire sequence of amino acids at positions 316..320 can be repeated one or more times.
MEGWSCIILFLVATATGVHSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKTWIYDTSKVASGVPYRFSGSGSGTSYSLTISSMEAEDAATYYCQQWSSNPLTFGAGTKLELKGSGSGHHHHHH

SEQ ID NO: 44 - protein, 386 aa, synthetic construct
  REGION 128..132: note = The entire sequence of amino acids at positions 128-132 can be repeated one or more times.
MEGWSCIILFLVATATGVHSGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLEGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKTWIYDTSKVASGVPYRFSGSGSGTSYSLTISSMEAEDAATYYCQQWSSNPLTFGAGTKLELKGSGSGHHHHHH

SEQ ID NO: 45 - protein, 4 aa, synthetic construct
GALA

(19) United States
(12) Patent Application Publication
(10) Pub. No.: US 2025/0333447 A1
(43) Pub. Date: Oct. 30, 2025

(54) CHIMERIC SMALL MOLECULES FOR LABELING PROTEINS WITH IMMUNOGENIC MOIETIES AND METHODS OF USE THEREOF

(71) Applicants: The Brigham and Women's Hospital, Inc., Boston, MA (US); The Broad Institute, Inc., Cambridge, MA (US); President and Fellows of Harvard College, Cambridge, MA (US)

(72) Inventors: Amit Choudhary, Boston, MA (US); Sameek Singh, Cambridge, MA (US); Kaushik Pal, Cambridge, MA (US); Endri Karaj, Cambridge, MA (US); Shaimaa Sindi, Cambridge, MA (US); Rajaiah Pergu, Cambridge, MA (US); Jeffrey Fung, Cambridge, MA (US); Basudeb Mondal, Cambridge, MA (US); Santosh Chaudhary, Cambridge, MA (US)

(21) Appl. No.: 19/175,181

(22) Filed: Apr. 10, 2025

Related U.S. Application Data
(63) Continuation of application No. PCT/US2023/076508, filed on Oct. 10, 2023.
(60) Provisional application No. 63/414,828, filed on Oct. 10, 2022.
(60) Provisional application No. 63/486,403, filed on Feb. 22, 2023.
(60) Provisional application No. 63/471,178, filed on Jun. 5, 2023.
(60) Provisional application No. 63/534,994, filed on Aug. 28, 2023.
(60) Provisional application No. 63/539,051, filed on Sep. 18, 2023.

(51) Int. Cl. C07K 14/00; A61K 35/17; A61K 40/11; A61P 37/04; C07K 14/74; C07K 16/18; C07K 16/28; C12N 5/078

(52) U.S. Cl. CPC C07K 14/001; A61K 35/17; A61K 40/11; A61P 37/04; C07K 14/70539; C07K 16/18; C07K 16/2809; C12N 5/0634; A61K 2239/13; C07K 2319/30; C12N 2510/00

(57) ABSTRACT

Chimeric small molecules comprising an immunogenic display moiety and methods of using the chimeric small molecules to label proteins with the immunogenic display moiety for MHC display on the surface of a cell or to label cell surface proteins with the immunogenic display moiety for display on the surface of a cell, thereby inducing an immune response.

### CROSS-REFERENCE TO RELATED APPLICATIONS

This application is a continuation application of PCT/US2023/076508, filed Oct. 10, 2023, which claims the benefit of U.S. Provisional Application No. 63/414,828 filed Oct. 10, 2022, U.S. Provisional Application No. 63/486,403 filed Feb. 22, 2023, U.S. Provisional Application No. 63/471,178 filed Jun. 5, 2023, U.S. Provisional Application No. 63/534,994 filed Aug. 28, 2023, and U.S. Provisional Application No. 63/539,051 filed Sep. 18, 2023. The entire contents of the above-identified applications are hereby fully incorporated herein by reference.

### STATEMENT REGARDING FEDERALLY SPONSORED RESEARCH

This invention was made with government support under Grant Nos. GM137606 and AI162662, awarded by the National Institutes of Health, and under Grant No. HR0011-21-2-0010, awarded by the Defense Advanced Research Projects Agency. The government has certain rights in the invention.

### REFERENCE TO AN ELECTRONIC SEQUENCE LISTING

The contents of the electronic sequence listing (“BROD-5620US_ST26.xml”; Size is 75,789 bytes and it was created on Apr. 2, 2025) is herein incorporated by reference in its entirety.

### TECHNICAL FIELD

The subject matter disclosed herein is generally directed to chimeric small molecules comprising a protein binding moiety and an immunogenic display moiety, wherein the protein binding moiety facilitates labeling of a protein with the immunogenic display moiety, and wherein major histocompatibility complex (MHC) display of a fragment of the protein labeled with the immunogenic display moiety induces an immune response.

### SUMMARY

In an aspect, the present invention provides an immune cell recruiting chimeric small molecule comprising a target protein binding moiety and an immunogenic display moiety connected via one or more linker molecules, and optionally an electrophilic reactive group. wherein the protein binding moiety facilitates labeling of an amino acid of a protein, via the electrophilic reactive group, with the immunogenic display moiety. In one example embodiment, the chimeric small molecule is according to the formula A-L₁-E-B or A-L₁-E-L₂-B or A-E-L₁-B, where A is the target protein binding moiety; B is the immunogenic display moiety; L₁ and L₂ are each a linker; and E is an electrophilic reactive group. In one example embodiment, the immunogenic display moiety is configured to bind with (i) a cell surface of a natural or an engineered immune cell, or (ii) bifunctional bridge molecule comprising a first binding moiety that binds the immunogenic display moiety and a second binding moiety that binds the surface of a natural or engineered immune cell. In one example embodiment, the natural or an engineered immune cell is a CAR T cell, T cell or an NK cell. In one example embodiment, the target protein is a disease-specific protein, optionally an oncogenic-specific protein. In one example embodiment, the protein binding moiety is a KRAS^{G12C}, EGFR, pan-EGFR, ITK, FGFR4, JAK3, RIP1, MEK1/2, CDK, AKT, TAK, INK, BMX, LIMK, IRE1, IRE2, ABL1, EphA2 receptor, a human dipeptidyl peptidase IV/CD26, a HER2 receptor, a prostate-specific membrane antigen (PSMA), a folate receptor, or a somatostatin binding moiety. In one example embodiment, the amino acid is lysine or cysteine.

In an aspect the present invention provides a bifunctional immune cell engager comprising a first binding moiety capable of binding the immunogenic display moiety and a second binding moiety capable of binding a cell surface receptor of a natural or engineered immune cell. In one example embodiment, the immune cell is a CD8 T cell, a CD4 T cell, a NK cell, a CAR T cell, or an engineered tumor infiltrating lymphocyte (TIL). In one example embodiment, the cell surface receptor is CD3, CD19, CD20, CD22, CD30, CD33, CD38, CD79B, or SLAMF7. In one example embodiment, the immunogenic display moiety of the chimeric small molecule and the first binding moiety of the bifunctional immune cell engager together comprise a click chemistry reagent pair. In one example embodiment, a binding domain of the second binding moiety is masked such that the second binding moiety is incapable of binding a cell surface receptor of a natural or engineered immune cell, and wherein the click chemistry reaction of the click chemistry reagent pair unmasks the binding domain of the second binding moiety, such that the second binding moiety is capable of binding the cell surface receptor of the natural or engineered immune cell. In one example embodiment, the immunogenic display moiety is a tetrazine moiety, or a tetracyclooctene (TCO) moiety, and the first binding moiety is a corresponding TCO moiety, or tetrazine moiety. In one example embodiment, the immunogenic display moiety is a Halo Tag ligand and the first binding moiety is a Halo Tag protein. In one example embodiment, the immunogenic display moiety is an E3 ligase ligand, and the first binding moiety is an E3 ligase ligand binding moiety of a CRBN protein, or an antibody or antibody fragment to an E3 ligase ligand. In one example embodiment, the first binding moiety is an antibody, a scFV fragment, or a nanobody directed against the immunogenic display moiety. In one example embodiment, the bifunctional immune cell engager is a BiTE, wherein the first binding moiety is a first antibody variable region the binds the immunogenic display moiety and the second binding moiety is a second antibody variable region that binds a cell surface receptor on an immune cell.

In an aspect, the present invention provides a method of inducing immune response, comprising: delivering the immune cell recruiting chimeric small molecule to a subject in need thereof; labeling one or more target polypeptides of one or more target proteins with the immunogenic display moiety by the immune cell recruiting chimeric small molecule; and displaying the one more target polypeptides labeled with the immunogenic display moiety on the cell surface via a Major Histocompatibility Complex (MHC) molecule. In one example embodiment, the method further comprises eliciting an immune response by binding of the immunogenic display moiety to a natural or an engineered immune cell, thereby activating the immune cell. In one example embodiment, the method further comprising eliciting an immune response by administering the bifunctional immune cell engager wherein the first binding moiety of the bifunctional immune cell engager binds the immunogenic display moiety displayed on the surface of the cell and the second binding moiety of the bifunctional immune cell engager binds a cell surface receptor of a natural or engineered immune cell thereby activating the natural or engineered immune cell. In one example embodiment, two or more different target proteins are labeled with the same immunogenic display moiety, whereby each target polypeptide of each different target protein is recognized by the same natural or engineered immune cell. In one example embodiment, the chimeric small molecule that labels the two or more different target proteins is the same molecule or different molecules.

In an aspect, the present invention provides a method of labeling cell surface polypeptides, comprising: delivering the chimeric small molecule to a cell; and labeling one or more target cell surface polypeptides with the immunogenic display moiety by the chimeric small molecule. In one example embodiment, the method further comprises eliciting an immune response by binding of the immunogenic display moiety to a natural or an engineered immune cell, thereby activating the natural or engineered immune cell. In one example embodiment, the method further comprises eliciting an immune response by administering the bifunctional immune cell engager to the cell surface, wherein the first binding moiety of the bifunctional immune cell engager binds the immunogenic display moiety and the second binding moiety of the bifunctional immune cell engager binds a cell surface receptor of a natural or engineered immune cell, thereby activating the natural or engineered immune cell. In one example embodiment, two or more different target cell surface polypeptides are labeled with the same immunogenic display moiety, whereby each different target cell surface polypeptide is recognized by the same natural or engineered immune cell. In one example embodiment, the chimeric small molecule that labels the two or more different cell surface polypeptides is the same chimeric small molecule or different chimeric small molecules. In one example embodiment, the target protein is a disease-specific protein, optionally an oncogenic-specific protein. In one example embodiment, the target protein is KRASG12C, EGFR, pan-EGFR, ITK, FGFR4, JAK3, RIP1, MEK1/2, CDK, AKT, TAK, JNK, BMX, LIMK, IRE1, IRE2, ABL1, EphA2 receptor, a human dipeptidyl peptidase IV/CD26, a HER2 receptor, a prostate-specific membrane antigen (PSMA), a folate receptor, or somatostatin. In one example embodiment, the target cell surface polypeptide is a disease-specific polypeptide, optionally an oncogenic-specific polypeptide. In one example embodiment, the target cell surface polypeptide is a prostate-specific membrane antigen (PSMA), a folate receptor, a somatostatin receptor, a human dipeptidyl peptidase IV/CD26, a HER2 receptor, or EGFR polypeptide.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1—Major histocompatibility complex (MHC) display mediated recruitment of T cells to cancer cells.

FIG. 2—Construct design for Hybrid-BiTE (SEQ ID NOS: 43-44).

FIG. 3—Compounds for human leukocyte antigen (HLA) display: KRAS (G12C) with HaloTAG, dinitrophenyl (DNP), and FKPB12^{F36V}.

FIG. 4—MHC display of potential immunogenic agents (e.g., HaloTAG) by targeting KRAS G12C.

FIG. 5—Proof-of-concept data by targeting KRAS G12C.

FIG. 6—MCH display of potential immunogenic agents (e.g., phospho-antigens) by targeting KRAS G12C.

FIG. 7—In-vitro covalent labeling of KRAS G12C.

FIG. 8—Generalization to kinases dysregulated in cancer.

FIG. 9—Generalization to kinases dysregulated in cancer (continued).

FIG. 10—Molecules developed to target cysteine in kinases.

FIG. 11—Molecules developed to target lysine in kinases.

FIG. 12—Example T-cell activation assay.

FIG. 13A-13B—Test results for PK1335.

FIG. 14—Schematic of MHC display mediated recruitment of T cells to cancer cells and test results for chimeric molecules targeting various target proteins.

FIG. 15—Test results for chimeric molecules targeting KRAS (Cys) having different target binding groups or different linker groups.

FIG. 16—T-cell recruiting chimeras (TRCs) for extracellular targets.

FIG. 17—Example molecules that target the extracellular domain of oncoproteins.

FIG. 18—Example molecules that target the intracellular domain of kinases involved in cancer.

FIG. 19—General Schematic of a chimeric molecule.

FIG. 20—Example target binder groups for various target proteins; and example chimeric molecules for the shown target binders.

FIG. 21—Example cleavable linker groups for targeting cysteine or lysine amine acid groups and an example lysing targeting molecule.

FIG. 22—Example connecting linker groups.

FIG. 23—Example molecules with different connecting linker groups.

FIG. 24—Example display functional groups and example molecules for the shown display functional groups.

FIG. 25—Example of chemical trigger of an inert Anti-CD3 into an active Anti-CD3 via removal of a masking group.

FIG. 26A-26G—Development of T-cell recruiting chimera for KRAS protein and T-cell Recruitment via Inside-out Covalent Labelling (TRICL) assay (FIG. 26A) Design and synthesis of T-cell recruitment chimeras comprised of a KRAS G12C binder and HaloTag ligand fixed at the ends and linked with various linkers. (FIG. 26B) A scheme of the TRICL assay where a bifunctional chimera covalently installs the HaloTag ligand on the KRAS(G12C) protein inside the cell and after proteasomal digestion the peptide along with HaloTag ligand displayed outside the cell on the MHC, a bifunctional protein covalently reacts with displayed HLA and recruits and activates T-cell via Anti-CD3 (on protein) and CD3 recognition (on T-cell surface). (FIG. 26C) Best linker is identified via bioluminescence based TRICL assay using KRAS(G12C) cell (MIA-PaCa2) and IL-2 luciferase (IL-2 Luc) reporter T-cell (Jurkat). Normalized with DMSO treatment value. (FIG. 26D) The specificity of the target protein is tested using KRAS G12C and G12D containing cells, an insignificant activation was observed with G12D cell compared to the G12C. Normalized with DMSO treatment value. (FIG. 26E) Bifunctional protein linked with G4S or 3(G4S) were tested with the best chimeric molecule (10) which shows that longer linker works better. (FIG. 26F) Chemical structures of the covalent KRAS(G12C) inhibitor Sotorasib (10S) and a bifunctional chimera with a BTK binder (11). (FIG. 26G) TRICL assay with 11 shows no T-cell activation whereas 10 in presence of 10S shows reduced T-cell activation, normalized with DMSO treatment value.

FIG. 27A-27D—T-cell recruitment via labelling intracellular kinase (FIG. 27A) Chemical structures of the kinase targeting bifunctional chimeric molecules. (FIG. 27B) TRICL assay by targeting intracellular kinase showing different extent of activation (KRAS as the reference here) in different cells (Target:cell-FGFR:AN3CA, BTK:Raji, KRAS:MIA-PaCa2, JAK3:RS4-11, ITK:Jurkat, EGFR:A431). (FIG. 27C) The FGFR targeting chimeric molecule showing different T-cell activation efficiency in different cells, which express different level of FGFR. (FIG. 27D) TRICL assay with all chimeric molecules and various cell lines which either express the target or not, shows various T-cell activation efficiency corroborating the target expression.

FIG. 28—Chimeras for enhancing protein function by cargo transfer or release.

FIG. 29—Chimeras for recruiting immune system.

FIG. 30—Bispecific T-cell Engager (BiTE): Contemporary and emerging applications.

FIG. 31—Challenges with the current design.

FIG. 32—T-cell recruiting chimeras (TRCs) for intracellular targets.

FIG. 33—Three designs for T-cell recruiting chimeras (TRCs) for intracellular targets.

FIG. 34—Library generation and description of T cell activation assay.

FIG. 35—Linker type (rigid vs. flexible, long vs short) impacts T cell activation.

FIG. 36—T cell activation is specific for KRAS C12C (not G12D or when BTK binder is used).

FIG. 37—EGFR mutant selective binder results in selective T cell activation compared to wild type (wt) EGFR cells.

FIG. 38—Rapid generalization to other targets using other clinically approved inhibitors.

FIG. 39—Degree of T cell activation is dependent on target expression (haplotype independent).

FIG. 40—T-cell recruiting chimeras (TRCs) for extracellular targets.

FIG. 41—A T cell recruitment platform using clinically approved inhibitors.

FIG. 42—Cell-specific release of phosphoantigens or other cargos.

FIG. 43A-43D—(FIG. 43A) A contemporary approach to haptenizing an oncogene (e.g., KRAS^{G12C}) using a covalent drug (box). Here the T cell engager (i.e., BiTE) binds to MHC:haptenated peptide complex on cancer cell and CD3 on T cell to induce proximity between cancer and T cell. (FIG. 43B) A Haptenizing chimeras (HaCs) platform wherein the covalent drug tags the oncogene with a bio-orthogonal group (e.g., tetrazine) that can react with a T cell engager to induce proximity between cancer and T cells. (FIG. 43C) A PROTAC-based HaC platform where HaC induces degradation and haptenization of the oncogene. (FIG. 4D) A HaC platform for extracellular targets.

FIG. 44A-44H—(FIG. 44A) A library of HaCs for KRAS^{G12C}. (FIG. 44B) Schematic of luciferase-based assay for monitoring T-cell activation. (FIG. 44C) T-cell activation by the different HaCs. (FIG. 44D) Linker optimization of the T cell engager showing 3G4S linker yielding higher activation. (FIG. 44E) T-cell activation by a HaC for KRAS^{G12C} in various cancer cells (MIA PaCa-2 only expresses KRAS^{G12C}). (FIG. 44F) Selective T-cell activation by a HaC in KRAS^{G12C} but not KRAS^{G12D} cells. (FIG. 44G) Structures of Sororasib 6 and a BTK HaC 7. (FIG. 44H) No T-cell activation was observed by BTK HaC in MIA PaCa-2 cells and T-cell activation by HaC 5a is competed by Sotorasib.

FIG. 45A-45E—(FIG. 45A) Structures of HaCs for ITK, JAK3, FGFR and EGFR. (FIG. 45B) Generalization of the platform to other targets. (FIG. 45C) EGFR (T790M/L858R) targeting HaC 11 selectively activates T-cell only with the double mutated cell line (H1975) and not with the wild type (A431). (FIG. 45D) HaC 10 leads to higher T-cell activation in AN3CA cells. (FIG. 45E). Relative expression levels of FGFR1.

FIG. 46A-46E—(FIG. 46A) Schematic presentation of T-cell recruitment based on the biorthogonal reaction of tetrazine with TCO. (FIG. 46B) Structure of tetrazine-based HaC targeting FGFR (20). (FIG. 46C) 21 activates leads to T-cell activation after reaction with TCO conjugated anti-CD3 antibody. (FIG. 46D) Structure of BTK PROTAC 21. (FIG. 46E) Co-treatment of BTK HaC 7 (20 uM) with 21 (0 or 10 uM) provides enhanced T-Cell activation.

FIG. 47A-47F—(FIG. 47A) General composition of the designed HaCs. (FIG. 47B) Structures of KRAS^{G12C} binder and EGFR^{T790M}. (FIG. 47C) Structures of different reactive groups organized from the most reactive (top) to the least reactive (bottom). The reaction rates are predicted from the corresponding β-amino acrylamides. (FIG. 47D) Chemical structures for different flexible and rigid linkers. (FIG. 47E) Structures of tetrazine analogs with varying reactivity. (FIG. 47F) Generation of T-cell engager via conjugation of anti-CD3 scFv with TCO via NHS chemistry (top) or via Sortase conjugation (bottom).

FIG. 48A-48F—(FIG. 48A) Competitive activity-based protein profiling for quantitative analysis of POI labeling by HaCs. (FIG. 48B) Schematic for TAP assay. (FIG. 48C) Schematic for differential scanning fluorimetry (DSF). (D) Schematic for proximity ligation assay (PLA). (FIG. 48E) Schematic of T-cell activation by Luciferase reporter assay. Luciferase expression is initiated after T-cell receptor (TCR) cascade activation. (FIG. 48F) Schematic of cancer cell killing by PMBCs.

FIG. 49A-49D—(FIG. 49A) General composition of PROTAC based HaCs. (FIG. 49B) Chemical structure of the BTK binder based on Ibrutinib. (FIG. 49C) Chemical structures of Lenalidomide and Pomalidomide E3-Ligase binders. (FIG. 49D) Chemical structures of PROTAC linkers based on BTK degraders.

FIG. 50A-50F—(FIG. 50A) Reaction of NASA 12 and alkyl 13 with lysine coumarin (aminolysis) or with water (hydrolysis). (FIG. 50B) Alkylation with of N-acetyl-Sulfonamides with different groups provides NASA analogs with tunable reactivity. (FIG. 50C) Alkylation with small, hydrophobic and electron withdrawing groups provides analogs with enhanced hydrolytic stability. (FIG. 50D) Structure of the PSMA based HaC with the optimized NASA warhead. (FIG. 50E) Crystal of PSMA with inhibitor (PDB 2XEJ). LC-Ms/Ms of PSMA with compound 19 showed selective labeling of K537. (FIG. 50F) Compound 19 leads to T-cell activation only on PSMA positive prostate cancer cell lines.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a” “an”, and “the” include both singular and plural referents unless the context clearly dictates otherwise.

The term “optional” or “optionally” means that the subsequent described event, circumstance, or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

Embodiments disclosed herein provide compositions and methods for eliciting targeted immune responses against cells expressing select target proteins. Compositions comprising a chimeric bi-functional small molecule (referred to herein “immune cell recruiting chimeric small molecule”) that can bind the target protein via a target protein binding moiety and label the target protein with an immunogenic display moiety. The target protein binding moiety and the immunogenic display moiety are connected by a linker, specifically a linker comprising one or more electrophilic reactive groups that can facilitate transfer of the immunogenic display moiety to the surface of the target protein. Fragments of the labeled target protein comprising the immunogenic display moiety are then displayed on the cell surface via natural cellular degradation of the target protein and MHC-mediated display of the resulting fragments. The immune recruiting chimeric small molecules may also be used to label cell surface expressed proteins with the immunogenic display moiety and in such embodiment degradation and MHC-mediated display is not necessarily required. In one embodiment, the immunogenic display moiety may bind directly to a surface of a natural or engineered immune cell, such as a T cell or a natural killer (NK) cell, leading to activation of the immune cell against the cell expressing the target protein. In another embodiment, an additional bifunctional molecule (referred to herein as an “immune cell engager” or “bifunctional immune cell engager”) capable of binding the immunogenic display moiety and the surface of a natural or engineered immune cell, is provided and thereby activates the immune cell against the cell expressing the target protein. Different cell phenotypes, including disease-specific phenotypes, are often marked by the expression of specific proteins or mutated forms of proteins. Accordingly, the compositions and methods provided herein a targeted way to induce immune responses against specific target cells marked by expression of phenotype-specific proteins, including disease-specific proteins.

The immune cell recruiting chimeric small molecule is modular in design and can be rapidly adapted to new target proteins without requiring building of a new molecule from the ground up. For example, the same immunogenic display moiety may be used with any target binding moiety requiring only a change in the target binding moiety to re-purpose the molecule for labeling of different target proteins. The ability to keep the immunogenic display moiety constant for a given molecule design also allows for the design of a single immune cell engager molecule that can be used with multiple different immune cell recruiting chimeric small molecules each comprising different target protein binding moieties but having the same immunogenic display moiety. The small molecule nature of the immune cell recruiting molecules also increases cell penetration and delivery. The designs of the immune cell recruiting chimeric small molecules also allow targeting of intracellular proteins and are not limited to extracellular proteins as are some current therapeutic modalities. As demonstrated herein, the ability to activate an immune response using the immune cell recruiting chimeric small molecules and immune cell engager molecules is haplotype independent, and therefore not by impacted by genetic variation in HLA sub-types across a patient population, or variations in the strength of the immune response elicited by different HLA sub-types.

### Immune Cell Recruiting Chimeric Small Molecules

In one example embodiment, the chimeric small molecule has the following formula: A-L1-E-B or A-L1-E-L2-B or A-E-L1-B, wherein A is a target protein binding moiety; B is an immunogenic moiety, e.g., an immunogenic display moiety; L1 and L2 are each a linker; and E is an electrophilic reactive group. The electrophilic reactive group of the chimeric small molecule may be designed to react with a moiety on the protein, for example, on an amino acid of the protein (e.g., via a Michael Addition reaction between a cysteine or lysine amino acid and an electrophilic group connected to the protein binding moiety). The electrophilic reactive group can be advantageously designed to react with a moiety in proximity to the binding site of the target protein binding moiety on the target protein. The reaction of the electrophilic reactive group with a moiety on the target protein, for example, a nucleophilic group disposed on the target protein, can allow the labeling or binding of the target protein with the immunogenic display moiety. Such binding of an immunogenic display moiety to the target protein can result in the formation of peptide fragments of the target protein comprising said immunogenic display moiety after the target protein is degraded via cell protein turnover and degradation processes.

### Target Protein Binding Moiety

The target protein binding moiety can comprise any small molecule capable of binding to a target protein. The target binding moiety may be an allosteric binder, i.e., binding to the target protein at a site other than the active site of the target protein. The target binding moiety may also bind to the active site of the target protein. The target protein binding moiety may target one or more different protein targets, or target one or more locations on the same target protein.

The target protein binding moiety is chosen based on the target protein. Ideally the protein will be specific for a disease phenotype of interest. For example, there are many proteins that are expressed only in tumor cells of different types of cancer. See, e.g. Zhou et al. “Proteomic signatures of 16 major types of human cancer reveal universal and cancer-type-specific proteins for the identification of potential therapeutic targets” J. Hematol Oncol 13, 170 (2020) (which assay the proteomic signatures of 16 major types of human cancer revealing a number of cancer-type-specific proteins). Accordingly, depending on the disease phenotype of interest, the target protein can be selected that provides the desired level of specificity for the disease phenotype, and a target binding moiety selected accordingly. It should be noted that the target protein may be expressed in more than one tissue type but can also be selected on the basis of overexpression in the disease phenotype, which can allow for selective dosing of the immune cell recruiting chimeric small molecule to take advantage of the higher concentration of the target protein in the specific cell type for which eliciting of an immune response is desired.

Advantageously, the target binding moiety can be an activator or inhibitor of the target protein. A target binding moiety may be chosen based on high abundance of the target protein in a target cell; available crystal structure and characterization of the target protein active sites or allosteric sites; target binding moieties with low residence time at the binding site; the ability of the target to accommodate a bio-orthogonal group, e.g. a small biorthogonal handle, without affecting binding potency and/or residence time; and/or target proteins with a high density of amino acids with nucleophilic side chains, e.g. serines/threonines/tyrosines/lysines close to the binding pocket; Linker length may be tuned, allowing labeling of the immunogenic display moiety with increased distance from binding pocket, allowing modification to be targeted to locations, for example, at amino acid residues, farther away from the binding pocket. For example, a longer linker length can be utilized when a bioconjugation reaction is desirable further away from a binding pocket but optimized for a length that still allows the target binding moiety, once bound to a target substrate in close proximity to the binding pocket of the kinase. Tuning linker length may also include a level of flexibility or rigidity depending on desired configuration of the target binding moiety for modifications of amino acid residues. A shorter linker length may allow for modification within the binding pocket which may be desirable for some applications.

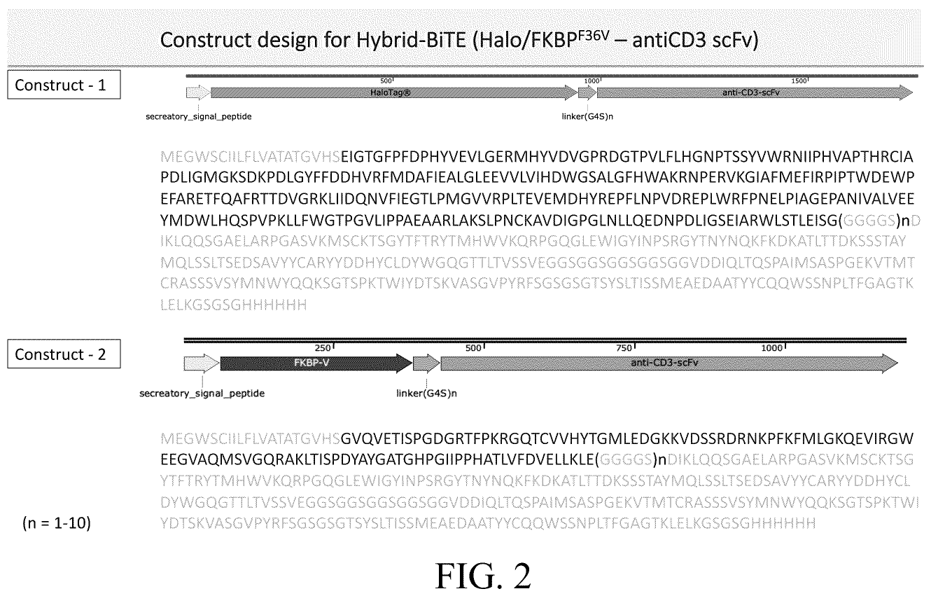

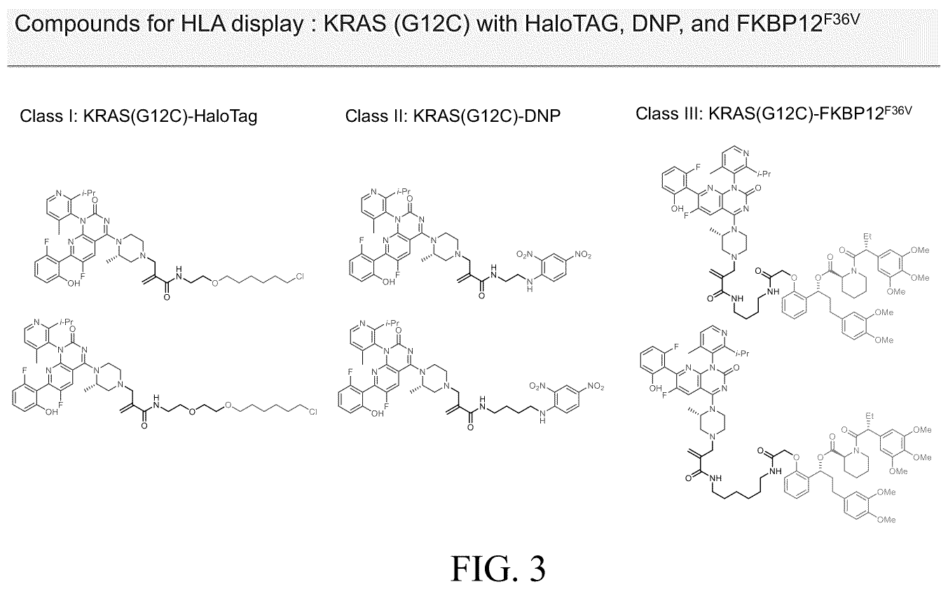

In an embodiment, the target binding moiety is an allosteric modulator. Considerations in selecting a target binding moiety may include allosteric signaling, which may include changes associated with networks of non-covalently interacting protein residues, conformational selection, and induced fit with both spatial and temporal aspects. In one embodiment, the target binding moiety may be an allosteric activator or inhibitor of the kinase. Allosteric activators or inhibitors may be discovered computationally. In one example method, high quality drug targets are acquired. Then allosteric site prediction is performed using methods such as perturbation response scanning (PRS) combined with all-atom molecular dynamics (MD) and dynamic residue networks (DRN). Allosteric modulators are then identified using methods such as homology modeling, docking, or essential dynamics. An illustration of this process can be found in FIGS. 2 and 3 of Amamuddy S., et al. Integrated Computational Approaches and Tools for Allosteric Drug Discovery, 21 IJMS, 847 (2020), incorporated herein by reference.

The target binding moiety may be chosen in part based on its half-life. In one example embodiment, the target binding moiety may be chosen based in part on its half-life relative to the half-life of the target protein. In an embodiment, the half-life of the target binding moiety is 2, 3, 4, or 5 times shorter than that of the target protein. Without being bound by a particular theory, design of a chimera small molecule with a half-life of the target binding moiety shorter than that of the kinase may allow for desirable reaction kinetics when the target protein is labeled by the via the electrophilic reactive group. The half-life of the target binding moiety and the target protein generally relates to the time required for the concentration of the target binding moiety or target protein to decrease to half of its initial concentration. In one embodiment, the half-life may measure the time it takes to degrade half of the molecules initially measured in a sample, which may comprise a cell, cells, tissue, organoid, or mammal, for example. In one embodiment, the half-life of the kinase and the target binding moiety is measured in the same or similar conditions, for example, in a same cell type, tissue, or organism. In one example embodiment, the measurement of half-life can be measured in a same sample or system that has a particular phenotype, genotype, disease or condition to be studied, treated and/or evaluated.

Measurement of the half-life of the target binding moiety may be determined, for example, by dissociation t_{1/2} or receptor occupancy t_{1/2}, describing the average time needed to liberate half of the initially occupied target proteins under conditions in where association of the target protein binding moiety or its rebinding can take place. Dissociation that requires a target protein conformational change or binding pocket size may play a factor in the residence time and can be considered when selected the target protein binding moiety. See, e.g. Roskoski R Jr. Classification of small molecule protein kinase inhibitors based upon the structures of their drug-enzyme complexes. Pharmacol Res. 2016; 103:26-48. doi:10.1016/j.phrs.2015.10.021.

The time a compound resides on its target, e.g., residence time, may be used. See, Willemsen-Seegers N, Uitdehaag J C M, Prinsen M B W, et al. Compound Selectivity and Target Residence Time of Kinase Inhibitors Studied with Surface Plasmon Resonance. J Mol Biol. 2017; 429(4):574-586. doi:10.1016/j.jmb.2016.12.019, for discussion and identification of residence time and parameters of exemplary kinase binding moieties, incorporated herein in its entirety, and in particular Table 1,3A-3B, 4A-4C, S3 and S4, for teachings to tyrosine kinase inhibitors, EGFR inhibitors, ponatinib to a variety of kinases, particular kinases and their associated inhibitors, Aurora A and B kinase inhibitors, and P13k lipid kinase inhibitors. Elimination half-life may also be utilized alone or in conjunction with residence time evaluation. Additional pharmacodynamics and pharmacokinetics may also be considered in the evaluation of half-life for the kinase binding moiety. Half-life may be modeled. See, e.g. Callegari D, Lodola A, Pala D, et al. Metadynamics Simulations Distinguish Short- and Long-Residence-Time Inhibitors of Cyclin-Dependent Kinase 8 [published correction appears in J Chem Inf Model. 2017 Feb. 27; 57(2):386]. J Chem Inf Model. 2017; 57(2):159-169. doi:10.1021/acs.jcim.6b00679, incorporated herein by reference.

The target binding moiety may also be selected based on a measurement of half-life of the target protein, approaches measuring half-life such as mass spectrometry-based proteomics such as SILAC (stable isotope labeling by amino acids in cell culture)-based proteomics, see, e.g. Matheison et al., Nature Communications volume 9, Article number: 689 (2018), may be used. High throughput proteomics may be used to estimate a target protein half-life in a particular tissue and/or cell, or further predictive modeling may be used to predict such target protein half-life in tissue from cellular properties, see, e.g., Rahman M, Sadygov R G Predicting the protein half-life in tissue from its cellular properties. PLoS ONE 12(7): e0180428. doi.org/10.1371/journal.pone.0180428 (2017).

The following provide examples of further, non-limiting, protein binding moieties to various target proteins of interest in oncology and infectious disease contexts and the use of which will discussed further in the Methods of Use section below. In one example embodiment, a protein binding moiety is an oncoprotein binding moiety, including but not limited to a KRAS binding moiety (e.g., G12C, or G13C). In one example embodiment, a protein binding moiety is a kinase binding moiety, including, but not limited to, a tyrosine kinase binding moiety. In one example embodiment, a target kinase is selected from EGFR (e.g., EGFR, pan-EGFR), FGFRs, JAK3, ITK, CDK, AKT, TAK, INK, BMX, LIMK, IRE1, IRE2, RIP1, MEK1/2, ABL1, EphA2.

In one example embodiment, the target protein is an extracellular protein, e.g., a prostate-specific membrane antigen (PSMA), a folate receptor, a somatostatin receptor, a human dipeptidyl peptidase IV/CD26, a HER2 receptor, or EGFR. In one example embodiment, a target protein is NRAS (e.g., G12C), FGFR3 (e.g., R248C, S249C, G370C, or Y373C), TP53 (e.g., Y220C, or R273C), IDH1 (e.g., R132C), GNAS (e.g., R201C), FBXW7 (e.g., R465C), CTNNB1 (e.g., S33C, or S37C), or DNMT3A (e.g., R882) protein.

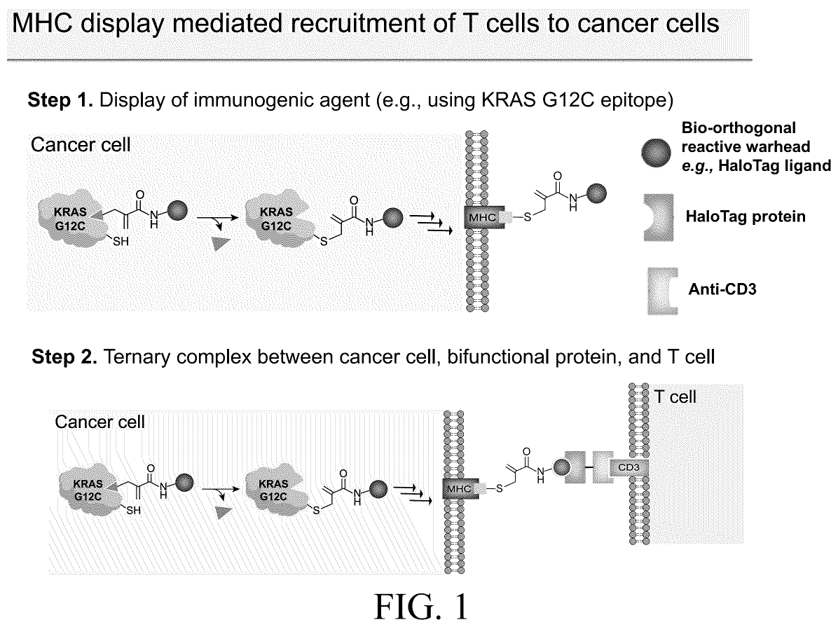

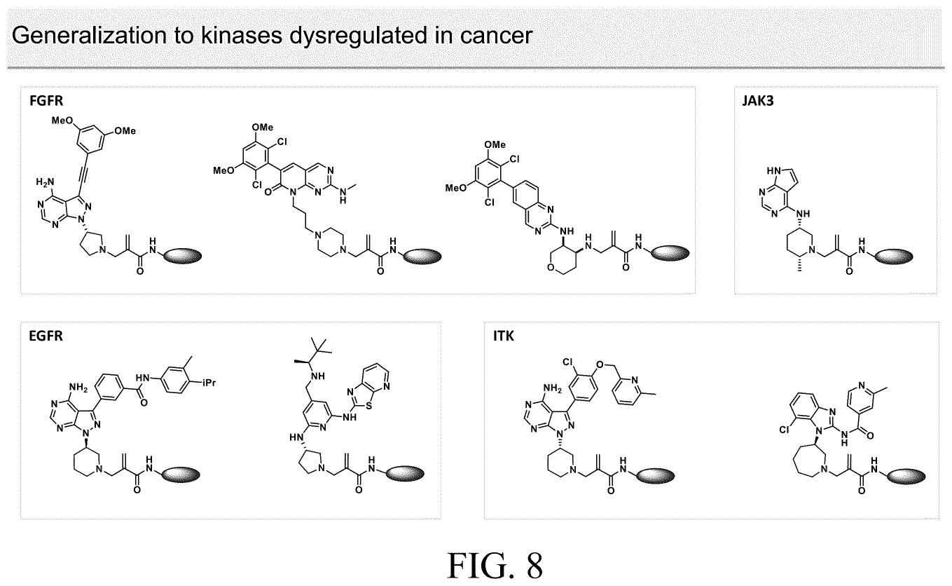

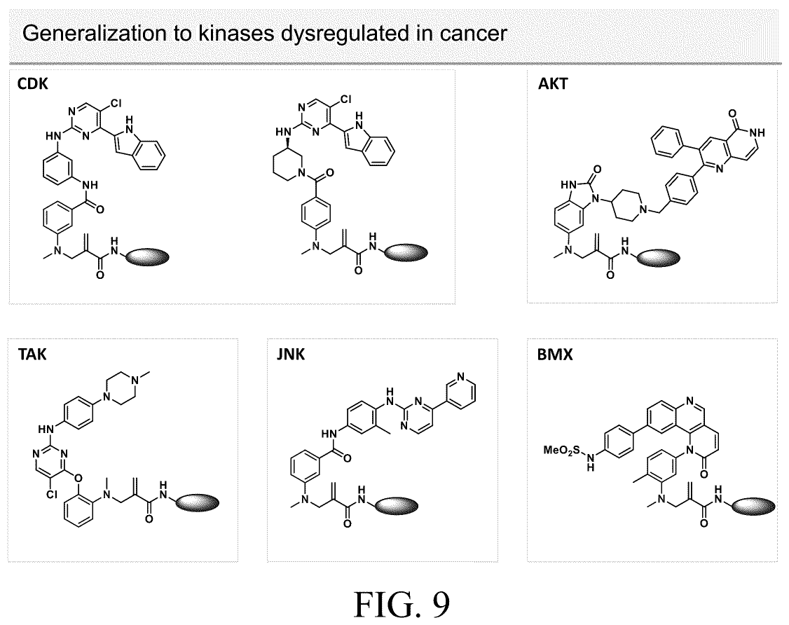

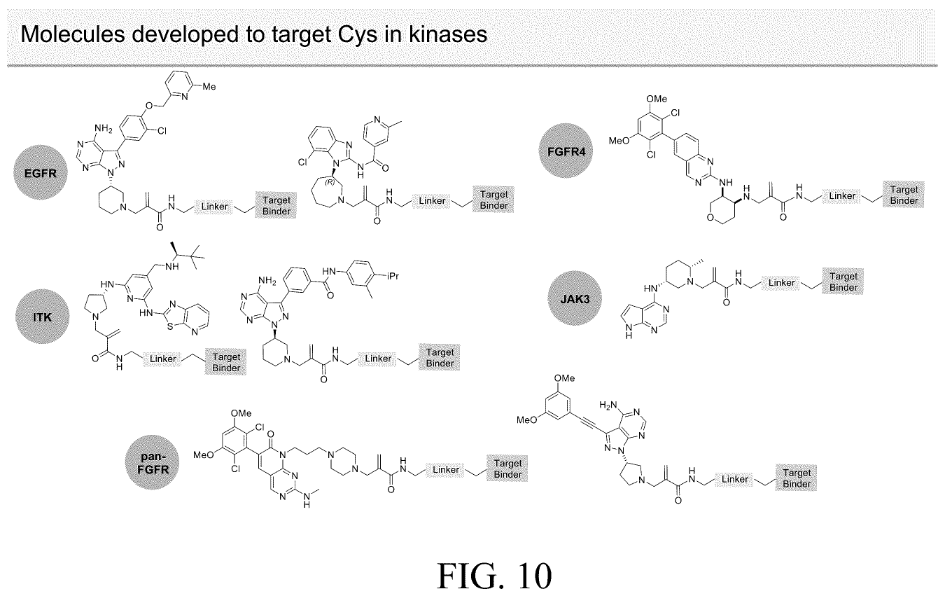

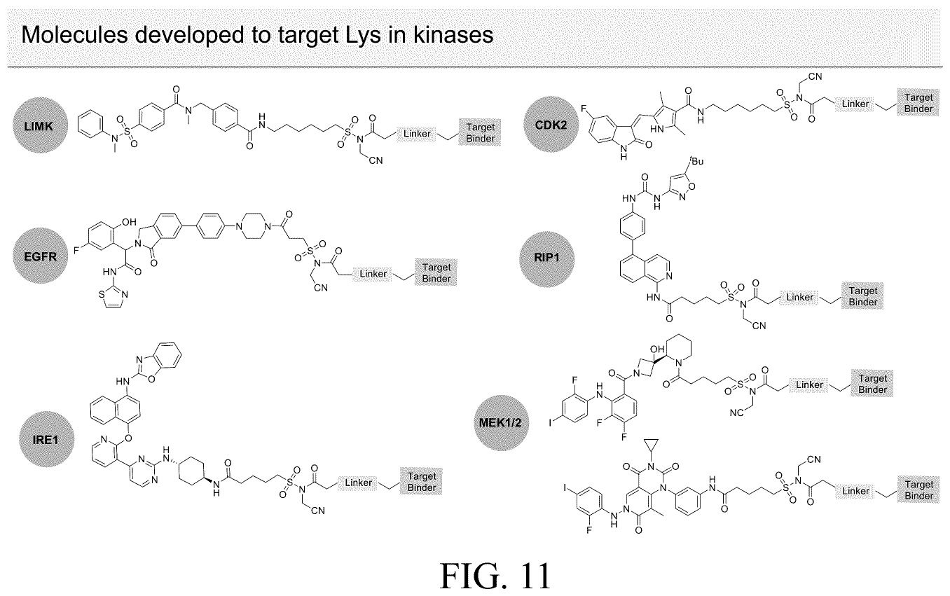

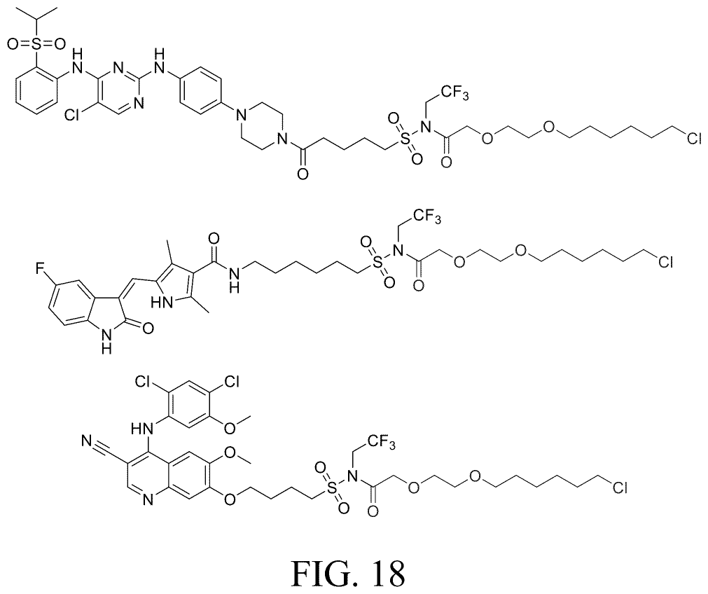

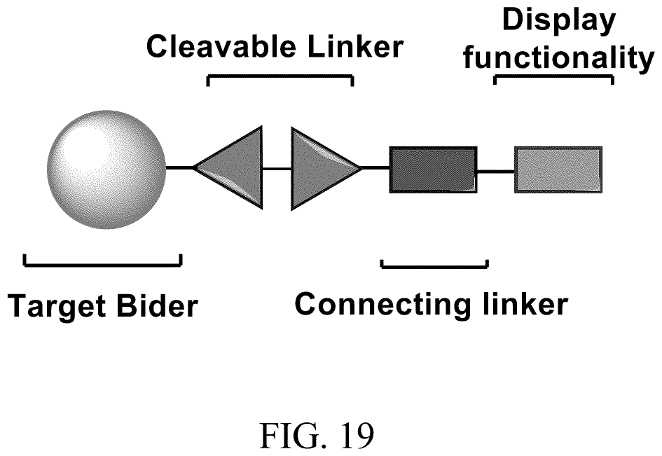

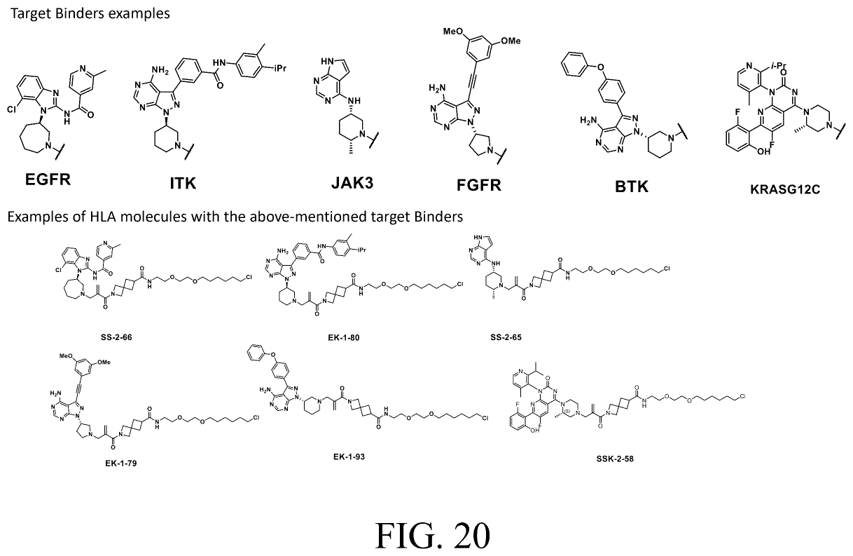

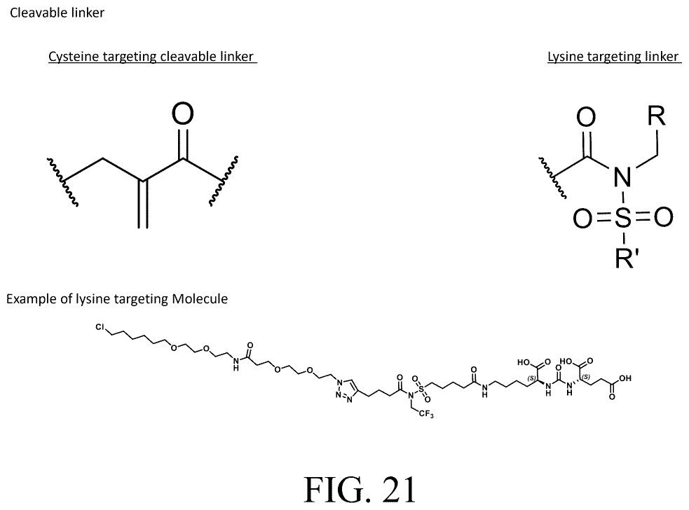

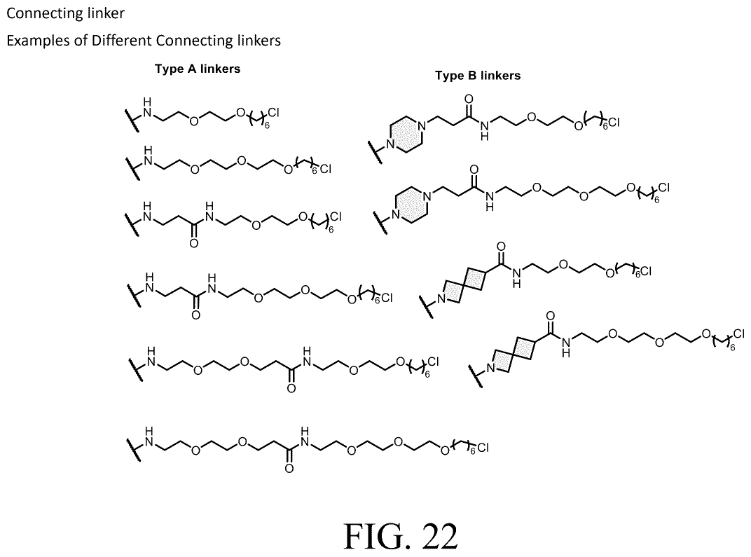

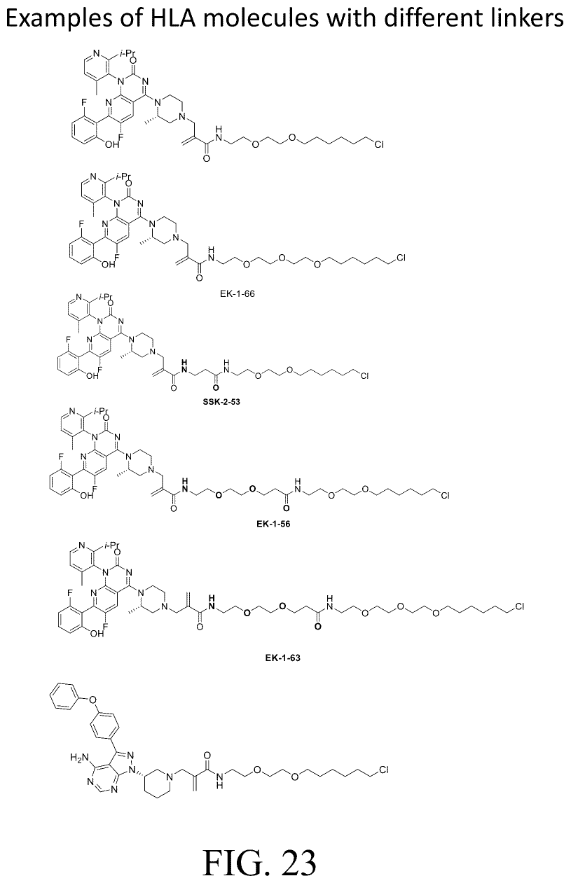

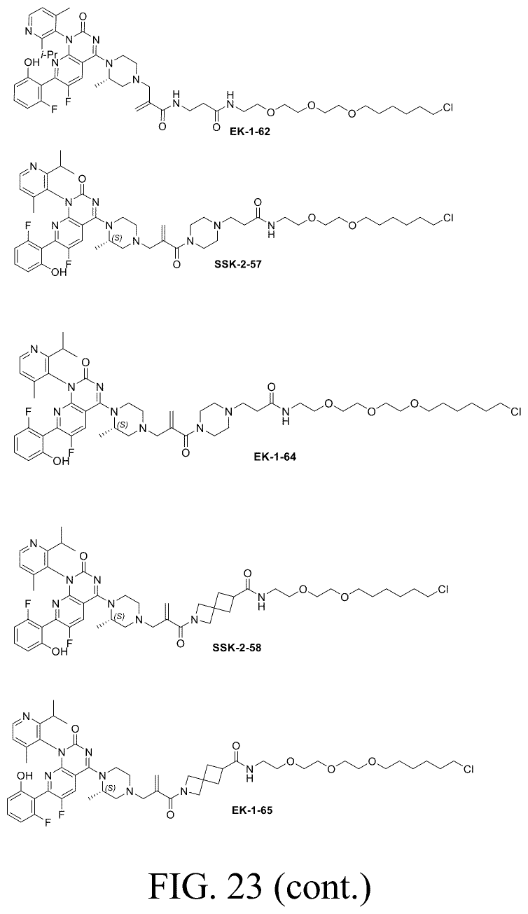

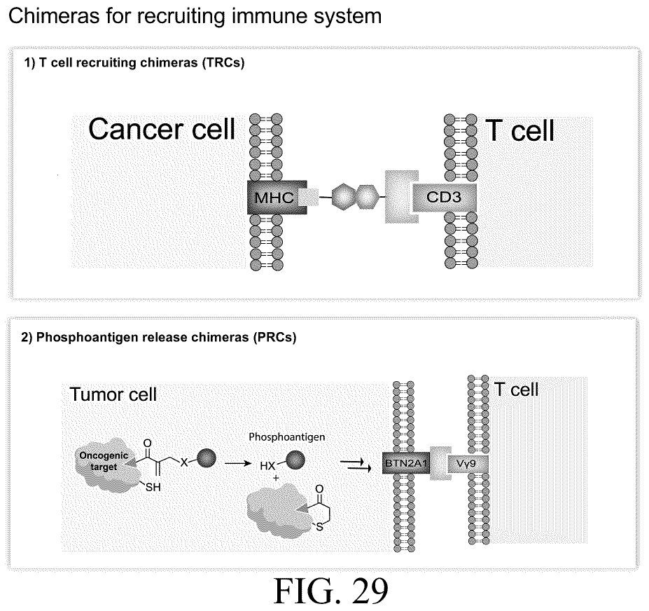

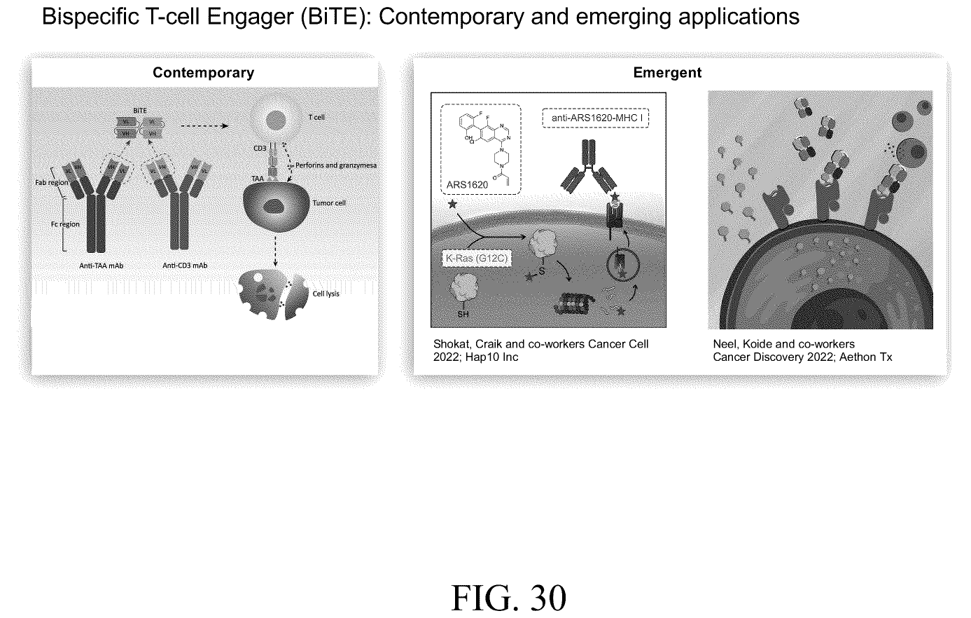

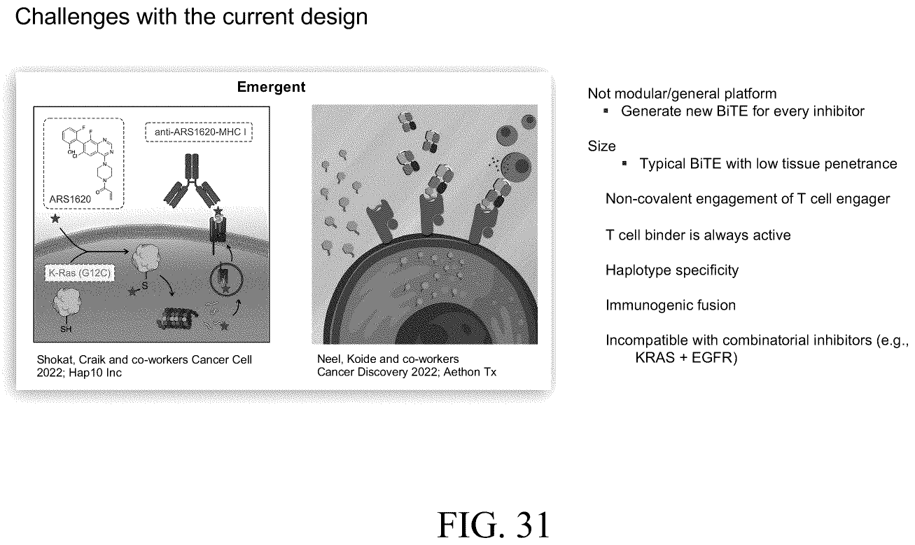

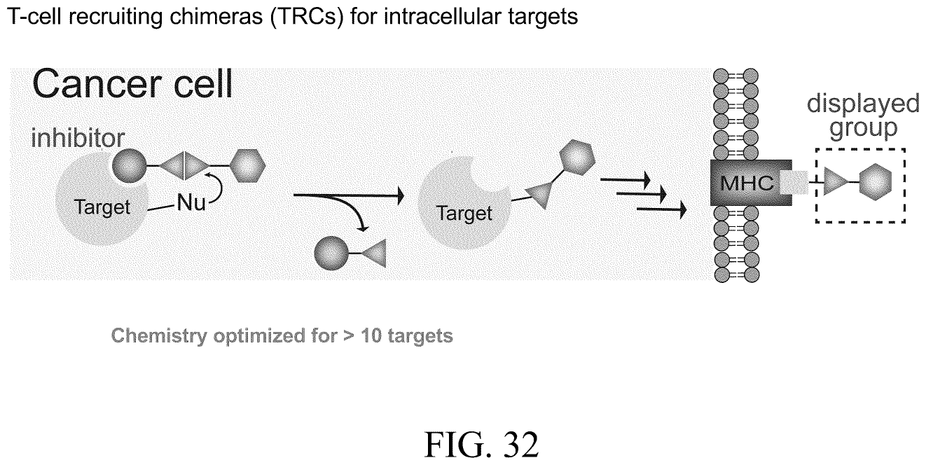

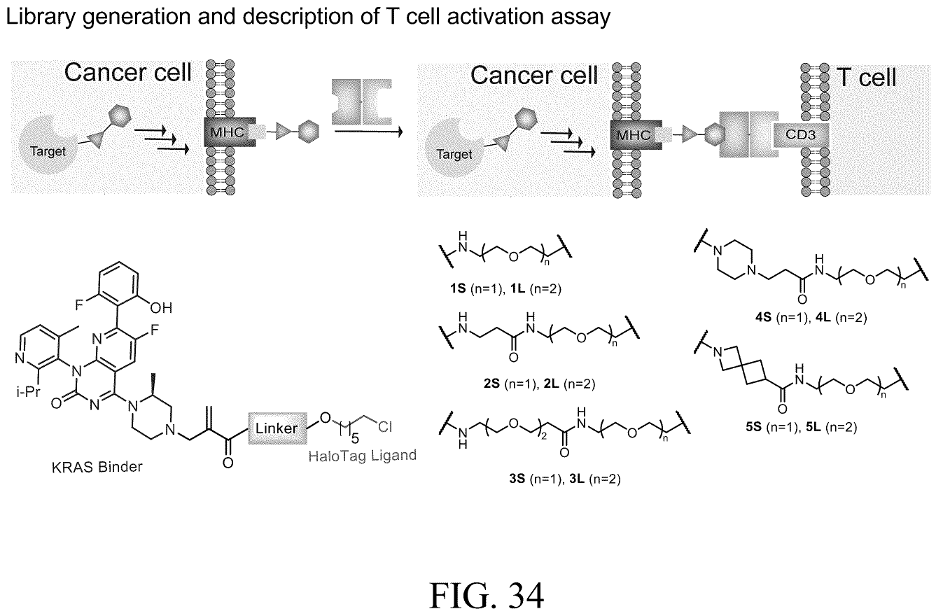

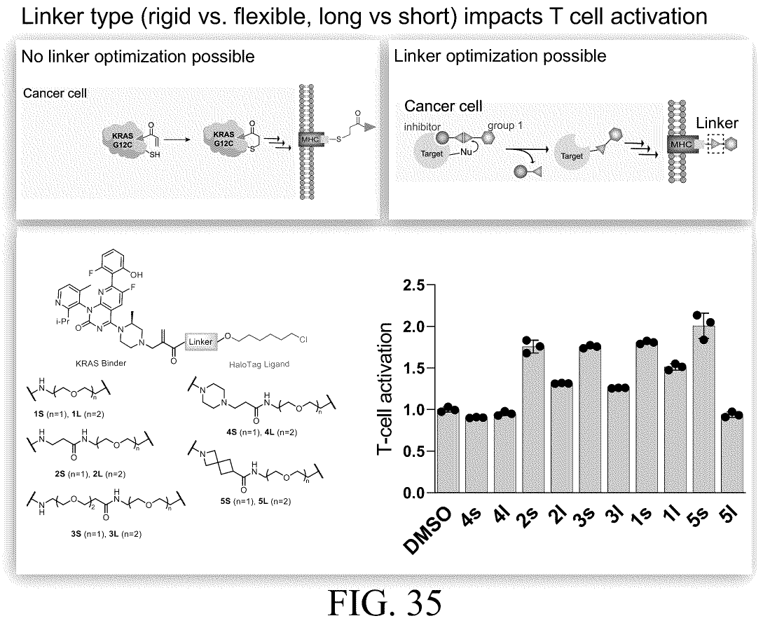

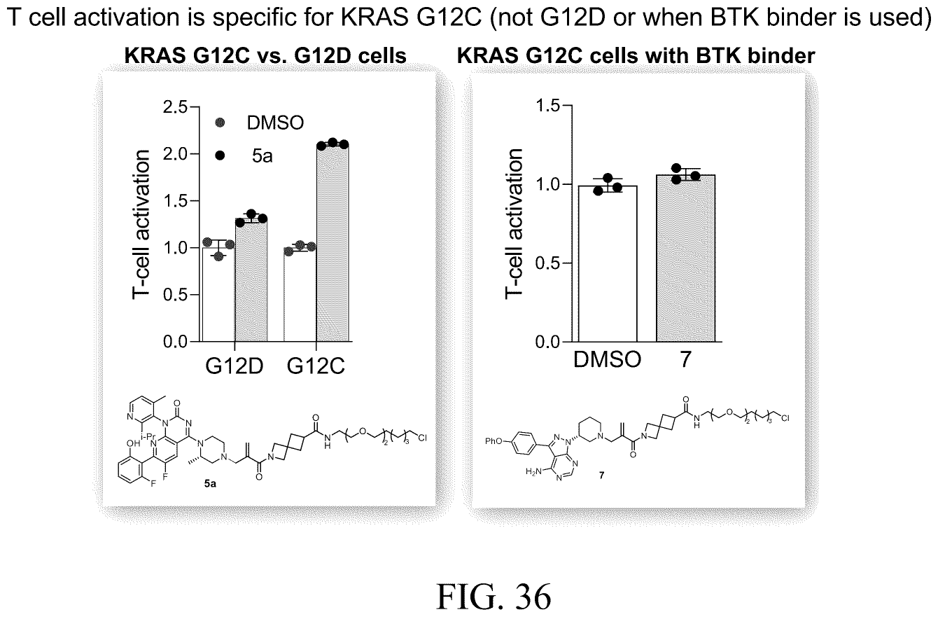

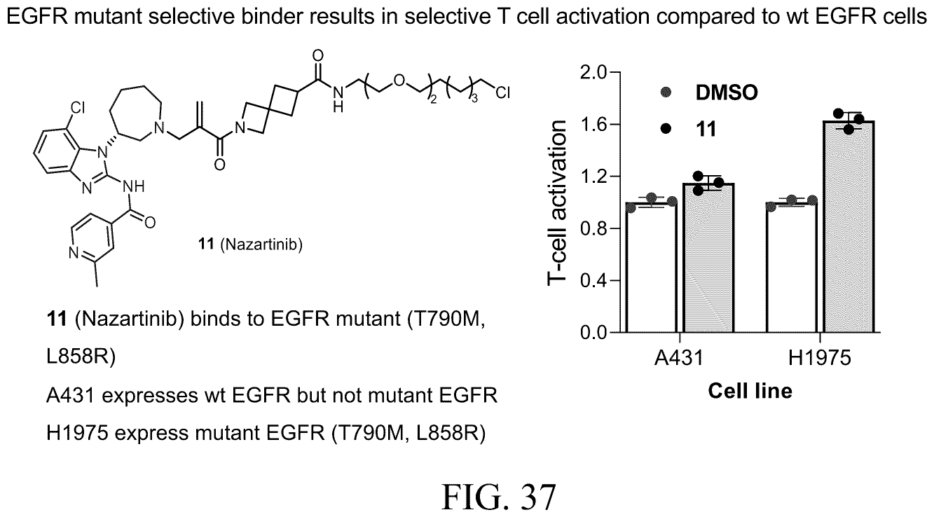

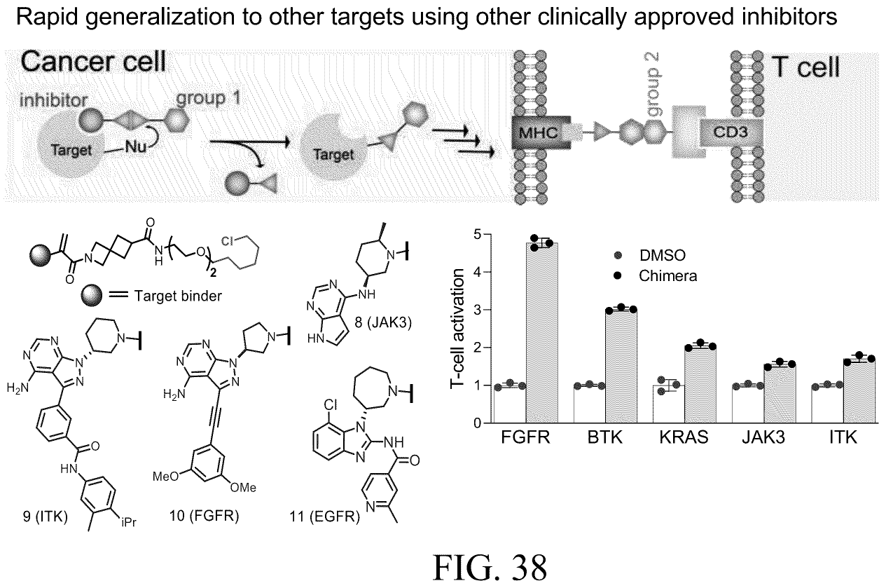

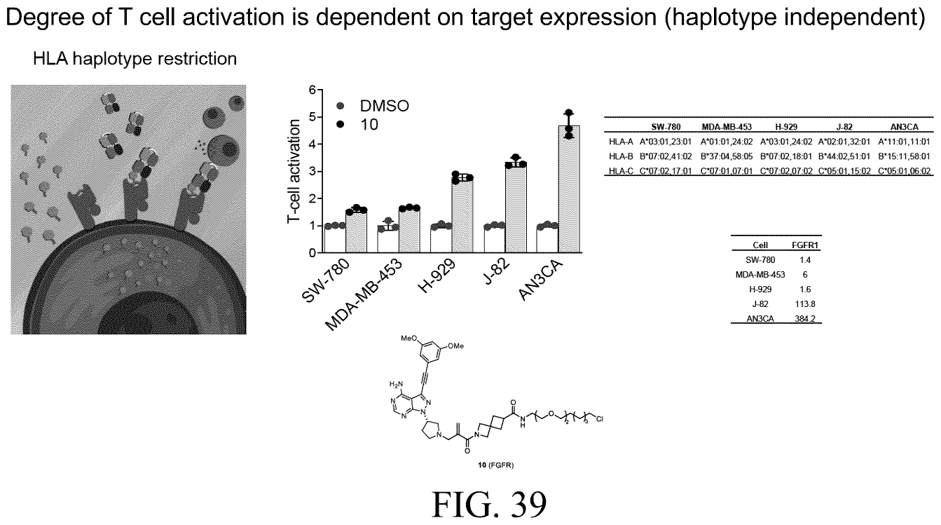

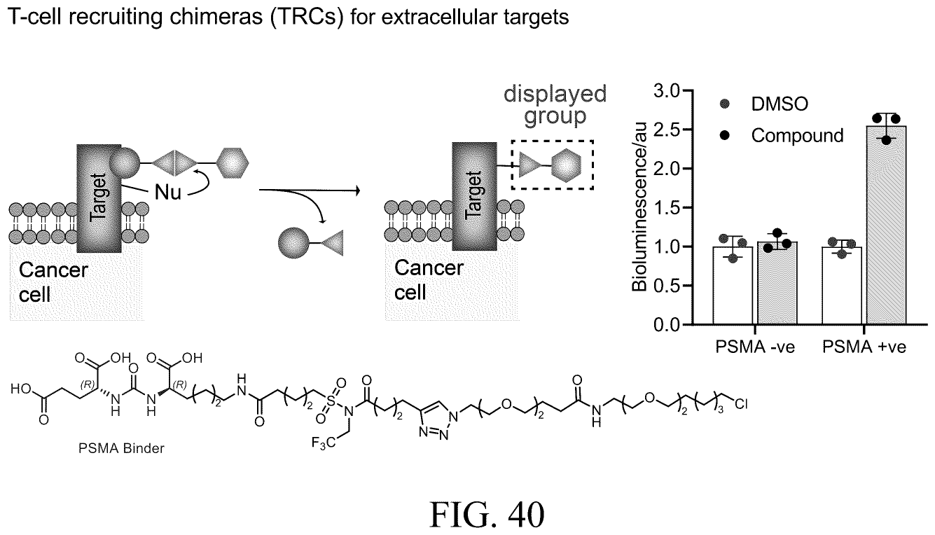

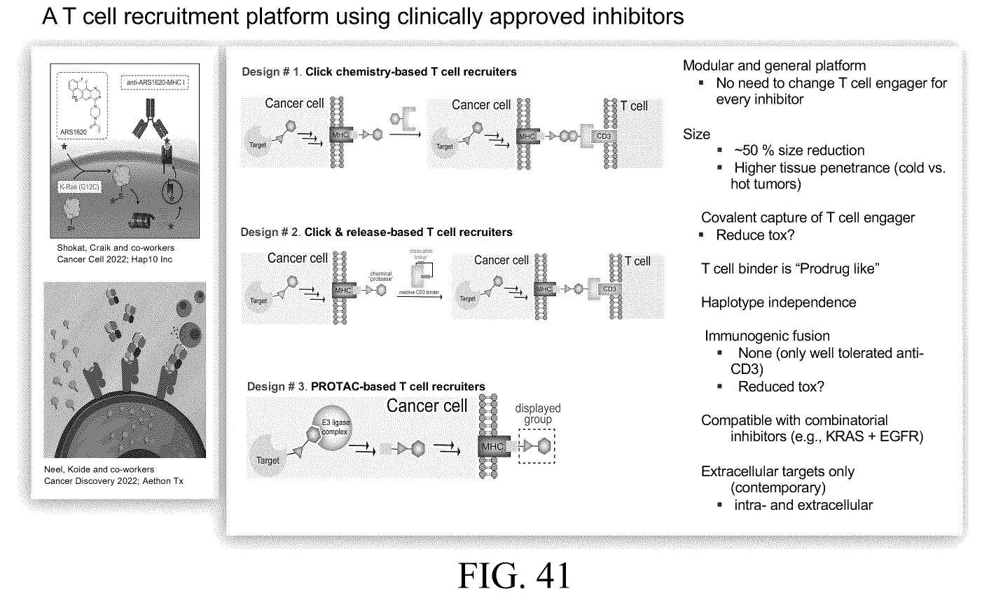

In one example embodiment, the chimeric small molecule can comprise a target protein binding moiety of any one of the chimeric small molecules of FIGS. 1-42. In one example embodiment, the target protein binding moiety is a variant-specific target protein agent (e.g., an inhibitor, a targeted degrader, a non-covalent inhibitor, a molecular glue inhibitor), or a sub-group, fragment, derivative, homologue, or orthologue thereof. In one example embodiment, the target protein binding moiety is a pan-target protein inhibitor (e.g., a RAS^{MULTI} molecular glue inhibitor or a pan-target protein degrader), or a sub-group, fragment, derivative, homologue, or orthologue thereof. In one example embodiment, the target protein binding moiety is an on-state inhibitor (e.g., a target protein inhibitor), or a sub-group, fragment, derivative, homologue, or orthologue thereof.

### KRAS Binding Moieties

In one example embodiment, the protein binding moiety is or comprises a KRAS binding moiety. In one example embodiment, the Kras binding moiety is a Kras inhibitor, e.g., a Kras inhibiting drug molecule, e.g., a variant-specific KRAs agent (e.g., a KRAS-G12D targeted degrader, a KRAS-G12D inhibitor, a non-covalent KRAS-G12D inhibitor, a KRAS-G12D molecular glue inhibitor, a KRAS-G13C molecular glue inhibitor), a pan-KRAS inhibitor (e.g., a RAS^{MULTI} molecular glue inhibitor, or a pan-KRAS degrader), an on-state KRAS inhibitor (e.g., a RAS^{MULTI} inhibitor, a KRAS-G12C inhibitor, a KRAS-G12D inhibitor, a KRAS-Q61H, a KRAS-G13C, a KRAS-G12R inhibitor, a KRAS-G12V inhibitor, a G13D inhibitor, a KRAS-Q61X inhibitor), or a sub-group, fragment, derivative, homologue, or orthologue thereof.

In one example embodiment, the KRAS binding moiety comprises (or is) ARS-1620 (e.g., Hap10, Inc.), sotorasib (e.g., Amgen), adagrasib (e.g., Mirati), opnurasib (e.g., Novartis), divarasib (e.g., Genentech/Roche), garsorasib (e.g., InvestiveBio), JAB-21822 (e.g., Jacobio), YL-15293 (e.g., Yingli), IBI251 (e.g., Innovent Biologics), RMC-6291 (e.g., Revolution Medicines), LY3537982 (e.g., Lilly/Loxo), MK-1084 (e.g., Merck), BI 1823911 (e.g., Boehringer Ingelheim), D3S-001 (e.g., D3 Bio (Wuxi)), ASP3082 (e.g., Astellas), HRS-4642 (e.g., Jiangsu Hengui Medicine), MRTX1133 (e.g., Mirati), RMC-9804 (e.g., Revolution Medicines), RMC-8839 (e.g., Revolution Medicines), BI-KRASG12D (e.g., Boehringer Ingelheim), JAB-22000 (e.g., Jacobio), ERAS-4 (e.g., Erasca), RMC-6236 (e.g., Revolution Mediciones), FMC-376 (e.g., Frontier Medicines), RMC-0708 (e.g., Revolution Medicines), or BBO-8520 (e.g., BridgeBio), or a sub-group, fragment, derivative, homologue, or orthologue thereof.

In one example embodiment, the KRAS binding moiety comprises or has the following structure:

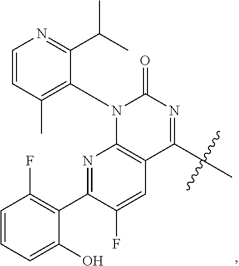 [SMILES: *c1nc(=O)n(-c2c(C)ccnc2C(C)C)c2nc(-c3c(O)cccc3F)c(F)cc12]

or an analog or derivative thereof.

In an example embodiment, a chimeric small molecule comprising a KRAS binding moiety comprises or has the formula:

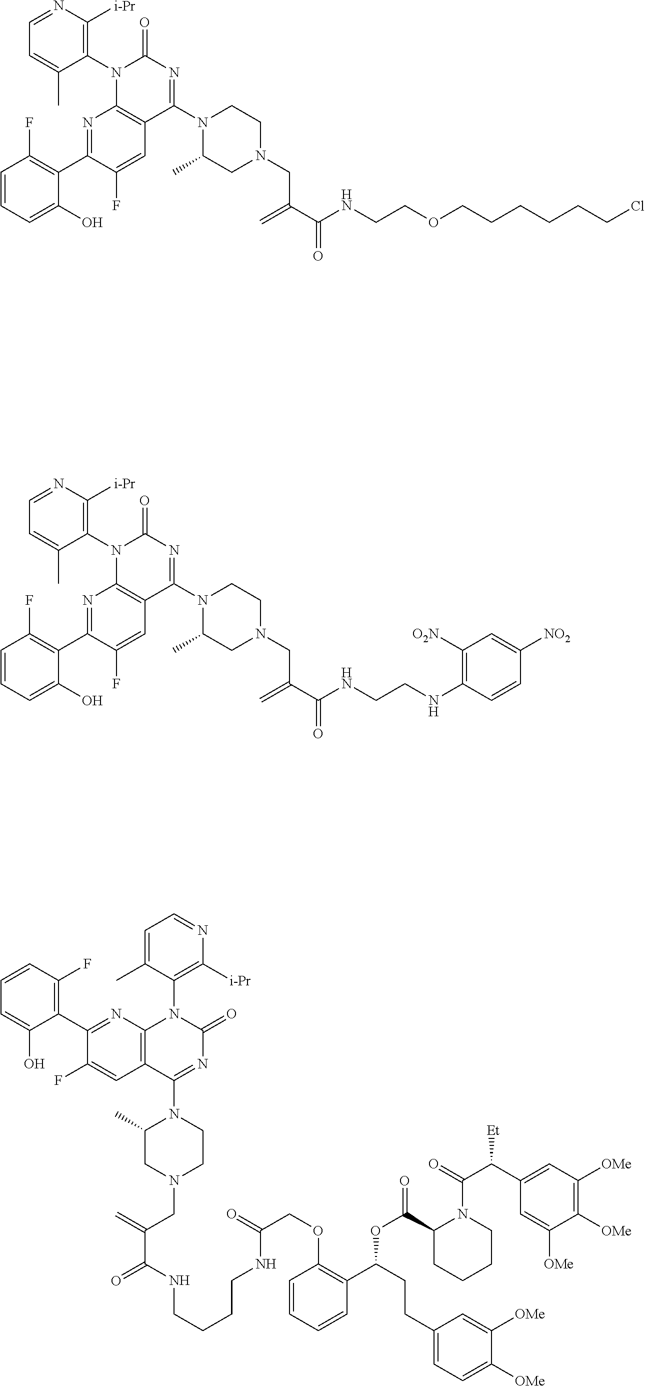 [SMILES: C=C(CN1CCN(c2nc(=O)n(-c3c(C)ccnc3C(C)C)c3nc(-c4c(O)cccc4F)c(F)cc23)[C@@H](C)C1)C(=O)NCCOCCCCCCCl ; C=C(CN1CCN(c2nc(=O)n(-c3c(C)ccnc3C(C)C)c3nc(-c4c(O)cccc4F)c(F)cc23)[C@@H](C)C1)C(=O)NCCNc1ccc([N+](=O)[O-])cc1[N+](=O)[O-] ; C=C(CN1CCN(c2nc(=O)n(-c3c(C)ccnc3C(C)C)c3nc(-c4c(O)cccc4F)c(F)cc23)[C@@H](C)C1)C(=O)NCCCCNC(=O)COc1ccccc1[C@@H](CCc1ccc(OC)c(OC)c1)OC(=O)[C@@H]1CCCCN1C(=O)[C@H](CC)c1cc(OC)c(OC)c(OC)c1]

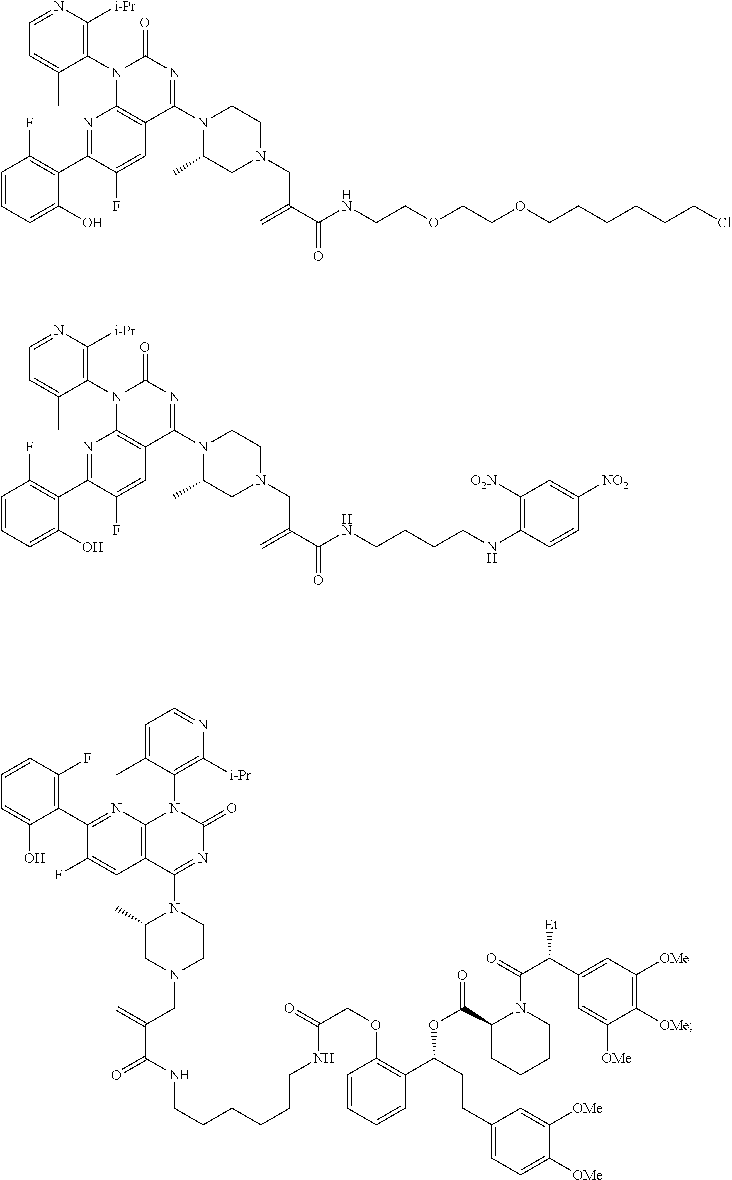 [SMILES: C=C(CN1CCN(c2nc(=O)n(-c3c(C)ccnc3C(C)C)c3nc(-c4c(O)cccc4F)c(F)cc23)[C@@H](C)C1)C(=O)NCCOCCOCCCCCCCl ; C=C(CN1CCN(c2nc(=O)n(-c3c(C)ccnc3C(C)C)c3nc(-c4c(O)cccc4F)c(F)cc23)[C@@H](C)C1)C(=O)NCCCCNc1ccc([N+](=O)[O-])cc1[N+](=O)[O-] ; C=C(CN1CCN(c2nc(=O)n(-c3c(C)ccnc3C(C)C)c3nc(-c4c(O)cccc4F)c(F)cc23)[C@@H](C)C1)C(=O)NCCCCCCNC(=O)COc1ccccc1[C@@H](CCc1ccc(OC)c(OC)c1)OC(=O)[C@@H]1CCCCN1C(=O)[C@H](CC)c1cc(OC)c(OC)c(OC)c1]

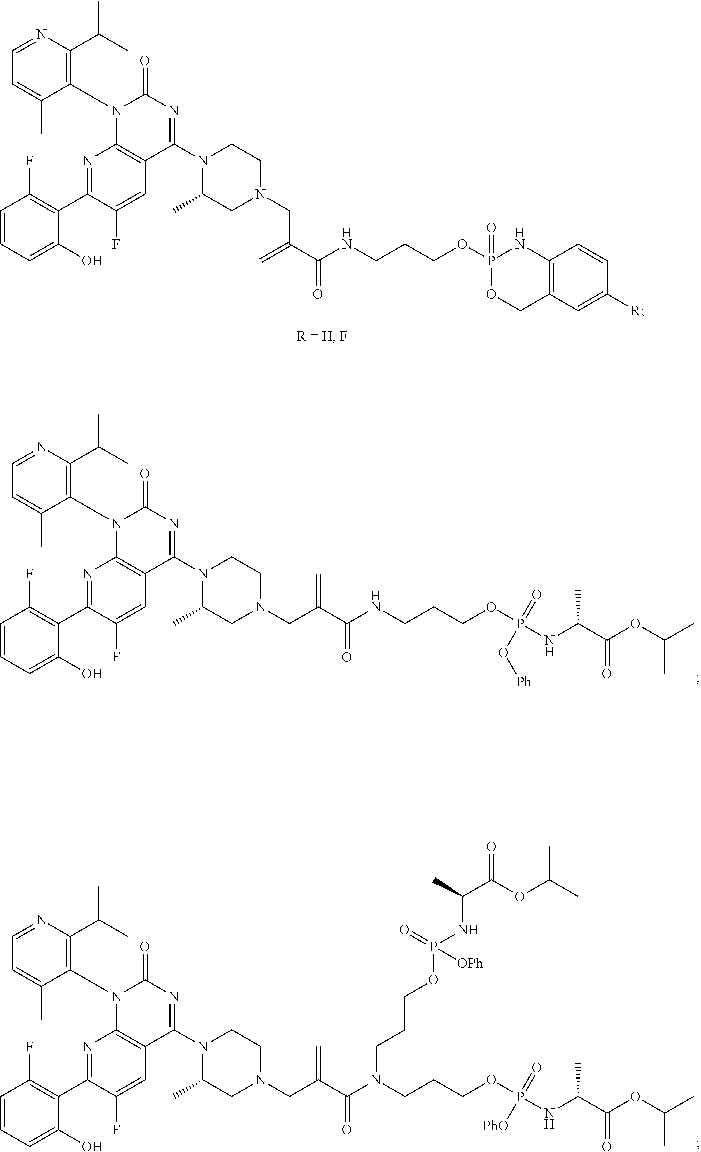

or an analog or derivative thereof.

In one example embodiment, the KRAS binding moiety comprises or has the following structure:

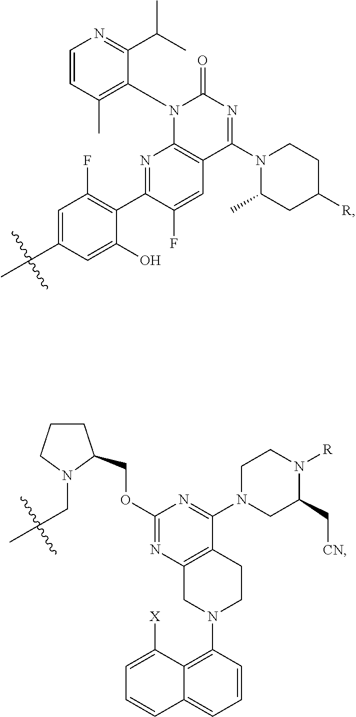

or an analog or derivative thereof, wherein R is a covalent warhead (an electrophilic reactive group that can form a direct covalent bond with a nucleophilic amino acid of a protein); X is the formula

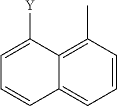

and Y is selected from the group consisting of: H, alkane, alkene, alkyne, amine, nitrile, nitro, ether, alcohol, thiol, sulfone, sulfonate, halogen, carbonyl, acyl, ketone, carboxylate ester, amide, enone, anhydride, imide, cyclic hydrocarbon, an unsaturated cyclic hydrocarbon, a heterocycle, one or more fused rings thereof, or an aliphatic halide such as —OCF₂C1.

In one example embodiment, the protein binding moiety is or comprises a hydrogen bond surrogate (HBS) Son of Sevenless (SOS) peptide mimics (PM). In one example embodiment, the HBS—SOS-PM is HBS 1-7 according to the sequences: XFE*GIYRTDILRTEEGN-NH2 (SEQ ID NO: 1); XFE*GIYRTELLKAEEAN-NH2 (SEQ ID NO: 2); XFE*GIYRLELLKAEEAN-NH2 (SEQ ID NO: 3); XFE*GIYRLELLK-NH2 (SEQ ID NO: 4); XFE*AIYRLELLKAEEAN-NH2 (SEQ ID NO: 5); XFE*GIYRLELLKAibEEAibN-NH2 (SEQ ID NO: 6); and XAE*GIYRLELLKAEAAA-NH2 (SEQ ID NO: 7), respectively, wherein X denotes a 4-pentenoic acid residue and the asterisk (*) denotes N-allyl residue (*G, N-allylglycine). In one example embodiment, the protein binding moiety is or comprises a KRAS binding molecule HB3 according to the formula: XFE*GIYRLELLKAEEAN-NH2 (SEQ ID NO: 3). In one example embodiment, the protein binding moiety is or comprises a KRAS binding molecule HB7 according to the formula: XAE*GIYRLELLKAEAAA-NH2 (SEQ ID NO: 8). See Nickerson et al., An Orthosteric Inhibitor of the RAS-SOS Interaction, doi: 10.1016/B978-O-12-420146-0.00002-0 incorporated herein by reference in its entirety with specific mention of Table 2.1.

In one example embodiment, the protein binding moiety comprises or is a KRAS binding molecule according to the formula:

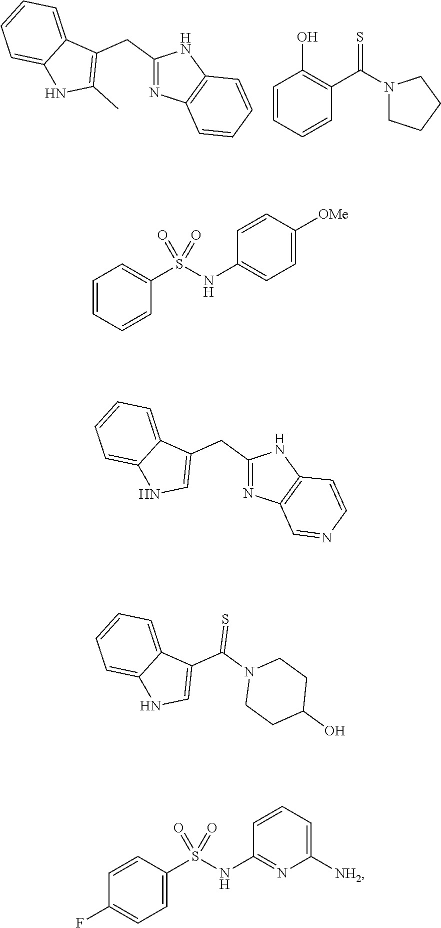

or an analog or derivative thereof. In one example embodiment, the protein binding moiety comprises or is a KRAS binding moiety according to the formula:

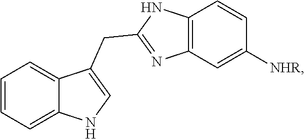

or an analog or derivative thereof, wherein R may be H, Gly, Ala, (3-Ala, Val, Ile, Pro, or any other feasible substituent known in the art. In one example embodiment, the protein binding moiety is or comprises a KRAS binding moiety, e.g., an indole, phenol, sulfonamide, or any modified version thereof. See Sun et al., Angew Chem Int Ed Engl. 2012 Jun. 18; 51(25): 6140-6143. doi: 10.1002/anie.201201358, herein incorporated by reference in its entirety.

In one example embodiment, the protein binding moiety is or comprises a KRAS binding molecule according to the formula:

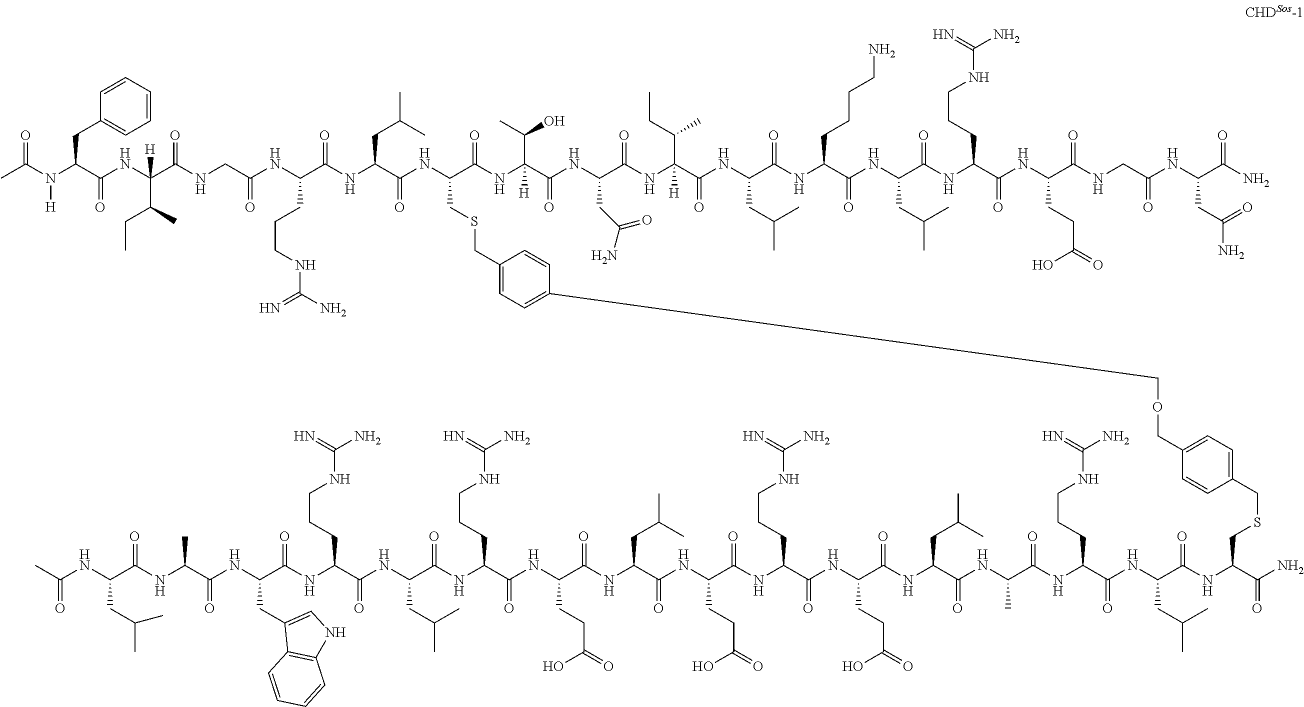

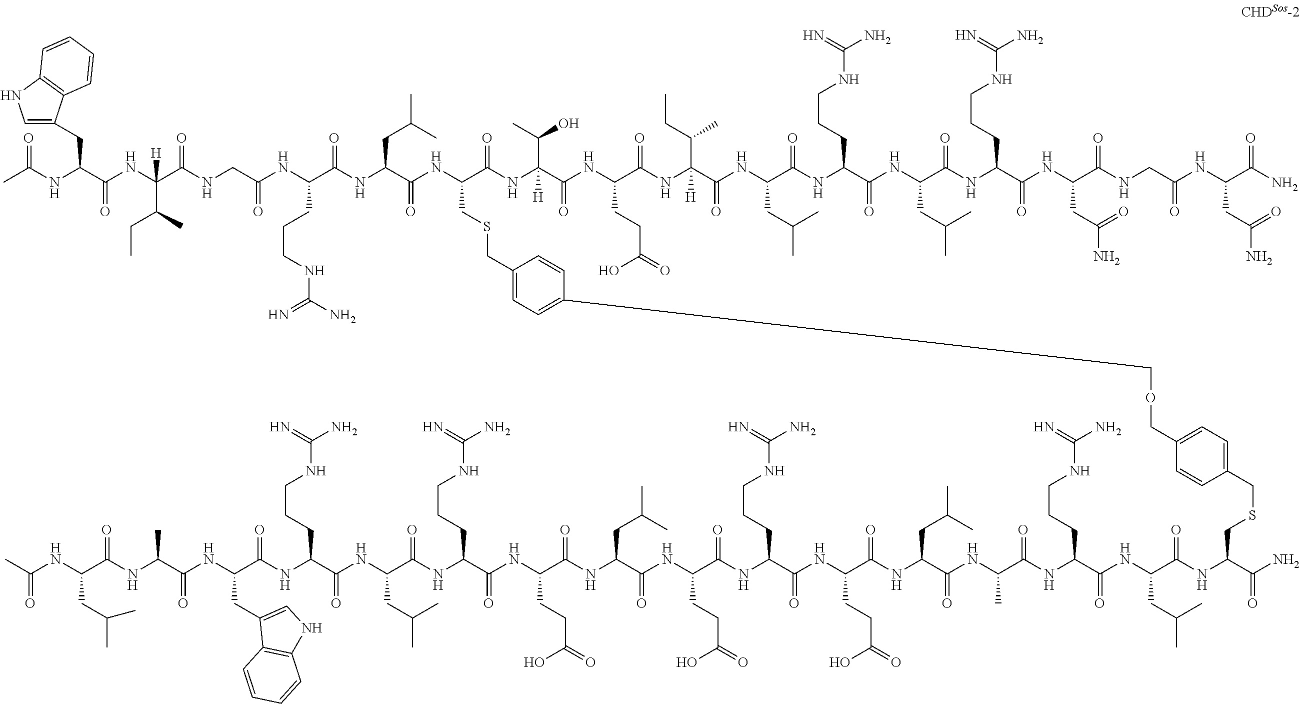

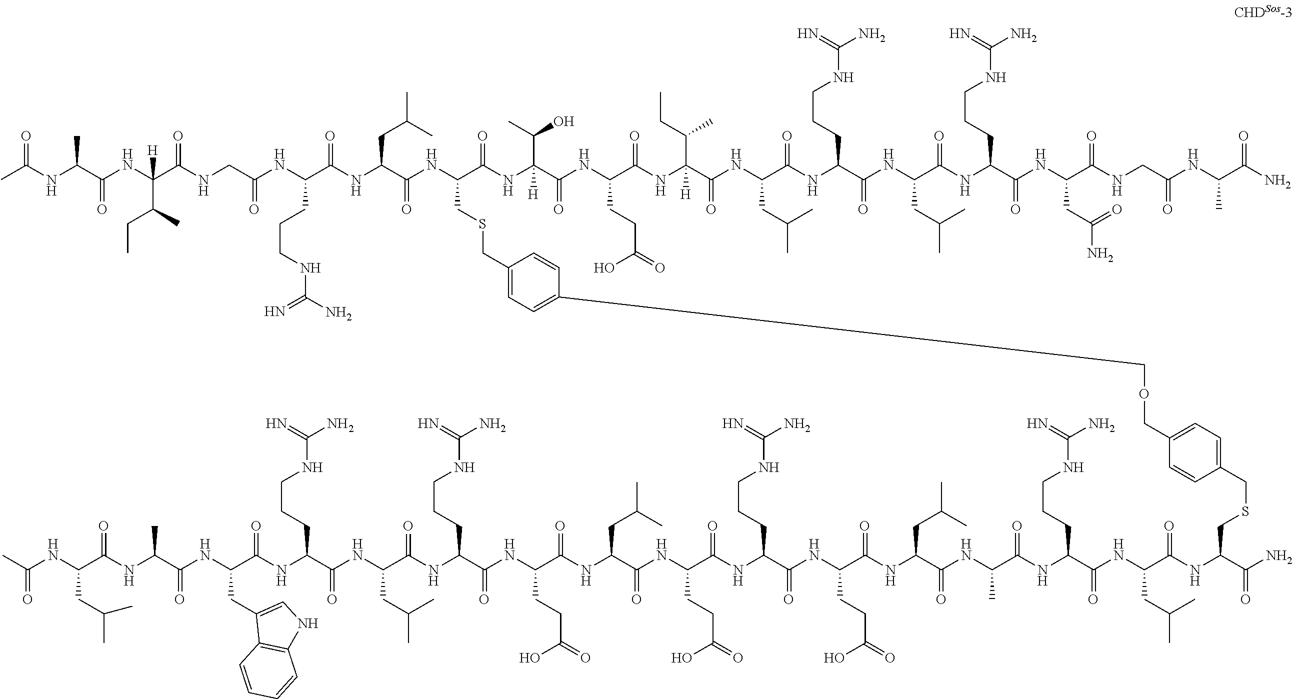

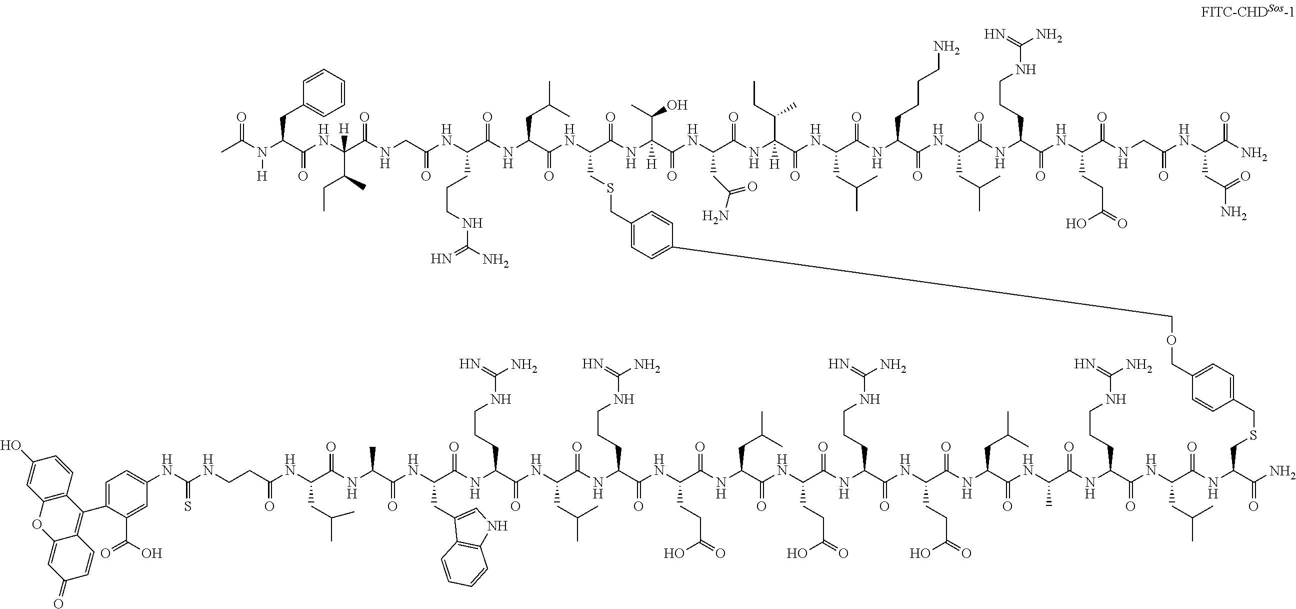

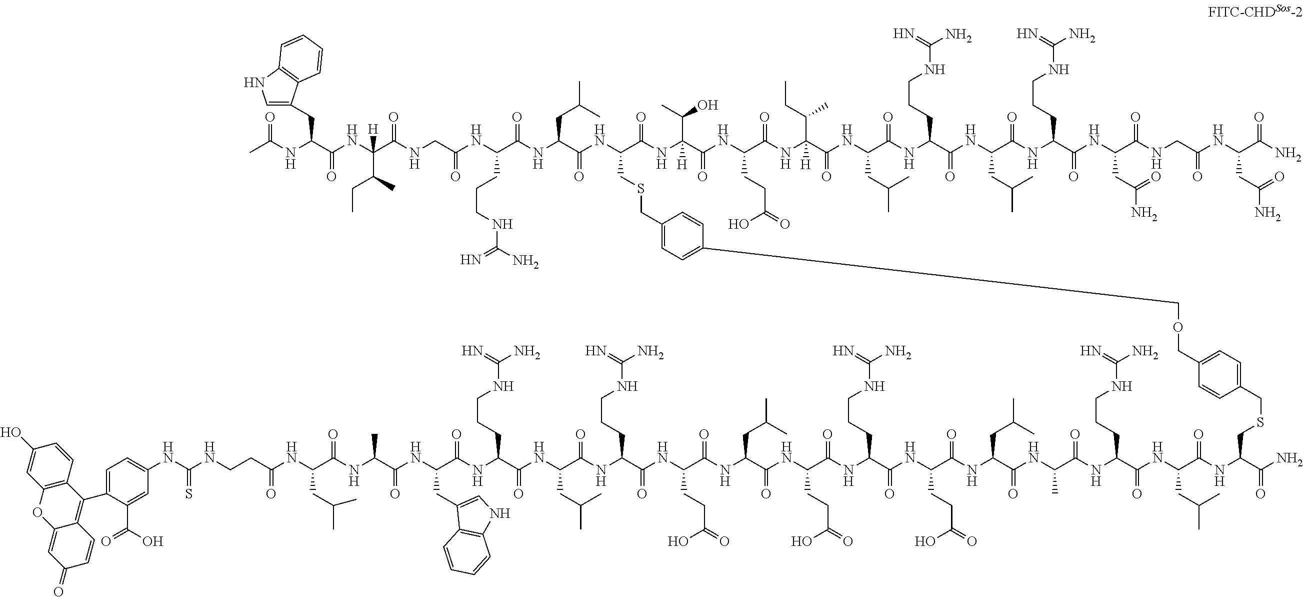

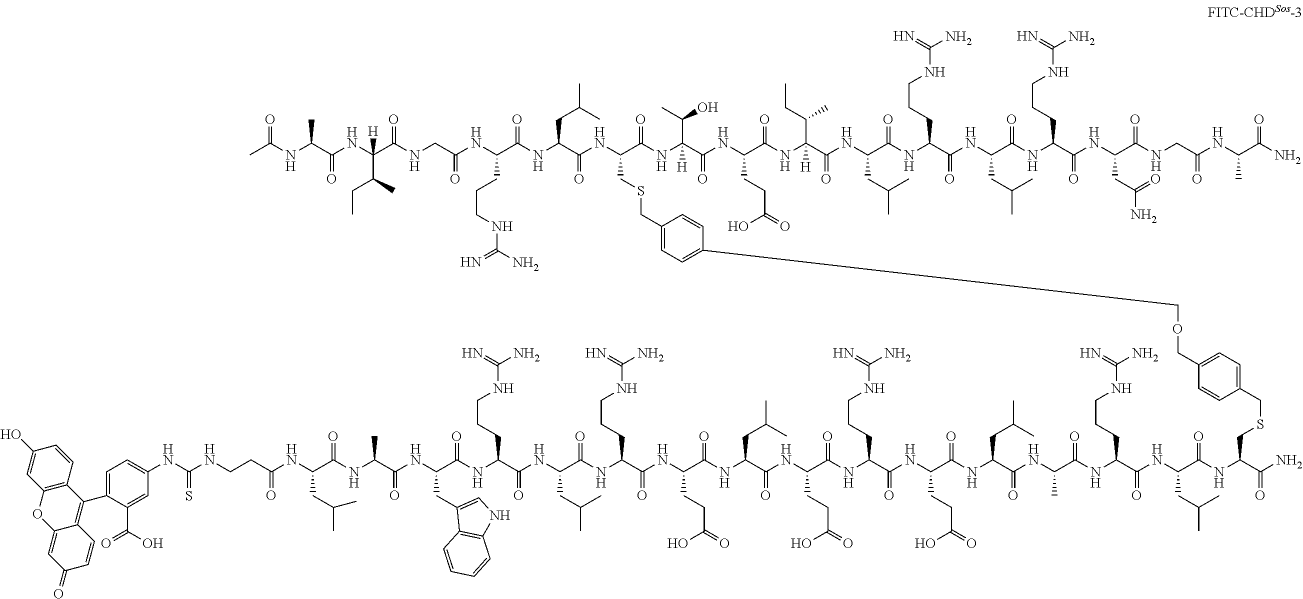

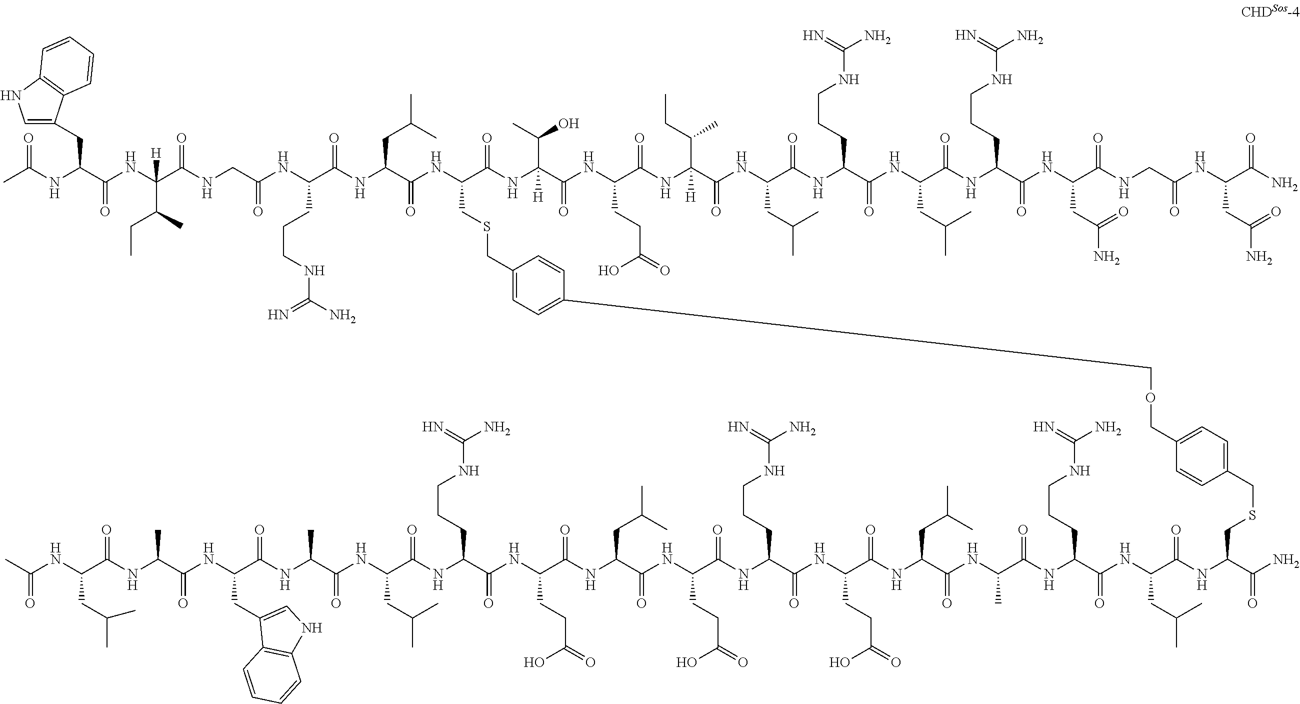

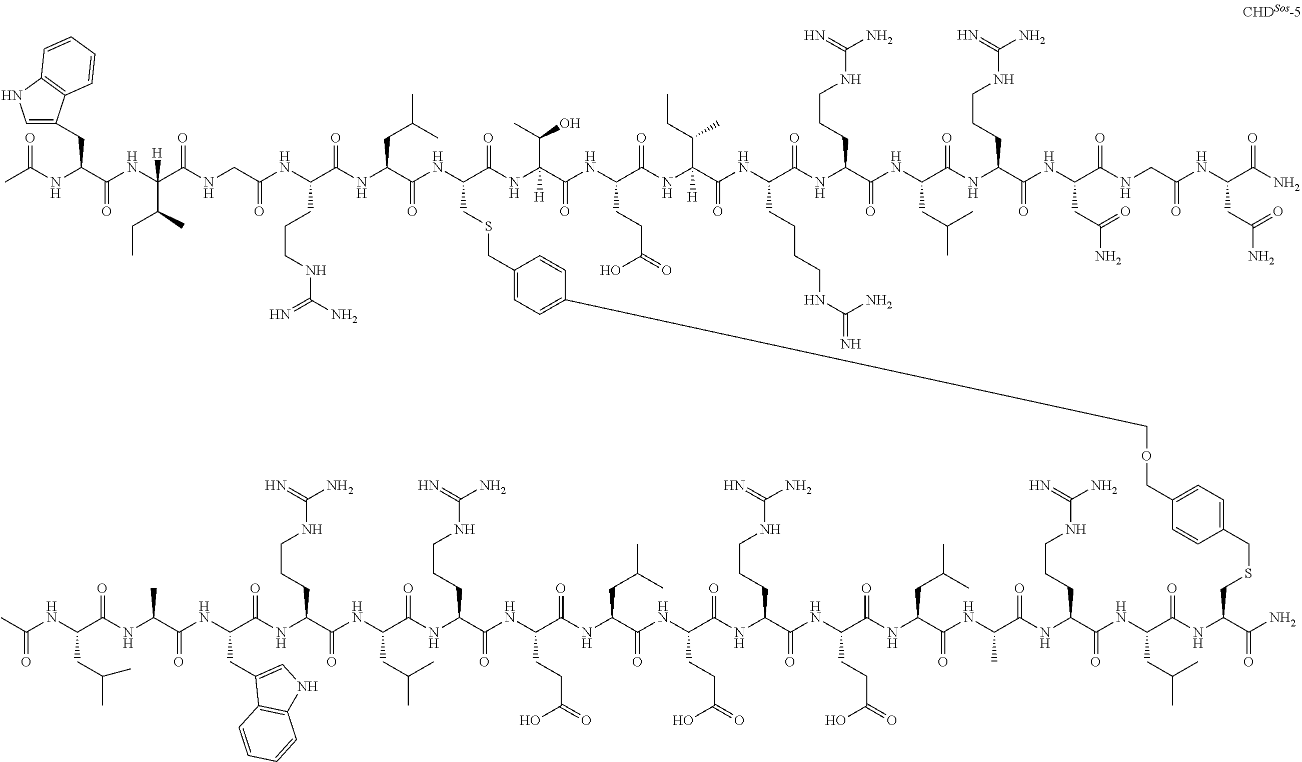

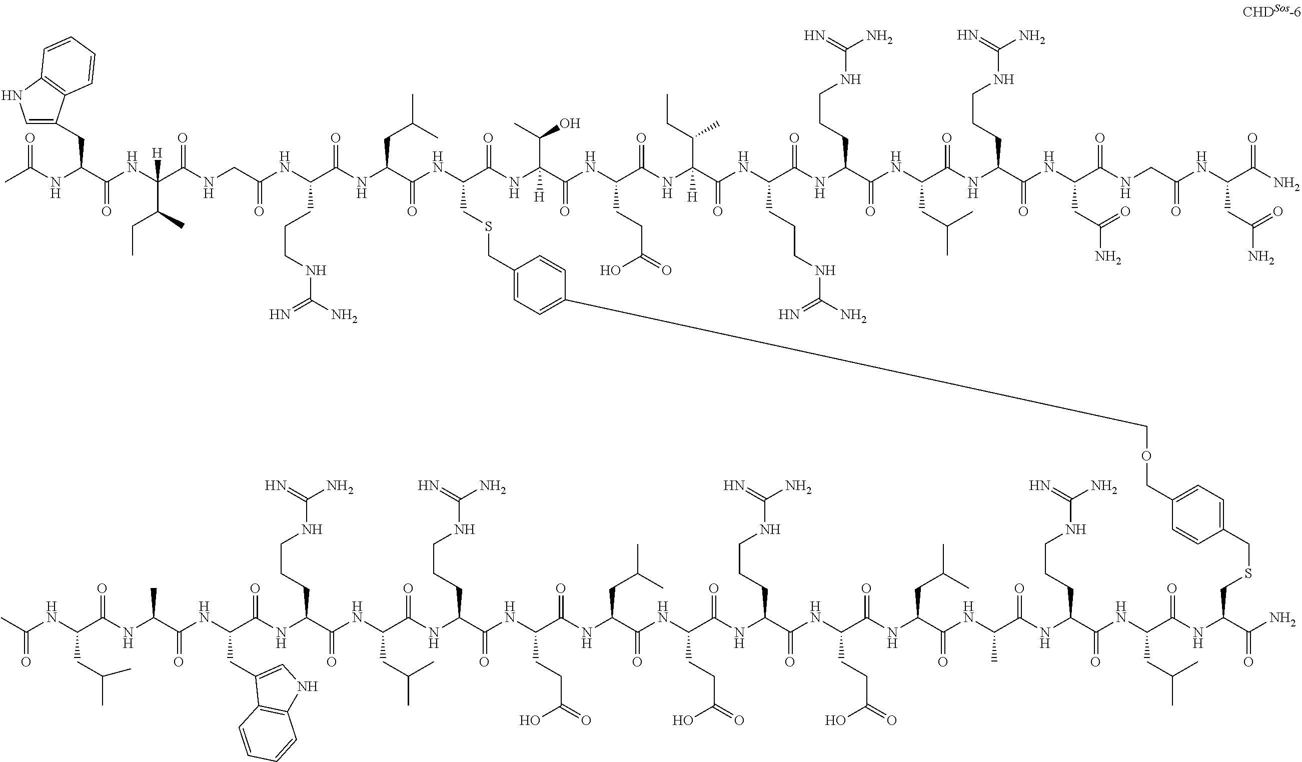

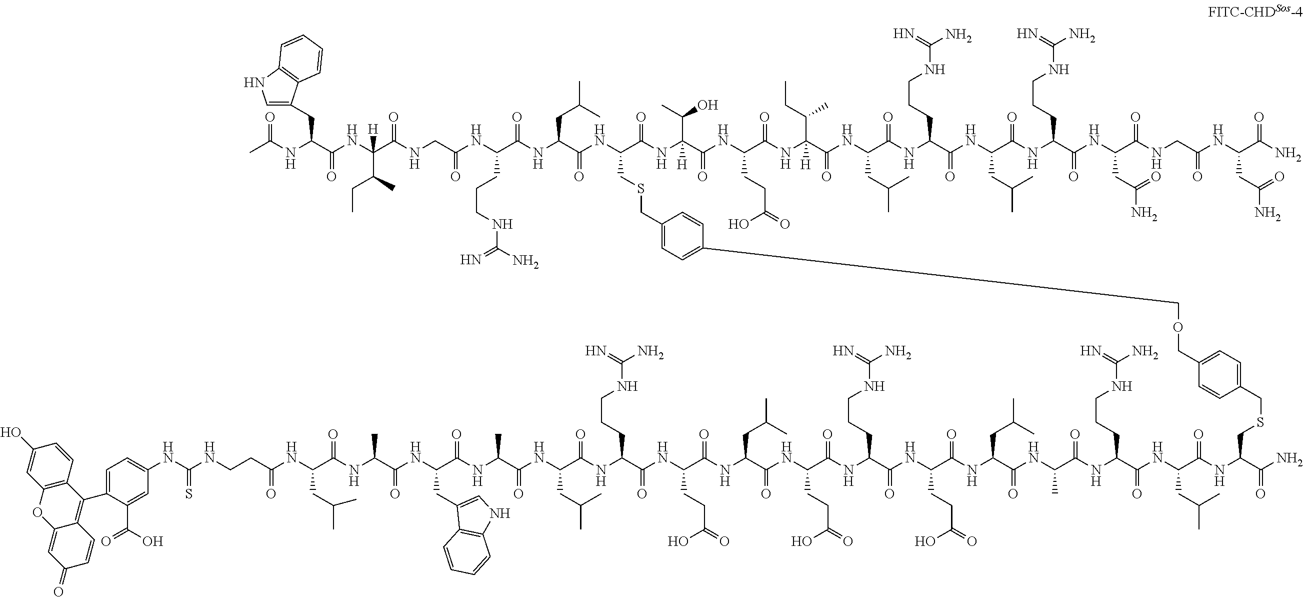

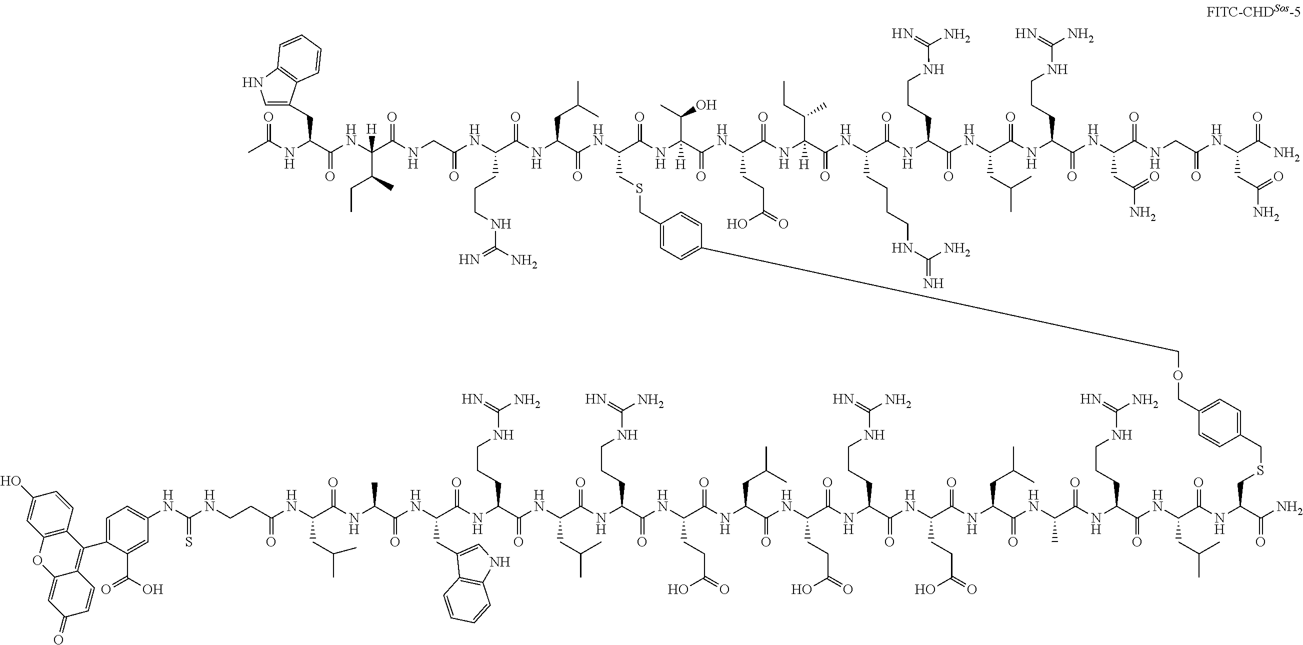

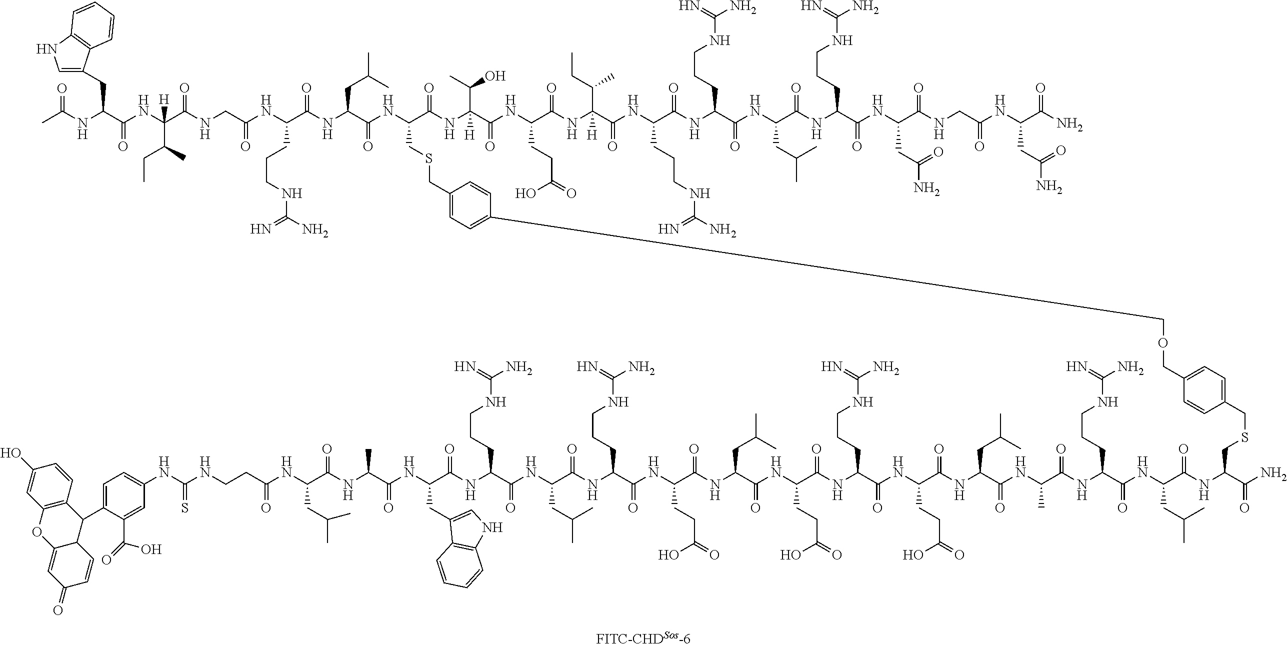

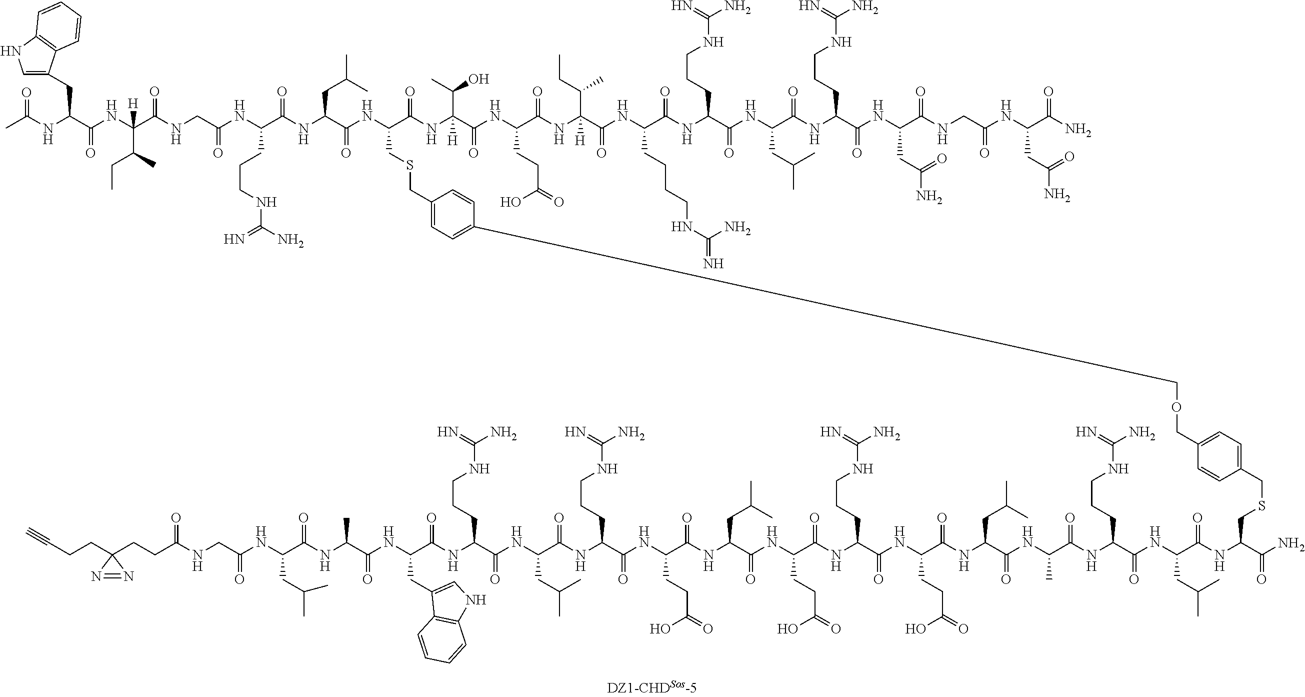

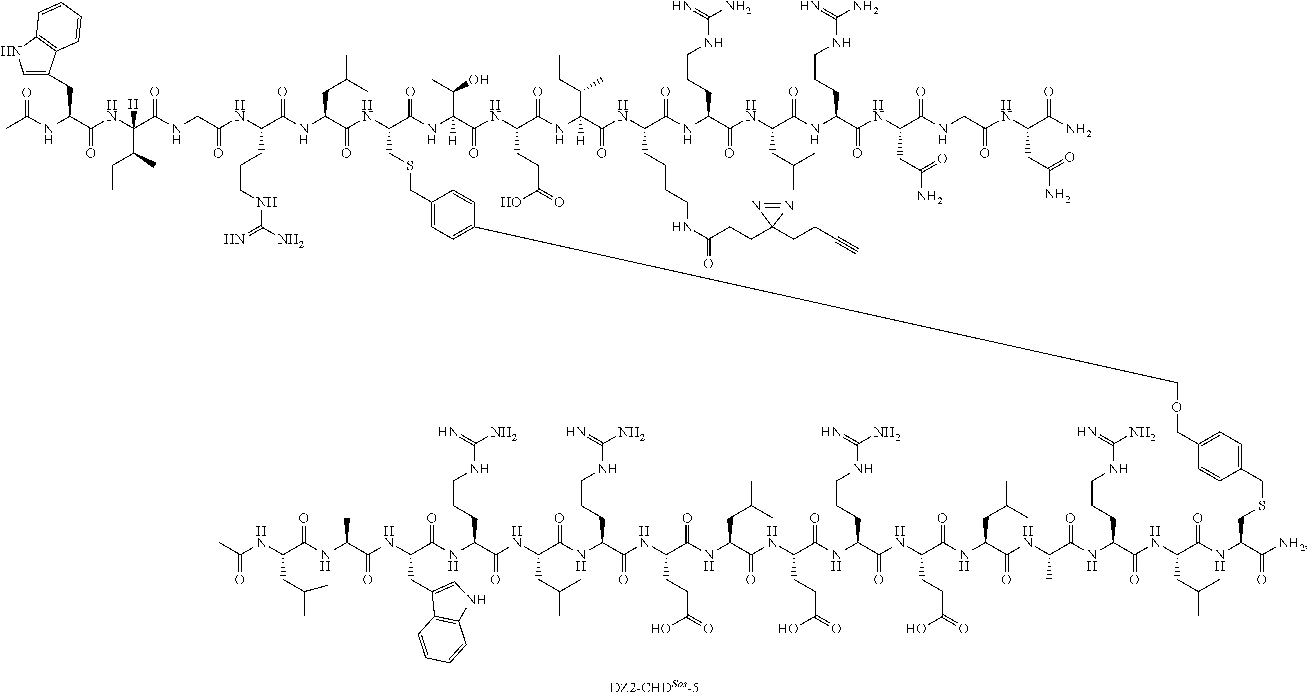

or an analog or derivative thereof. In one example embodiment, the protein binding moiety is or comprises a SOS peptide mimic according to the formula: Ac-FIGRLCTEILKLREGN-NH2 (SEQ ID NO: 9); Ac-LAWRLRELERELARLC-NH2 (SEQ ID NO: 10); Ac-WIGRLCTEILRLRNGN-NH2 (SEQ ID NO: 11); Ac-LAWRLRELERELARLC-NH2 (SEQ ID NO: 12); Ac-AIGRLCTEILRLRNGA-NH2 (SEQ ID NO: 13); Ac-LAWRLRELERELARLC-NH2 (SEQ ID NO: 14); Ac-WIGRLCTEILRLRNGN-NH2 (SEQ ID NO: 15); Ac-LAWALRELERELARLC-NH2 (SEQ ID NO: 16); Ac-WIGRLCTEIR^{H}RLRNGN-NH2 (SEQ ID NO: 17); Ac-LAWRLRELERELARLC-NH2 (SEQ ID NO: 18); Ac-WIGRLCTEIRRLRNGN-NH2 (SEQ ID NO: 19); Ac-LAWRLRELERELARLC-NH2 (SEQ ID NO: 20); Ac-WIGRLCTEILRLRNGN-NH2 (SEQ ID NO: 21); Ac-LAWRLRELERELARLC-NH2 (SEQ ID NO: 22); Ac-FIGRLCTEILKLREGN-NH2 (SEQ ID NO: 23); FITC-AβLAWRLRELERELARLC-NH2 (SEQ ID NO: 24); Ac-WIGRLCTEILRLRNGN-NH2 (SEQ ID NO: 25); FITC-APLAWRLRELERELARLC-NH2 (SEQ ID NO: 26); Ac-AIGRLCTEILRLRNGA-NH2 (SEQ ID NO: 27); FITC-APLAWRLRELERELARLC-NH2 (SEQ ID NO: 28); Ac-WIGRLCTEILRLRNGN-NH2 (SEQ ID NO: 29); FITC-AβLAWALRELERELARLC-NH2 (SEQ ID NO: 30); Ac-WIGRLCTEIR^{H}RLRNGN-NH2 (SEQ ID NO: 31); FITC-AβLAWRLRELERELARLC-NH2 (SEQ ID NO: 32); Ac-WIGRLCTEIRRLRNGN-NH2 (SEQ ID NO: 33); FITC-AβLAWRLRELERELARLC-NH2 (SEQ ID NO: 34); Ac-WIGRLCTEIR^{H}RLRNGN-NH2 (SEQ ID NO: 35); DZ-GLAWRLRELERELARLC-NH2 (SEQ ID NO: 36); Ac-WIGRLCTEIK(DZ)RLRNGN-NH2 (SEQ ID NO: 37); or Ac-LAWRLRELERELARLC-NH2 (SEQ ID NO: 38), wherein R^{H} is L-homoarginine; Aβ is L-β-alanine; DZ is diazirine photocrosslinker; and FITC is 5-fluorescein isothiocyanate linked via thiourea bond to N-terminal amine. See Hong et al., PNAS May 4, 2021 118 (18) e2101027118; doi:10.1073/pnas.2101027118, herein incorporated by reference in its entirety with specific mention of Table S2.

In one example embodiment, the protein binding moiety is or comprises a KRAS binding molecule according to the formula:

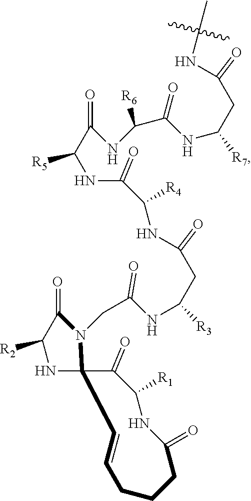

or any analog or derivative thereof, wherein the R groups may be any substituent known in the art. In one example embodiment, R4 is an electrophilic group. In one example embodiment the R4 is

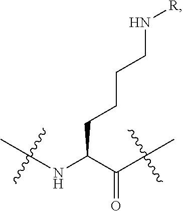

where R is H,

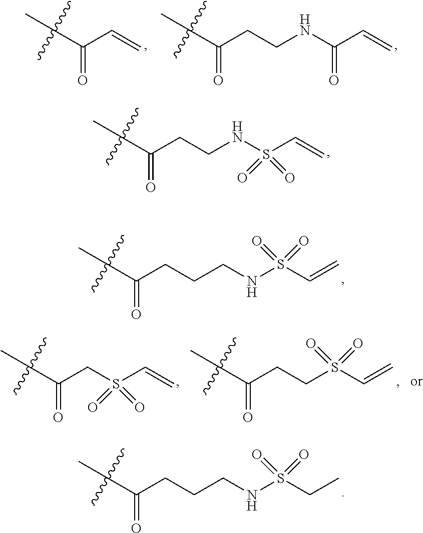

See Yoo et al., ACS Chem. Biol. 2020, 15, 6, 1604-1612, incorporated herein by reference in their entirety.

### FKPB Binding Moieties

In another example embodiment, the protein binding moiety is or comprises an FK506-binding protein (FKBP) binding moiety, or an analog or derivative thereof. The FKBP may be FKBP12, which binds to intracellular calcium release channels and TGF-b type I receptor. In one example embodiment, the FKBP protein binding moiety is or comprises an FKBP12^{F36V} protein binding moiety. In another example embodiment, the protein binding moiety has the following formula:

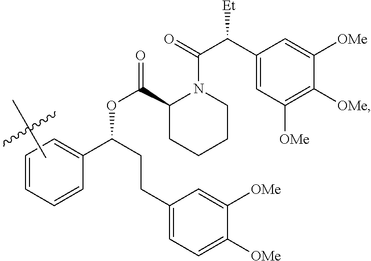

or an analog or derivative thereof.

Tyrosine phosphorylation on FGFR1 can trigger signaling cascade to induce PI3K/AKT/mTOR signaling and increased transcription of G-CSF, a blood growth factor. See, e.g., Turner et al, Nature Reviews Cancer 2010.

In one example embodiment, the molecule is capable of activating FGFR1/mTOR/G-CSF signaling in a dose-dependent manner.

### EGFR Binding Moieties

In one example embodiment, the protein binding moiety is or comprises an EGFR binding moiety. Epidermal growth factor receptor (EGFR) is a tyrosine kinase receptor belonging to the ErbB family of receptors that mediates cell growth, differentiation and repair in cells, especially non-cancerous cells, but EGF is overexpressed in certain cells such as many solid tumors, including colorectal, non-small-cell lung cancer, squamous cell carcinoma of the ovary, kidney, head, pancreas, neck, and prostate, and especially breast cancer.

In one embodiment, the protein binding molecule is an EGFR binding molecule of the formula:

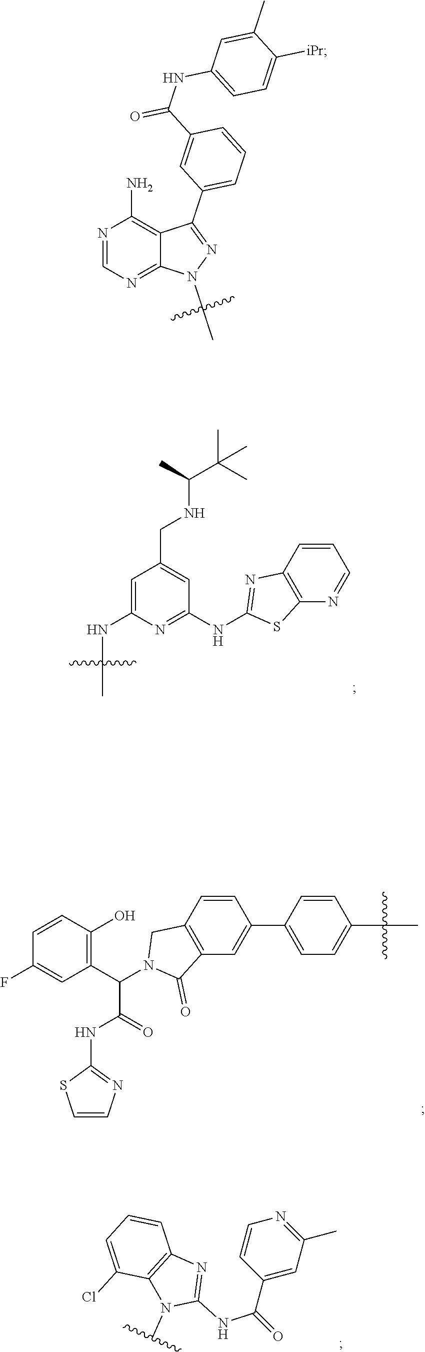 [SMILES: *n1nc(-c2cccc(C(=O)Nc3ccc(C(C)C)c(C)c3)c2)c2c(N)ncnc21 ; *Nc1cc(CN[C@@H](C)C(C)(C)C)cc(Nc2nc3cccnc3s2)n1 ; *c1ccc(-c2ccc3c(c2)C(=O)N(C(C(=O)Nc2nccs2)c2cc(F)ccc2O)C3)cc1 ; *n1c(NC(=O)c2ccnc(C)c2)nc2cccc(Cl)c21]

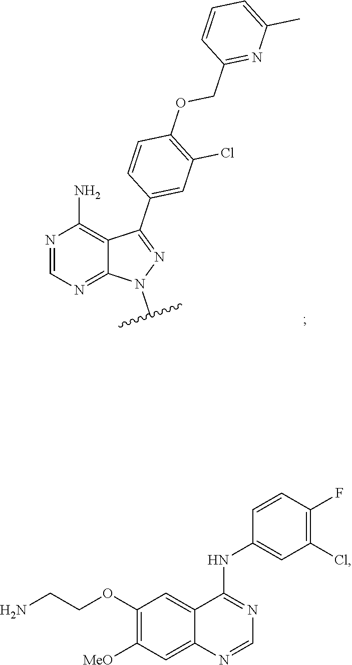 [SMILES: *n1nc(-c2ccc(OCc3cccc(C)n3)c(Cl)c2)c2c(N)ncnc21 ; COc1cc2ncnc(Nc3ccc(F)c(Cl)c3)c2cc1OCCN]

or an analog or derivative thereof.

In one example embodiment, a chimeric small molecule comprising a EGFR binding moiety has the formula:

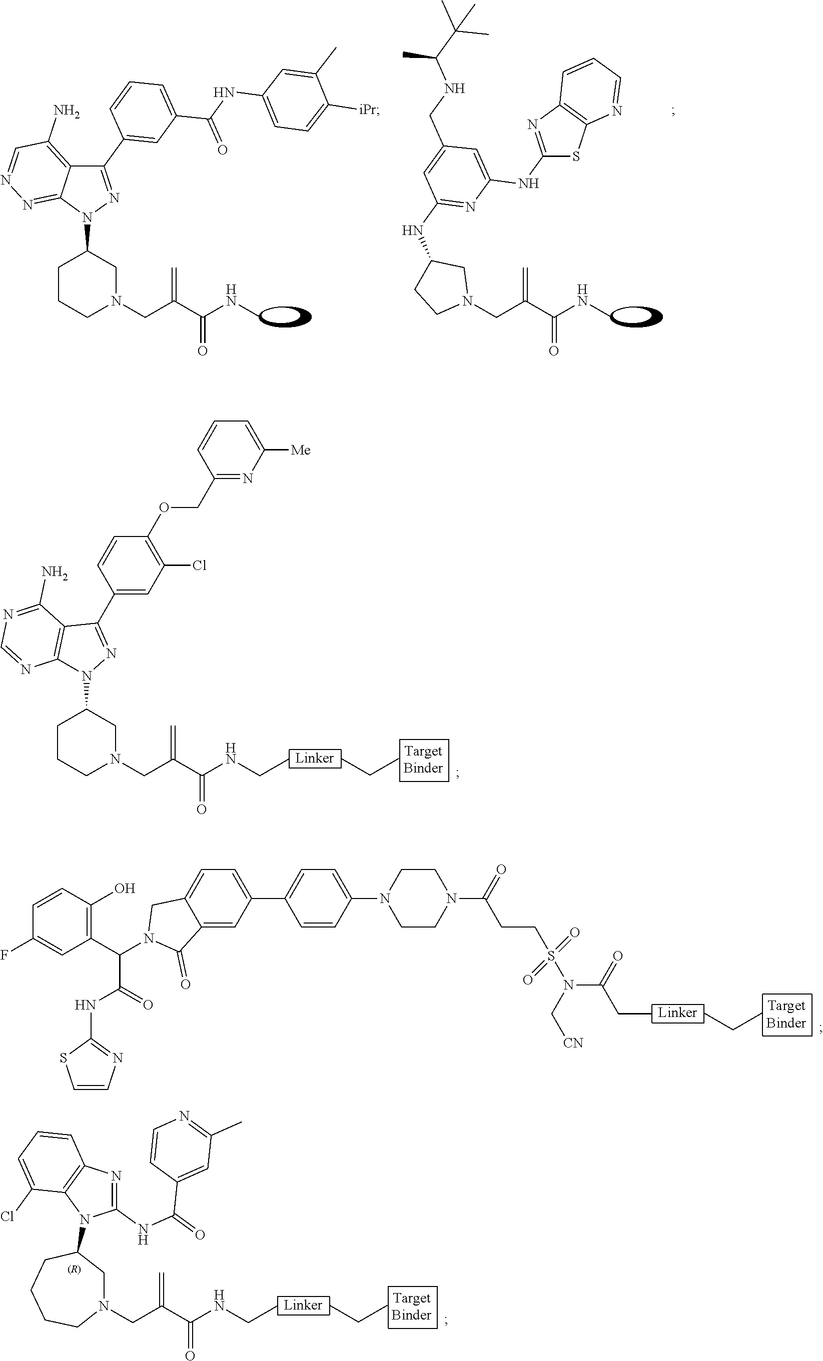

or an analog or derivative thereof, wherein the oval is the immunogenic display moiety and optionally a linker connecting the immunogenic display moiety to the electrophilic reactive group via a linker.

### FGFR Binding Moieties

In one example embodiment, the protein binding moiety is or comprises an FGFR binding moiety. FGFRs (fibroblast growth factor receptors) are a family of tyrosine kinase receptors. In one example embodiment, the FGFR is pan-FGFR, FGFR4, FGFR1, or FGFR3. In one example embodiment, the FGFR binding moiety is general to all FGFR. In one example embodiment, the protein binding molecule is specific for a particular FGFR.

In one example embodiment, the FGFR binding moiety is or comprises according to the formula:

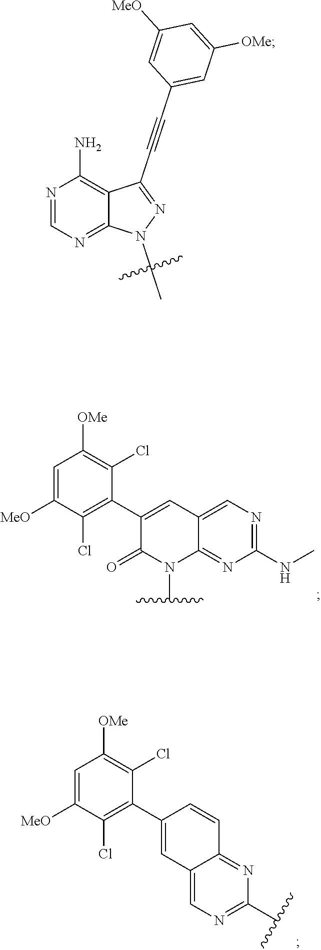 [SMILES: *n1nc(C#Cc2cc(OC)cc(OC)c2)c2c(N)ncnc21 ; *n1c(=O)c(-c2c(Cl)c(OC)cc(OC)c2Cl)cc2cnc(NC)nc21 ; *c1ncc2cc(-c3c(Cl)c(OC)cc(OC)c3Cl)ccc2n1]

or an analog or derivative thereof.

In one example embodiment, a chimeric small molecule comprising a FGFR binding moiety has the formula:

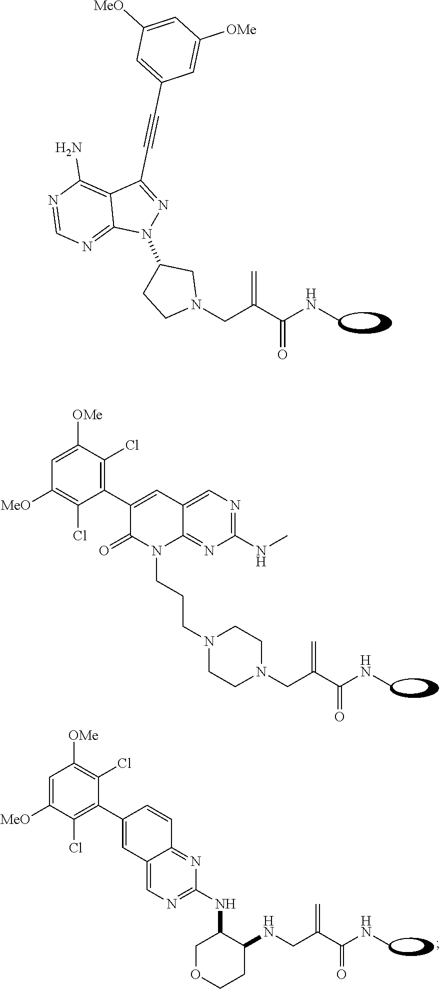 [SMILES: C=C(CN1CC[C@H](n2nc(C#Cc3cc(OC)cc(OC)c3)c3c(N)ncnc32)C1)C(=O)NC ; C=C(CN1CCN(CCCn2c(=O)c(-c3c(Cl)c(OC)cc(OC)c3Cl)cc3cnc(NC)nc32)CC1)C(=O)NC ; C=C(CN[C@H]1CCOC[C@H]1Nc1ncc2cc(-c3c(Cl)c(OC)cc(OC)c3Cl)ccc2n1)C(=O)NC]

or an analog or derivative thereof, wherein the oval is the immunogenic display moiety and optionally a linker connecting the immunogenic display moiety to the electrophilic reactive group via a linker.

In one example embodiment, the target protein binding moiety inhibits FGFR1 fusion proteins. In one example embodiment, the FGFR1 fusion protein inhibitor is Dovitinib, also known as TKI258, according to the formula:

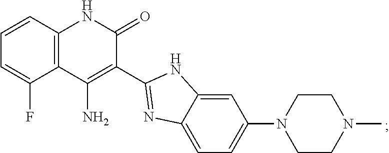 [SMILES: CN1CCN(c2ccc3nc(-c4c(N)c5c(F)cccc5[nH]c4=O)[nH]c3c2)CC1]

or an analog or derivative thereof.

### JAK Binding Moieties

In one example embodiment, the protein binding moiety is or comprises a JAK (Janus Kinase) binding moiety. JAKs are a family of tyrosine kinases. In one example embodiment, the JAK binding moiety is general to all JAK. In one example embodiment, the protein binding molecule is specific for a particular JAK. In one example embodiment, the JAK binding moiety is specific for JAK3. In one example embodiment, the JAK protein binding moiety comprises or is according to the formula:

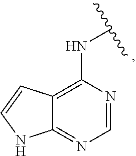 [SMILES: *Nc1ncnc2[nH]ccc12]

or an analog or derivative thereof.

In one example embodiment, a chimeric small molecule comprising a JAK binding moiety has the formula:

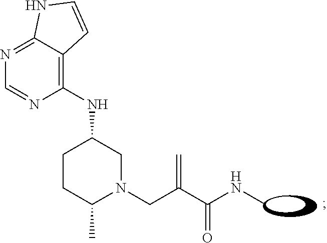 [SMILES: C=C(CN1C[C@@H](Nc2ncnc3[nH]ccc23)CC[C@H]1C)C(=O)NC]

or an analog or derivative thereof, wherein the oval is the immunogenic display moiety and optionally a linker connecting the immunogenic display moiety to the electrophilic reactive group via a linker.

### ITK Binding Moieties

In one example embodiment, the protein binding moiety is or comprises a ITK (IL-2-inducible tyrosine kinase) binding moiety. ITK belongs to the Tec family of kinases. In one example embodiment, the ITK protein binding moiety comprises or is according to the formula:

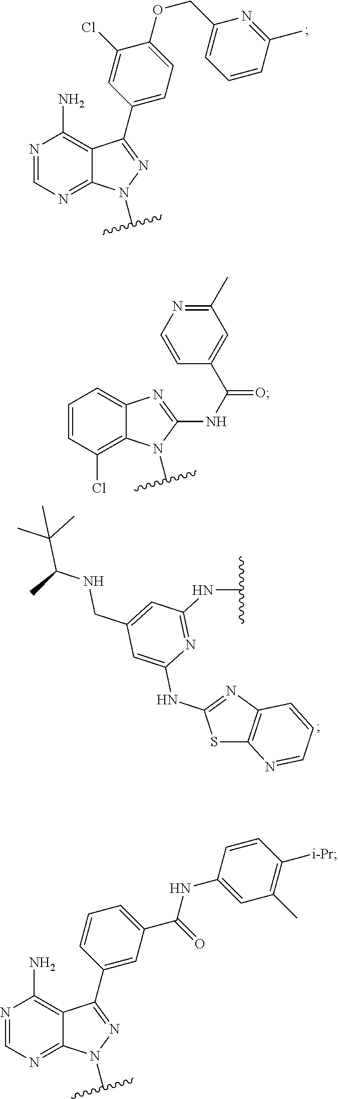

or an analog or derivative thereof.

In one example embodiment, a chimeric small molecule comprising a ITK binding moiety has the formula:

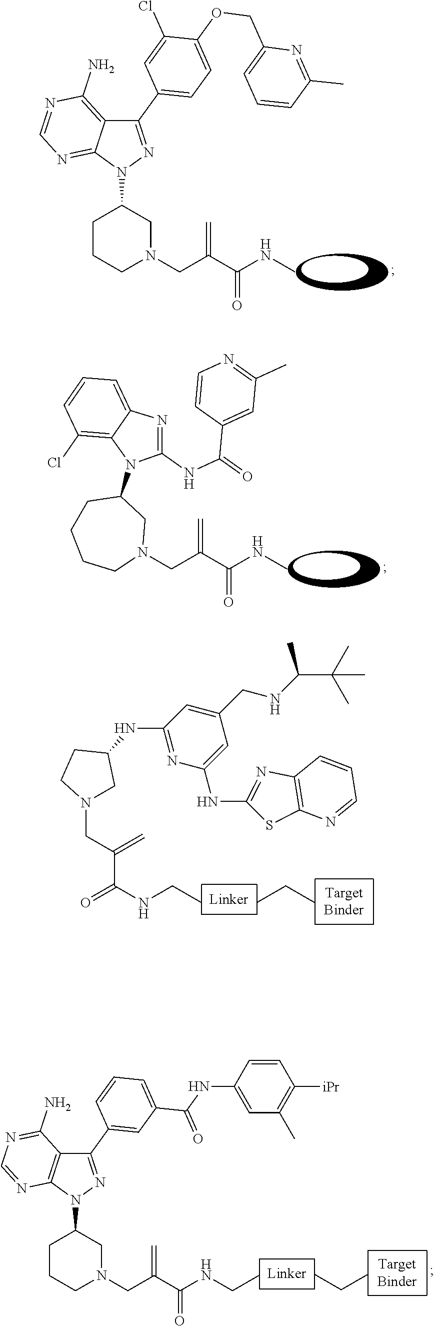

or an analog or derivative thereof, wherein the oval is the immunogenic display moiety and optionally a linker connecting the immunogenic display moiety to the electrophilic reactive group via a linker.

### CDK Binding Moieties

In one example embodiment, the protein binding moiety is or comprises a CDK (cyclin-dependent kinase) binding moiety. In one example embodiment, the protein binding molecule is specific for a particular CDK. In one example embodiment, the CDK binding moiety is specific for CDK2. In one example embodiment, the CDK protein binding moiety is or comprises according to the formula:

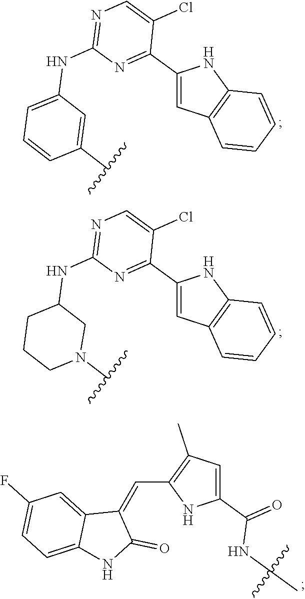

or an analog or derivative thereof.

In one example embodiment, a chimeric small molecule comprising a CDK binding moiety has the formula:

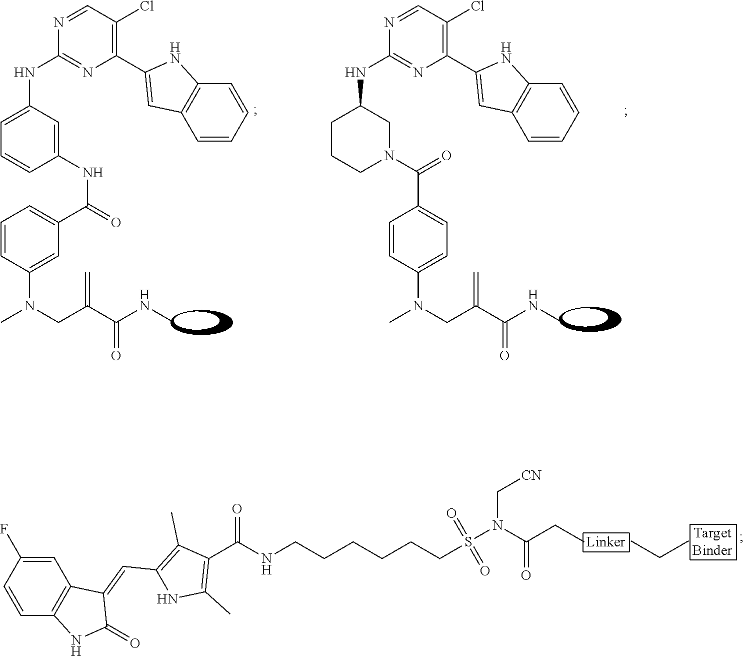

or an analog or derivative thereof, wherein the oval is the immunogenic display moiety and optionally a linker connecting the immunogenic display moiety to the electrophilic reactive group via a linker.

### AKT Binding Moieties

In one example embodiment, the protein binding moiety is a or comprises protein kinase B (PKB, also known as AKT) binding moiety. AKTs are a family of serine/threonine-specific protein kinases. In one example embodiment, the AKT binding moiety is general to all AKT. In one example embodiment, the protein binding molecule is specific for a particular AKT. In one example embodiment, the AKT protein binding moiety comprises or is according to the formula:

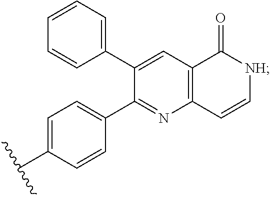 [SMILES: *c1ccc(-c2nc3cc[nH]c(=O)c3cc2-c2ccccc2)cc1]

or an analog or derivative thereof.

In one example embodiment, a chimeric small molecule comprising a AKT binding moiety has the formula:

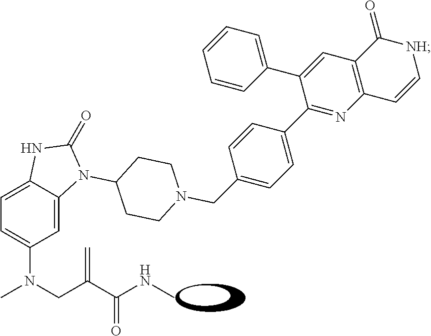 [SMILES: C=C(CN(C)c1ccc2[nH]c(=O)n(C3CCN(Cc4ccc(-c5nc6cc[nH]c(=O)c6cc5-c5ccccc5)cc4)CC3)c2c1)C(=O)NC]

or an analog or derivative thereof, wherein the oval is the immunogenic display moiety and optionally a linker connecting the immunogenic display moiety to the electrophilic reactive group via a linker.

### TAK1 Binding Moieties

In one example embodiment, the protein binding moiety comprises or is a transforming growth factor-β (TGF-β)-activated kinase 1 (TAK1) binding moiety. TAK1 is a member of the MAPK kinase kinase (MAPKKK) family. In one example embodiment, the TAK1 protein binding moiety comprises or is according to the formula:

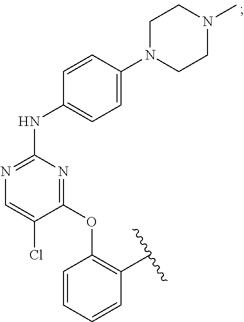 [SMILES: *c1ccccc1Oc1nc(Nc2ccc(N3CCN(C)CC3)cc2)ncc1Cl]

or an analog or derivative thereof.

In one example embodiment, a chimeric small molecule comprising a TAK1 binding moiety has the formula:

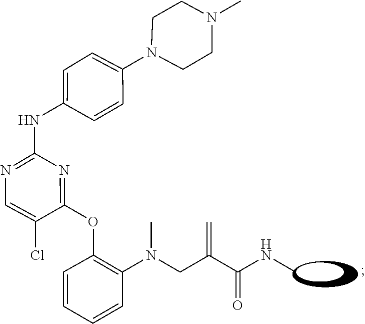 [SMILES: C=C(CN(C)c1ccccc1Oc1nc(Nc2ccc(N3CCN(C)CC3)cc2)ncc1Cl)C(=O)NC]

or an analog or derivative thereof, wherein the oval is the immunogenic display moiety and optionally a linker connecting the immunogenic display moiety to the electrophilic reactive group via a linker.

### JNK Binding Moieties

In one example embodiment, the protein binding moiety comprises or is a c-Jun N-terminal kinase (INK) binding moiety. JNKs are a family of INSERT. In one example embodiment, the JNK binding moiety is general to all JNK. In one example embodiment, the protein binding molecule is specific for a particular JNK. In one example embodiment, the JNK protein binding moiety comprises or is according to the formula:

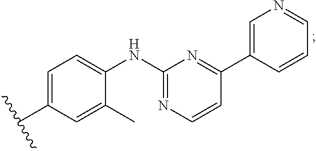 [SMILES: *c1ccc(Nc2nccc(-c3cccnc3)n2)c(C)c1]

or an analog or derivative thereof.

In one example embodiment, a chimeric small molecule comprising a JNK binding moiety has the formula:

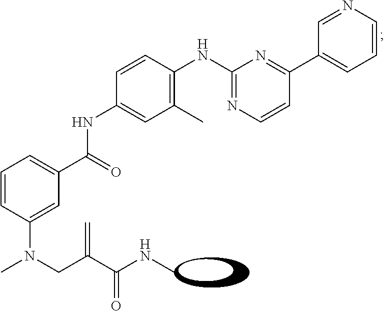 [SMILES: C=C(CN(C)c1cccc(C(=O)Nc2ccc(Nc3nccc(-c4cccnc4)n3)c(C)c2)c1)C(=O)NC]

or an analog or derivative thereof, wherein the oval is the immunogenic display moiety and optionally a linker connecting the immunogenic display moiety to the electrophilic reactive group via a linker.

### BMX Binding Moieties

In one example embodiment, the protein binding moiety comprises or is a bone marrow tyrosine kinase on chromosome X (BMX) binding moiety. BMX belongs to the Tec family of kinases. In one example embodiment, the BMX protein binding moiety comprises or is according to the formula:

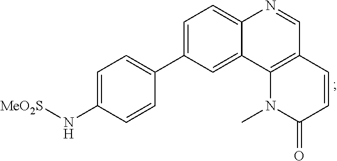 [SMILES: Cn1c(=O)ccc2cnc3ccc(-c4ccc(NS(C)(=O)=O)cc4)cc3c21]

or an analog or derivative thereof.

In one example embodiment, a chimeric small molecule comprising a BMX binding moiety has the formula:

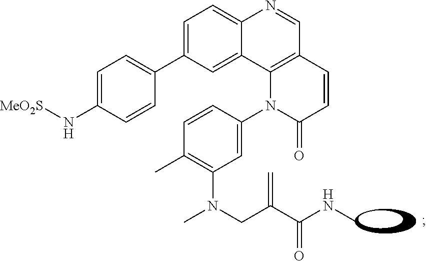 [SMILES: C=C(CN(C)c1cc(-n2c(=O)ccc3cnc4ccc(-c5ccc(NS(C)(=O)=O)cc5)cc4c32)ccc1C)C(=O)NC]

or an analog or derivative thereof, wherein the oval is the immunogenic display moiety and optionally a linker connecting the immunogenic display moiety to the electrophilic reactive group via a linker.

### LIMK Binding Moieties

In one example embodiment, the protein binding moiety comprises or is a LIM kinase (LIMK) binding moiety. LIMKs are a family of actin-binding kinases. In one example embodiment, the LIMK binding moiety is general to all LIMK. In one example embodiment, the protein binding molecule is specific for a particular LIMK. In one example embodiment, the LIMK binding moiety is specific for LIMK1 or LIMK2. In one example embodiment, the LIMK protein binding moiety comprises or is according to the formula:

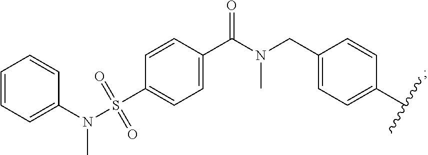 [SMILES: *c1ccc(CN(C)C(=O)c2ccc(S(=O)(=O)N(C)c3ccccc3)cc2)cc1]

or an analog or derivative thereof.

In one example embodiment, a chimeric small molecule comprising a BMX binding moiety has the formula:

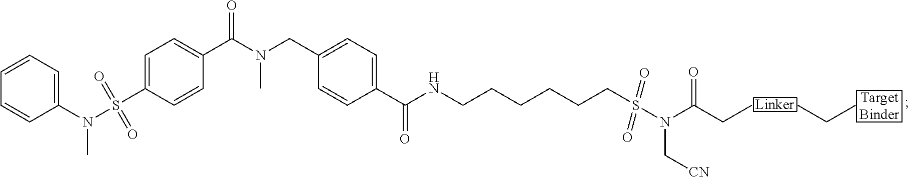

or an analog or derivative thereof.

### IRE1 Binding Moieties

In one example embodiment, the protein binding moiety comprises or is an inositol-requiring enzyme kinase (IRE1) binding moiety. In one example embodiment, the IRE1 protein binding moiety comprises or is according to the formula:

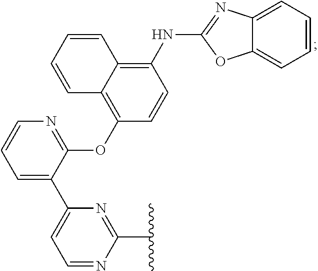 [SMILES: *c1nccc(-c2cccnc2Oc2ccc(Nc3nc4ccccc4o3)c3ccccc23)n1]

or an analog or derivative thereof.

In one example embodiment, a chimeric small molecule comprising a BMX binding moiety has the formula:

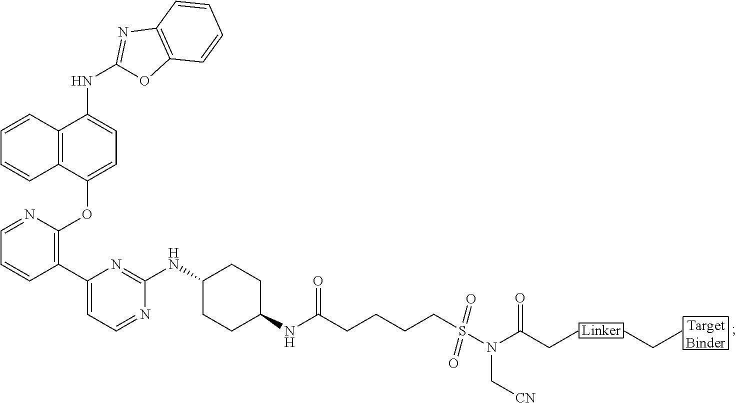

or an analog or derivative thereof.

### RIP Binding Moieties

In one example embodiment, the protein binding moiety comprises or is a receptor-interacting kinase (RIP) binding moiety. RIPs are a family of threonine/serine protein kinases. In one example embodiment, the RIP binding moiety is general to all RIP. In one example embodiment, the protein binding molecule is specific for a particular RIP. In one example embodiment, the RIP binding moiety is specific for RIP1. In one example embodiment, the RIP protein binding moiety comprises or is according to the formula:

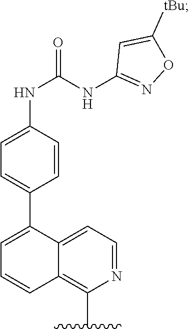 [SMILES: *c1nccc2c(-c3ccc(NC(=O)Nc4cc(C(C)(C)C)on4)cc3)cccc12]

or an analog or derivative thereof.

In one example embodiment, a chimeric small molecule comprising a RIP binding moiety has the formula:

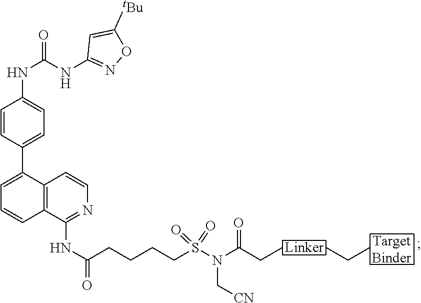

or an analog or derivative thereof.

### MEK Binding Moieties

In one example embodiment, the protein binding moiety comprises or is a mitogen-activated protein kinase kinase (MAPKK, also known as MEK) binding moiety. The MEKs, MEK1 and MEK2, are dual-specificity kinase enzymes which phosphorylate mitogen-activated protein kinase (MAPK). In one example embodiment, the MEK binding moiety is general to both MEK (collectively referred to as MEK1/2). In one example embodiment, the protein binding molecule is specific for a particular MEK. In one example embodiment, the MEK protein binding moiety comprises or is according to the formula:

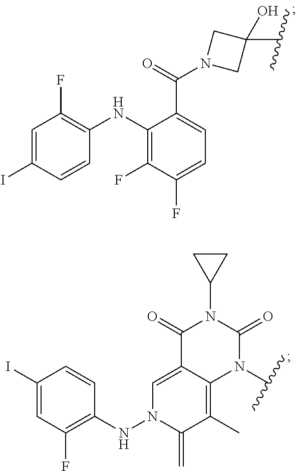

or an analog or derivative thereof.

In one example embodiment, a chimeric small molecule comprising a BMX binding moiety has the formula:

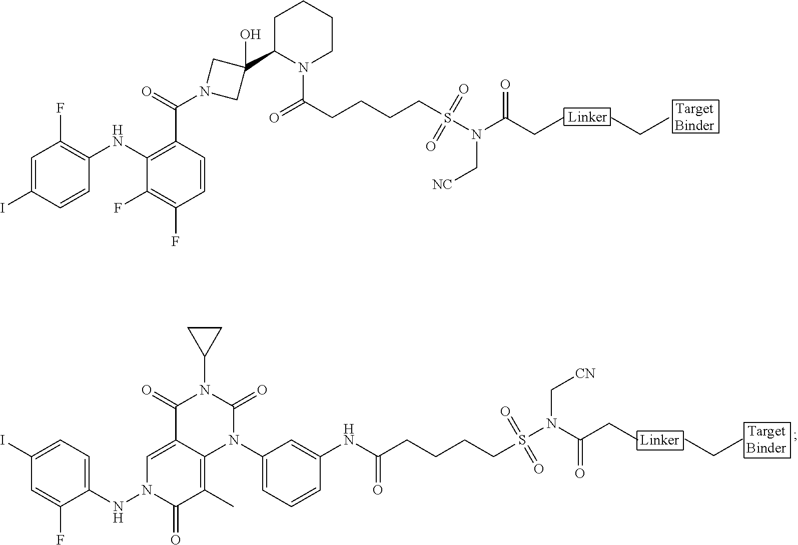

or an analog or derivative thereof.

### HSP90 Binding Moieties

Heat Shock Protein 90 (Hsp90) is an ATP dependent molecular chaperone that with its co-chaperones modulates proteins involved in cell cycle control and signal transduction. Like many ATP dependent proteins, the protein undergoes a functional cycle that is linked to its ATPase cycle.

In an embodiment, the HSP90 binding molecule comprises or is

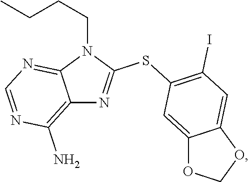 [SMILES: CCCCn1c(Sc2cc3c(cc2I)OCO3)nc2c(N)ncnc21]

or analog or derivative thereof.

Additional HSP90 binders include geldanamycin and derivatives thereof, including Tanspimycin (IC50 of 5 nM in cell free assay), according to the formula;

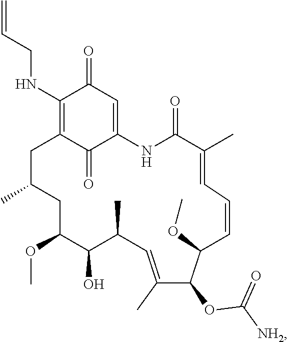 [SMILES: C=CCNC1=C2C[C@@H](C)C[C@H](OC)[C@H](O)[C@@H](C)/C=C(\C)[C@H](OC(N)=O)[C@@H](OC)/C=C\C=C(/C)C(=O)NC(=CC1=O)C2=O]

Alvespimycin (IC50 of 62 nM in cell-free assay) according to the formula;

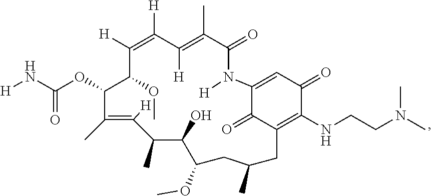 [SMILES: CO[C@H]1/C=C\C=C(/C)C(=O)NC2=CC(=O)C(NCCN(C)C)=C(C[C@@H](C)C[C@H](OC)[C@H](O)[C@@H](C)/C=C(\C)[C@@H]1OC(N)=O)C2=O]

EC141 according to the formula;

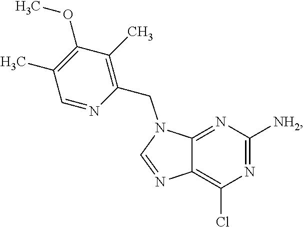 [SMILES: COc1c(C)cnc(Cn2cnc3c(Cl)nc(N)nc32)c1C]

Novobiocin according to the formula

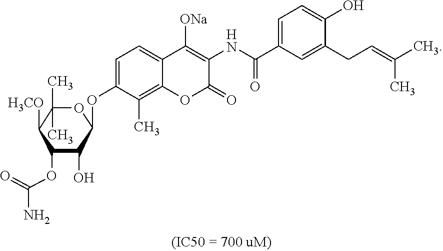

Novobiocin analogs can also be utilized and as described in Hall et al., J Med Chem. 2016 Feb. 11; 59(3): 925-933; doi: 10.1021/acs.jmedchem.5b01354, incorporated by reference, which can be used as a MAPK signaling disruptor.

### BTK Binding Moieties

In an example embodiment, the target binding moiety comprises or is Bruton's Tyrosine Kinase (BTK), a protein involved in multiple signaling cascades and is widely expressed in B cells. First, BTK is a cytoplasmic protein and thus available for interactions with enzymes.

In an embodiment, the BTK binding molecule comprises or is a formula selected from the group consisting of,

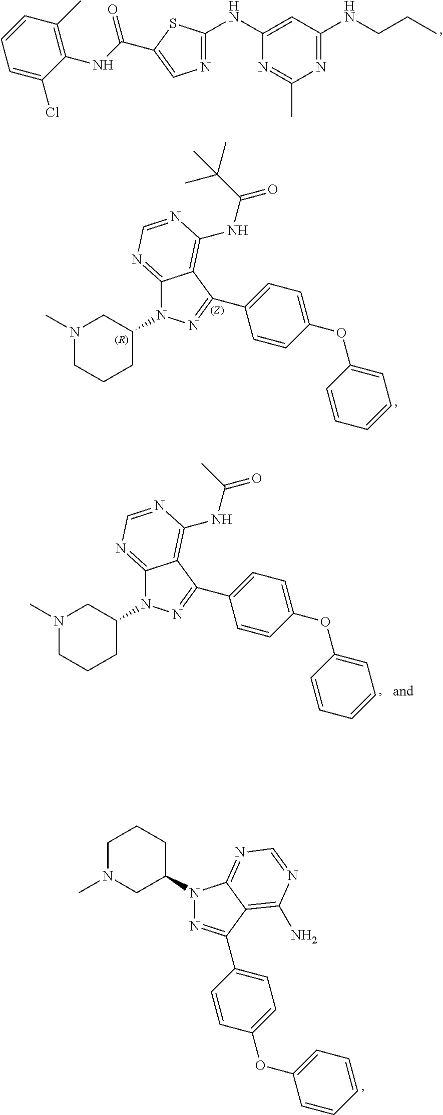

or an analog or derivative thereof.

### MDM2 Binding Moieties

In an embodiment, the target protein binding moiety comprises or is an MDM2 binding moiety according to

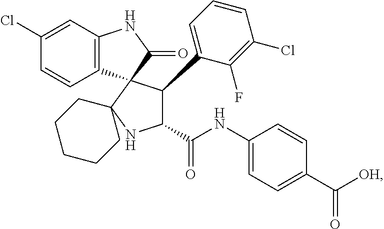 [SMILES: O=C(O)c1ccc(NC(=O)[C@@H]2NC3(CCCCC3)[C@@]3(C(=O)Nc4cc(Cl)ccc43)[C@H]2c2cccc(Cl)c2F)cc1]

or an analog or derivative or thereof, or any combination thereof.

### BRD4 Binding Moieties

In an embodiment, the target protein binding moiety comprises or is a BRD4 binding moiety selected from the group consisting of

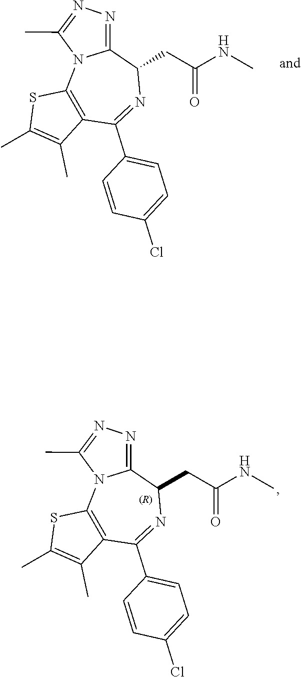

or an analog or derivative thereof.

### PtpA, PtpB Binding Moieties

In one example embodiment, target protein binding moiety comprises or is a PtpA binding moiety according to the formula

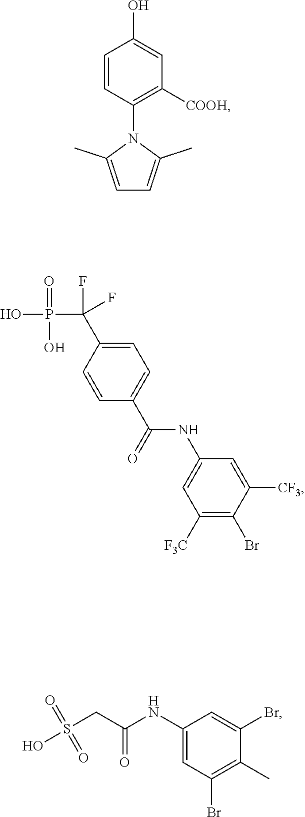

or any analog or derivative thereof.

In preferred embodiments, the PtpB binding moiety comprises or is according to the formula

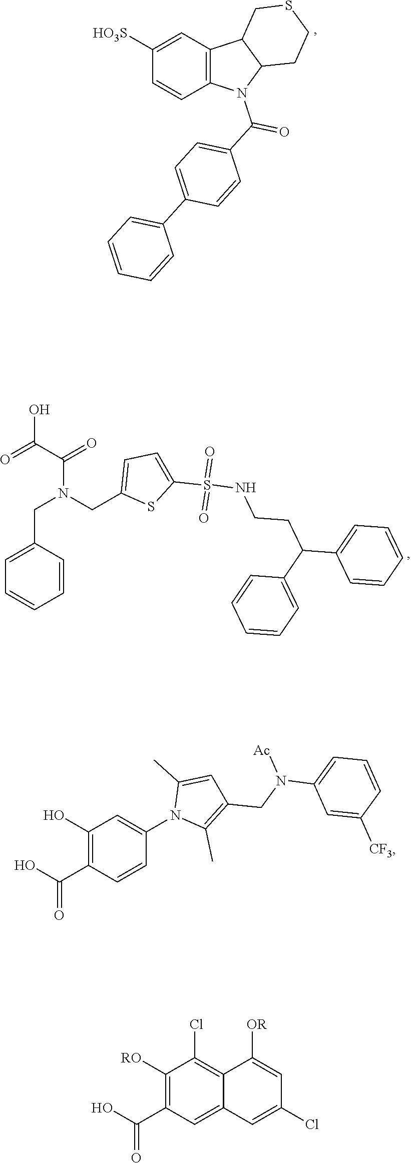

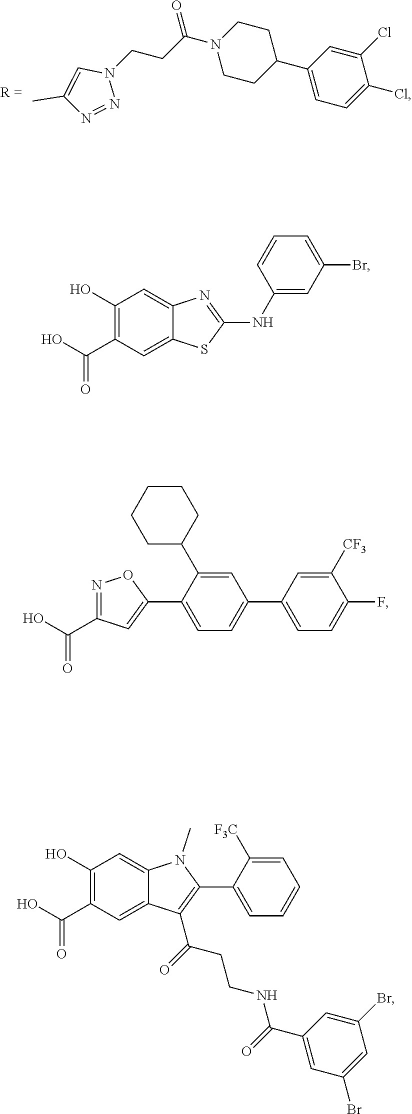 [SMILES: Cc1cn(CCC(=O)N2CCC(c3ccc(Cl)c(Cl)c3)CC2)nn1 ; O=C(O)c1cc2sc(Nc3cccc(Br)c3)nc2cc1O ; O=C(O)c1cc(-c2ccc(-c3ccc(F)c(C(F)(F)F)c3)cc2C2CCCCC2)on1 ; Cn1c(-c2ccccc2C(F)(F)F)c(C(=O)CCNC(=O)c2cc(Br)cc(Br)c2)c2cc(C(=O)O)c(O)cc21]

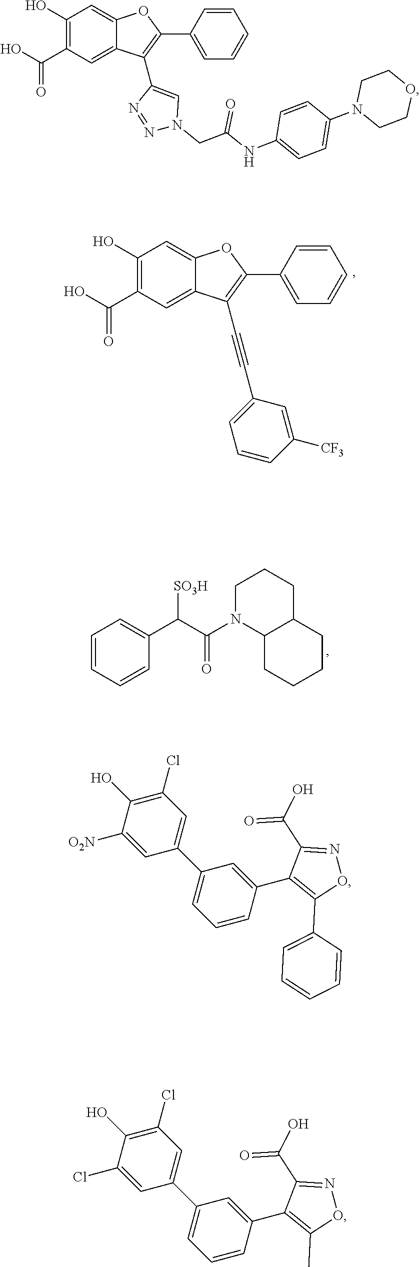 [SMILES: O=C(Cn1cc(-c2c(-c3ccccc3)oc3cc(O)c(C(=O)O)cc23)nn1)Nc1ccc(N2CCOCC2)cc1 ; O=C(O)c1cc2c(C#Cc3cccc(C(F)(F)F)c3)c(-c3ccccc3)oc2cc1O ; O=C(C(c1ccccc1)S(=O)(=O)O)N1CCCC2CCCCC21 ; O=C(O)c1noc(-c2ccccc2)c1-c1cccc(-c2cc(Cl)c(O)c([N+](=O)[O-])c2)c1 ; Cc1onc(C(=O)O)c1-c1cccc(-c2cc(Cl)c(O)c(Cl)c2)c1]

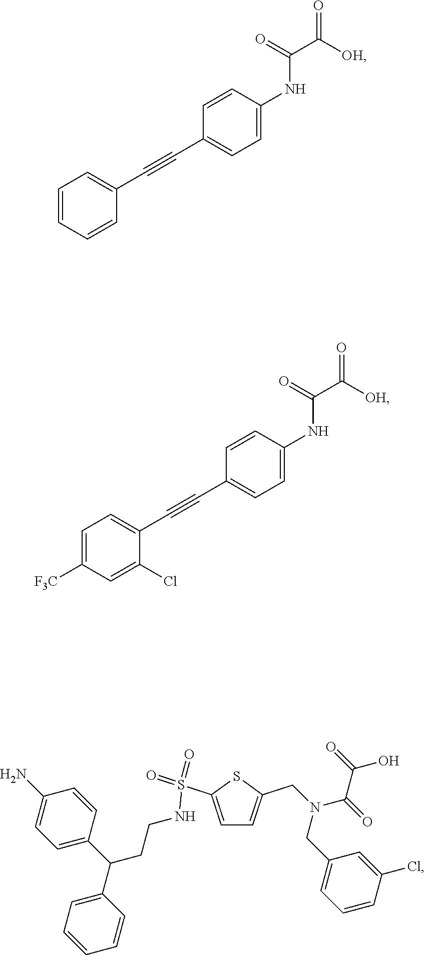 [SMILES: O=C(O)C(=O)Nc1ccc(C#Cc2ccccc2)cc1 ; O=C(O)C(=O)Nc1ccc(C#Cc2ccc(C(F)(F)F)cc2Cl)cc1 ; Nc1ccc(C(CCNS(=O)(=O)c2ccc(CN(Cc3cccc(Cl)c3)C(=O)C(=O)O)s2)c2ccccc2)cc1]

or any analog or derivative thereof.

### SapM Binding Moieties

In one example embodiment, the target protein binding moiety comprises or is a SapM binding moiety. In an example embodiment, the SapM binding moiety contains a trihydroxy-benzene group. In an example embodiment, the SapM binding moiety comprises of a benzylidenemalononitrile scaffold. In one example embodiment, the SapM binding moiety has the formula.

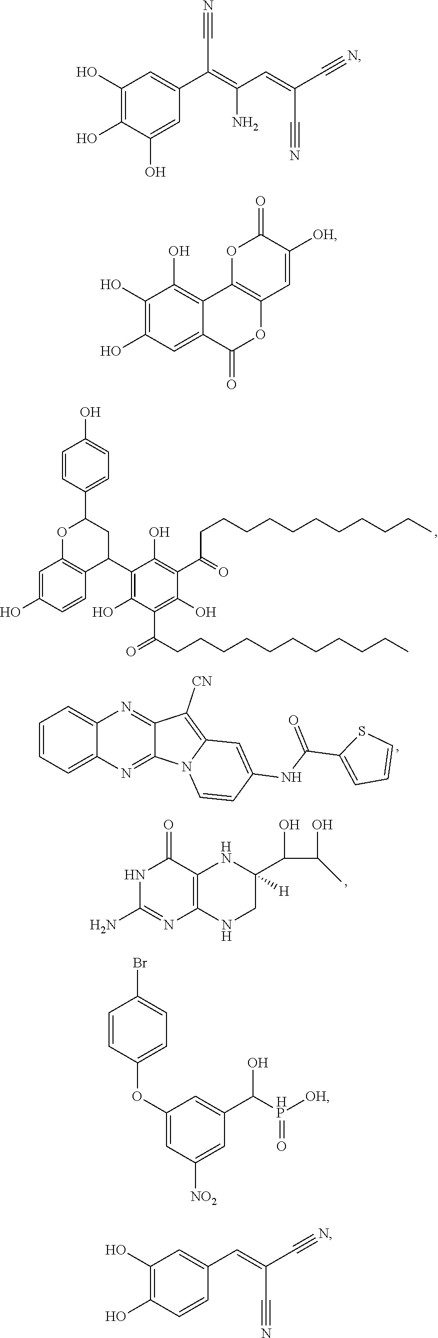

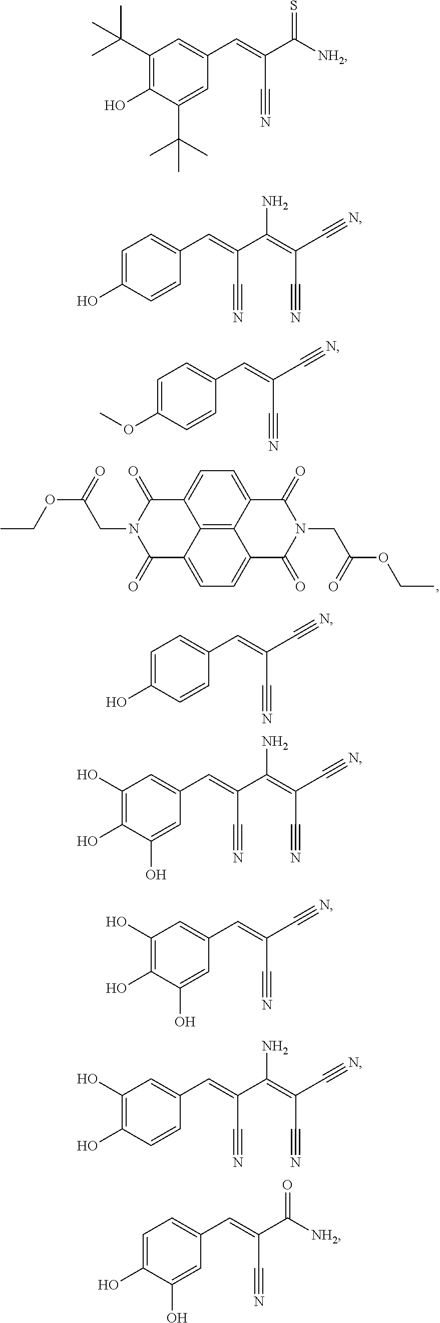

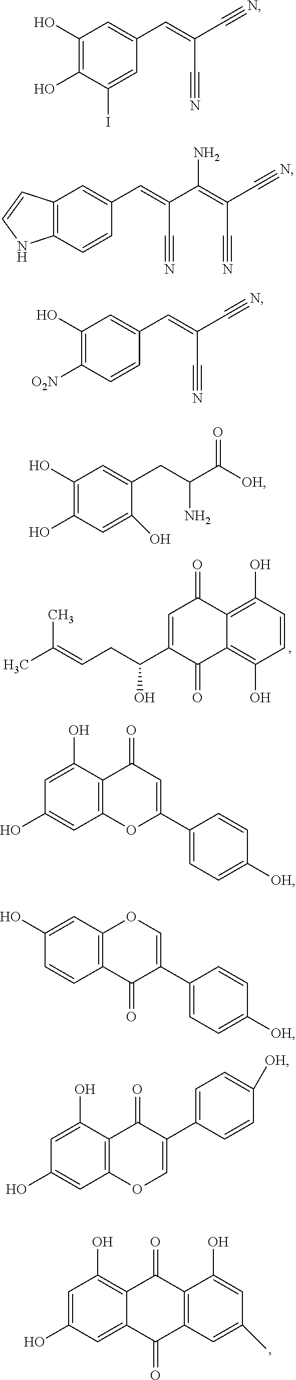

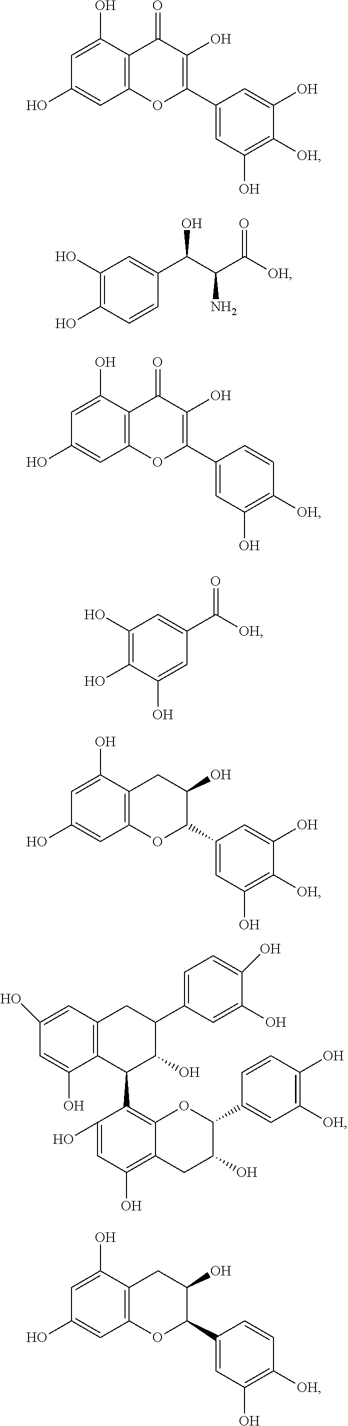

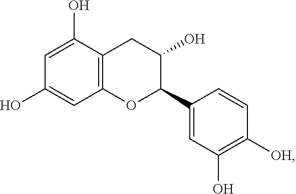 [SMILES: Oc1cc(O)c2c(c1)O[C@H](c1ccc(O)c(O)c1)[C@@H](O)C2]

or an analog or derivative thereof. In one example embodiment the SapM binding moiety comprises or is L-ascorbic acid (L-AC) and 2-phospho-L-ascorbic acid (2P-AC).

### UMPK Binding Moieties

In one example embodiment the target binding moiety comprises or is a UMPK and any derivative thereof identified in US Patent Application US US20090209022, herein incorporated by reference.

### Colistin Binding Moieties

In preferred embodiments, the PsA associated target protein binding moiety comprises or is a polymyxin. In an embodiment, the polymyxin is polymyxin B or polymyxin E (Colistin),

which has the formula:

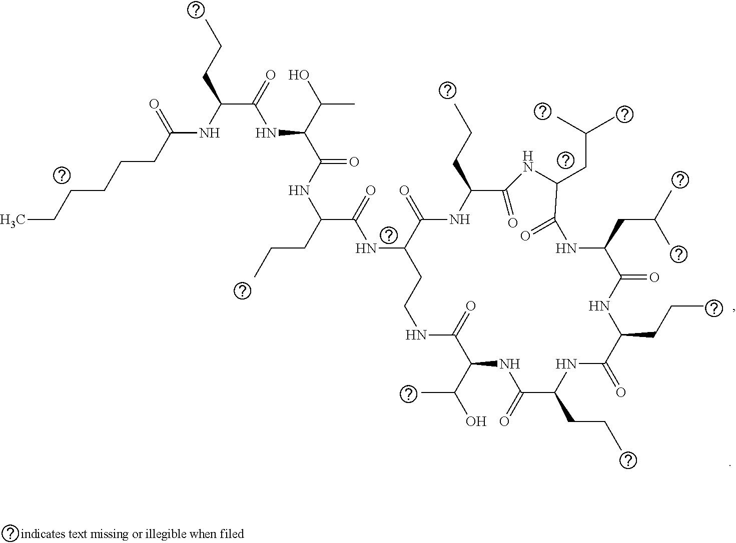 [SMILES: CCCCCCC(=O)N[C@@H](CCC)C(=O)N[C@H](C(=O)NC(CCC)C(=O)NC1CCNC(=O)[C@H](C(C)O)NC(=O)[C@H](CCC)NC(=O)[C@H](CCC)NC(=O)[C@H](CC(C)C)NC(=O)C(CC(C)C)NC(=O)[C@H](CCC)NC1=O)C(C)O]

or an analog or derivative thereof.

### PSMA Binding Moieties

In certain example embodiments, the target protein binding moiety is a PSMA binding moiety. In certain embodiments, the PSMA binding moiety comprises (or is)

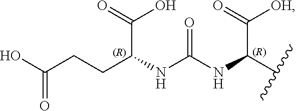

or an analog or derivative thereof.

### Electrophilic Reactive Group

In one embodiment, the chimeric small molecules or binding moieties thereof as disclosed herein may be modified to include an electrophilic reactive group. In one embodiment, the electrophilic reactive group is located between the target protein binding moiety (A) and a linker (L, L1, or L2) attached to the immunogenic display moiety (B). In one embodiment, the electrophilic reactive group is located between the target protein binding moiety (A) and a linker (L, L1, or L2) attached to the immunogenic display moiety (B). In an embodiment, the electrophilic reactive group is attached to each of the target protein binding moiety (A) and the immunogenic display moiety (B) via a first linker (L1) and a second linker (L2), respectively. In an embodiment, the electrophilic reactive group is attached to the target protein binding moiety (A) and the immunogenic display moiety (B), where either the attachment to A or to B is an indirect attachment via a linker (L).

An electrophilic reactive group, as used herein, is typically a functional group that can form a reversible or irreversible bond with a nucleophilic functional group. In one example embodiment, the electrophilic reactive group allows for the chimeric small molecule, including the immunogenic display moiety and/or the protein binding moiety, to attach to the target protein. Upon attaching to the electrophilic reactive group, the protein is now tagged with at least the immunogenic display moiety or the protein binding moiety. In one example embodiment, the molecules or binding moieties may be modified at an electrophilic reactive group to reduce or lessen the strength of covalent binding capabilities of an electrophilic reactive group, or to increase the binding affinity or strength of binding of an electrophilic reactive group as desired according to the application. In an embodiment, a binding molecule may be chosen that would create irreversible covalent binding at a target. When used in a chimeric small molecule, such tight bonding may be less desirable. Thus, modification of such electrophilic reactive group would be desirable and can be modified to reduce the interaction, see, e.g. sciencedirect.com/science/article/pii/S0968089618320807. In particular, reactivity can be designed to allow for covalent binding at the target, with reversible or irreversible properties, depending on desired functionality. In an embodiment, the electrophilic reactive group is designed to react with an amino acid side chain reactive group. The amino acid side chain reactive group may be nucleophilic. The nucleophilic amino acid side chain reactive group may comprise arginine, lysine, histidine, cysteine, aspartic acid, glutamic acid and tyrosine. In one preferred embodiment, the electrophilic reactive group reacts with lysine. An exemplary database that can aid in identification for protein ligand interaction around the binding site is described in Du et al, Nucleic Acids Research, Volume 49, Issue D1, 8 Jan. 2021, Pages D1122-D1129, incorporated herein by reference, with the database, CovalentInDB accessible at cadd.zju.edu.cn/cidb/. The approach can be used with any design of the electrophilic reactive group for molecules as disclosed herein.

In one example embodiment, the electrophilic group is a covalent warhead (a reactive group capable of forming a covalent bond with a nucleophilic amino acid of a protein) attached to a protein binding moiety, such that labeling occurs via covalent bonding between a protein binding moiety and a protein amino acid (e.g., via a Michael Addition reaction between a cysteine or lysine amino acid and the electrophilic group).

In one example embodiment, the electrophilic reactive group comprises (or is) one of

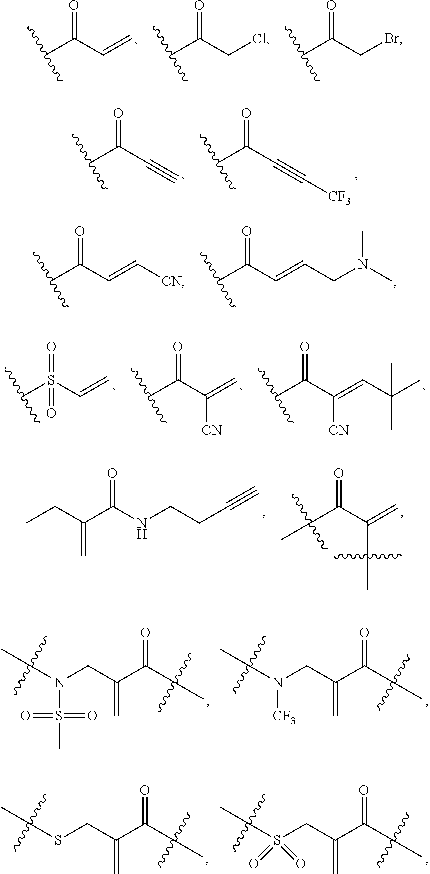

or an analog or derivative thereof. In one example embodiment, the electrophilic group comprises (or is)

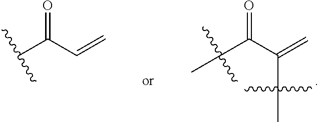

In one example embodiment, the electrophilic group is directly or indirectly (via a linker) to each of an immunogenic display moiety and a protein binding moiety, such that labeling occurs via covalent bonding between the immunogenic display moiety and a protein amino acid (e.g., via a Michael Addition reaction or an addition-elimination between a cysteine or lysine amino acid and the electrophilic group) or such that release occurs via covalent bonding between the linker and a protein amino acid.

In one example embodiment, the electrophilic reactive group comprises (or is)

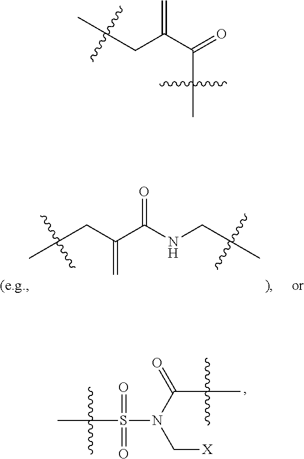

where X is an electron withdrawing group, optionally a —CN group. In one example embodiment, the electrophilic reactive group comprises (or is)

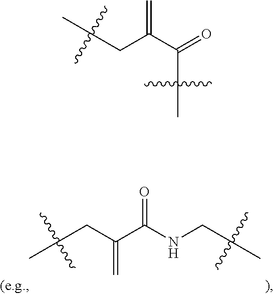

and labeling with an immunogenic display moiety occurs via covalent bonding between the immunogenic display moiety and a cysteine amino acid sulfur group (e.g., addition-elimination reaction). In one example embodiment, the electrophilic reactive group comprises (or is)

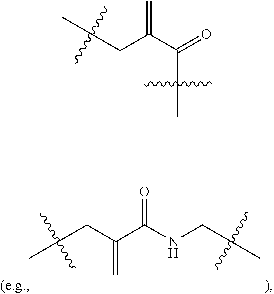

and release of an immunogenic display moiety occurs via covalent bonding between the linker and a cysteine amino acid sulfur group (e.g., addition-elimination reaction). In one example embodiment, the electrophilic reactive group comprises (or is)

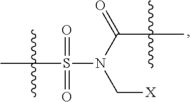

where X is an electron withdrawing group, optionally a —CN group, and labeling occurs via covalent bonding between the immunogenic display moiety and a lysine amino acid amine group (e.g., addition-elimination reaction).
N-acyl-N-alkyl sulfonimide (NASA)

In one example embodiment, the electrophilic reactive group is an N-acyl N-alkyl sulfonamide (NASA) electrophilic reactive group or an analog or derivative thereof. NASA chemistry may be used to accomplish the design of the electrophilic reactive group. NASA chemistry is generally described in Nat Commun 9, 1870 (2018), incorporated herein by reference. In one example embodiment, an immunogenic display moiety (B) can be directly attached to an N-acyl N-alkyl sulfonamide (NASA) electrophilic reactive group or indirectly attached to a NASA via a linker group. In one example embodiment, the NASA electrophilic reactive group directly or indirectly attached to the immunogenic display moiety (B) is further directly attached to a protein binding moiety or indirectly attached to the protein binding moiety via a linker group. Upon non-covalent binding of the protein binding moiety to a protein, the NASA will chemically react with a proximal lysine. The NASA-modified protein binding moiety then disassociates from the immunogenic display moiety leaving behind the immunogenic display moiety covalently attached to the protein (attached directly or via a linker). In an example embodiment, NASA chemistry is used to label the protein with an immunogenic display moiety. Accordingly, an embodiment comprises methods of making compositions disclosed herein using NASA chemistry, and as further described in the examples.

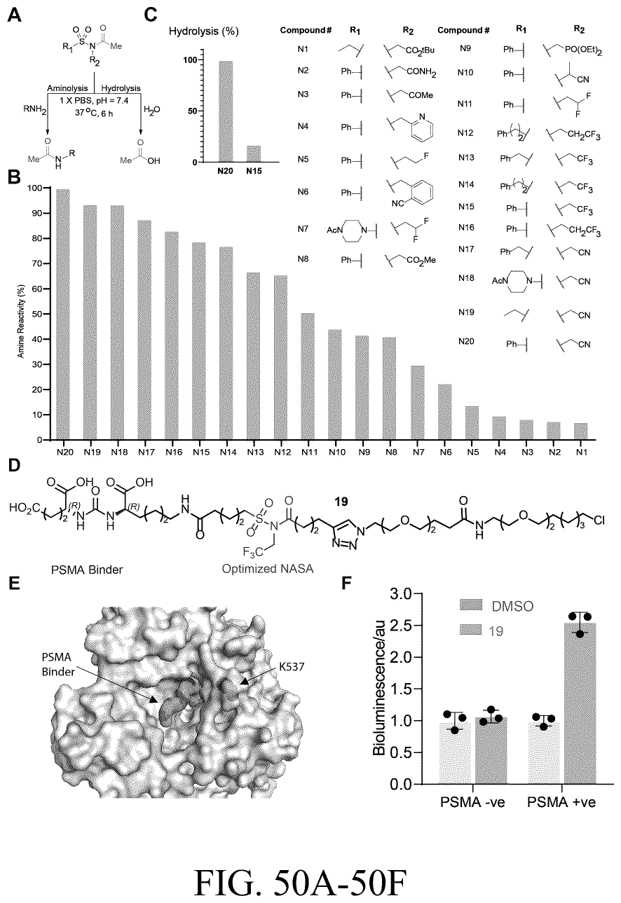

In an example embodiment, a NASA analogue comprises (or has) the formula:

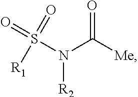

where R₁ and/or R₂ is/are independently selected from R₁ and/R₂ moieties of any one of FIGS. 1-50, e.g., FIG. 50. In an example embodiment, a NASA analogue comprises (or has) the formula of any NASA analogue of any one of FIGS. 1-50, e.g., FIG. 50. In an example embodiment, a NASA analogue comprises (or has) the formula of, or independently comprises any R₁ and/or R₂ of, any one of the following.

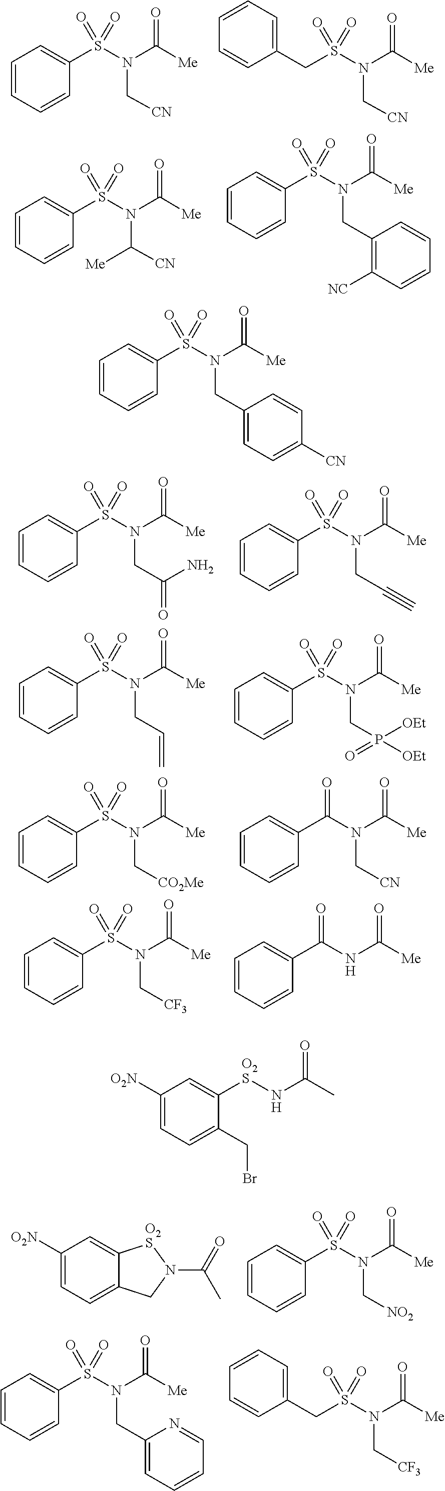

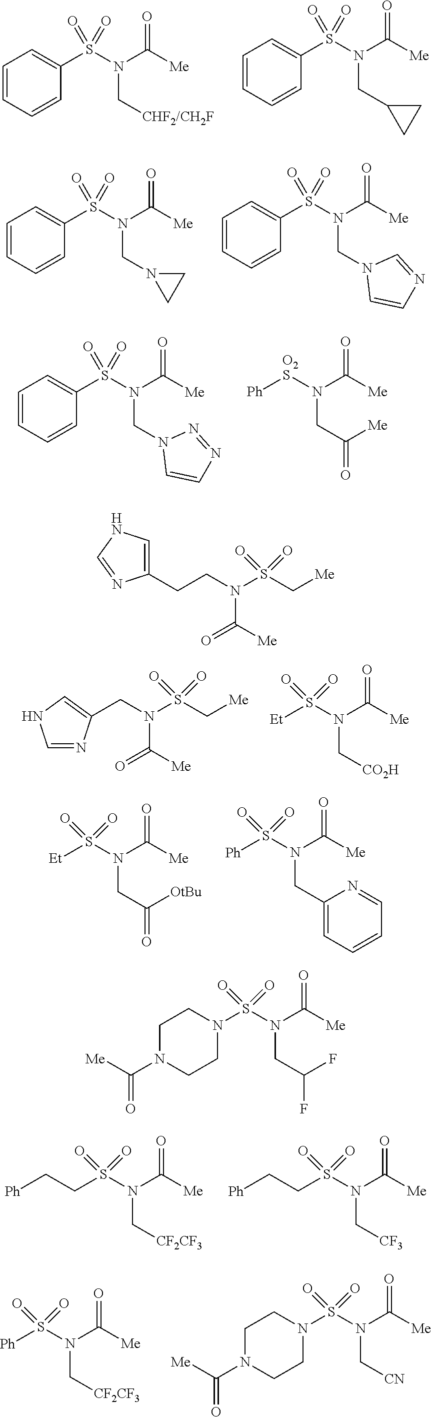

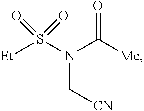 [SMILES: CCS(=O)(=O)N(CC#N)C(C)=O]

or an analog or derivative thereof. In one example embodiment, the protein binding moiety is directly or indirectly (via a linker group) attached to the electrophilic reactive group via any ring position of the NASA. In one example embodiment, the immunogenic display moiety is directly or indirectly (via a linker group) attached to the electrophilic reactive group via the acyl group.

### Dibromophenyl Benzoate

In one example embodiment, the electrophilic reactive group is dibromophenyl benzoate (DB) or an analog or derivative thereof. DB can be used to functionalize a linker by reacting with a nucleophile located on an enzyme. The dibromophenyl group acts as the leaving group facilitating the reaction while the benzoate stabilizes the now attached moiety. DB chemistry is generally described in Takaoka et al. Chem. Sci., (2015), 6, 3217-3224, incorporated herein by reference. In one example embodiment, a linker connecting a protein binding moiety and an immunogenic display moiety is functionalized with DB to label a target protein with the immunogenic display moiety. In one example embodiment, the protein binding moiety is directly or indirectly (via a linker group) attached to the electrophilic reactive group via the dibromophenyl group, and the immunogenic display moiety is directly or indirectly (via a linker group) attached to the electrophilic reactive group via the benzoate group.

### N-Sulfonyl Pyridone

In preferred embodiments, the electrophilic reactive group is N-sulfonyl pyridone (SP). SP can be used to functionalize a linker by undergoing sulfonylation with a nucleophile located on an enzyme. In a preferred embodiment, a linker connecting an enzyme binding moiety and protein binding moiety is functionalized with SP to label a target enzyme with the protein binding moiety. SP chemistry is generally described in K. Matsuo et al. Angew. Chem. Int. Ed. 2018, 57, 659 incorporated herein by reference. In one example embodiment, the protein binding moiety is directly or indirectly (via a linker group) attached to the electrophilic reactive group via the pyridone group, and the immunogenic display moiety is directly or indirectly (via a linker group) attached to the electrophilic reactive group via the sulfonyl benzene group.

In one example embodiment, the electrophilic reactive group comprises one of

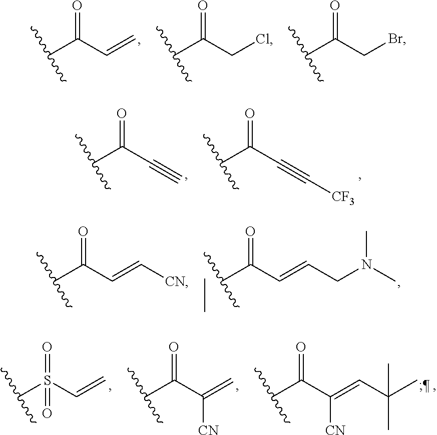

or an analog or derivative thereof.

In one example embodiment, the electrophilic reactive group is of the formula:

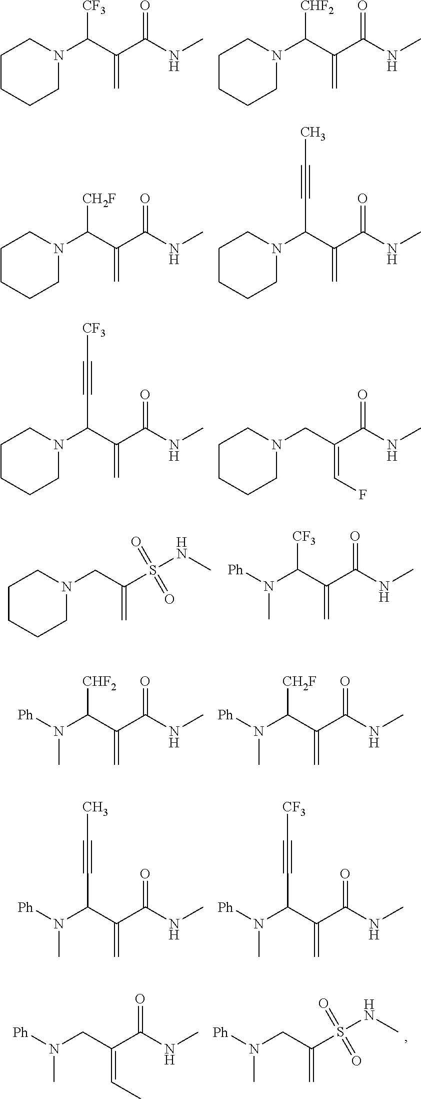

an analog or derivative thereof.

### Photo-Reactive Group

In one example embodiment, the electrophilic reactive group is a photo-reactive group. In one embodiment, the photo-reactive group is a photoactivated cell-surface reactive group. In another embodiment, the photoactivated cell-surface reactive group is a benzophenone, azide, or diazirine, wherein the group is activated to become a carbon-centered radical, nitrene, or carbene, respectively. In another embodiment, the photo-reactive group is a thienyl-substituted alpha-ketoamide, see e.g., Ota, E., et al. “Thienyl-Substituted α-Ketoamide: A Less Hydrophobic Reactive Group for Photo-Affinity Labeling.” ACS Chem. Biol. 2018, 13 (4), 876-880.

### Linker

A linker (also referred to herein as a linking moiety or a linker group or a linker molecule) is a bifunctional or multifunctional moiety that can be used to link one or more of a protein binding moiety to an electrophilic reactive group, a protein binding moiety to an immunogenic display moiety, or an electrophilic reactive group to an immunogenic display moiety. A multifunctional linker can further be used to link more than one (e.g., two or more) immunogenic moieties to a chimeric small molecule. In some embodiments, the linker has a functionality capable of reacting with the moieties for covalent attachment. The linker moiety is preferably a chemical linker moiety and is represented in the formulas of the present invention as L. In one example embodiment, the linker moiety L refers to a series of connected linker moieties (e.g., one or more repeats, e.g., 1, 2, 3, 4, 5, 6, 7, 8, or more repeats, of the same linker moiety and/or one or more different linker moieties) which may be utilized to facilitate or improve spacing, conformation, and/or performance of the molecules. The linker described herein may refer to L or to both L1 and L2, or L1 and L2 may be different linkers described herein. When more than one linker is used in a chimeric small molecule, the linkers may be the same or different from each other.

In one example embodiment, the linker may be represented with an exit vector. In one example embodiment, the exit vector may be represented independently of the linker. Exit vector parameters can be identified in part based on average orientation of a substituent attached to a variation point which can be generated using chemoinformatics software. An exit vector may comprise outgoing bonds from a chemical moiety. In an embodiment, the exit vector is provided as bonds on the linker or from the binding moiety, providing conformation of attachment between the linker and the activator moiety and/or the localizing moiety.

### Exit Vectors

One or more exit vectors may be utilized with the molecules described herein. In certain embodiments, the linker or protein binding moiety may be represented with an exit vector comprised in the linker or protein binding moiety. In an embodiment, the exit vector may be represented independently of the linker or protein binding moiety. Exit vector parameters can be identified in part based on average orientation of a substituent attached to a variation point which can be generated using chemoinformatics software. An exit vector may comprise outgoing bonds from a chemical moiety. In an embodiment, the exit vector is provided as bonds on the linker or from an Abl binding moiety, providing conformation of attachment between the linker and the Abl binding moiety and/or the second Abl binding moiety. The exit vector may also be represented independent of the linker of the formulas detailed herein. In an embodiment, the exit vector is comprised in W.

In an embodiment, the bond is chosen to be energetically favorable, preferably increasing binding affinity. The exit vector may be adjusted depending on the linker utilized in the molecules. In embodiments, the exit vector is a chemical moiety or bond that facilitates stereochemical protrusion that may further facilitate subsequent coupling, bonding and/or accessibility.

In one example embodiment, the protein binding moiety has an adapter or reactive handle, both used herein interchangeably. The reactive handle comprises the group on the protein binding moiety that attaches to the linker. In one example embodiment, the reactive handle can perform click chemistry, amide coupling chemistry, crosslinking chemistry, alkylation, or sulfonation chemistry. (See e.g., Nwe, K.; Brechbiel, M. W. Growing Applications of “Click Chemistry” for Bioconjugation in Contemporary Biomedical Research. Cancer Biotherapy and Radiopharmaceuticals, 2009, 24, 289-302.). Examples of click chemistry reactions include, but are not limited to: [3+2]cycloadditions, e.g., Huisgen 1,3-dipolar cycloaddition, e.g., Cu(I)-catalyzed azide-alkyne cycloaddition (CuAAC), thiol-ene reaction, Diels-Alder reaction, inverse electron demand Diels-Alder reaction, [4+1]cycloadditions between isonitriles (isocyanides) and tetrazines, nucleophilic substitution, e.g., to small strained rings (e.g., epoxy and aziridines), carbonyl-chemistry-like formation of ureas, carbon-carbon double bonds addition reactions (e.g., dihydroxylation or alkynes in the thiol-yne reaction), sulfur (VI) fluoride exchange. Strain-promoted azide-alkyne cycloaddition (SPAAC), Strain-promoted alkyne-nitrone cycloaddition (SPANC), and reaction oof trans-cycloalkenes (usually cyclooctenes) and other strained alkenes, e.g., oxanorbornadiene, with azides, tetrazines and tetrazoles.

In one example embodiment, the bond is chosen to be energetically favorable, preferably increasing binding affinity. The exit vector may be adjusted depending on the linker utilized in the molecules. In one example embodiment, the exit vector is a chemical moiety or bond that facilitates stereochemical protrusion that may further facilitate subsequent coupling, bonding and/or accessibility.

### Linker Structures

In an example embodiment, a chimeric small molecule can comprise one or more linkers of any one of the chimeric small molecules of FIGS. 1-50. In an embodiment, L comprises (or is) —[CH₂]ₙ—, —[CH₂CH₂O]ₙ—, —NH—CH₂—[CH₂OCH₂]ₙ—CH₂—, —NH—CH₂CH₂—C(═O)—NH—CH₂—[CH₂OCH₂]ₙ—CH₂, —NH—CH₂—[CH₂OCH₂]ₙ—CH₂—NH—CH₂—[CH₂OCH₂]ₙ—CH₂, —OC(═O)—N(Me)-CH₂—[CH₂OCH₂]ₙ—CH₂—, where n=1-10, an analog or derivative thereof, or any combination thereof.

In an embodiment, L comprises (or is) a rigid linker, the structure of which may comprise (or be):

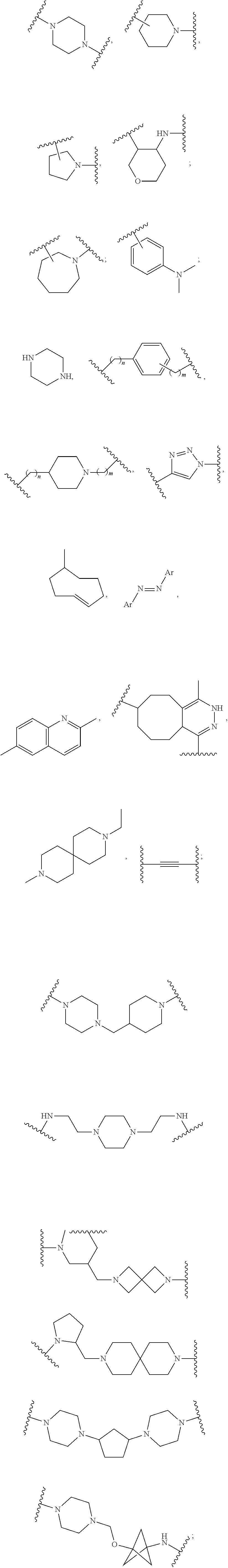

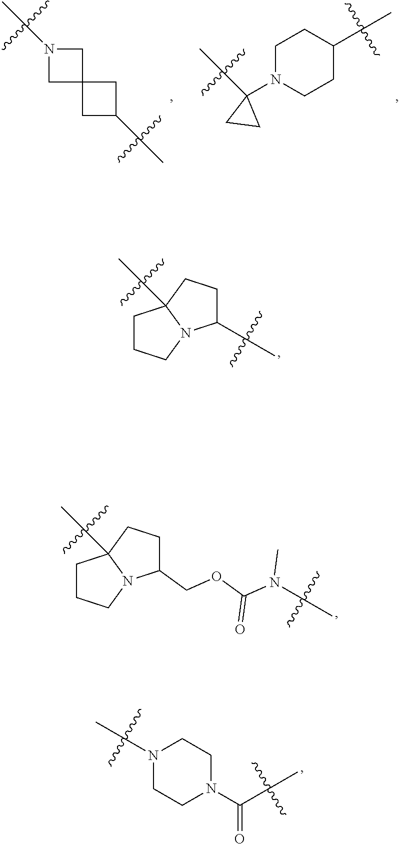 [SMILES: *C1CC2(C1)CN(*)C2 ; *C1CCN(C2(*)CC2)CC1 ; *C1CCC2(*)CCCN12 ; *N(C)C(=O)OCC1CCC2(*)CCCN12 ; *C(=O)N1CCN(*)CC1]

an analog or derivative thereof; or any combination thereof, and wherein any atom in within a ring may substituted for C, N O, S; the linkers may bond to one or more PEG molecules before bonding to A and optionally B; and m and n may be independently selected from 0 to 6. In one example embodiment, a rigid linker L is attached to each of a protein binding moiety and an electrophilic reactive group. In certain example embodiments, the linker L comprises (or is).

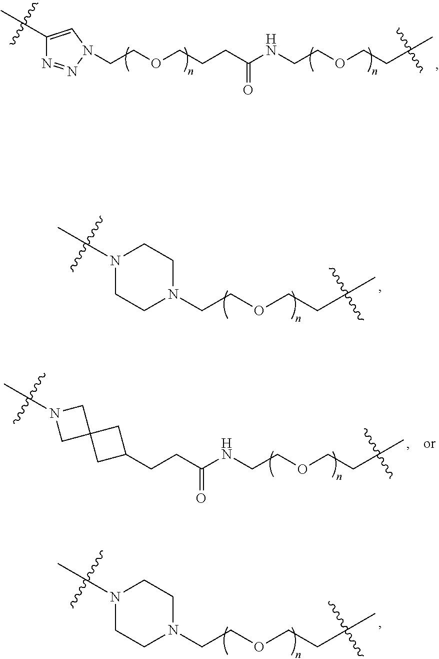

wherein n=1-10, e.g., n=1 or 2, an analog or derivative thereof, or any combination thereof.

In preferred example embodiments, the linker L has one covalent attachment point to a protein binding moiety and two covalent attachment points to an immunogenic moiety. A covalent attachment point may be any single, double, triple, or quadruple bond between one component of the chimeric small molecule and another. In preferred example embodiments, the linker is attached to a protein binding moiety, i.e. A, and an immunogenic display moiety, i.e., B, according to the formula

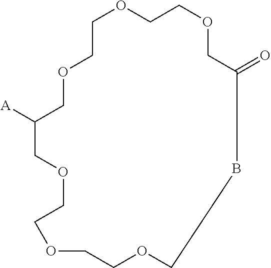

In one example embodiment, the PEG compounds in the previously mentioned linker can be substituted for any linker mentioned herein. In one example embodiment, the previously mentioned linker is optimized for physiochemical properties, such as solubility and/or permeability, and/or pharmacokinetic properties, such as microsomal stability or target binding.

In one example embodiment, the protein binding moiety or the immunogenic display moiety has an adapter or reactive handle, both used herein interchangeably. The reactive handle comprises the group on the protein binding moiety or the immunogenic display moiety that attaches to the linker. In one example embodiment, the reactive handle can perform click chemistry, amide coupling chemistry, crosslinking chemistry, alkylation, or sulfonation chemistry.

Additional Electrophilic Group and/or Linker Groups

In one example embodiment, an exemplary electrophilic group and/or an exemplary linker group is comprised in a structure shown below:

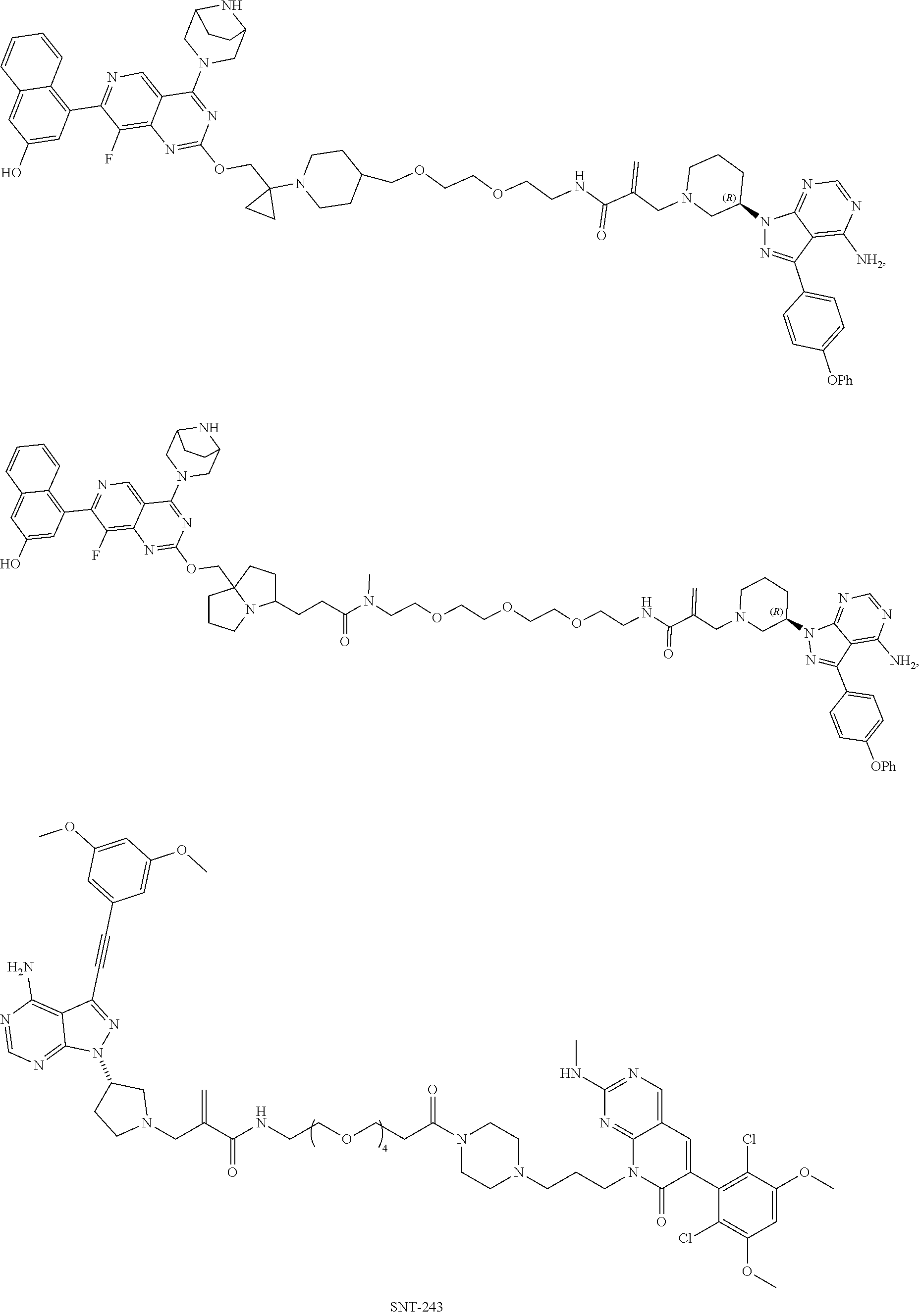

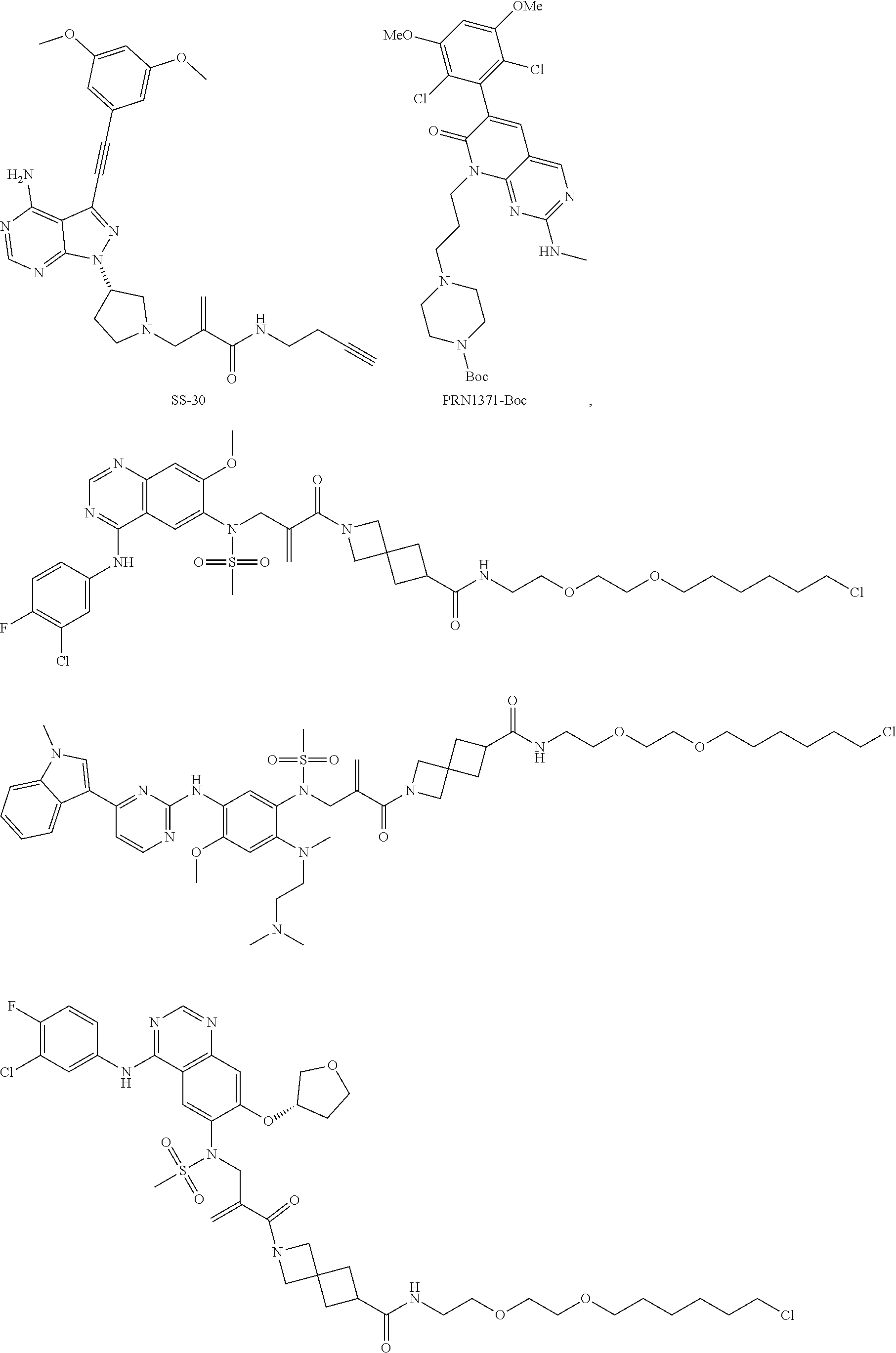 [SMILES: C#CCCNC(=O)C(=C)CN1CC[C@H](n2nc(C#Cc3cc(OC)cc(OC)c3)c3c(N)ncnc32)C1 ; CNc1ncc2cc(-c3c(Cl)c(OC)cc(OC)c3Cl)c(=O)n(CCCN3CCN(C(=O)OC(C)(C)C)CC3)c2n1 ; "SS-30" ; C=C(CN(c1cc2c(Nc3ccc(F)c(Cl)c3)ncnc2cc1OC)S(C)(=O)=O)C(=O)N1CC2(CC(C(=O)NCCOCCOCCCCCCCl)C2)C1 ; C=C(CN(c1cc(Nc2nccc(-c3cn(C)c4ccccc34)n2)c(OC)cc1N(C)CCN(C)C)S(C)(=O)=O)C(=O)N1CC2(CC(C(=O)NCCOCCOCCCCCCCl)C2)C1 ; C=C(CN(c1cc2c(Nc3ccc(F)c(Cl)c3)ncnc2cc1O[C@H]1CCOC1)S(C)(=O)=O)C(=O)N1CC2(CC(C(=O)NCCOCCOCCCCCCCl)C2)C1]

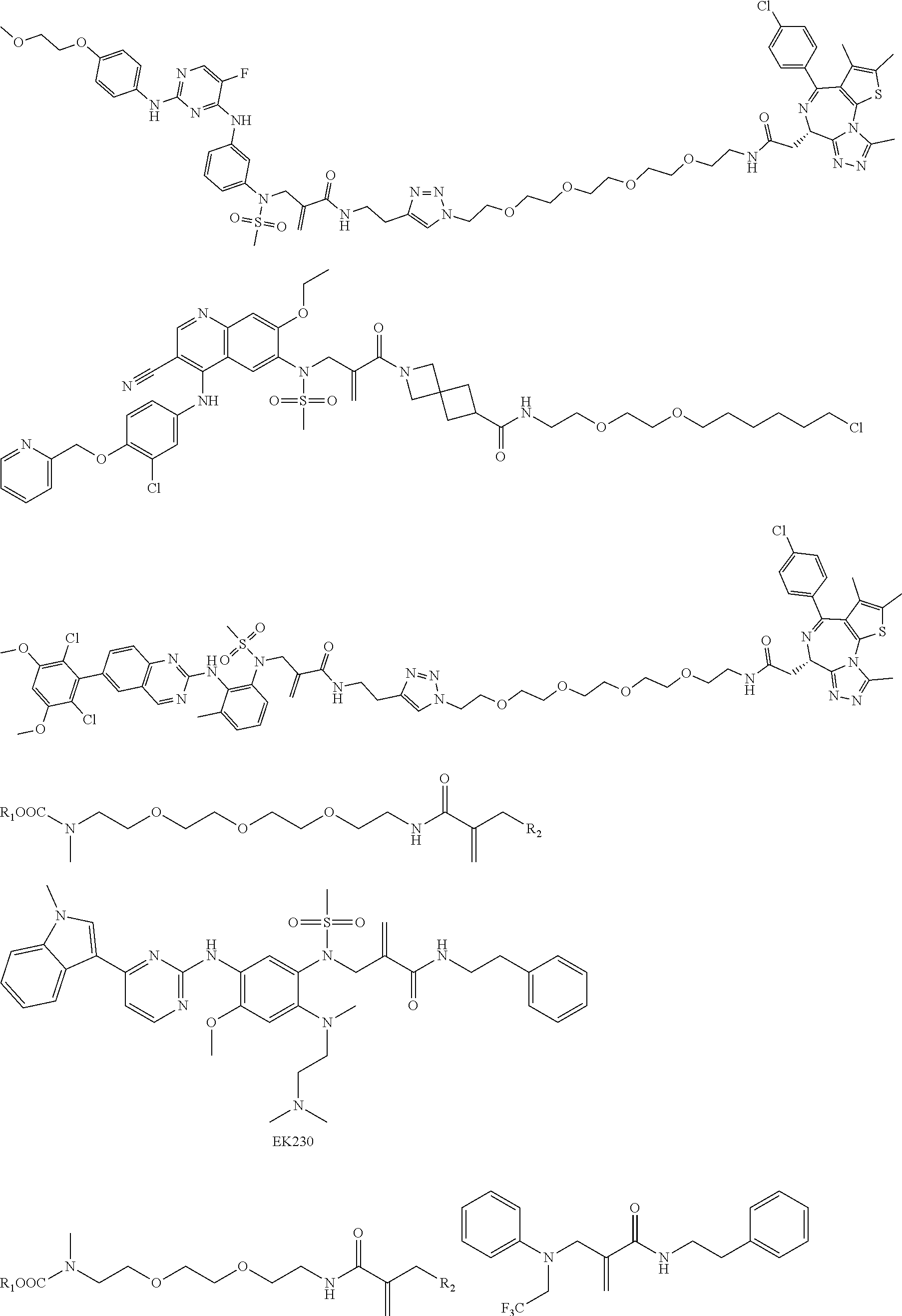

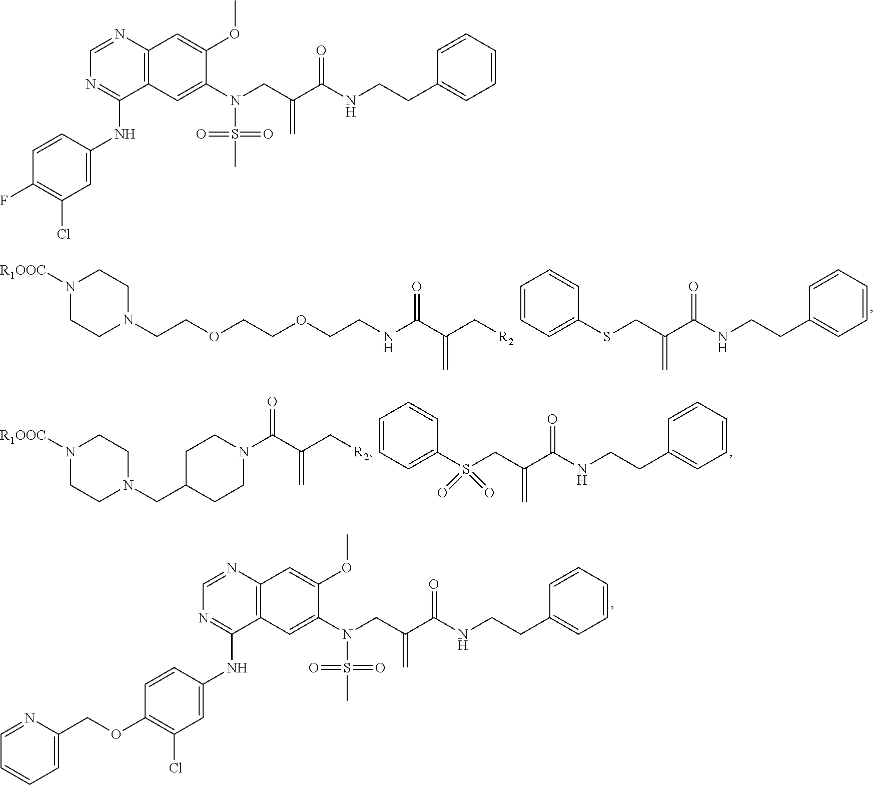

or an analog or derivative thereof.

### Direct Targeting of Immune Cells by Immunogenic Display Moieties

In one embodiment, the immunogenic display moiety may be configured such that it is directly recognized by an immune cell receptor. In one example embodiment, the MHC system of a cell is induced to present peptides carrying said immunogenic display moiety for direct targeting by an immune cell as disclosed herein, such as an engineered T cell having a receptor corresponding to the immunogenic display moiety. In one example embodiment, the immunogenic display moiety is an antigen moiety designed to target an engineered immune cell comprising a receptor for said antigen moiety. In one example embodiment, the engineered immune cell is a CAR-T cell as disclosed herein.

### Indirect Targeting of Immune Cells by Immunogenic Display Moieties

In another embodiment, the immunogenic display moiety is a binding partner of a binding pair, where the cognate binding partner is located on an immune cell engager molecule, discussed in further detail below. In one embodiment, the immunogenic display moiety may be a small molecule that is bound by an antibody or antibody fragment, or a peptide, or capable of engaging in a click chemistry reaction with another molecule. In another embodiment, the immunogenic display moiety may be a peptide that is bound by an antibody or antibody fragment, a small molecule, or another peptide. In another embodiment, the immunogenic display moiety is a small molecule capable of engaging in a click chemistry reaction with another molecule.

### Small Molecule Based Immunogenic Display Moieties

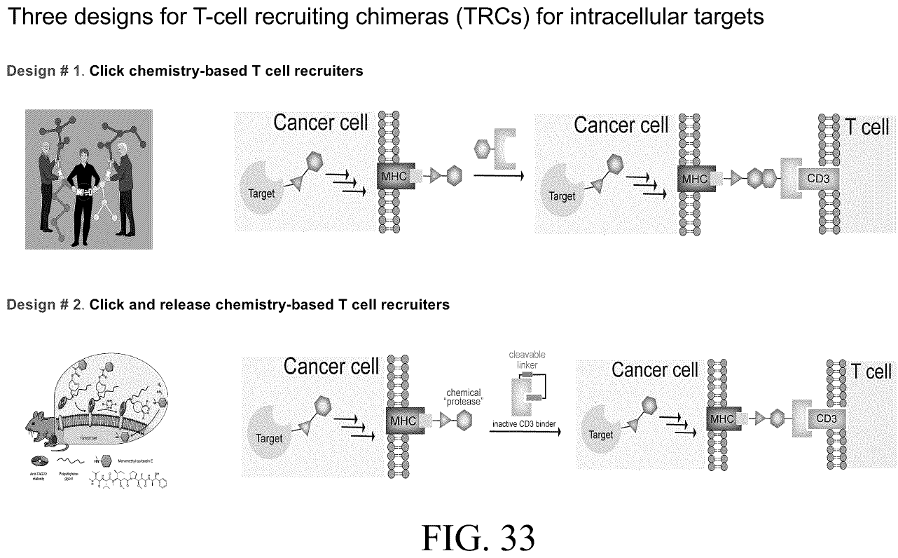

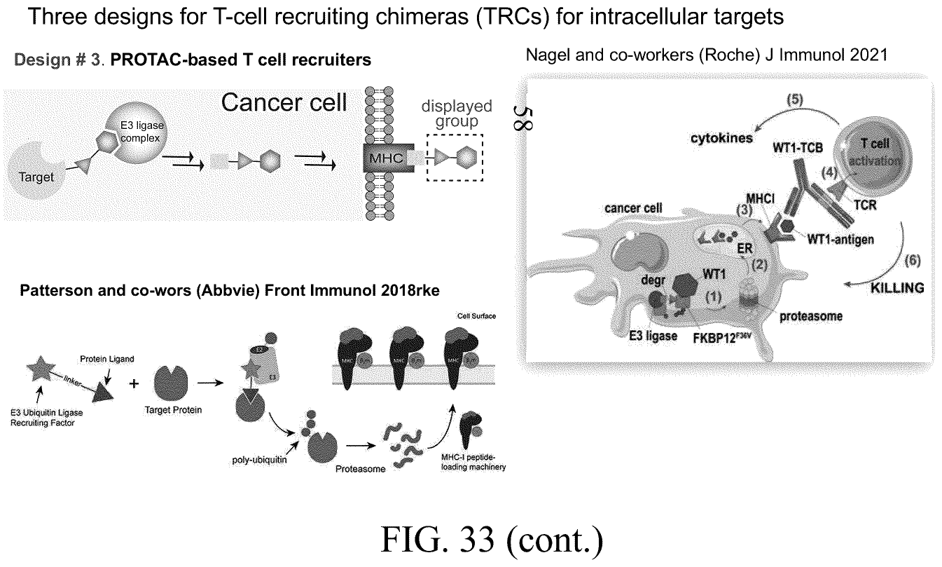

In one example embodiment, an immunogenic display moiety is a small molecule antigen capable of binding to a corresponding antibody or antibody fragment or peptide. In one example embodiment, the antibody or antibody fragment or peptide is a moiety of an immune cell receptor. In one example embodiment, the antibody or antibody fragment or peptide is a moiety of an immune cell engager, e.g., a T-cell engager, further comprising an immune cell binding moiety, e.g., a T-cell binding moiety. In one example embodiment, the small molecule antigen is a 2.4-dinitrophenyl (DNP) motif (see, e.g., FIG. 3). In one example embodiment, the small molecule antigen is any target protein binding moiety as disclosed herein, e.g., FKBP12^{F36V} (see, e.g., FIG. 3), where the corresponding target protein, e.g., FKBP12^{F36V}, is a moiety of an immune cell or an immune cell engager disclosed herein. In one example embodiment, the small molecule is a chemical “protease” which is capable of binding to an immune cell engager, e.g., a T-cell engager, further comprising an inactive immune cell binding moiety, e.g., an inactive T-cell binding moiety, and where the chemical “protease” can cleave a cleavable linker to activate the immune cell binding moiety (see, e.g., FIG. 33).

### Click Chemistry

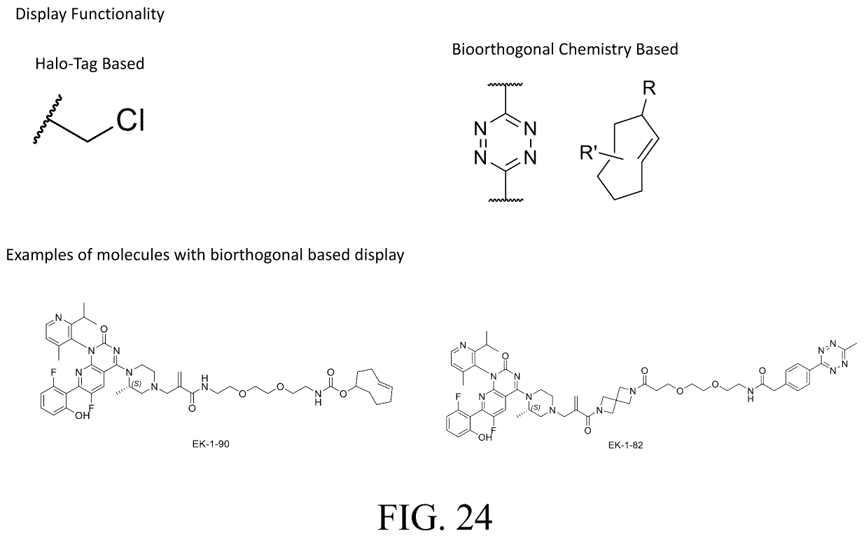

In one example embodiment, the small molecule immunogenic display moiety is a click chemistry ligand, e.g., HaloTag ligand, e.g., a chloroalkane ligand, capable of connecting via a click chemistry reaction to a click chemistry receptor, e.g., a HaloTag protein, of an immune cell engager, e.g., a T-cell engager, further comprising an immune cell binding moiety, e.g., a T-cell binding moiety. In one example embodiment, the click chemistry ligand is a tetrazine ligand (or a TCO ligand) capable of connecting via a click chemistry reaction to a transcyclooctene (TCO) receptor (or a tetrazine receptor) (see, e.g., FIG. 24).

In one example embodiment, a click chemistry reaction is a click chemistry reaction disclosed by New et al, incorporated by reference herein (See e.g., Nwe, K.; Brechbiel, M. W. Growing Applications of “Click Chemistry” for Bioconjugation in Contemporary Biomedical Research. Cancer Biotherapy and Radiopharmaceuticals, 2009, 24, 289-302.). Examples of click chemistry reactions include, but are not limited to: [3+2]cycloadditions, e.g., Huisgen 1,3-dipolar cycloaddition, e.g., Cu(I)-catalyzed azide-alkyne cycloaddition (CuAAC), thiol-ene reaction, Diels-Alder reaction, inverse electron demand Diels-Alder reaction, [4+1]cycloadditions between isonitriles (isocyanides) and tetrazines, nucleophilic substitution, e.g., to small strained rings (e.g., epoxy and aziridines), carbonyl-chemistry-like formation of ureas, carbon-carbon double bonds addition reactions (e.g., dihydroxylation or alkynes in the thiol-yne reaction), sulfur (VI) fluoride exchange. Strain-promoted azide-alkyne cycloaddition (SPAAC), Strain-promoted alkyne-nitrone cycloaddition (SPANC), and reaction oof trans-cycloalkenes (usually cyclooctenes) and other strained alkenes, e.g., oxanorbornadiene, with azides, tetrazines and tetrazoles.

### Peptide Based Immunogenic Display Moieties

In one example embodiment, an immunogenic display moiety is a peptide antigen capable of binding to a corresponding antibody or antibody fragment. In one example embodiment, the antibody or antibody fragment is a moiety of an immune cell receptor. In one example embodiment, the antibody or antibody fragment is a moiety of an immune cell engager, e.g., a T-cell engager, further comprising an immune cell binding moiety, e.g., a T-cell binding moiety. In one example embodiment, In one example embodiment, the peptide based immunogenic display moiety is a click chemistry receptor, e.g., a HaloTag protein, capable of connecting to an immune cell engager, e.g., a T-cell engager, further comprising an immune cell binding moiety, e.g., a T-cell binding moiety, via a click chemistry reaction with a click chemistry ligand, e.g., a HaloTag ligand, e.g., a chloroalkane ligand, of the immune cell engager, e.g., the T-cell engager.

Immunogenic Display Moieties for Use with Bifunctional Immune Cell Engager

In one embodiment, the immunogenic display moiety may be selected for use with a second bifunctional immune cell engager molecule. The immune cell engager comprises a first binding moiety that can bind to the immunogenic display moiety and a second binding moiety that binds to a cell surface receptor of the immune cell to be activated (immune cell binding moiety). The use of an immune cell engager molecule further enhances the modularity of the compositions and methods described herein because a single immune cell engage can be used with multiple different immune cell recruiting chimera molecules each having a different target binding moiety but the same immunogenic display moiety recognized by a single immune cell engager molecule design. The use of immune cell engagers also overcomes the limitations of MHC haplotype variability within a patient population noted above.

The immunogenic display binding moiety may be any molecule capable of binding the immunogenic display moiety or may be an antibody, scFV fragment, or nanobody directed against the immunogenic display moiety and connected to the immune cell binding moiety using a linker.

### Example Immunogenic Display Moiety Structures

Chimeric molecules may be assembled using any combination of the above protein binding moieties, linkers, electrophilic activation groups, and immunogenic moieties. The following description provides, by way of reference only, certain chimeric small molecules that can be generated according to the design principles and examples moieties provided above. In an example embodiment, the chimeric small molecule has the formula of any one of the molecules of FIGS. 1-50.

In one example embodiment, the chimeric small molecule comprises a single immunogenic display moiety. In one example embodiment, the chimeric small molecule comprises two or more same or different immunogenic moieties (e.g., via attachment to a multifunctional linker).

In one example embodiment, the antigen display moiety is a HaloTAG group (e.g., moiety comprises (or is) —(CH₂)₃₋₆—Cl, e.g., —O(CH₂)₆—Cl), a dinitrophenyl (e.g., 2,4-dinitrophenyl) group, or a FKBP12^{F36V} protein binding moiety.

In one example embodiment, the antigen moiety is a phospho-antigen moiety or any precursor thereof. In one example embodiment, the phospho-antigen moiety or precursor thereof is selected from

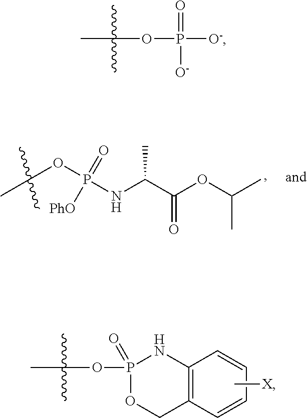

where X is hydrogen or a halogen (e.g., a fluorine).

In one embodiment, the immune cell engager molecule may be a bispecific T-cell engager (BiTE), capable of binding the immunogenic display moiety and a receptor on the surface of the immune cell.

BiTEs that bind to CD3 on T cells are known and the CD3 binding portion thereof may be used as a CD3 binding portion of the immune cell engages disclosed herein. Suurs et al. J Nucl Med. 2020 61(11):1594-1601; Goebler and Bargou, “Blinatumomab: a CD19/CD3 bispecific T cell engager (BiTE) with unique anti-tumor efficacy” Leuk Lymphoma 2016, 57(5):1021-31. In addition to CD8+ cytotoxic T cells, BiTES that bind CD4+ T helper cells and T regulatory cells are also known and may be used as the immune cell binding moiety of the immune cell engager molecule of the present invention. Suryadevara et al. “Are BiTES the missing link in cancer therapy?” Oncoimmunology 2015; 4(6): e1008339. Other configurations such as simultaneous multiple interaction T cell engagers (SMITEs), trispecific killer engagers (TriKEs), and BiTE-expressing chimeric antigen receptor (CAR) T cells (CART.BiTEs) may also be used as the immune cell binding moiety of the immune cell engagers disclosed here. Goebeler and Bargou “T cell-engaging therapies—BiTEs and beyond” Nature Rev Clin Oncol 2020 17: 418-434.

### FKBP12F^{36V}

In one example embodiment, the immunogenic display moiety is an FK506-binding protein (FKBP) binding moiety. The FKBP may be FKBP12, which binds to intracellular calcium release channels and TGF-b type I receptor. In one example embodiment, the FKBP protein binding moiety is an FKBP12^{F36V} protein binding moiety. In another example embodiment, the FKBP protein binding moiety is selected from

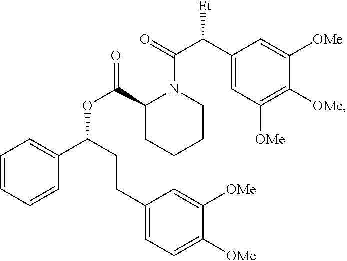 [SMILES: CC[C@@H](C(=O)N1CCCC[C@H]1C(=O)O[C@H](CCc1ccc(OC)c(OC)c1)c1ccccc1)c1cc(OC)c(OC)c(OC)c1]

or an analog or derivative thereof.

Tyrosine phosphorylation on FGFR1 can trigger signaling cascade to induce PI3K/AKT/mTOR signaling and increased transcription of G-CSF, a blood growth factor. See, e.g., Turner et al, Nature Reviews Cancer 2010. In one example embodiment, the molecule is capable of activating FGFR1/mTOR/G-CSF signaling in a dose-dependent manner.

### Proteolysis-Targeting Chimeras (PROTACS)

Proteolysis Targeting Chimeras (PROTACs), a class of heterobifunctional molecules composed of a protein of interest ligand connected to an E3 ligase ligand. PROTACs, which recruit target proteins to E3 ligases for ubiquitination and proteosome degradation, are emerging as a novel therapeutic modality for targeted protein degradation. Typical E3 ligase ligands include Immunomodulatory Drugs (IMiDs), including, but not limited to, Thalidomide, Pomalidomide, Lenalidomide, and analogs thereof, which induce proximity between cereblon (CRBN), a component of E3 ubiquitin ligase, and proteins with Zinc-finger (ZF) motifs. A diverse number of IMiD analogs can be prepared with various linkers (exit vectors) and structurally and/or stereochemically diverse modifications for use in PROTACs.

In an embodiment, an immunogenic display moiety of the present disclosure is also an E3 ligase ligand, e.g., IMiD, capable of recruiting an E3 ligase such as CRBN to the target protein for ubiquitination and degradation of the target protein. In various example embodiments, a chimeric small molecule of the present disclosure both labels a target protein with the immunogenic display moiety and brings the target protein into proximity to an E3 ligase such as CRBN, for degradation, such that the immunogenic display moiety is presented at a cell surface by an MHC system. In certain example embodiments, the MHC presentation of the immunogenic display moiety at the cell surface is enhanced by the ability of the immunogenic display moiety to recruit an E3 ligase to the target protein, as compared to the use of an immunogenic display moiety which is not capable of E3 ligase recruitment. See Massafra et al. Proteolysis-Targeting Chimeras Enhance T Cell Bispecific Antibody-Driven T Cell Activation and Effector Function through Increased MHC Class I Antigen Presentation in Cancer Cells (2021). J Immunol; 207 (2): 493-504. Doi.org/10.4049/jimmunol.2000252 (stating that treating human breast cancer cells with a PROTAC targeting mutant-selective FKBP12^{F36V} increased the MHC presentation of WT1 antigens). In certain example embodiments, the E3 ligase ligand is selected from any PROTAC in the PROTAC-DB 2.0 database, academic.oup.com/nar/article/51/D1/D1367/6775390, or in any other publicly available PROTAC database. In certain example embodiments, the E3 ligase ligand is selected from any E3 ligase ligand disclosed in International Patent Publication WO2023/081400A1 to Choudhary. In one example embodiment, an immune cell engager, e.g., a T-cell engager, comprises a moiety capable of binding to an E3 ligase ligand of an immunogenic display moiety. In one example embodiment, a moiety capable of binding to an E3 ligase ligand is an E3 ligase ligand binding moiety of a CRBN protein, or an antibody or antibody fragment to an E3 ligase ligand. In one example embodiments, any one of the chimeric small molecules disclosed herein comprises an immunogenic display moiety comprising an E3 ligase ligand and/or is prepared by a method as disclosed herein.

### Bifunctional Immune Cell Engager Molecules

Bifunctional immune cell engager molecules comprise a moiety that functions as the cognate binding partner of the immunogenic display moiety of the chimeric small molecule and a second binding moiety (e.g. an immune cell binding moiety). The cognate binding partner of the immunogenic display moiety and the immune cell binding moiety may be fused or linked together. In one example embodiment, the immune cell binding moiety is an antibody, a nanobody, an antigen binding fragment, a BiTE, or the like, to a receptor of the immune cell, e.g., an anti-CD3 scFV.

In certain example embodiments, activation of an immune cell (e.g., a T-cell) with a bifunctional immune cell engager molecule is prevented by protecting the bifunctional cell binding moiety with a masking agent (e.g., a cleavable linker). Only upon reaction of the bifunctional compound with a chimeric small molecule at a surface of a cell will the masking agent be released, activating the immune cell binding moiety and allowing recruitment of an immune cell to the surface of the cell displaying the immunogenic display moiety.

In certain embodiments, click-chemistry occurs between the display functionality attached to the protein of interest and a masking small molecule attached to the second binding moiety. A click-chemistry reaction between the immunogenic display moiety of the chimeric small molecule and, for example, the first binding moiety of the bifunctional immune cell engager will lead to a click chemistry reaction that unmask the second binding moiety (i.e. the immune cell binding moiety) allowing it to recruit an immune cell. In certain embodiments, the click chemistry will result in a molecule that unmasks an anti-CD3. In one embodiment, t, the bifunctional immune cell engager is designed to recognize the new molecule that results from the click-chemistry reaction between the immunogenic display functionality and the masking molecule. In certain embodiments, the immunogenic display functionality acts as a “chemical protease” for a cleavable linker attached to the bifunctional immune cell engager, and upon cleavage of the linker, the bifunctional immune cell engager is capable of activating the immune cell binding group and allowing recruiting of an immune cell.

### Antibodies

In one embodiment, the first binding moiety, the second binding moiety, or both may comprise an antibody or an antigen binding fragment thereof. The term “antibody” is used interchangeably with the term “immunoglobulin” herein, and includes intact antibodies, fragments of antibodies, e.g., Fab, F(ab′)2 fragments, and intact antibodies and fragments that have been mutated either in their constant and/or variable region (e.g., mutations to produce chimeric, partially humanized, or fully humanized antibodies, as well as to produce antibodies with a desired trait, e.g., enhanced binding and/or reduced FcR binding). The term “fragment” refers to a part or portion of an antibody or antibody chain comprising fewer amino acid residues than an intact or complete antibody or antibody chain. Fragments can be obtained via chemical or enzymatic treatment of an intact or complete antibody or antibody chain. Fragments can also be obtained by recombinant means. Exemplary fragments include Fab, Fab′, F(ab′)2, Fabc, Fd, dAb, V_{HH} and scFv and/or Fv fragments.

As used herein, a preparation of antibody protein having less than about 50% of non-antibody protein (also referred to herein as a “contaminating protein”), or of chemical precursors, is considered to be “substantially free.” 40%, 30%, 20%, 10% and more preferably 5% (by dry weight), of non-antibody protein, or of chemical precursors is considered to be substantially free. When the antibody protein or biologically active portion thereof is recombinantly produced, it is also preferably substantially free of culture medium, i.e., culture medium represents less than about 30%, preferably less than about 20%, more preferably less than about 10%, and most preferably less than about 5% of the volume or mass of the protein preparation.

The term “antigen-binding fragment” refers to a polypeptide fragment of an immunoglobulin or antibody that binds antigen or competes with intact antibody (i.e., with the intact antibody from which they were derived) for antigen binding (i.e., specific binding). As such these antibodies or fragments thereof are included in the scope of the invention, provided that the antibody or fragment binds specifically to a target molecule.

It is intended that the term “antibody” encompass any Ig class or any Ig subclass (e.g. the IgG1, IgG2, IgG3, and IgG4 subclasses of IgG) obtained from any source (e.g., humans and non-human primates, and in rodents, lagomorphs, caprines, bovines, equines, ovines, etc.).

The term “Ig class” or “immunoglobulin class”, as used herein, refers to the five classes of immunoglobulin that have been identified in humans and higher mammals, IgG, IgM, IgA, IgD, and IgE. The term “Ig subclass” refers to the two subclasses of IgM (H and L), three subclasses of IgA (IgA1, IgA2, and secretory IgA), and four subclasses of IgG (IgG1, IgG2, IgG3, and IgG4) that have been identified in humans and higher mammals. The antibodies can exist in monomeric or polymeric form; for example, 1 gM antibodies exist in pentameric form, and IgA antibodies exist in monomeric, dimeric, or multimeric form.

The term “IgG subclass” refers to the four subclasses of immunoglobulin class IgG-IgG1, IgG2, IgG3, and IgG4 that have been identified in humans and higher mammals by the heavy chains of the immunoglobulins, V1-γ4, respectively. The term “single-chain immunoglobulin” or “single-chain antibody” (used interchangeably herein) refers to a protein having a two-polypeptide chain structure consisting of a heavy and a light chain, said chains being stabilized, for example, by interchain peptide linkers, which has the ability to specifically bind antigen. The term “domain” refers to a globular region of a heavy or light chain polypeptide comprising peptide loops (e.g., comprising 3 to 4 peptide loops) stabilized, for example, by β pleated sheet and/or intrachain disulfide bond. Domains are further referred to herein as “constant” or “variable”, based on the relative lack of sequence variation within the domains of various class members in the case of a “constant” domain, or the significant variation within the domains of various class members in the case of a “variable” domain. Antibody or polypeptide “domains” are often referred to interchangeably in the art as antibody or polypeptide “regions”. The “constant” domains of an antibody light chain are referred to interchangeably as “light chain constant regions”, “light chain constant domains”, “CL” regions or “CL” domains. The “constant” domains of an antibody heavy chain are referred to interchangeably as “heavy chain constant regions”, “heavy chain constant domains”, “CH” regions or “CH” domains). The “variable” domains of an antibody light chain are referred to interchangeably as “light chain variable regions”, “light chain variable domains”, “VL” regions or “VL” domains). The “variable” domains of an antibody heavy chain are referred to interchangeably as “heavy chain constant regions”, “heavy chain constant domains”, “VH” regions or “VH” domains).

The term “region” can also refer to a part or portion of an antibody chain or antibody chain domain (e.g., a part or portion of a heavy or light chain or a part or portion of a constant or variable domain, as defined herein), as well as more discrete parts or portions of said chains or domains. For example, light and heavy chains or light and heavy chain variable domains include “complementarity determining regions” or “CDRs” interspersed among “framework regions” or “FRs”, as defined herein.

The term “conformation” refers to the tertiary structure of a protein or polypeptide (e.g., an antibody, antibody chain, domain, or region thereof). For example, the phrase “light (or heavy) chain conformation” refers to the tertiary structure of a light (or heavy) chain variable region, and the phrase “antibody conformation” or “antibody fragment conformation” refers to the tertiary structure of an antibody or fragment thereof.

The term “antibody-like protein scaffolds” or “engineered protein scaffolds” broadly encompasses proteinaceous non-immunoglobulin specific-binding agents, typically obtained by combinatorial engineering (such as site-directed random mutagenesis in combination with phage display or other molecular selection techniques). Usually, such scaffolds are derived from robust and small soluble monomeric proteins (such as Kunitz inhibitors or lipocalins) or from a stably folded extra-membrane domain of a cell surface receptor (such as protein A, fibronectin or the ankyrin repeat).

Such scaffolds have been extensively reviewed in Binz et al. (Engineering novel binding proteins from nonimmunoglobulin domains. Nat Biotechnol 2005, 23:1257-1268), Gebauer and Skerra (Engineered protein scaffolds as next-generation antibody therapeutics. Curr Opin Chem Biol. 2009, 13:245-55), Gill and Damle (Biopharmaceutical drug discovery using novel protein scaffolds. Curr Opin Biotechnol 2006, 17:653-658), Skerra (Engineered protein scaffolds for molecular recognition. J Mol Recognit 2000, 13:167-187), and Skerra (Alternative non-antibody scaffolds for molecular recognition. Curr Opin Biotechnol 2007, 18:295-304), and include without limitation affibodies, based on the Z-domain of staphylococcal protein A, a three-helix bundle of 58 residues providing an interface on two of its alpha-helices (Nygren, Alternative binding proteins: Affibody binding proteins developed from a small three-helix bundle scaffold. FEBS J 2008, 275:2668-2676); engineered Kunitz domains based on a small (ca. 58 residues) and robust, disulphide-crosslinked serine protease inhibitor, typically of human origin (e.g. LACI-D1), which can be engineered for different protease specificities (Nixon and Wood, Engineered protein inhibitors of proteases. Curr Opin Drug Discov Dev 2006, 9:261-268); monobodies or adnectins based on the 10th extracellular domain of human fibronectin III (10Fn3), which adopts an Ig-like beta-sandwich fold (94 residues) with 2-3 exposed loops, but lacks the central disulphide bridge (Koide and Koide, Monobodies: antibody mimics based on the scaffold of the fibronectin type III domain. Methods Mol Biol 2007, 352:95-109); anticalins derived from the lipocalins, a diverse family of eight-stranded beta-barrel proteins (ca. 180 residues) that naturally form binding sites for small ligands by means of four structurally variable loops at the open end, which are abundant in humans, insects, and many other organisms (Skerra, Alternative binding proteins: Anticalins harnessing the structural plasticity of the lipocalin ligand pocket to engineer novel binding activities. FEBS J 2008, 275:2677-2683); DARPins, designed ankyrin repeat domains (166 residues), which provide a rigid interface arising from typically three repeated beta-turns (Stumpp et al., DARPins: a new generation of protein therapeutics. Drug Discov Today 2008, 13:695-701); avimers (multimerized LDLR-A module) (Silverman et al., Multivalent avimer proteins evolved by exon shuffling of a family of human receptor domains. Nat Biotechnol 2005, 23:1556-1561); and cysteine-rich knottin peptides (Kolmar, Alternative binding proteins: biological activity and therapeutic potential of cystine-knot miniproteins. FEBS J 2008, 275:2684-2690).

“Specific binding” of an antibody means that the antibody exhibits appreciable affinity for a particular antigen or epitope and, generally, does not exhibit significant cross reactivity. “Appreciable” binding includes binding with an affinity of at least 25 M. Antibodies with affinities greater than 1×107 M−1 (or a dissociation coefficient of 1 M or less or a dissociation coefficient of 1 nm or less) typically bind with correspondingly greater specificity. Values intermediate of those set forth herein are also intended to be within the scope of the present invention and antibodies of the invention bind with a range of affinities, for example, 100 nM or less, 75 nM or less, 50 nM or less, 25 nM or less, for example 10 nM or less, 5 nM or less, 1 nM or less, or in embodiments 500 pM or less, 100 pM or less, 50 pM or less or 25 pM or less. An antibody that “does not exhibit significant crossreactivity” is one that will not appreciably bind to an entity other than its target (e.g., a different epitope or a different molecule). For example, an antibody that specifically binds to a target molecule will appreciably bind the target molecule but will not significantly react with non-target molecules or peptides. An antibody specific for a particular epitope will, for example, not significantly crossreact with remote epitopes on the same protein or peptide. Specific binding can be determined according to any art-recognized means for determining such binding. Preferably, specific binding is determined according to Scatchard analysis and/or competitive binding assays.

As used herein, the term “affinity” refers to the strength of the binding of a single antigen-combining site with an antigenic determinant. Affinity depends on the closeness of stereochemical fit between antibody combining sites and antigen determinants, on the size of the area of contact between them, on the distribution of charged and hydrophobic groups, etc. Antibody affinity can be measured by equilibrium dialysis or by the kinetic BIACORE™ method. The dissociation constant, Kd, and the association constant, Ka, are quantitative measures of affinity.

As used herein, the term “monoclonal antibody” refers to an antibody derived from a clonal population of antibody-producing cells (e.g., B lymphocytes or B cells) which is homogeneous in structure and antigen specificity. The term “polyclonal antibody” refers to a plurality of antibodies originating from different clonal populations of antibody-producing cells which are heterogeneous in their structure and epitope specificity but which recognize a common antigen. Monoclonal and polyclonal antibodies may exist within bodily fluids, as crude preparations, or may be purified, as described herein.

The term “binding portion” of an antibody (or “antibody portion”) includes one or more complete domains, e.g., a pair of complete domains, as well as fragments of an antibody that retain the ability to specifically bind to a target molecule. It has been shown that the binding function of an antibody can be performed by fragments of a full-length antibody. Binding fragments are produced by recombinant DNA techniques, or by enzymatic or chemical cleavage of intact immunoglobulins. Binding fragments include Fab, Fab′, F(ab′)2, Fabc, Fd, dAb, Fv, single chains, single-chain antibodies, e.g., scFv, and single domain antibodies.

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, FR residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable regions correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin.

Examples of portions of antibodies or epitope-binding proteins encompassed by the present definition include: (i) the Fab fragment, having VL, CL, VH and CH1 domains; (ii) the Fab′ fragment, which is a Fab fragment having one or more cysteine residues at the C-terminus of the CH1 domain; (iii) the Fd fragment having VH and CH1 domains; (iv) the Fd′ fragment having VH and CH1 domains and one or more cysteine residues at the C-terminus of the CHI domain; (v) the Fv fragment having the VL and VH domains of a single arm of an antibody; (vi) the dAb fragment (Ward et al., 341 Nature 544 (1989)) which consists of a VH domain or a VL domain that binds antigen; (vii) isolated CDR regions or isolated CDR regions presented in a functional framework; (viii) F(ab′)2 fragments which are bivalent fragments including two Fab′ fragments linked by a disulphide bridge at the hinge region; (ix) single chain antibody molecules (e.g., single chain Fv; scFv) (Bird et al., 242 Science 423 (1988); and Huston et al., 85 PNAS 5879 (1988)); (x) “diabodies” with two antigen binding sites, comprising a heavy chain variable domain (VH) connected to a light chain variable domain (VL) in the same polypeptide chain (see, e.g., EP 404,097; WO 93/11161; Hollinger et al., 90 PNAS 6444 (1993)); (xi) “linear antibodies” comprising a pair of tandem Fd segments (VH-Ch1-VH-Ch1) which, together with complementary light chain polypeptides, form a pair of antigen binding regions (Zapata et al., Protein Eng. 8(10):1057-62 (1995); and U.S. Pat. No. 5,641,870).

As used herein, a “blocking” antibody or an antibody “antagonist” is one which inhibits or reduces biological activity of the antigen(s) it binds. In certain embodiments, the blocking antibodies or antagonist antibodies or portions thereof described herein completely inhibit the biological activity of the antigen(s).

Antibodies may act as agonists or antagonists of the recognized polypeptides. For example, the present invention includes antibodies which disrupt receptor/ligand interactions either partially or fully. The invention features both receptor-specific antibodies and ligand-specific antibodies. The invention also features receptor-specific antibodies which do not prevent ligand binding but prevent receptor activation. Receptor activation (i.e., signaling) may be determined by techniques described herein or otherwise known in the art. For example, receptor activation can be determined by detecting the phosphorylation (e.g., tyrosine or serine/threonine) of the receptor or of one of its down-stream substrates by immunoprecipitation followed by western blot analysis. In specific embodiments, antibodies are provided that inhibit ligand activity or receptor activity by at least 95%, at least 90%, at least 85%, at least 80%, at least 75%, at least 70%, at least 60%, or at least 50% of the activity in absence of the antibody.

The invention also features receptor-specific antibodies which both prevent ligand binding and receptor activation as well as antibodies that recognize the receptor-ligand complex. Likewise, encompassed by the invention are neutralizing antibodies which bind the ligand and prevent binding of the ligand to the receptor, as well as antibodies which bind the ligand, thereby preventing receptor activation, but do not prevent the ligand from binding the receptor. Further included in the invention are antibodies which activate the receptor. These antibodies may act as receptor agonists, i.e., potentiate or activate either all or a subset of the biological activities of the ligand-mediated receptor activation, for example, by inducing dimerization of the receptor. The antibodies may be specified as agonists, antagonists or inverse agonists for biological activities comprising the specific biological activities of the peptides disclosed herein. The antibody agonists and antagonists can be made using methods known in the art. See, e.g., PCT publication WO 96/40281; U.S. Pat. No. 5,811,097; Deng et al., Blood 92(6):1981-1988 (1998); Chen et al., Cancer Res. 58(16):3668-3678 (1998); Harrop et al., J. Immunol. 161(4):1786-1794 (1998); Zhu et al., Cancer Res. 58(15):3209-3214 (1998); Yoon et al., J. Immunol. 160(7):3170-3179 (1998); Prat et al., J. Cell. Sci. III (Pt2):237-247 (1998); Pitard et al., J. Immunol. Methods 205(2):177-190 (1997); Liautard et al., Cytokine 9(4):233-241 (1997); Carlson et al., J. Biol. Chem. 272(17):11295-11301 (1997); Taryman et al., Neuron 14(4):755-762 (1995); Muller et al., Structure 6(9):1153-1167 (1998); Bartunek et al., Cytokine 8(1):14-20 (1996).

The antibodies as defined for the present invention include derivatives that are modified, i.e., by the covalent attachment of any type of molecule to the antibody such that covalent attachment does not prevent the antibody from generating an anti-idiotypic response. For example, but not by way of limitation, the antibody derivatives include antibodies that have been modified, e.g., by glycosylation, acetylation, pegylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

Simple binding assays can be used to screen for or detect agents that bind to a target protein, or disrupt the interaction between proteins (e.g., a receptor and a ligand). Because certain targets of the present invention are transmembrane proteins, assays that use the soluble forms of these proteins rather than full-length protein can be used, in some embodiments. Soluble forms include, for example, those lacking the transmembrane domain and/or those comprising the IgV domain or fragments thereof which retain their ability to bind their cognate binding partners. Further, agents that inhibit or enhance protein interactions for use in the compositions and methods described herein, can include recombinant peptido-mimetics.

Detection methods useful in screening assays include antibody-based methods, detection of a reporter moiety, detection of cytokines as described herein, and detection of a gene signature as described herein.

Another variation of assays to determine binding of a receptor protein to a ligand protein is through the use of affinity biosensor methods. Such methods may be based on the piezoelectric effect, electrochemistry, or optical methods, such as ellipsometry, optical wave guidance, and surface plasmon resonance (SPR).

### Aptamers

In one embodiment, the first binding moiety, the second binding moiety, or both may comprise an aptamer. Nucleic acid aptamers are nucleic acid species that have been engineered through repeated rounds of in vitro selection or equivalently, SELEX (systematic evolution of ligands by exponential enrichment) to bind to various molecular targets such as small molecules, proteins, nucleic acids, cells, tissues and organisms. Nucleic acid aptamers have specific binding affinity to molecules through interactions other than classic Watson-Crick base pairing. Aptamers are useful in biotechnological and therapeutic applications as they offer molecular recognition properties similar to antibodies. In addition to their discriminate recognition, aptamers offer advantages over antibodies as they can be engineered completely in a test tube, are readily produced by chemical synthesis, possess desirable storage properties, and elicit little or no immunogenicity in therapeutic applications. In certain embodiments, RNA aptamers may be expressed from a DNA construct. In other embodiments, a nucleic acid aptamer may be linked to another polynucleotide sequence. The polynucleotide sequence may be a double stranded DNA polynucleotide sequence. The aptamer may be covalently linked to one strand of the polynucleotide sequence. The aptamer may be ligated to the polynucleotide sequence. The polynucleotide sequence may be configured, such that the polynucleotide sequence may be linked to a solid support or ligated to another polynucleotide sequence.

Aptamers, like peptides generated by phage display or monoclonal antibodies (“mAbs”), are capable of specifically binding to selected targets and modulating the target's activity, e.g., through binding, aptamers may block their target's ability to function. A typical aptamer is 10-15 kDa in size (30-45 nucleotides), binds its target with sub-nanomolar affinity, and discriminates against closely related targets (e.g., aptamers will typically not bind other proteins from the same gene family). Structural studies have shown that aptamers are capable of using the same types of binding interactions (e.g., hydrogen bonding, electrostatic complementarity, hydrophobic contacts, steric exclusion) that drives affinity and specificity in antibody-antigen complexes.

Aptamers have a number of desirable characteristics for use in research and as therapeutics and diagnostics including high specificity and affinity, biological efficacy, and excellent pharmacokinetic properties. In addition, they offer specific competitive advantages over antibodies and other protein biologics. Aptamers are chemically synthesized and are readily scaled as needed to meet production demand for research, diagnostic or therapeutic applications. Aptamers are chemically robust. They are intrinsically adapted to regain activity following exposure to factors such as heat and denaturants and can be stored for extended periods (>1 yr) at room temperature as lyophilized powders. Not being bound by a theory, aptamers bound to a solid support or beads may be stored for extended periods.

Oligonucleotides in their phosphodiester form may be quickly degraded by intracellular and extracellular enzymes such as endonucleases and exonucleases. Aptamers can include modified nucleotides conferring improved characteristics on the ligand, such as improved in vivo stability or improved delivery characteristics. Examples of such modifications include chemical substitutions at the ribose and/or phosphate and/or base positions. SELEX identified nucleic acid ligands containing modified nucleotides are described, e.g., in U.S. Pat. No. 5,660,985, which describes oligonucleotides containing nucleotide derivatives chemically modified at the 2′ position of ribose, 5 position of pyrimidines, and 8 position of purines, U.S. Pat. No. 5,756,703 which describes oligonucleotides containing various 2′-modified pyrimidines, and U.S. Pat. No. 5,580,737 which describes highly specific nucleic acid ligands containing one or more nucleotides modified with 2′-amino (2′-NH2), 2′-fluoro (2′-F), and/or 2′-O-methyl (2′-OMe) substituents. Modifications of aptamers may also include modifications at exocyclic amines, substitution of 4-thiouridine, substitution of 5-bromo or 5-iodo-uracil; backbone modifications, phosphorothioate or allyl phosphate modifications, methylations, and unusual base-pairing combinations such as the isobases isocytidine and isoguanosine. Modifications can also include 3′ and 5′ modifications such as capping. As used herein, the term phosphorothioate encompasses one or more non-bridging oxygen atoms in a phosphodiester bond replaced by one or more sulfur atoms. In further embodiments, the oligonucleotides comprise modified sugar groups, for example, one or more of the hydroxyl groups is replaced with halogen, aliphatic groups, or functionalized as ethers or amines. In one embodiment, the 2′-position of the furanose residue is substituted by any of an O-methyl, O-alkyl, 0-allyl, S-alkyl, S-allyl, or halo group. Methods of synthesis of 2′-modified sugars are described, e.g., in Sproat, et al., Nucl. Acid Res. 19:733-738 (1991); Cotten, et al, Nucl. Acid Res. 19:2629-2635 (1991); and Hobbs, et al, Biochemistry 12:5138-5145 (1973). Other modifications are known to one of ordinary skill in the art. In certain embodiments, aptamers include aptamers with improved off-rates as described in International Patent Publication No. WO 2009012418, “Method for generating aptamers with improved off-rates,” incorporated herein by reference in its entirety. In certain embodiments aptamers are chosen from a library of aptamers. Such libraries include, but are not limited to, those described in Rohloff et al., “Nucleic Acid Ligands With Protein-like Side Chains: Modified Aptamers and Their Use as Diagnostic and Therapeutic Agents,” Molecular Therapy Nucleic Acids (2014) 3, e201. Aptamers are also commercially available (see, e.g., SomaLogic, Inc., Boulder, Colorado). In certain embodiments, the present invention may utilize any aptamer containing any modification as described herein,

### Antibody Drug Conjugates

In one example embodiment the bifunctional immune cell engager is an antibody drug conguate. The term “antibody-drug-conjugate” or “ADC” refers to a binding protein, such as an antibody or antigen binding fragment thereof, chemically linked to one or more chemical drug(s) (also referred to herein as agent(s)) that may optionally be therapeutic or cytotoxic agents. In a preferred embodiment, an ADC includes an antibody, a small molecule, and a linker that enables attachment or conjugation of the small molecule to the antibody. An ADC typically has anywhere from 1 to 8 drugs conjugated to the antibody, including drug loaded species of 2, 4, 6, or 8. In the context of the present invention the cytotoxic or therapeutic agent may be, or be replaced with, a molecule capable of binding to the immunogenic display moiety of the immune recruiting chimera. Conversely the antibody or antigen binding fragment may bind the immunogenic display moiety and the small molecule portion of the ADC may bind a cell surface receptor of a target immune cell.

In certain embodiments, the ADC specifically binds to a gene product expressed on the cell surface of a tumor cell. By means of an example, an agent, such as an antibody, capable of specifically binding to a gene product expressed on the cell surface of the tumor cells may be conjugated with a therapeutic or effector agent for targeted delivery of the therapeutic or effector agent to the immune cells.

Examples of such therapeutic or effector agents include immunomodulatory classes as discussed herein, such as without limitation a toxin, drug, radionuclide, cytokine, lymphokine, chemokine, growth factor, tumor necrosis factor, hormone, hormone antagonist, enzyme, oligonucleotide, siRNA, RNAi, photoactive therapeutic agent, anti-angiogenic agent and pro-apoptotic agent.

Non-limiting examples of drugs that may be included in the ADCs are mitotic inhibitors (e.g., maytansinoid DM4), antitumor antibiotics, immunomodulating agents, vectors for gene therapy, alkylating agents, antiangiogenic agents, antimetabolites, boron-containing agents, chemoprotective agents, hormones, antihormone agents, corticosteroids, photoactive therapeutic agents, oligonucleotides, radionuclide agents, topoisomerase inhibitors, tyrosine kinase inhibitors, and radiosensitizers.

Example toxins include ricin, abrin, alpha toxin, saporin, ribonuclease (RNase), DNase I, Staphylococcal enterotoxin-A, pokeweed antiviral protein, gelonin, diphtheria toxin, Pseudomonas exotoxin, or Pseudomonas endotoxin.

Example radionuclides include 103mRh, 103Ru, 105Rh, 105Ru, 107Hg, 109Pd, 109Pt, 111Ag, 111In, 113mIn 119Sb, 11C, 121mTe, 122mTe, 125I, 125mTe, 1261, 1311, 1331, 13N, 142Pr, 143Pr, 149Pm, 152Dy, 153Sm, 15O, 161Ho, 161Tb, 165Tm, 166Dy, 166Ho, 167Tm, 168Tm, 169Er, 169Yb, 177Lu, 186Re, 188Re, 189mOs, 189Re, 192Ir, 194Ir, 197Pt, 198Au, 199Au, 201T1, 203Hg, 211At, 211Bi, 211Pb, 212Bi, 212Pb, 213Bi, 215Po, 217At, 219Rn, 221Fr, 223Ra, 224Ac, 225Ac, 225Fm, 32P, 33P, 47Sc, 51Cr, 57Co, 58Co, 59Fe, 62Cu, 67Cu, 67Ga, 75Br, 75Se, 76Br, 77As, 77Br, 80mBr, 89Sr, 90Y, 95Ru, 97Ru, 99Mo or 99mTc. Preferably, the radionuclide may be an alpha-particle-emitting radionuclide.

Example enzymes include malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase or acetylcholinesterase. Such enzymes may be used, for example, in combination with prodrugs that are administered in relatively non-toxic form and converted at the target site by the enzyme into a cytotoxic agent. In other alternatives, a drug may be converted into less toxic form by endogenous enzymes in the subject but may be reconverted into a cytotoxic form by the therapeutic enzyme.

### Bi-Specific Antibody

In one example embodiment, the bifunctional immune cell engager is a bi-specific antibody, e.g., bi-specific antibodies (bsAb) or BiTEs, that bind two antigens (see, e.g., Suurs et al., A review of bispecific antibodies and antibody constructs in oncology and clinical challenges. Pharmacol Ther. 2019 September; 201:103-119; and Huehls, et al., Bispecific T cell engagers for cancer immunotherapy. Immunol Cell Biol. 2015 March; 93(3): 290-296). The bi-specific antigen-binding construct includes two antigen-binding polypeptide constructs, e.g., antigen binding domains, wherein at least one polypeptide construct specifically binds to a tumor surface protein. In some embodiments, the antigen-binding construct is derived from known antibodies or antigen-binding constructs. In some embodiments, the antigen-binding polypeptide constructs comprise two antigen binding domains that comprise antibody fragments. In some embodiments, the first antigen binding domain and second antigen binding domain each independently comprises an antibody fragment selected from the group of: an scFv, a Fab, and an Fc domain. The antibody fragments may be the same format or different formats from each other. For example, in some embodiments, the antigen-binding polypeptide constructs comprise a first antigen binding domain comprising an scFv and a second antigen binding domain comprising a Fab. In some embodiments, the antigen-binding polypeptide constructs comprise a first antigen binding domain and a second antigen binding domain, wherein both antigen binding domains comprise an scFv. In some embodiments, the first and second antigen binding domains each comprise a Fab. In some embodiments, the first and second antigen binding domains each comprise an Fc domain. Any combination of antibody formats is suitable for the bi-specific antibody constructs disclosed herein.

In certain embodiments, immune cells can be engaged to tumor cells. In certain embodiments, tumor cells are targeted with a bsAb having affinity for both the tumor and a payload. In certain embodiments, two targets are disrupted on a tumor cell by the bsAb (e.g., any two of CLDN3, CLDN7, CLDN4, EPCAM, TACSTD2, MAL2, LSR, CD9, SPINT2, TM4SF1, TMEM205, TNFRSF12A and CD47). By means of an example, an agent, such as a bi-specific antibody, capable of specifically binding to a gene product expressed on the cell surface of the immune cells (e.g., CD3, CD8, CD28, CD16) and a tumor cell (e.g., CLDN3, CLDN7, and/or CLDN4) may be used for targeting polyfunctional immune cells to tumor cells. Immune cells targeted to a tumor may include T cells or Natural Killer cells.

### Target Immune Cell

The target immune cell may be a natural or engineered immune cell with a receptor specific to the immunogenic display moiety. The target substrate may be a natural or engineered immune cell with an antigen specific to a bifunctional protein comprising a first antibody for binding the immunogenic display moiety and a second antibody for binding the immune cell. The target immune cell may be positive (+) for or negative (−) for given markers (e.g., cell surface receptors). In certain example embodiments, an immunogenic display moiety or an immune cell engager can target one or more of the positive markers of a target immune cell.

The target immune cell may be a human target immune cell. The human target immune cell may be a Hematopoietic stem cell (HSC) (e.g., CD34+, CD38−, CD45RA−, CD49+, or CD90/Thy1+), a Multi-potent progenitor (MPP) (e.g., CD34+, CD38−, CD45RA−, or CD90/Thy1-), a Common lymphoid progenitor (CLP) (e.g., CD34+, CD38+, CD10+, or CD45RA+), a Common myeloid progenitor (CMP) (e.g., CD34+, CD38+, CD7−, CD10−, CD45RA−, CD90/Thy1−, CD135+), a Megakaryocyte-erythroid progenitor (MEP) (e.g., CD34+, CD38+, CD7−, CD10−, CD45RA−, CD135−, IL3Rα−), a Granulocyte-monocyte progenitor (GMP), (e.g., CD34+, CD38+, CD10−, CD45RA+, CD123+, or CD135+), a Natural killer cell* (e.g., CD3−, CD56+, CD94+, NKp46+), a T-cell*, (e.g., CD3+), a B-cell*, (e.g., CD19+), a Monocyte* (e.g., CD14+), aMacrophage* (e.g., CD11b+, CD68+, CD163+), aDendritic cell* (e.g., CD11c+, HLA-DR+), aNeutrophil (e.g., CD11b+, CD16+, CD18+, CD32+, CD44+, CD55+), aEosinophil (e.g., CD45+, CD125+, CD193+, F4/80+, Siglec-8+), a Basophil (e.g., CD19−, CD22+, CD45low, CD123+), a Mast cell (e.g., CD32+, CD33+, CD117+, CD203c+, FcεRI+), a Erythrocyte (e.g., CD235a+), a Megakaryocyte (e.g., CD41b+, CD42a+, CD42b+, CD61+) or, a Platelet (e.g., CD41+, CD42a+, CD42b+, CD61+), where * markers given for these cell types are common to all subsets (e.g., T-cell markers are common to killer, helper, and regulatory T cells). See Hoover-Plow J, G. Y. Challenges for heart disease stem cell therapy. Vasc. Health Risk Manag. 8, 99-113 (2012); Warr, M. R., Pietras, E. M. & Passegué, E. Mechanisms controlling hematopoietic stem cell functions during normal hematopoiesis and hematological malignancies. Wiley Interdiscip. Rev. Syst. Biol. Med. 3, 681-701 (2011).; Weissman, I. L. & Shizuru, J. A. The origins of the identification and isolation of hematopoietic stem cells, and their capability to induce donor-specific transplantation tolerance and treat autoimmune diseases. Blood 112, 3543-3553 (2008).; Challen, G. a., Boles, N., Lin, K. K. & Goodell, M. a. Mouse Hematopoietic Stem Cell Identification and Analysis. Cytom. A 75, 14-24 (2009).; Sudo, T. et al. in Stem Cell Biology in Normal Life and Diseases (InTech, 2013). doi:10.5772/54474; Notta, F. et al. Isolation of Single Human Hematopoietic Stem Cells Capable of Long Term Multilineage Engraftment. Science 333, 218-221 (2011).; Doulatov, S. et al. Revised map of the human progenitor hierarchy shows the origin of macrophages and dendritic cells in early lymphoid development. Nat. Immunol. 11, 585-93 (2010).; Majeti, R., Park, C. Y. & Weissman, I. L. Identification of a Hierarchy of Multipotent Hematopoietic Progenitors in Human Cord Blood. Cell Stem Cell 1, 635-645 (2007).; Goardon, N. et al. Coexistence of LMPP-like and GMP-like leukemia stem cells in acute myeloid leukemia. Cancer Cell 19, 138-152 (2011).; Welner, R. S., Pelayo, R. & Kincade, P. W. Evolving views on the genealogy of B cells. Nat. Rev. Immunol. 8, 95-106 (2008).; Doulatov, S., Notta, F., Laurenti, E. & Dick, J. E. Hematopoiesis: A human perspective. Cell Stem Cell 10, 120-136 (2012).; Mori, Y., Chen, J. Y., Pluvinage, J. V., Seita, J. & Weissman, I. L. Prospective isolation of human erythroid lineage-committed progenitors. Proc. Natl. Acad. Sci. 112, 9638-9643 (2015).; Chang, Y., Bluteau, D., Debili, N. & Vainchenker, W. From hematopoietic stem cells to platelets. J. Thromb. Haemost. 5, 318-327 (2007).; Novershtern, N. et al. Densely interconnected transcriptional circuits control cell states in human hematopoiesis. Cell 144, 296-309 (2011).; Lee, J. et al. Restricted dendritic cell and monocyte progenitors in human cord blood and bone marrow. J. Exp. Med. 212, 385-99 (2015).; Montaldo, E. et al. Human NK cell receptors/markers: A tool to analyze NK cell development, subsets and function. Cytom. Part A 83, 702-713 (2013).; Chen, Q. et al. Delineation of Natural Killer Cell Differentiation from Myeloid Progenitors in Human. Sci. Rep. 5, 15118 (2015).; Colucci, F., Caligiuri, M. a & Di Santo, J. P. What does it take to make a natural killer?Nat. Rev. Immunol. 3, 413-425 (2003).; Farag, S. S. & Caligiuri, M. A. Human natural killer cell development and biology. Blood Rev. 20, 123-137 (2006).; Flow Cytometry. (Humana Press, 2007). doi:10.1007/978-1-59745-451-3; Galon, J. et al. Type, density, and location of immune cells within human colorectal tumors predict clinical outcome. Science 313, 1960-4 (2006).; Finak, G. et al. Standardizing Flow Cytometry Immunophenotyping Analysis from the Human ImmunoPhenotyping Consortium. Sci. Rep. 6, 20686 (2016).; Kaminski, D. A., Wei, C., Qian, Y., Rosenberg, A. F. & Sanz, I. Advances in human B cell phenotypic profiling. Front. Immunol. 3, 1-15 (2012).; Orlic, D., Fischer, R., Nishikawa, S., Nienhuis, A. W. & Bodine, D. M. Purification and characterization of heterogeneous pluripotent hematopoietic stem cell populations expressing high levels of c-kit receptor. Blood 82, 762-70 (1993).; Bendall, S. C. et al. Single-Cell Trajectory Detection Uncovers Progression and Regulatory Coordination in Human B Cell Development. Cell 157, 714-725 (2014).; Wood, B. Multicolor Immunophenotyping: Human Immune System Hematopoiesis. Methods Cell Biol. 75, 559-576 (2004).; Yang, J., Zhang, L., Yu, C., Yang, X.-F. & Wang, H. Monocyte and macrophage differentiation: circulation inflammatory monocyte as biomarker for inflammatory diseases. Biomark. Res. 2, 1 (2014).; Murray, P. J. & Wynn, T. A. Protective and pathogenic functions of macrophage subsets. Nat. Rev. Immunol. 11, 723-37 (2011).; Pilling, D., Fan, T., Huang, D., Kaul, B. & Gomer, R. H. Identification of markers that distinguish monocyte-derived fibrocytes from monocytes, macrophages, and fibroblasts. PLoS One 4, 31-33 (2009).; Merad, M., Sathe, P., Helft, J., Miller, J. & Mortha, A. The dendritic cell lineage: ontogeny and function of dendritic cells and their subsets in the steady state and the inflamed setting. Annu. Rev. Immunol. 31, 563-604 (2013). Elghetany, M. & Elghetany, M. Surface Antigen Changes during Normal Neutrophilic Development: A Critical Review. Blood Cells, Mol. Dis. 28, 260-274 (2002).; Mantovani, A., Cassatella, M. A., Costantini, C. & Jaillon, S. Neutrophils in the activation and regulation of innate and adaptive immunity. Nat. Rev. Immunol. 11, 519-531 (2011).; Behnen, M. et al. Immobilized Immune Complexes Induce Neutrophil Extracellular Trap Release by Human Neutrophil Granulocytes via Fc RIIIB and Mac-1. J. Immunol. 193, 1954-1965 (2014).; Lee, J. J. et al. Human versus mouse eosinophils: ‘That which we call an eosinophil, by any other name would stain as red’. J. Allergy Clin. Immunol. 130, 572-584 (2012).; Mori, Y. et al. Identification of the human eosinophil lineage-committed progenitor: revision of phenotypic definition of the human common myeloid progenitor. J. Exp. Med. 206, 183-193 (2009).; Rothenberg, M. E. & Hogan, S. P. The eosinophil. Annu. Rev. Immunol. 24, 147-74 (2006).; Han, X. et al. Immunophenotypic study of basophils by multiparameter flow cytometry. Arch. Pathol. Lab. Med. 132, 813-819 (2008).; Cromheecke, J. L., Nguyen, K. T. & Huston, D. P. Emerging role of human basophil biology in health and disease. Curr. Allergy Asthma Rep. 14, 43-45 (2014).; Hauswirth, A. W. et al. Expression of cell surface antigens on mast cells: mast cell phenotyping. Methods Mol. Biol. 315, 77-90 (2006).; Sanchez-Munoz, L., Teodósio, C., Morgado, J. M. & Escribano, L. Immunophenotypic characterization of bone marrow mast cells in mastocytosis and other mast cell disorders. Methods Cell Biol. 103, 333-59 (2011).; Murphy, K. Janeway's Immunobiology. (Garland Science, 2012).; Rodriguez-Perales, S. et al. Truncated RUNX1 protein generated by a novel t(1;21)(p32;q22) chromosomal translocation impairs the proliferation and differentiation of human hematopoietic progenitors. Oncogene 35, 1-10 (2015).; Deutsch, V. R. & Tomer, A. Megakaryocyte development and platelet production. Br. J. Haematol. 134, 453-466 (2006).; Van Velzen, J. F., Laros-Van Gorkom, B. A. P., Pop, G. A. M. & Van Heerde, W. L. Multicolor flow cytometry for evaluation of platelet surface antigens and activation markers. Thromb. Res. 130, 92-98 (2012).; Blom B, Spits H. Development of human lymphoid cells. Annu Rev Immunol. 2006; 24:287-320. Dahlin J S, Hallgren J. Mast Cell Progenitors: Origin, Development, and Migration to Tissues. Mol Immunol. 2015; 63: 9-17. Doulatov S, Notta F, Eppert K, Nguyen L T, Ohashi P S, Dick J E. Revised map of the human progenitor hierarchy shows the origin of macrophages and dendritic cells in early lymphoid development. Nat Immunol. 2010; 11(7):585-93.; Terry R L, Miller S D. Molecular control of monocyte development. Cell Immunol. 2014; 291(1-2):16-21, each incorporated by reference herein in its entirety.

In one example embodiment, the target immune cell may express a CD20 cell surface receptor. See Vlaming, M., Bilemjian, V., Freile, J. Á. et al. CD20 positive CD8 T cells are a unique and transcriptionally-distinct subset of T cells with distinct transmigration properties. Sci Rep 11, 20499 (2021). doi.org/10.1038/s41598-021-00007-0, incorporated by reference herein in its entirety. In one example embodiment, the target immune cell may express a CD30 cell surface receptor. See, van der Weyden, C., Pileri, S., Feldman, A. et al. Understanding CD30 biology and therapeutic targeting: a historical perspective providing insight into future directions. Blood Cancer J. 7, e603 (2017). doi.org/10.1038/bcj.2017.85 incorporated by reference herein in its entirety. In one example embodiment, the target immune cell may express a CD79B cell surface receptor. See Huse K, Bai B, Hilden V I, Bollum L K, Vitsveen T K, Munthe L A, Smeland E B, Irish J M, Walchli S, Myklebust J H. Mechanism of CD79A and CD79B Support for IgM+B Cell Fitness through B Cell Receptor Surface Expression. J Immunol. 2022 Nov. 15; 209(10):2042-2053. doi: 10.4049/jimmunol.2200144. PMID: 36426942; PMCID: PMC9643646, incorporated by reference herein in its entirety. In one example embodiment, the target immune cell may express a SLAM family cell surface receptor, e.g., a SLAMF7 cell surface receptor. See Farhangnia Pooya, Ghomi Shamim Mollazadeh, Mollazadehghomi Shabnam, Nickho Hamid, Akbarpour Mahzad, Delbandi Ali-Akbar, SLAM-family receptors come of age as a potential molecular target in cancer immunotherapy. Frontiers in Immunology, 14 (2023). www.frontiersin.org/articles/10.3389/fimmu.2023.1174138. DOI=10.3389/fimmu.2023.1174138, incorporated by reference herein in its entirety.

### Immunogenic Cargo Moiety

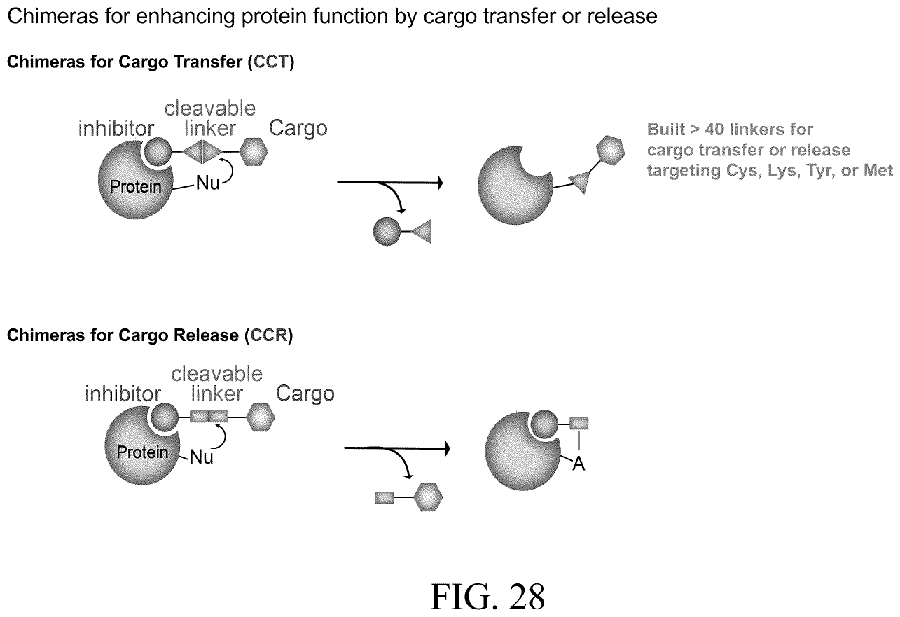

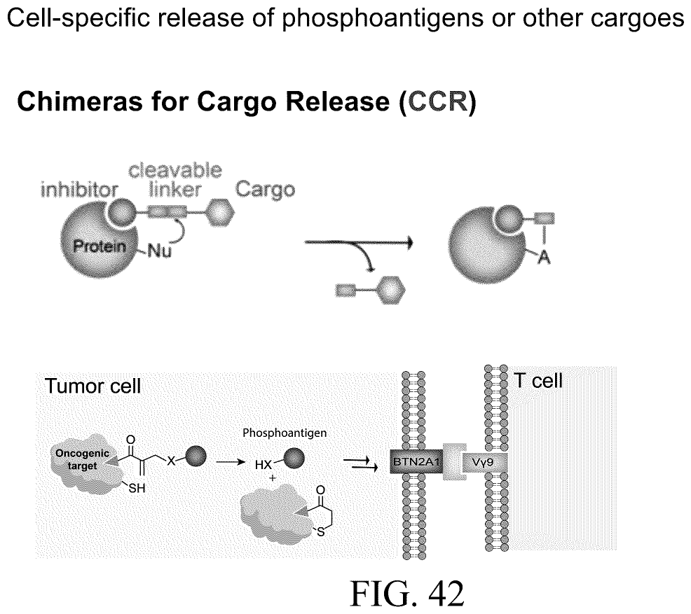

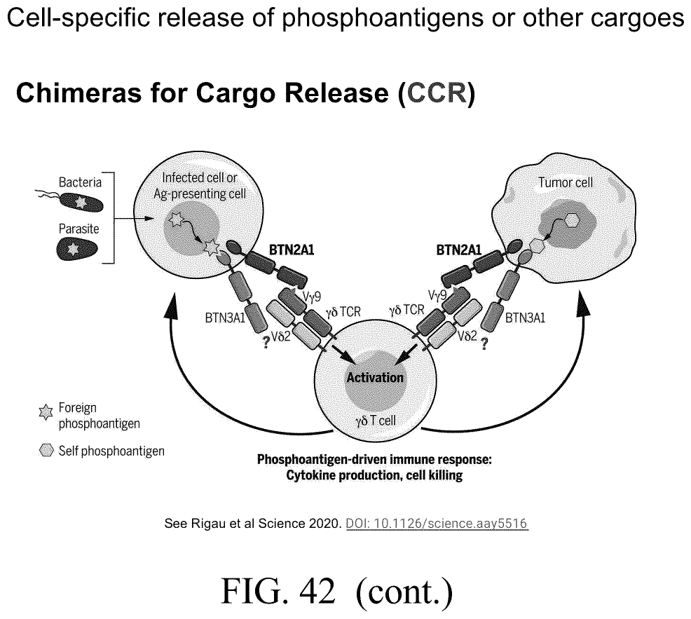

In certain example embodiments, the cleavable linker of a chimeric small molecule covalently bonds to an amino acid of a target protein within a cell, thus releasing the immunogenic cargo moiety within the cell. In certain example embodiments, the released immunogenic cargo moiety indirectly activates immune cell, e.g., phosphoantigens or precursors thereof which are capable of indirect activation of T cells via activation of cell surface molecules. See FIGS. 28 and 42; see also Rigau et al. Butyrophilin 2A1 is essential for phosphoantigen reactivity by γδ T cells (2020), Science, Vol. 367, doi.org/10.1126/science.aay5516 (stating that γδ T cells recognize a phosphoantigen (pAg) via activation of cell surface molecules BTN2A1 and BTN3A1, which co-bind the Vγ9Vδ2 TCR in response to the pAg). In one example embodiment, the phosphoantigen moiety or precursor thereof is selected from

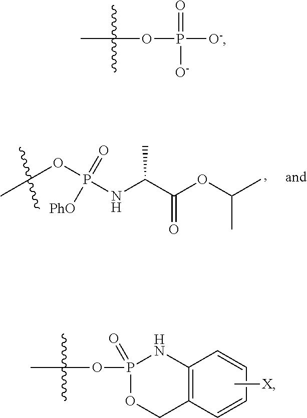

or an analog or derivative thereof, where X is hydrogen or a halogen (e.g., a fluorine).

In certain example embodiments, an immunogenic cargo moiety (or chimeric small molecule comprising said immunogenic cargo moiety), has the structure or a portion thereof of any immunogenic display moiety (or any immune cell recruiting chimeric small molecule comprising said immunogenic display moiety) of the present disclosure. In certain example embodiments, any method of delivering the immune cell recruiting chimeric small molecule and/or labeling one or more target polypeptides of one or more target proteins with the immunogenic display moiety by the immune cell recruiting chimeric small molecule of the present disclosure can be used to deliver said chimeric small molecule comprising said immunogenic cargo moiety and/or labeling one or more target polypeptides of one or more target proteins with said immunogenic cargo moiety.

### Methods of Making Chimeric Small Molecules

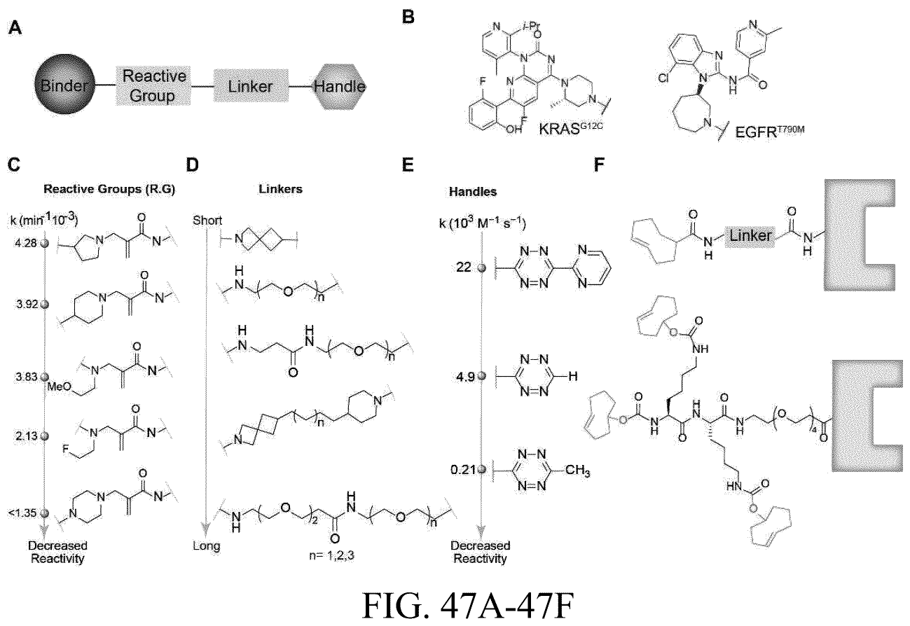

In an aspect, the present invention provides methods of making chimeric small molecules disclosed herein, e.g., chimeric small molecules capable of engaging a T cell engager for recruitment of a T cell. In an example embodiment, a chimeric small molecule comprises at least one of each of 4 components (e.g., FIG. 47A): 1) Any target protein binding moiety A disclosed herein, 2) Any electrophilic reactive group E disclosed herein, e.g., capable of group transfer reaction, 3) Any linker group (L, L1, and/or L2) disclosed herein, and 4) Any immunogenic display moiety B disclosed herein, e.g., an immunogenic display group capable of binding to, e.g., via a click chemistry reaction, a T-cell engager for recruitment of a T cell to a cell displaying said immunogenic display group. In certain example embodiments, the immunogenic display group comprises one component of a click chemistry pair, and a T-cell engager comprises the partner component of the click chemistry pair. In certain example embodiments, click chemistry reactions are optimized for reactivity and in vivo stability.

In an example embodiment, methods of making chimeric small molecules comprise synthesizing one or more chimeric small molecules each comprising the 4 components disclosed herein. In certain example embodiments, methods of making chimeric small molecules comprises synthesizing a library of chimeric small molecules, each comprising a different combination of the 4 components disclosed herein. In certain example embodiments, each chimeric small molecule in a library has a different target protein binding moiety while comprising a common immunogenic display moiety capable of binding to the same T-cell engager. In certain example embodiments, methods of making chimeric small molecules comprise synthesizing or providing one or more separate components of a chimeric small molecule and attaching the prepared or provided components together to form the chimeric small molecule. In an example embodiment, one or more separate components are synthesized directly onto one or more other prepared or provided components to form the chimeric small molecule.

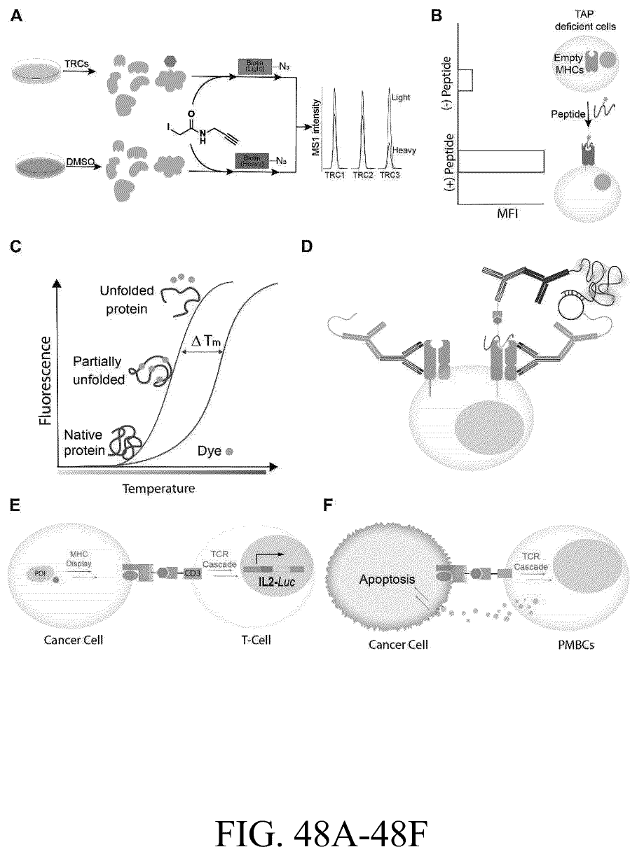

In certain example embodiments, methods of making chimeric small molecules comprises optimizing one or more of the components of a chimeric small molecule, e.g., linker length, rigidity, and stoichiometry. In certain example embodiments, chimeric small molecules are optimized for target protein binding. In certain example embodiments, a library of chimeric small molecules will comprise various target protein binding moieties attached to different electrophilic reactive groups. Thus, the chimeric small molecules will have different binding affinities for their corresponding target protein, e.g., target oncogene, and different reactivities. Both these differences can affect the stoichiometry of labeling of the target oncogene. In certain example embodiments, the degree of labeling of a target protein may be quantified by intact mass spectroscopy (MS) as well as by quantitative mass spectroscopy in cells (e.g., FIG. 48A). For the intact mass spectroscopy, purified target proteins may be incubated with the corresponding chimeric small molecules or DMSO and degree of labeling can be analyzed by intact LC-MS. The chimeric small molecules with the best biochemical labeling may be subject to competitive labeling experiments using an Activity-based protein profiling approach, which has been used extensively to profile covalent inhibitors. Briefly, cells treated with chimeric small molecules or a vehicle will be lysed and labeled with a broad reactive ABPP-alkyne probe, followed by click chemistry with heavy or light-labeled biotin-azide. After enrichment and proteolytic digestions by trypsin, the isotopically labeled peptides will be analyzed by liquid-chromatography-high-resolution MS to determine their ratio to quantify the extent of covalent modification.

In certain example embodiments, chimeric small molecules are optimized for MHC binding. In certain example embodiments, peptides for different HLA alleles (HLA-A*02:01 and HLA-A*03:01) are synthesized and modified by chimeric small molecules biochemically. The affinity of the modified peptides for MHC may be evaluated both biochemically and on cells. For biochemical evaluation, MHC binding may be quantified by measuring the thermal stability of MHC-peptide complexes by differential scanning fluorimetry (DSF) (e.g., FIG. 48B) following previously reported procedures (4). For cellular evaluation, T2 cells may be used that are deficient in transporter associated with antigen processing (TAP) protein (e.g., FIG. 48C). This results in the generation of empty MHCs, which form stable complexes only with exogenously supplied cognate peptides (20, 21)—the cells bearing such peptides can be detected using FACS sorting with TCO-bearing dye. Finally, the reported proximity-ligation assay (PLA) (e.g., FIG. 48D) may be used, which allows the detection and localization of proteins/peptides with single-molecule resolution.

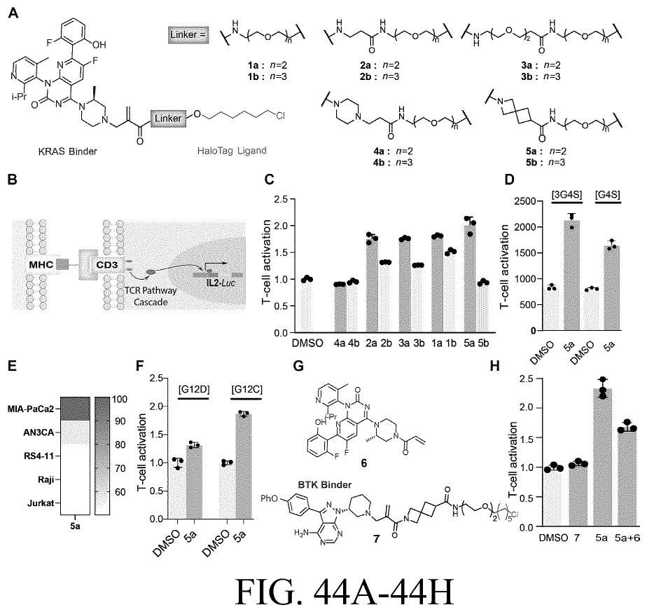

In certain example embodiments, chimeric small molecules are optimized for T-cell activation. To confirm that immunogenic displayed moieties of chimeric small molecules can recruit and activate T-cells, a luciferase reporter assay disclosed herein (see, e.g., FIGS. 44B, 48E) may be used. Whether T-cell activation leads to cytotoxicity may be valudated using peripheral blood mononuclear cells (PBMCs) (see, e.g., FIG. 48F). Here, a published protocol (4) may be used where the target cells bearing a fluorescent marker may be treated with HaCs. In parallel, human PBMCs may be thawed and cultured overnight in complete media. The target cells may then be washed gently three times with complete media and cocultured with PBMCs in the presence of a T-cell engager as disclosed herein. Subsequently, the number of cancer cells may be quantified using the Operetta confocal imager.

In certain example embodiments, chimeric small molecules may be evaluated in vivo. In certain example embodiments, critical physicochemical (e.g., solubility, permeability) and pharmacokinetic (e.g., microsomal stability, plasma binding) properties may be measured for downstream development. Ideal characteristics may include solubility >50 μM in PBS buffer; plasma stability, with >75% parent molecule remaining after 1-hour incubation in human plasma; membrane permeability, as measured by the Caco-2 permeability assay; and liver microsome stability, such that >50% parent molecule remains after 1-hour incubation in human liver microsomes. In certain example embodiments, maximum tolerable dose studies may be performed after identifying the ideal candidate and formulations. All assays are available at various Contract Research Organizations (CROs).

Alternative in vivo efficacy studies may be used. These efficacy studies follow the reported procedures involving quantifying the tumor burden and other associated metrics. Two groups of tumor-bearing mice receive four cycles of chimeric small molecules as disclosed herein at a 0.33 mmol kg-1 dose and a T-cell engager as disclosed herein. Two groups of mice are injected with four cycles of chimeric small molecules at the same dose followed by vehicle and, finally, two more groups of mice receive four cycles of either the T-cell engager or vehicle. The animals are randomly grouped, monitored daily by experienced biotechnicians, and removed from the study in case of poor physical condition (e.g., discomfort, reduced motility). Also, the animals re removed from the study in the cases of excessive body weight loss (>20% with respect to baseline or >15% in two consecutive measurements) or when tumors reach a 1 cm3 size. If these conditions do not occur, the animals will be maintained in the study for up to two months, after which they will be euthanized and selected organs will be harvested and formaldehyde-fixed for histopathology.

### Methods of Making T-Cell Engagers

In an aspect, the present invention provides methods of making T-cell engagers disclosed herein, e.g., T-cell engagers capable of binding to immunogenic display moieties as disclosed herein, e.g., via a click chemistry reaction, for recruitment of a T cell to the cell displaying the immunogenic display moiety. In an example embodiment, methods of making T-cell engagers comprises synthesizing a T cell engager capable of binding to each of a library of chimeric small molecules comprising a same immunogenic display moiety. In certain example embodiments, the immunogenic display group comprises one component of a click chemistry pair, and a T-cell engager comprises the partner component of the click chemistry pair.

In certain example embodiments, a T-cell engager comprises at least one of each of 3 components (e.g., FIG. 47F): 1) Any moiety capable of binding an immunogenic display moiety as disclosed herein, 2) Any T-cell binding moiety as disclosed herein, and 3) Any linker group (L, L1, and/or L2) disclosed herein. In one example embodiment, methods of making T-cell engagers comprise synthesizing one or more T-cell engagers each comprising the 4 components disclosed herein. In certain example embodiments, methods of making chimeric small molecules comprises synthesizing a library of T-cell engagers each comprising the 4 components disclosed herein. In certain example embodiments, each T-cell engager in a library has a same or different T-cell binding moiety while comprising a common moiety capable of binding a common immunogenic display moiety. In certain example embodiments, methods of making T-cell engagers comprises optimizing parameters of one or more components of a T-cell engager, e.g., linker length, rigidity, and stoichiometry.

### Methods of Using Chimeric Small Molecules

In an aspect, the present invention provides for methods of inducing an immune response using chimeric small molecules of the present invention. In one example embodiment, the methods comprise using said chimeric small molecules to label a target protein with an immunogenic display moiety. In one example embodiment, the methods label an intracellular target protein with the immunogenic display moiety. Peptide fragments of said target protein comprising said immunogenic display moiety are capable of being displayed by the MHC system of a cell on the surface of the cell. In one embodiment, the methods label an extracellular protein with the immunogenic display moiety. In one embodiment, recognition of said immunogenic display moiety at the cell surface induces an immune response from an immune cell.

In one embodiment, a method of inducing an immune response comprises: delivering the chimeric small molecule of any of the preceding embodiments to a cell or a subject in need thereof, labeling one or more target polypeptides with an immunogenic display moiety by the chimeric small molecule; and displaying the one more target polypeptides labeled with the immunogenic display moiety on the cell surface via a Major Histocompatibility Complex (MHC) molecule. In one example embodiment, a method is a method of labeling a protein on the surface of the cell, the method comprising: delivering the chimeric small molecule of any of the preceding embodiments to a cell; and labeling one or more target cell surface polypeptides with an immunogenic display moiety by the chimeric small molecule, thereby displaying the target cell surface polypeptides labeled with the immunogenic display moiety on the cell surface.

In one embodiment, the method of any one of the preceding embodiments further comprises eliciting an immune response. In one example embodiment, the immune response comprises recognition of the immunogenic display moiety by a natural or an engineered immune cell. In one embodiment, the method of any one of the preceding embodiments further comprises delivering the bifunctional molecule to the cell surface, and the immune response comprises recognition of the immunogenic display moiety by the first group of the bifunctional molecule and recognition of a natural or engineered immune cell by the second group of the bifunctional molecule.

In one embodiment, according to the method of any one of the preceding embodiments, two or more different proteins are labeled with the same immunogenic display moiety, whereby each protein is recognized by the same engineered immune cell. In one example embodiment, the chimeric small molecule that labels the two or more different proteins is the same molecule or different molecules.

### Immune System and Antigen Presentation

The immune system can be classified into two functional subsystems: the innate and the acquired immune system. The innate immune system is the first line of defense against infections, and most potential pathogens are rapidly neutralized by this system before they can cause, for example, a noticeable infection. The acquired immune system reacts to molecular structures, referred to as antigens, of the intruding organism. There are two types of acquired immune reactions, which include the humoral immune reaction and the cell-mediated immune reaction. In the humoral immune reaction, antibodies secreted by B cells into bodily fluids bind to pathogen-derived antigens, leading to the elimination of the pathogen through a variety of mechanisms, e.g., complement-mediated lysis. In the cell-mediated immune reaction, T-cells capable of destroying other cells are activated. For example, if proteins associated with a disease are present in a cell, they are fragmented proteolytically to peptides within the cell. Specific cell proteins then attach themselves to the antigen or peptide formed in this manner and transport them to the surface of the cell, where they are presented to the molecular defense mechanisms, in particular T-cells, of the body. Cytotoxic T cells recognize these antigens and kill the cells that harbor the antigens.

The molecules that transport and present peptides on the cell surface are referred to as proteins of the major histocompatibility complex (MHC). MHC proteins are classified into two types, referred to as MHC class I and MHC class II. The structures of the proteins of the two MHC classes are very similar; however, they have very different functions. Proteins of MHC class I are present on the surface of almost all cells of the body, including most tumor cells. MHC class I proteins are loaded with antigens that usually originate from endogenous proteins or from pathogens present inside cells and are then presented to naive or cytotoxic T-lymphocytes (CTLs). MHC class II proteins are present on dendritic cells, B-lymphocytes, macrophages and other antigen-presenting cells. They mainly present peptides, which are processed from external antigen sources, i.e. outside of the cells, to T-helper (Th) cells. Most of the peptides bound by the MHC class I proteins originate from cytoplasmic proteins produced in the healthy host cells of an organism itself, and do not normally stimulate an immune reaction. Accordingly, cytotoxic T-lymphocytes that recognize such self-peptide-presenting MHC molecules of class I are deleted in the thymus (central tolerance) or, after their release from the thymus, are deleted or inactivated, i.e. tolerized (peripheral tolerance). MHC molecules are capable of stimulating an immune reaction when they present peptides to non-tolerized T-lymphocytes. Cytotoxic T-lymphocytes have both T-cell receptors (TCR) and CD8 molecules on their surface. T-Cell receptors are capable of recognizing and binding peptides complexed with the molecules of MHC class I. Each cytotoxic T-lymphocyte expresses a unique T-cell receptor which is capable of binding specific MHC/peptide complexes.

The peptide antigens attach themselves to the molecules of MHC class I by competitive affinity binding within the endoplasmic reticulum, before they are presented on the cell surface. Here, the affinity of an individual peptide antigen is directly linked to its amino acid sequence and the presence of specific binding motifs in defined positions within the amino acid sequence. If the sequence of such a peptide is known, it is possible to manipulate the immune system against diseased cells using, for example, peptide vaccines. The human leukocyte antigen (HLA) system is a gene complex encoding the major histocompatibility complex (MHC) proteins in humans.

By “proteins or molecules of the major histocompatibility complex (MHC)”, “MHC molecules”, “MHC proteins” or “HLA proteins” is thus meant proteins capable of binding peptides resulting from the proteolytic cleavage of protein antigens and representing potential T-cell epitopes, transporting them to the cell surface and presenting them there to specific cells, in particular cytotoxic T-lymphocytes or T-helper cells. MHC molecules of class I consist of a heavy chain and a light chain and are capable of binding a peptide of about 8 to 11 amino acids, but usually 9 or 10 amino acids, if this peptide has suitable binding motifs, and presenting it to cytotoxic T-lymphocytes. The peptide bound by the MHC molecules of class I originates from an endogenous protein antigen. The heavy chain of the MHC molecules of class I is preferably an HLA-A, HLA-B or HLA-C monomer, and the light chain is 0-2-microglobulin (B2M).

MHC molecules of class II consist of an α-chain and a β-chain and are capable of binding a peptide of about 15 to 24 amino acids if this peptide has suitable binding motifs, and presenting it to T-helper cells. The peptide bound by the MHC molecules of class II usually originates from an extracellular of exogenous protein antigen. The α-chain and the β-chain are in particular HLA-DR, HLA-DQ and HLA-DP monomers.

Subject specific HLA alleles or HLA genotype of a subject may be determined by any method known in the art. In preferred embodiments, HLA genotypes are determined by any method described in International Patent Application number PCT/US2014/068746, published Jun. 11, 2015 as WO2015085147. Briefly, the methods include determining polymorphic gene types that may comprise generating an alignment of reads extracted from a sequencing data set to a gene reference set comprising allele variants of the polymorphic gene, determining a first posterior probability or a posterior probability derived score for each allele variant in the alignment, identifying the allele variant with a maximum first posterior probability or posterior probability derived score as a first allele variant, identifying one or more overlapping reads that aligned with the first allele variant and one or more other allele variants, determining a second posterior probability or posterior probability derived score for the one or more other allele variants using a weighting factor, identifying a second allele variant by selecting the allele variant with a maximum second posterior probability or posterior probability derived score, the first and second allele variant defining the gene type for the polymorphic gene, and providing an output of the first and second allele variant.

### Anti-Tumor Immunity

In one example embodiment, the method of any one of the preceding embodiments further comprises preventing the recurrence of tumors, including, but not limited to, tumors of the protein labeled with the immunogenic display moiety. See Canon et al., The clinical KRAS (G12C) inhibitor AMG 510 drives anti-tumor immunity (2019), Nature, Vol. 575, pages 217-223 (indicating that treatment with AMG 510 increased cell surface expression of MHC class 1 antigens on KRAS (G12C) cells, as well as inhibited subsequent growth of tumor cells upon subsequent challenge with KRAS (G12C) and KRAS (G12D) tumor cells.).

### Adoptive Cell Transfer

As used herein, “ACT”, “adoptive cell therapy” and “adoptive cell transfer” may be used interchangeably. In certain embodiments, Adoptive cell therapy (ACT) can refer to the transfer of cells to a patient with the goal of transferring the functionality and characteristics into the new host by engraftment of the cells (see, e.g., Mettananda et al., Editing an α-globin enhancer in primary human hematopoietic stem cells as a treatment for β-thalassemia, Nat Commun. 2017 Sep. 4; 8(1):424). As used herein, the term “engraft” or “engraftment” refers to the process of cell incorporation into a tissue of interest in vivo through contact with existing cells of the tissue. Adoptive cell therapy (ACT) can refer to the transfer of cells, most commonly immune-derived cells (e.g., T cells or NK cells), back into the same patient or into a new recipient host with the goal of transferring the immunologic functionality and characteristics into the new host. If possible, use of autologous cells helps the recipient by minimizing GVHD issues. The adoptive transfer of autologous tumor infiltrating lymphocytes (TIL) (Zacharakis et al., (2018) Nat Med. 2018 June; 24(6):724-730; Besser et al., (2010) Clin. Cancer Res 16 (9) 2646-55; Dudley et al., (2002) Science 298 (5594): 850-4; and Dudley et al., (2005) Journal of Clinical Oncology 23 (10): 2346-57.) or genetically re-directed peripheral blood mononuclear cells (Johnson et al., (2009) Blood 114 (3): 535-46; and Morgan et al., (2006) Science 314(5796) 126-9) has been used to successfully treat patients with advanced solid tumors, including melanoma, metastatic breast cancer and colorectal carcinoma, as well as patients with CD19-expressing hematologic malignancies (Kalos et al., (2011) Science Translational Medicine 3 (95): 95ra73). In certain embodiments, allogenic cells immune cells are transferred (see, e.g., Ren et al., (2017) Clin Cancer Res 23 (9) 2255-2266). As described further herein, allogenic cells can be edited to reduce alloreactivity and prevent graft-versus-host disease. Thus, use of allogenic cells allows for cells to be obtained from healthy donors and prepared for use in patients as opposed to preparing autologous cells from a patient after diagnosis.

Aspects of the invention involve the adoptive transfer of immune system cells, such as T cells or NK cells, specific for selected antigens, such as tumor associated antigens or tumor specific neoantigens (see, e.g., Maus et al., 2014, Adoptive Immunotherapy for Cancer or Viruses, Annual Review of Immunology, Vol. 32: 189-225; Rosenberg and Restifo, 2015, Adoptive cell transfer as personalized immunotherapy for human cancer, Science Vol. 348 no. 6230 pp. 62-68; Restifo et al., 2015, Adoptive immunotherapy for cancer: harnessing the T cell response. Nat. Rev. Immunol. 12(4): 269-281; and Jenson and Riddell, 2014, Design and implementation of adoptive therapy with chimeric antigen receptor-modified T cells. Immunol Rev. 257(1): 127-144; and Rajasagi et al., 2014, Systematic identification of personal tumor-specific neoantigens in chronic lymphocytic leukemia. Blood. 2014 Jul. 17; 124(3):453-62).

In certain embodiments, an antigen (such as a tumor antigen) to be targeted in adoptive cell therapy (such as particularly CAR or TCR T-cell therapy) of a disease (such as particularly of tumor or cancer) may be selected from a group consisting of: MR1 (see, e.g., Crowther, et al., 2020, Genome-wide CRISPR-Cas9 screening reveals ubiquitous T cell cancer targeting via the monomorphic MHC class I-related protein MR1, Nature Immunology volume 21, pages 178-185), B cell maturation antigen (BCMA) (see, e.g., Friedman et al., Effective Targeting of Multiple BCMA-Expressing Hematological Malignancies by Anti-BCMA CAR T Cells, Hum Gene Ther. 2018 Mar. 8; Berdeja J G, et al. Durable clinical responses in heavily pretreated patients with relapsed/refractory multiple myeloma: updated results from a multicenter study of bb2121 anti-Bcma CAR T cell therapy. Blood. 2017; 130:740; and Mouhieddine and Ghobrial, Immunotherapy in Multiple Myeloma: The Era of CAR T Cell Therapy, Hematologist, May-June 2018, Volume 15, issue 3); PSA (prostate-specific antigen); prostate-specific membrane antigen (PSMA); PSCA (Prostate stem cell antigen); Tyrosine-protein kinase transmembrane receptor ROR1; fibroblast activation protein (FAP); Tumor-associated glycoprotein 72 (TAG72); Carcinoembryonic antigen (CEA); Epithelial cell adhesion molecule (EPCAM); Mesothelin; Human Epidermal growth factor Receptor 2 (ERBB2 (Her2/neu)); Prostase; Prostatic acid phosphatase (PAP); elongation factor 2 mutant (ELF2M); Insulin-like growth factor 1 receptor (IGF-1R); gplOO; BCR-ABL (breakpoint cluster region-Abelson); tyrosinase; New York esophageal squamous cell carcinoma 1 (NY-ESO-1); κ-light chain, LAGE (L antigen); MAGE (melanoma antigen); Melanoma-associated antigen 1 (MAGE-A1); MAGE A3; MAGE A6; legumain; Human papillomavirus (HPV) E6; HPV E7; prostein; survivin; PCTA1 (Galectin 8); Melan-A/MART-1; Ras mutant; TRP-1 (tyrosinase related protein 1, or gp75); Tyrosinase-related Protein 2 (TRP2); TRP-2/INT2 (TRP-2/intron 2); RAGE (renal antigen); receptor for advanced glycation end products 1 (RAGE1); Renal ubiquitous 1, 2 (RU1, RU2); intestinal carboxyl esterase (iCE); Heat shock protein 70-2 (HSP70-2) mutant; thyroid stimulating hormone receptor (TSHR); CD123; CD171; CD19; CD20; CD22; CD26; CD30; CD33; CD44v7/8 (cluster of differentiation 44, exons 7/8); CD53; CD92; CD100; CD148; CD150; CD200; CD261; CD262; CD362; CS-1 (CD2 subset 1, CRACC, SLAMF7, CD319, and 19A24); C-type lectin-like molecule-1 (CLL-1); ganglioside GD3 (aNeu5Ac(2-8)aNeu5Ac(2-3)bDGalp(1-4)bDGlcp(1-1)Cer); Tn antigen (Tn Ag); Fms-Like Tyrosine Kinase 3 (FLT3); CD38; CD138; CD44v6; B7H3 (CD276); KIT (CD117); Interleukin-13 receptor subunit alpha-2 (IL-13Ra2); Interleukin 11 receptor alpha (IL-11Ra); prostate stem cell antigen (PSCA); Protease Serine 21 (PRSS21); vascular endothelial growth factor receptor 2 (VEGFR2); Lewis(Y) antigen; CD24; Platelet-derived growth factor receptor beta (PDGFR-beta); stage-specific embryonic antigen-4 (SSEA-4); Mucin 1, cell surface associated (MUC1); mucin 16 (MUC16); epidermal growth factor receptor (EGFR); epidermal growth factor receptor variant III (EGFRvIII); neural cell adhesion molecule (NCAM); carbonic anhydrase IX (CAIX); Proteasome (Prosome, Macropain) Subunit, Beta Type, 9 (LMP2); ephrin type-A receptor 2 (EphA2); Ephrin B2; Fucosyl GM1; sialyl Lewis adhesion molecule (sLe); ganglioside GM3 (aNeu5Ac(2-3)bDGalp(1-4)bDGlcp(1-1)Cer); TGS5; high molecular weight-melanoma-associated antigen (HMWMAA); o-acetyl-GD2 ganglioside (OAcGD2); Folate receptor alpha; Folate receptor beta; tumor endothelial marker 1 (TEM1/CD248); tumor endothelial marker 7-related (TEM7R); claudin 6 (CLDN6); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); CT (cancer/testis (antigen)); melanoma cancer testis antigen-1 (MAD-CT-1); melanoma cancer testis antigen-2 (MAD-CT-2); Fos-related antigen 1; p53; p53 mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; Cyclin D1; v-myc avian myelocytomatosis viral oncogene neuroblastoma derived homolog (MYCN); Ras Homolog Family Member C (RhoC); Cytochrome P450 1B1 (CYP1B1); CCCTC-Binding Factor (Zinc Finger Protein)-Like (BORIS); Squamous Cell Carcinoma Antigen Recognized By T Cells-1 or 3 (SART1, SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint-1, -2, -3 or -4 (SSX1, SSX2, SSX3, SSX4); CD79a; CD79b; CD72; Leukocyte-associated immunoglobulin-like receptor 1 (LAIR1); Fc fragment of IgA receptor (FCAR); Leukocyte immunoglobulin-like receptor subfamily A member 2 (LILRA2); CD300 molecule-like family member f (CD300LF); C-type lectin domain family 12 member A (CLECI2A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); mouse double minute 2 homolog (MDM2); livin; alphafetoprotein (AFP); transmembrane activator and CAML Interactor (TACI); B-cell activating factor receptor (BAFF-R); V-Ki-ras2 Kirsten rat sarcoma viral oncogene homolog (KRAS); immunoglobulin lambda-like polypeptide 1 (IGLL1); 707-AP (707 alanine proline); ART-4 (adenocarcinoma antigen recognized by T4 cells); BAGE (B antigen; b-catenin/m, b-catenin/mutated); CAMEL (CTL-recognized antigen on melanoma); CAP1 (carcinoembryonic antigen peptide 1); CASP-8 (caspase-8); CDC27m (cell-division cycle 27 mutated); CDK4/m (cycline-dependent kinase 4 mutated); Cyp-B (cyclophilin B); DAM (differentiation antigen melanoma); EGP-2 (epithelial glycoprotein 2); EGP-40 (epithelial glycoprotein 40); Erbb2, 3, 4 (erythroblastic leukemia viral oncogene homolog-2, -3, 4); FBP (folate binding protein); fAchR (Fetal acetylcholine receptor); G250 (glycoprotein 250); GAGE (G antigen); GnT-V (N-acetylglucosaminyltransferase V); HAGE (helicose antigen); ULA-A (human leukocyte antigen-A); HST2 (human signet ring tumor 2); KIAA0205; KDR (kinase insert domain receptor); LDLR/FUT (low density lipid receptor/GDP L-fucose: b-D-galactosidase 2-a-L fucosyltransferase); L1CAM (L1 cell adhesion molecule); MC1R (melanocortin 1 receptor); Myosin/m (myosin mutated); MUM-1, -2, -3 (melanoma ubiquitous mutated 1, 2, 3); NA88-A (NA cDNA clone of patient M88); KG2D (Natural killer group 2, member D) ligands; oncofetal antigen (h5T4); p190 minor bcr-abl (protein of 190KD bcr-abl); Pml/RARa (promyelocytic leukaemia/retinoic acid receptor a); PRAME (preferentially expressed antigen of melanoma); SAGE (sarcoma antigen); TEL/AML1 (translocation Ets-family leukemia/acute myeloid leukemia 1); TPI/m (triosephosphate isomerase mutated); CD70; and any combination thereof.

In certain embodiments, an antigen to be targeted in adoptive cell therapy (such as particularly CAR or TCR T-cell therapy) of a disease (such as particularly of tumor or cancer) is a tumor-specific antigen (TSA).

In certain embodiments, an antigen to be targeted in adoptive cell therapy (such as particularly CAR or TCR T-cell therapy) of a disease (such as particularly of tumor or cancer) is a neoantigen.

In certain embodiments, an antigen to be targeted in adoptive cell therapy (such as particularly CAR or TCR T-cell therapy) of a disease (such as particularly of tumor or cancer) is a tumor-associated antigen (TAA).

In certain embodiments, an antigen to be targeted in adoptive cell therapy (such as particularly CAR or TCR T-cell therapy) of a disease (such as particularly of tumor or cancer) is a universal tumor antigen. In certain preferred embodiments, the universal tumor antigen is selected from the group consisting of: a human telomerase reverse transcriptase (hTERT), survivin, mouse double minute 2 homolog (MDM2), cytochrome P450 1B 1 (CYP1B), HER2/neu, Wilms' tumor gene 1 (WT1), livin, alphafetoprotein (AFP), carcinoembryonic antigen (CEA), mucin 16 (MUC16), MUC1, prostate-specific membrane antigen (PSMA), p53, cyclin (Dl), and any combinations thereof.

In certain embodiments, an antigen (such as a tumor antigen) to be targeted in adoptive cell therapy (such as particularly CAR or TCR T-cell therapy) of a disease (such as particularly of tumor or cancer) may be selected from a group consisting of: CD19, BCMA, CD70, CLL-1, MAGE A3, MAGE A6, HPV E6, HPV E7, WT1, CD22, CD171, ROR1, MUC16, and SSX2. In certain preferred embodiments, the antigen may be CD19. For example, CD19 may be targeted in hematologic malignancies, such as in lymphomas, more particularly in B-cell lymphomas, such as without limitation in diffuse large B-cell lymphoma, primary mediastinal b-cell lymphoma, transformed follicular lymphoma, marginal zone lymphoma, mantle cell lymphoma, acute lymphoblastic leukemia including adult and pediatric ALL, non-Hodgkin lymphoma, indolent non-Hodgkin lymphoma, or chronic lymphocytic leukemia. For example, BCMA may be targeted in multiple myeloma or plasma cell leukemia (see, e.g., 2018 American Association for Cancer Research (AACR) Annual meeting Poster: Allogeneic Chimeric Antigen Receptor T Cells Targeting B Cell Maturation Antigen). For example, CLL1 may be targeted in acute myeloid leukemia. For example, MAGE A3, MAGE A6, SSX2, and/or KRAS may be targeted in solid tumors. For example, HPV E6 and/or HPV E7 may be targeted in cervical cancer or head and neck cancer. For example, WT1 may be targeted in acute myeloid leukemia (AML), myelodysplastic syndromes (MDS), chronic myeloid leukemia (CML), non-small cell lung cancer, breast, pancreatic, ovarian or colorectal cancers, or mesothelioma. For example, CD22 may be targeted in B cell malignancies, including non-Hodgkin lymphoma, diffuse large B-cell lymphoma, or acute lymphoblastic leukemia. For example, CD171 may be targeted in neuroblastoma, glioblastoma, or lung, pancreatic, or ovarian cancers. For example, ROR1 may be targeted in ROR1+ malignancies, including non-small cell lung cancer, triple negative breast cancer, pancreatic cancer, prostate cancer, ALL, chronic lymphocytic leukemia, or mantle cell lymphoma. For example, MUC16 may be targeted in MUC16ecto+ epithelial ovarian, fallopian tube or primary peritoneal cancer. For example, CD70 may be targeted in both hematologic malignancies as well as in solid cancers such as renal cell carcinoma (RCC), gliomas (e.g., GBM), and head and neck cancers (HNSCC). CD70 is expressed in both hematologic malignancies as well as in solid cancers, while its expression in normal tissues is restricted to a subset of lymphoid cell types (see, e.g., 2018 American Association for Cancer Research (AACR) Annual meeting Poster: Allogeneic CRISPR Engineered Anti-CD70 CAR-T Cells Demonstrate Potent Preclinical Activity Against Both Solid and Hematological Cancer Cells).

Various strategies may for example be employed to genetically modify T cells by altering the specificity of the T cell receptor (TCR) for example by introducing new TCR a and R chains with selected peptide specificity (see U.S. Pat. No. 8,697,854; PCT Patent Publications: WO2003020763, WO2004033685, WO2004044004, WO2005114215, WO2006000830, WO2008038002, WO2008039818, WO2004074322, WO2005113595, WO2006125962, WO2013166321, WO2013039889, WO2014018863, WO2014083173; U.S. Pat. No. 8,088,379).

As an alternative to, or addition to, TCR modifications, chimeric antigen receptors (CARs) may be used in order to generate immunoresponsive cells, such as T cells or natural killer cells (NK), specific for selected targets, such as malignant cells, with a wide variety of receptor chimera constructs having been described (see U.S. Pat. Nos. 5,843,728; 5,851,828; 5,912,170; 6,004,811; 6,284,240; 6,392,013; 6,410,014; 6,753,162; 8,211,422; and, PCT Publication WO9215322).

In general, CARs are comprised of an extracellular domain, a transmembrane domain, and an intracellular domain, wherein the extracellular domain comprises an antigen-binding domain that is specific for a predetermined target (see, e.g., Gong Y, Klein Wolterink R G J, Wang J, Bos G M J, Germeraad W T V. Chimeric antigen receptor natural killer (CAR-NK) cell design and engineering for cancer therapy. J Hematol Oncol. 2021; 14(1):73; Guedan S, Calderon H, Posey A D Jr, Maus M V. Engineering and Design of Chimeric Antigen Receptors. Mol Ther Methods Clin Dev. 2018; 12:145-156; Petersen C T, Krenciute G. Next Generation CAR T Cells for the Immunotherapy of High-Grade Glioma. Front Oncol. 2019; 9:69; and Lu H, Zhao X, Li Z, Hu Y, Wang H. From CAR-T Cells to CAR-NK Cells: A Developing Immunotherapy Method for Hematological Malignancies. Front Oncol. 2021). While the antigen-binding domain of a CAR is often an antibody or antibody fragment (e.g., a single chain variable fragment, scFv), the binding domain is not particularly limited so long as it results in specific recognition of a target. For example, in some embodiments, the antigen-binding domain may comprise a receptor, such that the CAR is capable of binding to the ligand of the receptor. Alternatively, the antigen-binding domain may comprise a ligand, such that the CAR is capable of binding the endogenous receptor of that ligand.

The antigen-binding domain of a CAR is generally separated from the transmembrane domain by a hinge or spacer. The spacer is also not particularly limited, and it is designed to provide the CAR with flexibility. For example, a spacer domain may comprise a portion of a human Fc domain, including a portion of the CH3 domain, or the hinge region of any immunoglobulin, such as IgA, IgD, IgE, IgG, or IgM, or variants thereof. Furthermore, the hinge region may be modified so as to prevent off-target binding by FcRs or other potential interfering objects. For example, the hinge may comprise an IgG4 Fc domain with or without a S228P, L235E, and/or N297Q mutation (according to Kabat numbering) in order to decrease binding to FcRs. Additional spacers/hinges include, but are not limited to, CD4, CD8, and CD28 hinge regions.

The transmembrane domain of a CAR may be derived either from a natural or from a synthetic source. Where the source is natural, the domain may be derived from any membrane bound or transmembrane protein. Transmembrane regions of particular use in this disclosure may be derived from CD8, CD28, CD3, CD45, CD4, CD5, CD5, CD9, CD 16, CD22, CD33, CD37, CD64, CD80, CD86, CD 134, CD137, CD 154, TCR. Alternatively, the transmembrane domain may be synthetic, in which case it will comprise predominantly hydrophobic residues such as leucine and valine. Preferably a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the cytoplasmic signaling domain of the CAR. A glycine-serine doublet provides a particularly suitable linker.

Alternative CAR constructs may be characterized as belonging to successive generations. First-generation CARs typically consist of a single-chain variable fragment of an antibody specific for an antigen, for example comprising a VL linked to a VH of a specific antibody, linked by a flexible linker, for example by a CD8a hinge domain and a CD8a transmembrane domain, to the transmembrane and intracellular signaling domains of either CD3 (or FcRγ (scFv-CD3 (or scFv-FcRγ; see U.S. Pat. Nos. 7,741,465; 5,912,172; U.S. Pat. No. 5,906,936). Second-generation CARs incorporate the intracellular domains of one or more costimulatory molecules, such as CD28, OX40 (CD134), or 4-1BB (CD137) within the endodomain (for example scFv-CD28/OX40/4-1BB-CD3ζ; see U.S. Pat. Nos. 8,911,993; 8,916,381; 8,975,071; 9,101,584; 9,102,760; 9,102,761). Third-generation CARs include a combination of costimulatory endodomains, such a CD3ζ-chain, CD97, GDI 1a-CD18, CD2, ICOS, CD27, CD154, CDS, OX40, 4-1BB, CD2, CD7, LIGHT, LFA-1, NKG2C, B7-H3, CD30, CD40, PD-1, or CD28 signaling domains (for example scFv-CD28-4-1BB-CD3 (or scFv-CD28-OX40-CD3ζ; see U.S. Pat. Nos. 8,906,682; 8,399,645; 5,686,281; PCT Publication No. WO2014134165; PCT Publication No. WO2012079000). In certain embodiments, the primary signaling domain comprises a functional signaling domain of a protein selected from the group consisting of CD3 zeta, CD3 gamma, CD3 delta, CD3 epsilon, common FcR gamma (FCERIG), FcR beta (Fc Epsilon Rib), CD79a, CD79b, Fc gamma RIIa, DAP10, and DAP12. In certain preferred embodiments, the primary signaling domain comprises a functional signaling domain of CD3ζ or FcRγ. In certain embodiments, the one or more costimulatory signaling domains comprise a functional signaling domain of a protein selected, each independently, from the group consisting of: CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD160, CD19, CD4, CD8 alpha, CD8 beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Lyl08), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D. In certain embodiments, the one or more costimulatory signaling domains comprise a functional signaling domain of a protein selected, each independently, from the group consisting of: 4-1BB, CD27, and CD28. In certain embodiments, a chimeric antigen receptor may have the design as described in U.S. Pat. No. 7,446,190, comprising an intracellular domain of CD3ζ chain (such as amino acid residues 52-163 of the human CD3 zeta chain, as shown in SEQ ID NO: 14 of U.S. Pat. No. 7,446,190), a signaling region from CD28 and an antigen-binding element (or portion or domain; such as scFv). The CD28 portion, when between the zeta chain portion and the antigen-binding element, may suitably include the transmembrane and signaling domains of CD28 (such as amino acid residues 114-220 of SEQ ID NO: 10, full sequence shown in SEQ ID NO: 6 of U.S. Pat. No. 7,446,190; these can include the following portion of CD28 as set forth in Genbank identifier NM_006139 (sequence version 1, 2 or 3):

(SEQ ID NO: 39)
IEVMYPPPYLDNEKSNGTIIHVKGKHLCPSPLFPGPSKPFWVLVVVGGVL
ACYSLLVTVAFIIFWVRSKRSRLLHSDYMNMTPRRPGPTRKHYQPYAPPR
DFAAYRS)).

Alternatively, when the zeta sequence lies between the CD28 sequence and the antigen-binding element, intracellular domain of CD28 can be used alone (such as amino sequence set forth in SEQ ID NO: 9 of U.S. Pat. No. 7,446,190). Hence, certain embodiments employ a CAR comprising (a) a zeta chain portion comprising the intracellular domain of human CD3ζ chain, (b) a costimulatory signaling region, and (c) an antigen-binding element (or portion or domain), wherein the costimulatory signaling region comprises the amino acid sequence encoded by SEQ ID NO: 6 of U.S. Pat. No. 7,446,190.

Alternatively, costimulation may be orchestrated by expressing CARs in antigen-specific T cells, chosen so as to be activated and expanded following engagement of their native αβTCR, for example by antigen on professional antigen-presenting cells, with attendant costimulation. In addition, additional engineered receptors may be provided on the immunoresponsive cells, for example to improve targeting of a T-cell attack and/or minimize side effects.

By means of an example and without limitation, Kochenderfer et al., (2009) J Immunother. 32 (7): 689-702 described anti-CD19 chimeric antigen receptors (CAR). FMC63-28Z CAR contained a single chain variable region moiety (scFv) recognizing CD19 derived from the FMC63 mouse hybridoma (described in Nicholson et al., (1997) Molecular Immunology 34: 1157-1165), a portion of the human CD28 molecule, and the intracellular component of the human TCR-ζ molecule. FMC63-CD828BBZ CAR contained the FMC63 scFv, the hinge and transmembrane regions of the CD8 molecule, the cytoplasmic portions of CD28 and 4-1BB, and the cytoplasmic component of the TCR-ζ molecule. The exact sequence of the CD28 molecule included in the FMC63-28Z CAR corresponded to Genbank identifier NM_006139; the sequence included all amino acids starting with the amino acid sequence IEVMYPPPY (SEQ ID NO: 40) and continuing all the way to the carboxy-terminus of the protein. To encode the anti-CD19 scFv component of the vector, the authors designed a DNA sequence which was based on a portion of a previously published CAR (Cooper et al., (2003) Blood 101: 1637-1644). This sequence encoded the following components in frame from the 5′ end to the 3′ end: an XhoI site, the human granulocyte-macrophage colony-stimulating factor (GM-CSF) receptor α-chain signal sequence, the FMC63 light chain variable region (as in Nicholson et al., supra), a linker peptide (as in Cooper et al., supra), the FMC63 heavy chain variable region (as in Nicholson et al., supra), and a NotI site. A plasmid encoding this sequence was digested with XhoI and NotI. To form the MSGV-FMC63-28Z retroviral vector, the XhoI and NotI-digested fragment encoding the FMC63 scFv was ligated into a second XhoI and NotI-digested fragment that encoded the MSGV retroviral backbone (as in Hughes et al., (2005) Human Gene Therapy 16: 457-472) as well as part of the extracellular portion of human CD28, the entire transmembrane and cytoplasmic portion of human CD28, and the cytoplasmic portion of the human TCR-ζ molecule (as in Maher et al., 2002) Nature Biotechnology 20: 70-75). The FMC63-28Z CAR is included in the KTE-C19 (axicabtagene ciloleucel) anti-CD19 CAR-T therapy product in development by Kite Pharma, Inc. for the treatment of inter alia patients with relapsed/refractory aggressive B-cell non-Hodgkin lymphoma (NHL). Accordingly, in certain embodiments, cells intended for adoptive cell therapies, more particularly immunoresponsive cells such as T cells, may express the FMC63-28Z CAR as described by Kochenderfer et al. (supra). Hence, in certain embodiments, cells intended for adoptive cell therapies, more particularly immunoresponsive cells such as T cells, may comprise a CAR comprising an extracellular antigen-binding element (or portion or domain; such as scFv) that specifically binds to an antigen, an intracellular signaling domain comprising an intracellular domain of a CD3ζ chain, and a costimulatory signaling region comprising a signaling domain of CD28. Preferably, the CD28 amino acid sequence is as set forth in Genbank identifier NM_006139 (sequence version 1, 2 or 3) starting with the amino acid sequence IEVMYPPPY (SEQ ID NO: 40) and continuing all the way to the carboxy-terminus of the protein. The sequence is reproduced herein:

(SEQ ID NO: 39)
IEVMYPPPYLDNEKSNGTIIHVKGKHLCPSPLFPGPSKPFWVLVVVGGVL
ACYSLLVTVAFIIFWVRSKRSRLLHSDYMNMTPRRPGPTRKHYQPYAPPR
DFAAYRS.

Preferably, the antigen is CD19, more preferably the antigen-binding element is an anti-CD19 scFv, even more preferably the anti-CD19 scFv as described by Kochenderfer et al. (supra).

Additional anti-CD19 CARs are further described in WO2015187528. More particularly Example 1 and Table 1 of WO2015187528, incorporated by reference herein, demonstrate the generation of anti-CD19 CARs based on a fully human anti-CD19 monoclonal antibody (47G4, as described in US20100104509) and murine anti-CD19 monoclonal antibody (as described in Nicholson et al. and explained above). Various combinations of a signal sequence (human CD8-alpha or GM-CSF receptor), extracellular and transmembrane regions (human CD8-alpha) and intracellular T-cell signalling domains (CD28-CD3ζ; 4-1BB-CD3ζ; CD27-CD3ζ; CD28-CD27-CD3ζ, 4-1BB-CD27-CD3ζ; CD27-4-1BB-CD3ζ; CD28-CD27-FcεRI gamma chain; or CD28-FcεRI gamma chain) were disclosed. Hence, in certain embodiments, cells intended for adoptive cell therapies, more particularly immunoresponsive cells such as T cells, may comprise a CAR comprising an extracellular antigen-binding element that specifically binds to an antigen, an extracellular and transmembrane region as set forth in Table 1 of WO2015187528 and an intracellular T-cell signalling domain as set forth in Table 1 of WO2015187528. Preferably, the antigen is CD19, more preferably the antigen-binding element is an anti-CD19 scFv, even more preferably the mouse or human anti-CD19 scFv as described in Example 1 of WO2015187528. In certain embodiments, the CAR comprises, consists essentially of or consists of an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, or SEQ ID NO: 13 as set forth in Table 1 of WO2015187528.

By means of an example and without limitation, chimeric antigen receptor that recognizes the CD70 antigen is described in WO2012058460A2 (see also, Park et al., CD70 as a target for chimeric antigen receptor T cells in head and neck squamous cell carcinoma, Oral Oncol. 2018 March; 78:145-150; and Jin et al., CD70, a novel target of CAR T-cell therapy for gliomas, Neuro Oncol. 2018 Jan. 10; 20(1):55-65). CD70 is expressed by diffuse large B-cell and follicular lymphoma and also by the malignant cells of Hodgkins lymphoma, Waldenstrom's macroglobulinemia and multiple myeloma, and by HTLV-1- and EBV-associated malignancies. (Agathanggelou et al. Am. J. Pathol. 1995; 147: 1152-1160; Hunter et al., Blood 2004; 104:4881. 26; Lens et al., J Immunol. 2005; 174:6212-6219; Baba et al., J Virol. 2008; 82:3843-3852.) In addition, CD70 is expressed by non-hematological malignancies such as renal cell carcinoma and glioblastoma. (Junker et al., J Urol. 2005; 173:2150-2153; Chahlavi et al., Cancer Res 2005; 65:5428-5438) Physiologically, CD70 expression is transient and restricted to a subset of highly activated T, B, and dendritic cells.

By means of an example and without limitation, chimeric antigen receptor that recognizes BCMA has been described (see, e.g., US20160046724A1; WO2016014789A2; WO2017211900A1; WO2015158671A1; US20180085444A1; WO2018028647A1; US20170283504A1; and WO2013154760A1).

In certain embodiments, the immune cell may, in addition to a CAR or exogenous TCR as described herein, further comprise a chimeric inhibitory receptor (inhibitory CAR) that specifically binds to a second target antigen and is capable of inducing an inhibitory or immunosuppressive or repressive signal to the cell upon recognition of the second target antigen. In certain embodiments, the chimeric inhibitory receptor comprises an extracellular antigen-binding element (or portion or domain) configured to specifically bind to a target antigen, a transmembrane domain, and an intracellular immunosuppressive or repressive signaling domain. In certain embodiments, the second target antigen is an antigen that is not expressed on the surface of a cancer cell or infected cell or the expression of which is downregulated on a cancer cell or an infected cell. In certain embodiments, the second target antigen is an MHC-class I molecule. In certain embodiments, the intracellular signaling domain comprises a functional signaling portion of an immune checkpoint molecule, such as for example PD-1 or CTLA4. Advantageously, the inclusion of such inhibitory CAR reduces the chance of the engineered immune cells attacking non-target (e.g., non-cancer) tissues.

Alternatively, T-cells expressing CARs may be further modified to reduce or eliminate expression of endogenous TCRs in order to reduce off-target effects. Reduction or elimination of endogenous TCRs can reduce off-target effects and increase the effectiveness of the T cells (U.S. Pat. No. 9,181,527). T cells stably lacking expression of a functional TCR may be produced using a variety of approaches. T cells internalize, sort, and degrade the entire T cell receptor as a complex, with a half-life of about 10 hours in resting T cells and 3 hours in stimulated T cells (von Essen, M. et al. 2004. J. Immunol. 173:384-393). Proper functioning of the TCR complex requires the proper stoichiometric ratio of the proteins that compose the TCR complex. TCR function also requires two functioning TCR zeta proteins with ITAM motifs. The activation of the TCR upon engagement of its MHC-peptide ligand requires the engagement of several TCRs on the same T cell, which all must signal properly. Thus, if a TCR complex is destabilized with proteins that do not associate properly or cannot signal optimally, the T cell will not become activated sufficiently to begin a cellular response.

Accordingly, in some embodiments, TCR expression may eliminated using RNA interference (e.g., shRNA, siRNA, miRNA, etc.), CRISPR, or other methods that target the nucleic acids encoding specific TCRs (e.g., TCR-α and TCR-β) and/or CD3 chains in primary T cells. By blocking expression of one or more of these proteins, the T cell will no longer produce one or more of the key components of the TCR complex, thereby destabilizing the TCR complex and preventing cell surface expression of a functional TCR.

In some instances, CAR may also comprise a switch mechanism for controlling expression and/or activation of the CAR. For example, a CAR may comprise an extracellular, transmembrane, and intracellular domain, in which the extracellular domain comprises a target-specific binding element that comprises a label, binding domain, or tag that is specific for a molecule other than the target antigen that is expressed on or by a target cell. In such embodiments, the specificity of the CAR is provided by a second construct that comprises a target antigen binding domain (e.g., an scFv or a bispecific antibody that is specific for both the target antigen and the label or tag on the CAR) and a domain that is recognized by or binds to the label, binding domain, or tag on the CAR. See, e.g., WO 2013/044225, WO 2016/000304, WO 2015/057834, WO 2015/057852, WO 2016/070061, U.S. Pat. No. 9,233,125, US 2016/0129109. In this way, a T-cell that expresses the CAR can be administered to a subject, but the CAR cannot bind its target antigen until the second composition comprising an antigen-specific binding domain is administered.

Alternative switch mechanisms include CARs that require multimerization in order to activate their signaling function (see, e.g., US 2015/0368342, US 2016/0175359, US 2015/0368360) and/or an exogenous signal, such as a small molecule drug (US 2016/0166613, Yung et al., Science, 2015), in order to elicit a T-cell response. Some CARs may also comprise a “suicide switch” to induce cell death of the CAR T-cells following treatment (Buddee et al., PLoS One, 2013) or to downregulate expression of the CAR following binding to the target antigen (WO 2016/011210).

Alternative techniques may be used to transform target immunoresponsive cells, such as protoplast fusion, lipofection, transfection or electroporation. A wide variety of vectors may be used, such as retroviral vectors, lentiviral vectors, adenoviral vectors, adeno-associated viral vectors, plasmids or transposons, such as a Sleeping Beauty transposon (see U.S. Pat. Nos. 6,489,458; 7,148,203; 7,160,682; 7,985,739; 8,227,432), may be used to introduce CARs, for example using 2nd generation antigen-specific CARs signaling through CD3ζ and either CD28 or CD137. Viral vectors may for example include vectors based on HIV, SV40, EBV, HSV or BPV. In certain embodiments, inducible gene switches are used to regulate expression of a CAR or TCR (see, e.g., Chakravarti, Deboki et al. “Inducible Gene Switches with Memory in Human T Cells for Cellular Immunotherapy.” ACS synthetic biology vol. 8,8 (2019): 1744-1754).

Cells that are targeted for transformation may for example include T cells, Natural Killer (NK) cells, cytotoxic T lymphocytes (CTL), regulatory T cells, human embryonic stem cells, tumor-infiltrating lymphocytes (TIL) or a pluripotent stem cell from which lymphoid cells may be differentiated. T cells expressing a desired CAR may for example be selected through co-culture with γ-irradiated activating and propagating cells (AaPC), which co-express the cancer antigen and co-stimulatory molecules. The engineered CAR T-cells may be expanded, for example by co-culture on AaPC in presence of soluble factors, such as IL-2 and IL-21. This expansion may for example be carried out so as to provide memory CAR+ T cells (which may for example be assayed by non-enzymatic digital array and/or multi-panel flow cytometry). In this way, CAR T cells may be provided that have specific cytotoxic activity against antigen-bearing tumors (optionally in conjunction with production of desired chemokines such as interferon-γ). CAR T cells of this kind may for example be used in animal models, for example to treat tumor xenografts.

In certain embodiments, ACT includes co-transferring CD4+ Th1 cells and CD8+ CTLs to induce a synergistic antitumour response (see, e.g., Li et al., Adoptive cell therapy with CD4+ T helper 1 cells and CD8+ cytotoxic T cells enhances complete rejection of an established tumour, leading to generation of endogenous memory responses to non-targeted tumour epitopes. Clin Transl Immunology. 2017 October; 6(10): e160).

In certain embodiments, Th17 cells are transferred to a subject in need thereof. Th17 cells have been reported to directly eradicate melanoma tumors in mice to a greater extent than Th1 cells (Muranski P, et al., Tumor-specific Th17-polarized cells eradicate large established melanoma. Blood. 2008 Jul. 15; 112(2):362-73; and Martin-Orozco N, et al., T helper 17 cells promote cytotoxic T cell activation in tumor immunity. Immunity. 2009 Nov. 20; 31(5):787-98). Those studies involved an adoptive T cell transfer (ACT) therapy approach, which takes advantage of CD4+ T cells that express a TCR recognizing tyrosinase tumor antigen. Exploitation of the TCR leads to rapid expansion of Th17 populations to large numbers ex vivo for reinfusion into the autologous tumor-bearing hosts.

In certain embodiments, ACT may include autologous iPSC-based vaccines, such as irradiated iPSCs in autologous anti-tumor vaccines (see e.g., Kooreman, Nigel G. et al., Autologous iPSC-Based Vaccines Elicit Anti-tumor Responses In Vivo, Cell Stem Cell 22, 1-13, 2018, doi.org/10.1016/j.stem.2018.01.016).

Unlike T-cell receptors (TCRs) that are MHC restricted, CARs can potentially bind any cell surface-expressed antigen and can thus be more universally used to treat patients (see Irving et al., Engineering Chimeric Antigen Receptor T-Cells for Racing in Solid Tumors: Don't Forget the Fuel, Front. Immunol., 3 Apr. 2017, doi.org/10.3389/fimmu.2017.00267). In certain embodiments, in the absence of endogenous T-cell infiltrate (e.g., due to aberrant antigen processing and presentation), which precludes the use of TIL therapy and immune checkpoint blockade, the transfer of CAR T-cells may be used to treat patients (see, e.g., Hinrichs C S, Rosenberg S A. Exploiting the curative potential of adoptive T-cell therapy for cancer. Immunol Rev (2014) 257(1):56-71. doi:10.1111/imr.12132).

Approaches such as the foregoing may be adapted to provide methods of treating and/or increasing survival of a subject having a disease, such as a neoplasia, for example by administering an effective amount of an immunoresponsive cell comprising an antigen recognizing receptor that binds a selected antigen, wherein the binding activates the immunoresponsive cell, thereby treating or preventing the disease (such as a neoplasia, a pathogen infection, an autoimmune disorder, or an allogeneic transplant reaction).

In certain embodiments, the treatment can be administered after lymphodepleting pretreatment in the form of chemotherapy (typically a combination of cyclophosphamide and fludarabine) or radiation therapy. Initial studies in ACT had short lived responses and the transferred cells did not persist in vivo for very long (Houot et al., T-cell-based immunotherapy: adoptive cell transfer and checkpoint inhibition. Cancer Immunol Res (2015) 3(10):1115-22; and Kamta et al., Advancing Cancer Therapy with Present and Emerging Immuno-Oncology Approaches. Front. Oncol. (2017) 7:64). Immune suppressor cells like Tregs and MDSCs may attenuate the activity of transferred cells by outcompeting them for the necessary cytokines. Not being bound by a theory lymphodepleting pretreatment may eliminate the suppressor cells allowing the TILs to persist.

In one embodiment, the treatment can be administrated into patients undergoing an immunosuppressive treatment (e.g., glucocorticoid treatment). The cells, or population of cells, may be made resistant to at least one immunosuppressive agent due to the inactivation of a gene encoding a receptor for such immunosuppressive agent. In certain embodiments, the immunosuppressive treatment provides for the selection and expansion of the immunoresponsive T cells within the patient.

In certain embodiments, the treatment can be administered before primary treatment (e.g., surgery or radiation therapy) to shrink a tumor before the primary treatment. In another embodiment, the treatment can be administered after primary treatment to remove any remaining cancer cells.

In certain embodiments, immunometabolic barriers can be targeted therapeutically prior to and/or during ACT to enhance responses to ACT or CAR T-cell therapy and to support endogenous immunity (see, e.g., Irving et al., Engineering Chimeric Antigen Receptor T-Cells for Racing in Solid Tumors: Don't Forget the Fuel, Front. Immunol., 3 Apr. 2017, doi.org/10.3389/fimmu.2017.00267).

The administration of cells or population of cells, such as immune system cells or cell populations, such as more particularly immunoresponsive cells or cell populations, as disclosed herein may be carried out in any convenient manner, including by aerosol inhalation, injection, ingestion, transfusion, implantation, or transplantation. The cells or population of cells may be administered to a patient subcutaneously, intradermally, intratumorally, intranodally, intramedullary, intramuscularly, intrathecally, by intravenous or intralymphatic injection, or intraperitoneally. In some embodiments, the disclosed CARs may be delivered or administered into a cavity formed by the resection of tumor tissue (i.e. intracavity delivery) or directly into a tumor prior to resection (i.e. intratumoral delivery). In one embodiment, the cell compositions of the present invention are preferably administered by intravenous injection.

The administration of the cells or population of cells can consist of the administration of 104-109 cells per kg body weight, preferably 105 to 106 cells/kg body weight including all integer values of cell numbers within those ranges. Dosing in CAR T cell therapies may for example involve administration of from 106 to 109 cells/kg, with or without a course of lymphodepletion, for example with cyclophosphamide. The cells or population of cells can be administrated in one or more doses. In another embodiment, the effective amount of cells are administrated as a single dose. In another embodiment, the effective amount of cells are administrated as more than one dose over a period time. Timing of administration is within the judgment of managing physician and depends on the clinical condition of the patient. The cells or population of cells may be obtained from any source, such as a blood bank or a donor. While individual needs vary, determination of optimal ranges of effective amounts of a given cell type for a particular disease or conditions are within the skill of one in the art. An effective amount means an amount which provides a therapeutic or prophylactic benefit. The dosage administrated will be dependent upon the age, health and weight of the recipient, kind of concurrent treatment, if any, frequency of treatment and the nature of the effect desired.

In another embodiment, the effective amount of cells or composition comprising those cells are administrated parenterally. The administration can be an intravenous administration. The administration can be directly done by injection within a tumor.

To guard against possible adverse reactions, engineered immunoresponsive cells may be equipped with a transgenic safety switch, in the form of a transgene that renders the cells vulnerable to exposure to a specific signal. For example, the herpes simplex viral thymidine kinase (TK) gene may be used in this way, for example by introduction into allogeneic T lymphocytes used as donor lymphocyte infusions following stem cell transplantation (Greco, et al., Improving the safety of cell therapy with the TK-suicide gene. Front. Pharmacol. 2015; 6: 95). In such cells, administration of a nucleoside prodrug such as ganciclovir or acyclovir causes cell death. Alternative safety switch constructs include inducible caspase 9, for example triggered by administration of a small-molecule dimerizer that brings together two nonfunctional icasp9 molecules to form the active enzyme. A wide variety of alternative approaches to implementing cellular proliferation controls have been described (see U.S. Patent Publication No. 20130071414; PCT Patent Publication WO2011146862; PCT Patent Publication WO2014011987; PCT Patent Publication WO2013040371; Zhou et al. BLOOD, 2014, 123/25:3895-3905; Di Stasi et al., The New England Journal of Medicine 2011; 365:1673-1683; Sadelain M, The New England Journal of Medicine 2011; 365:1735-173; Ramos et al., Stem Cells 28(6):1107-15 (2010)).

In a further refinement of adoptive therapies, genome editing may be used to tailor immunoresponsive cells to alternative implementations, for example providing edited CAR T cells (see Poirot et al., 2015, Multiplex genome edited T-cell manufacturing platform for “off-the-shelf” adoptive T-cell immunotherapies, Cancer Res 75 (18): 3853; Ren et al., 2017, Multiplex genome editing to generate universal CAR T cells resistant to PD1 inhibition, Clin Cancer Res. 2017 May 1; 23(9):2255-2266. doi: 10.1158/1078-0432.CCR-16-1300. Epub 2016 Nov. 4; Qasim et al., 2017, Molecular remission of infant B-ALL after infusion of universal TALEN gene-edited CAR T cells, Sci Transl Med. 2017 Jan. 25; 9(374); Legut, et al., 2018, CRISPR-mediated TCR replacement generates superior anticancer transgenic T cells. Blood, 131(3), 311-322; Georgiadis et al., Long Terminal Repeat CRISPR-CAR-Coupled “Universal” T Cells Mediate Potent Anti-leukemic Effects, Molecular Therapy, In Press, Corrected Proof, Available online 6 Mar. 2018; and Roth, T. L. Editing of Endogenous Genes in Cellular Immunotherapies. Curr Hematol Malig Rep 15, 235-240 (2020)). Cells may be edited using any CRISPR system and method of use thereof as described herein. CRISPR systems may be delivered to an immune cell by any method described herein. In preferred embodiments, cells are edited ex vivo and transferred to a subject in need thereof. Immunoresponsive cells, CAR T cells or any cells used for adoptive cell transfer may be edited. Editing may be performed for example to insert or knock-in an exogenous gene, such as an exogenous gene encoding a CAR or a TCR, at a preselected locus in a cell (e.g. TRAC locus); to eliminate potential alloreactive T-cell receptors (TCR) or to prevent inappropriate pairing between endogenous and exogenous TCR chains, such as to knock-out or knock-down expression of an endogenous TCR in a cell; to disrupt the target of a chemotherapeutic agent in a cell; to block an immune checkpoint, such as to knock-out or knock-down expression of an immune checkpoint protein or receptor in a cell; to knock-out or knock-down expression of other gene or genes in a cell, the reduced expression or lack of expression of which can enhance the efficacy of adoptive therapies using the cell; to knock-out or knock-down expression of an endogenous gene in a cell, said endogenous gene encoding an antigen targeted by an exogenous CAR or TCR; to knock-out or knock-down expression of one or more MHC constituent proteins in a cell; to activate a T cell; to modulate cells such that the cells are resistant to exhaustion or dysfunction; and/or increase the differentiation and/or proliferation of functionally exhausted or dysfunctional CD8+ T-cells (see PCT Patent Publications: WO2013176915, WO2014059173, WO2014172606, WO2014184744, and WO2014191128).

In certain embodiments, editing may result in inactivation of a gene. By inactivating a gene, it is intended that the gene of interest is not expressed in a functional protein form. In a particular embodiment, the CRISPR system specifically catalyzes cleavage in one targeted gene thereby inactivating said targeted gene. The nucleic acid strand breaks caused are commonly repaired through the distinct mechanisms of homologous recombination or non-homologous end joining (NHEJ). However, NHEJ is an imperfect repair process that often results in changes to the DNA sequence at the site of the cleavage. Repair via non-homologous end joining (NHEJ) often results in small insertions or deletions (Indel) and can be used for the creation of specific gene knockouts. Cells in which a cleavage induced mutagenesis event has occurred can be identified and/or selected by well-known methods in the art. In certain embodiments, homology directed repair (HDR) is used to concurrently inactivate a gene (e.g., TRAC) and insert an endogenous TCR or CAR into the inactivated locus.

Hence, in certain embodiments, editing of cells (such as by CRISPR/Cas), particularly cells intended for adoptive cell therapies, more particularly immunoresponsive cells such as T cells, may be performed to insert or knock-in an exogenous gene, such as an exogenous gene encoding a CAR or a TCR, at a preselected locus in a cell. Conventionally, nucleic acid molecules encoding CARs or TCRs are transfected or transduced to cells using randomly integrating vectors, which, depending on the site of integration, may lead to clonal expansion, oncogenic transformation, variegated transgene expression and/or transcriptional silencing of the transgene. Directing of transgene(s) to a specific locus in a cell can minimize or avoid such risks and advantageously provide for uniform expression of the transgene(s) by the cells. Without limitation, suitable ‘safe harbor’ loci for directed transgene integration include CCR5 or AAVS1. Homology-directed repair (HDR) strategies are known and described elsewhere in this specification allowing to insert transgenes into desired loci (e.g., TRAC locus).

Further suitable loci for insertion of transgenes, in particular CAR or exogenous TCR transgenes, include without limitation loci comprising genes coding for constituents of endogenous T-cell receptor, such as T-cell receptor alpha locus (TRA) or T-cell receptor beta locus (TRB), for example T-cell receptor alpha constant (TRAC) locus, T-cell receptor beta constant 1 (TRBC1) locus or T-cell receptor beta constant 2 (TRBC1) locus. Advantageously, insertion of a transgene into such locus can simultaneously achieve expression of the transgene, potentially controlled by the endogenous promoter, and knock-out expression of the endogenous TCR. This approach has been exemplified in Eyquem et al., (2017) Nature 543: 113-117, wherein the authors used CRISPR/Cas9 gene editing to knock-in a DNA molecule encoding a CD19-specific CAR into the TRAC locus downstream of the endogenous promoter; the CAR-T cells obtained by CRISPR were significantly superior in terms of reduced tonic CAR signaling and exhaustion.

T cell receptors (TCR) are cell surface receptors that participate in the activation of T cells in response to the presentation of antigen. The TCR is generally made from two chains, a and β, which assemble to form a heterodimer and associates with the CD3-transducing subunits to form the T cell receptor complex present on the cell surface. Each α and β chain of the TCR consists of an immunoglobulin-like N-terminal variable (V) and constant (C) region, a hydrophobic transmembrane domain, and a short cytoplasmic region. As for immunoglobulin molecules, the variable region of the α and β chains are generated by V(D)J recombination, creating a large diversity of antigen specificities within the population of T cells. However, in contrast to immunoglobulins that recognize intact antigen, T cells are activated by processed peptide fragments in association with an MHC molecule, introducing an extra dimension to antigen recognition by T cells, known as MHC restriction. Recognition of MHC disparities between the donor and recipient through the T cell receptor leads to T cell proliferation and the potential development of graft versus host disease (GVHD). The inactivation of TCRα or TCRβ can result in the elimination of the TCR from the surface of T cells preventing recognition of alloantigen and thus GVHD. However, TCR disruption generally results in the elimination of the CD3 signaling component and alters the means of further T cell expansion.

Hence, in certain embodiments, editing of cells (such as by CRISPR/Cas), particularly cells intended for adoptive cell therapies, more particularly immunoresponsive cells such as T cells, may be performed to knock-out or knock-down expression of an endogenous TCR in a cell. For example, NHEJ-based or HDR-based gene editing approaches can be employed to disrupt the endogenous TCR alpha and/or beta chain genes. For example, gene editing system or systems, such as CRISPR/Cas system or systems, can be designed to target a sequence found within the TCR beta chain conserved between the beta 1 and beta 2 constant region genes (TRBC1 and TRBC2) and/or to target the constant region of the TCR alpha chain (TRAC) gene.

Allogeneic cells are rapidly rejected by the host immune system. It has been demonstrated that, allogeneic leukocytes present in non-irradiated blood products will persist for no more than 5 to 6 days (Boni, Muranski et al. 2008 Blood 1; 112(12):4746-54). Thus, to prevent rejection of allogeneic cells, the host's immune system usually has to be suppressed to some extent. However, in the case of adoptive cell transfer the use of immunosuppressive drugs also have a detrimental effect on the introduced therapeutic T cells. Therefore, to effectively use an adoptive immunotherapy approach in these conditions, the introduced cells would need to be resistant to the immunosuppressive treatment. Thus, in a particular embodiment, the present invention further comprises a step of modifying T cells to make them resistant to an immunosuppressive agent, preferably by inactivating at least one gene encoding a target for an immunosuppressive agent. An immunosuppressive agent is an agent that suppresses immune function by one of several mechanisms of action. An immunosuppressive agent can be, but is not limited to a calcineurin inhibitor, a target of rapamycin, an interleukin-2 receptor α-chain blocker, an inhibitor of inosine monophosphate dehydrogenase, an inhibitor of dihydrofolic acid reductase, a corticosteroid or an immunosuppressive antimetabolite. The present invention allows conferring immunosuppressive resistance to T cells for immunotherapy by inactivating the target of the immunosuppressive agent in T cells. As non-limiting examples, targets for an immunosuppressive agent can be a receptor for an immunosuppressive agent such as: CD52, glucocorticoid receptor (GR), a FKBP family gene member and a cyclophilin family gene member.

In certain embodiments, editing of cells (such as by CRISPR/Cas), particularly cells intended for adoptive cell therapies, more particularly immunoresponsive cells such as T cells, may be performed to block an immune checkpoint, such as to knock-out or knock-down expression of an immune checkpoint protein or receptor in a cell. Immune checkpoints are inhibitory pathways that slow down or stop immune reactions and prevent excessive tissue damage from uncontrolled activity of immune cells. In certain embodiments, the immune checkpoint targeted is the programmed death-1 (PD-1 or CD279) gene (PDCD1) (see, e.g., Rupp U, Schumann K, Roybal K T, et al. CRISPR/Cas9-mediated PD-1 disruption enhances anti-tumor efficacy of human chimeric antigen receptor T cells. Sci Rep. 2017; 7(1):737). In other embodiments, the immune checkpoint targeted is cytotoxic T-lymphocyte-associated antigen (CTLA-4). In additional embodiments, the immune checkpoint targeted is another member of the CD28 and CTLA4 Ig superfamily such as BTLA, LAG3, ICOS, PDL1 or KTR. In further additional embodiments, the immune checkpoint targeted is a member of the TNFR superfamily such as CD40, OX40, CD137, GITR, CD27 or TIM-3.

Additional immune checkpoints include Src homology 2 domain-containing protein tyrosine phosphatase 1 (SHP-1) (Watson H A, et al., SHP-1: the next checkpoint target for cancer immunotherapy?Biochem Soc Trans. 2016 Apr. 15; 44(2):356-62). SHP-1 is a widely expressed inhibitory protein tyrosine phosphatase (PTP). In T-cells, it is a negative regulator of antigen-dependent activation and proliferation. It is a cytosolic protein, and therefore not amenable to antibody-mediated therapies, but its role in activation and proliferation makes it an attractive target for genetic manipulation in adoptive transfer strategies, such as chimeric antigen receptor (CAR) T cells. Immune checkpoints may also include T cell immunoreceptor with Ig and ITIM domains (TIGIT/Vstm3/WUCAM/VSIG9) and VISTA (Le Mercier I, et al., (2015) Beyond CTLA-4 and PD-1, the generation Z of negative checkpoint regulators. Front. Immunol. 6:418).

WO2014172606 relates to the use of MT1 and/or MT2 inhibitors to increase proliferation and/or activity of exhausted CD8+ T-cells and to decrease CD8+ T-cell exhaustion (e.g., decrease functionally exhausted or unresponsive CD8+ immune cells). In certain embodiments, metallothioneins are targeted by gene editing in adoptively transferred T cells.

In certain embodiments, targets of gene editing may be at least one targeted locus involved in the expression of an immune checkpoint protein. Such targets may include, but are not limited to CTLA4, PPP2CA, PPP2CB, PTPN6, PTPN22, PDCD1, ICOS (CD278), PDL1, KIR, LAG3, HAVCR2, BTLA, CD160, TIGIT, CD96, CRTAM, LAIR1, SIGLEC7, SIGLEC9, CD244 (2B4), TNFRSF10B, TNFRSF10A, CASP8, CASP10, CASP3, CASP6, CASP7, FADD, FAS, TGFBRII, TGFRBRI, SMAD2, SMAD3, SMAD4, SMAD10, SKI, SKIL, TGIF1, IL10RA, ILIORB, HMOX2, IL6R, IL6ST, EIF2AK4, CSK, PAG1, SIT1, FOXP3, PRDM1, BATF, VISTA, GUCYlA2, GUCYlA3, GUCYlB2, GUCYlB3, MT1, MT2, CD40, OX40, CD137, GITR, CD27, SHP-1, TIM-3, CEACAM-1, CEACAM-3, or CEACAM-5. In preferred embodiments, the gene locus involved in the expression of PD-1 or CTLA-4 genes is targeted. In other preferred embodiments, combinations of genes are targeted, such as but not limited to PD-1 and TIGIT.

By means of an example and without limitation, WO2016196388 concerns an engineered T cell comprising (a) a genetically engineered antigen receptor that specifically binds to an antigen, which receptor may be a CAR; and (b) a disrupted gene encoding a PD-L1, an agent for disruption of a gene encoding a PD-L1, and/or disruption of a gene encoding PD-L1, wherein the disruption of the gene may be mediated by a gene editing nuclease, a zinc finger nuclease (ZFN), CRISPR/Cas9 and/or TALEN. WO2015142675 relates to immune effector cells comprising a CAR in combination with an agent (such as CRISPR, TALEN or ZFN) that increases the efficacy of the immune effector cells in the treatment of cancer, wherein the agent may inhibit an immune inhibitory molecule, such as PD1, PD-L1, CTLA-4, TIM-3, LAG-3, VISTA, BTLA, TIGIT, LAIR1, CD160, 2B4, TGFR beta, CEACAM-1, CEACAM-3, or CEACAM-5. Ren et al., (2017) Clin Cancer Res 23 (9) 2255-2266 performed lentiviral delivery of CAR and electro-transfer of Cas9 mRNA and gRNAs targeting endogenous TCR, 3-2 microglobulin (B2M) and PD1 simultaneously, to generate gene-disrupted allogeneic CAR T cells deficient of TCR, HLA class I molecule and PD1.

In certain embodiments, cells may be engineered to express a CAR, wherein expression and/or function of methylcytosine dioxygenase genes (TET1, TET2 and/or TET3) in the cells has been reduced or eliminated, such as by CRISPR, ZNF or TALEN (for example, as described in WO201704916).

In certain embodiments, editing of cells (such as by CRISPR/Cas), particularly cells intended for adoptive cell therapies, more particularly immunoresponsive cells such as T cells, may be performed to knock-out or knock-down expression of an endogenous gene in a cell, said endogenous gene encoding an antigen targeted by an exogenous CAR or TCR, thereby reducing the likelihood of targeting of the engineered cells. In certain embodiments, the targeted antigen may be one or more antigen selected from the group consisting of CD38, CD138, CS-1, CD33, CD26, CD30, CD53, CD92, CD100, CD148, CD150, CD200, CD261, CD262, CD362, human telomerase reverse transcriptase (hTERT), survivin, mouse double minute 2 homolog (MDM2), cytochrome P450 1B1 (CYP1B), HER2/neu, Wilms' tumor gene 1 (WT1), livin, alphafetoprotein (AFP), carcinoembryonic antigen (CEA), mucin 16 (MUC16), MUC1, prostate-specific membrane antigen (PSMA), p53, cyclin (D1), B cell maturation antigen (BCMA), transmembrane activator and CAML Interactor (TACI), and B-cell activating factor receptor (BAFF-R) (for example, as described in WO2016011210 and WO2017011804).

In certain embodiments, editing of cells (such as by CRISPR/Cas), particularly cells intended for adoptive cell therapies, more particularly immunoresponsive cells such as T cells, may be performed to knock-out or knock-down expression of one or more MHC constituent proteins, such as one or more HLA proteins and/or beta-2 microglobulin (B2M), in a cell, whereby rejection of non-autologous (e.g., allogeneic) cells by the recipient's immune system can be reduced or avoided. In preferred embodiments, one or more HLA class I proteins, such as HLA-A, B and/or C, and/or B2M may be knocked-out or knocked-down. Preferably, B2M may be knocked-out or knocked-down. By means of an example, Ren et al., (2017) Clin Cancer Res 23 (9) 2255-2266 performed lentiviral delivery of CAR and electro-transfer of Cas9 mRNA and gRNAs targeting endogenous TCR, β-2 microglobulin (B2M) and PD1 simultaneously, to generate gene-disrupted allogeneic CAR T cells deficient of TCR, HLA class I molecule and PD1.

In other embodiments, at least two genes are edited. Pairs of genes may include, but are not limited to PD1 and TCRα, PD1 and TCRβ, CTLA-4 and TCRα, CTLA-4 and TCRβ, LAG3 and TCRα, LAG3 and TCRβ, Tim3 and TCRα, Tim3 and TCRβ, BTLA and TCRα, BTLA and TCRβ, BY55 and TCRα, BY55 and TCRβ, TIGIT and TCRα, TIGIT and TCRβ, B7H5 and TCRα, B7H5 and TCRβ, LAIR1 and TCRα, LAIR1 and TCRβ, SIGLEC10 and TCRα, SIGLEC10 and TCRβ, 2B4 and TCRα, 2B4 and TCRβ, B2M and TCRα, B2M and TCRβ.

In certain embodiments, a cell may be multiply edited (multiplex genome editing) as taught herein to (1) knock-out or knock-down expression of an endogenous TCR (for example, TRBC1, TRBC2 and/or TRAC), (2) knock-out or knock-down expression of an immune checkpoint protein or receptor (for example PD1, PD-L1 and/or CTLA4); and (3) knock-out or knock-down expression of one or more MHC constituent proteins (for example, HLA-A, B and/or C, and/or B2M, preferably B2M).

Whether prior to or after genetic modification of the T cells, the T cells can be activated and expanded generally using methods as described, for example, in U.S. Pat. Nos. 6,352,694; 6,534,055; 6,905,680; 5,858,358; 6,887,466; 6,905,681; 7,144,575; 7,232,566; 7,175,843; 5,883,223; 6,905,874; 6,797,514; 6,867,041; and 7,572,631. T cells can be expanded in vitro or in vivo.

Immune cells may be obtained using any method known in the art. In one embodiment, allogenic T cells may be obtained from healthy subjects. In one embodiment T cells that have infiltrated a tumor are isolated. T cells may be removed during surgery. T cells may be isolated after removal of tumor tissue by biopsy. T cells may be isolated by any means known in the art. In one embodiment, T cells are obtained by apheresis. In one embodiment, the method may comprise obtaining a bulk population of T cells from a tumor sample by any suitable method known in the art. For example, a bulk population of T cells can be obtained from a tumor sample by dissociating the tumor sample into a cell suspension from which specific cell populations can be selected. Suitable methods of obtaining a bulk population of T cells may include, but are not limited to, any one or more of mechanically dissociating (e.g., mincing) the tumor, enzymatically dissociating (e.g., digesting) the tumor, and aspiration (e.g., as with a needle).

The bulk population of T cells obtained from a tumor sample may comprise any suitable type of T cell. Preferably, the bulk population of T cells obtained from a tumor sample comprises tumor infiltrating lymphocytes (TILs).

The tumor sample may be obtained from any mammal. Unless stated otherwise, as used herein, the term “mammal” refers to any mammal including, but not limited to, mammals of the order Logomorpha, such as rabbits; the order Carnivora, including Felines (cats) and Canines (dogs); the order Artiodactyla, including Bovines (cows) and Swines (pigs); or of the order Perssodactyla, including Equines (horses). The mammals may be non-human primates, e.g., of the order Primates, Ceboids, or Simoids (monkeys) or of the order Anthropoids (humans and apes). In some embodiments, the mammal may be a mammal of the order Rodentia, such as mice and hamsters. Preferably, the mammal is a non-human primate or a human. An especially preferred mammal is the human.

T cells can be obtained from a number of sources, including peripheral blood mononuclear cells (PBMC), bone marrow, lymph node tissue, spleen tissue, and tumors. In certain embodiments of the present invention, T cells can be obtained from a unit of blood collected from a subject using any number of techniques known to the skilled artisan, such as Ficoll separation. In one preferred embodiment, cells from the circulating blood of an individual are obtained by apheresis or leukapheresis. The apheresis product typically contains lymphocytes, including T cells, monocytes, granulocytes, B cells, other nucleated white blood cells, red blood cells, and platelets. In one embodiment, the cells collected by apheresis may be washed to remove the plasma fraction and to place the cells in an appropriate buffer or media for subsequent processing steps. In one embodiment of the invention, the cells are washed with phosphate buffered saline (PBS). In an alternative embodiment, the wash solution lacks calcium and may lack magnesium or may lack many if not all divalent cations. Initial activation steps in the absence of calcium lead to magnified activation. As those of ordinary skill in the art would readily appreciate a washing step may be accomplished by methods known to those in the art, such as by using a semi-automated “flow-through” centrifuge (for example, the Cobe 2991 cell processor) according to the manufacturer's instructions. After washing, the cells may be resuspended in a variety of biocompatible buffers, such as, for example, Ca-free, Mg-free PBS. Alternatively, the undesirable components of the apheresis sample may be removed, and the cells directly resuspended in culture media.

In another embodiment, T cells are isolated from peripheral blood lymphocytes by lysing the red blood cells and depleting the monocytes, for example, by centrifugation through a PERCOLL™ gradient. A specific subpopulation of T cells, such as CD28+, CD4+, CDC, CD45RA+, and CD45RO+ T cells, can be further isolated by positive or negative selection techniques. For example, in one preferred embodiment, T cells are isolated by incubation with anti-CD3/anti-CD28 (i.e., 3×28)-conjugated beads, such as DYNABEADS® M-450 CD3/CD28 T, or XCYTE DYNABEADS™ for a time period sufficient for positive selection of the desired T cells. In one embodiment, the time period is about 30 minutes. In a further embodiment, the time period ranges from 30 minutes to 36 hours or longer and all integer values there between. In a further embodiment, the time period is at least 1, 2, 3, 4, 5, or 6 hours. In yet another preferred embodiment, the time period is 10 to 24 hours. In one preferred embodiment, the incubation time period is 24 hours. For isolation of T cells from patients with leukemia, use of longer incubation times, such as 24 hours, can increase cell yield. Longer incubation times may be used to isolate T cells in any situation where there are few T cells as compared to other cell types, such in isolating tumor infiltrating lymphocytes (TIL) from tumor tissue or from immunocompromised individuals. Further, use of longer incubation times can increase the efficiency of capture of CD8+ T cells.

Enrichment of a T cell population by negative selection can be accomplished with a combination of antibodies directed to surface markers unique to the negatively selected cells. A preferred method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8.

Further, monocyte populations (i.e., CD14+ cells) may be depleted from blood preparations by a variety of methodologies, including anti-CD14 coated beads or columns, or utilization of the phagocytotic activity of these cells to facilitate removal. Accordingly, in one embodiment, the invention uses paramagnetic particles of a size sufficient to be engulfed by phagocytotic monocytes. In certain embodiments, the paramagnetic particles are commercially available beads, for example, those produced by Life Technologies under the trade name Dynabeads™. In one embodiment, other non-specific cells are removed by coating the paramagnetic particles with “irrelevant” proteins (e.g., serum proteins or antibodies). Irrelevant proteins and antibodies include those proteins and antibodies or fragments thereof that do not specifically target the T cells to be isolated. In certain embodiments, the irrelevant beads include beads coated with sheep anti-mouse antibodies, goat anti-mouse antibodies, and human serum albumin.

In brief, such depletion of monocytes is performed by preincubating T cells isolated from whole blood, apheresed peripheral blood, or tumors with one or more varieties of irrelevant or non-antibody coupled paramagnetic particles at any amount that allows for removal of monocytes (approximately a 20:1 bead:cell ratio) for about 30 minutes to 2 hours at 22 to 37 degrees C., followed by magnetic removal of cells which have attached to or engulfed the paramagnetic particles. Such separation can be performed using standard methods available in the art. For example, any magnetic separation methodology may be used including a variety of which are commercially available, (e.g., DYNAL® Magnetic Particle Concentrator (DYNAL MPC®)). Assurance of requisite depletion can be monitored by a variety of methodologies known to those of ordinary skill in the art, including flow cytometric analysis of CD14 positive cells, before and after depletion.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (i.e., leukemic blood, tumor tissue, etc). Such populations of cells may have therapeutic value and would be desirable to obtain. For example, using high concentration of cells allows more efficient selection of CD8+ T cells that normally have weaker CD28 expression.

In a related embodiment, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4+ T cells express higher levels of CD28 and are more efficiently captured than CD8+ T cells in dilute concentrations. In one embodiment, the concentration of cells used is 5×106/ml. In other embodiments, the concentration used can be from about 1×105/ml to 1×106/ml, and any integer value in between.

T cells can also be frozen. Wishing not to be bound by theory, the freeze and subsequent thaw step provides a more uniform product by removing granulocytes and to some extent monocytes in the cell population. After a washing step to remove plasma and platelets, the cells may be suspended in a freezing solution. While many freezing solutions and parameters are known in the art and will be useful in this context, one method involves using PBS containing 20% DMSO and 8% human serum albumin, or other suitable cell freezing media, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

T cells for use in the present invention may also be antigen-specific T cells. For example, tumor-specific T cells can be used. In certain embodiments, antigen-specific T cells can be isolated from a patient of interest, such as a patient afflicted with a cancer or an infectious disease. In one embodiment, neoepitopes are determined for a subject and T cells specific to these antigens are isolated. Antigen-specific cells for use in expansion may also be generated in vitro using any number of methods known in the art, for example, as described in U.S. Patent Publication No. US 20040224402 entitled, Generation and Isolation of Antigen-Specific T Cells, or in U.S. Pat. Nos. 6,040,177. Antigen-specific cells for use in the present invention may also be generated using any number of methods known in the art, for example, as described in Current Protocols in Immunology, or Current Protocols in Cell Biology, both published by John Wiley & Sons, Inc., Boston, Mass.

In a related embodiment, it may be desirable to sort or otherwise positively select (e.g. via magnetic selection) the antigen specific cells prior to or following one or two rounds of expansion. Sorting or positively selecting antigen-specific cells can be carried out using peptide-MHC tetramers (Altman, et al., Science. 1996 Oct. 4; 274(5284):94-6). In another embodiment, the adaptable tetramer technology approach is used (Andersen et al., 2012 Nat Protoc. 7:891-902). Tetramers are limited by the need to utilize predicted binding peptides based on prior hypotheses, and the restriction to specific HLAs. Peptide-MHC tetramers can be generated using techniques known in the art and can be made with any MHC molecule of interest and any antigen of interest as described herein. Specific epitopes to be used in this context can be identified using numerous assays known in the art. For example, the ability of a polypeptide to bind to MHC class I may be evaluated indirectly by monitoring the ability to promote incorporation of 125I labeled β2-microglobulin (β2m) into MHC class I/β2m/peptide heterotrimeric complexes (see Parker et al., J. Immunol. 152:163, 1994).

In one embodiment cells are directly labeled with an epitope-specific reagent for isolation by flow cytometry followed by characterization of phenotype and TCRs. In one embodiment, T cells are isolated by contacting with T cell specific antibodies. Sorting of antigen-specific T cells, or generally any cells of the present invention, can be carried out using any of a variety of commercially available cell sorters, including, but not limited to, MoFlo sorter (DakoCytomation, Fort Collins, Colo.), FACSAria™, FACSArray™, FACSVantage™, BD™ LSR II, and FACSCalibur™ (BD Biosciences, San Jose, Calif.).

In a preferred embodiment, the method comprises selecting cells that also express CD3. The method may comprise specifically selecting the cells in any suitable manner. Preferably, the selecting is carried out using flow cytometry. The flow cytometry may be carried out using any suitable method known in the art. The flow cytometry may employ any suitable antibodies and stains. Preferably, the antibody is chosen such that it specifically recognizes and binds to the particular biomarker being selected. For example, the specific selection of CD3, CD8, TIM-3, LAG-3, 4-1BB, or PD-1 may be carried out using anti-CD3, anti-CD8, anti-TIM-3, anti-LAG-3, anti-4-1BB, or anti-PD-1 antibodies, respectively. The antibody or antibodies may be conjugated to a bead (e.g., a magnetic bead) or to a fluorochrome. Preferably, the flow cytometry is fluorescence-activated cell sorting (FACS). TCRs expressed on T cells can be selected based on reactivity to autologous tumors. Additionally, T cells that are reactive to tumors can be selected for based on markers using the methods described in patent publication Nos. WO2014133567 and WO2014133568, herein incorporated by reference in their entirety. Additionally, activated T cells can be selected for based on surface expression of CD107a.

In one embodiment of the invention, the method further comprises expanding the numbers of T cells in the enriched cell population. Such methods are described in U.S. Pat. No. 8,637,307 and is herein incorporated by reference in its entirety. The numbers of T cells may be increased at least about 3-fold (or 4-, 5-, 6-, 7-, 8-, or 9-fold), more preferably at least about 10-fold (or 20-, 30-, 40-, 50-, 60-, 70-, 80-, or 90-fold), more preferably at least about 100-fold, more preferably at least about 1,000-fold, or most preferably at least about 100,000-fold. The numbers of T cells may be expanded using any suitable method known in the art. Exemplary methods of expanding the numbers of cells are described in patent publication No. WO 2003057171, U.S. Pat. No. 8,034,334, and U.S. Patent Application Publication No. 2012/0244133, each of which is incorporated herein by reference.

In one embodiment, ex vivo T cell expansion can be performed by isolation of T cells and subsequent stimulation or activation followed by further expansion. In one embodiment of the invention, the T cells may be stimulated or activated by a single agent. In another embodiment, T cells are stimulated or activated with two agents, one that induces a primary signal and a second that is a co-stimulatory signal. Ligands useful for stimulating a single signal or stimulating a primary signal and an accessory molecule that stimulates a second signal may be used in soluble form. Ligands may be attached to the surface of a cell, to an Engineered Multivalent Signaling Platform (EMSP), or immobilized on a surface. In a preferred embodiment both primary and secondary agents are co-immobilized on a surface, for example a bead or a cell. In one embodiment, the molecule providing the primary activation signal may be a CD3 ligand, and the co-stimulatory molecule may be a CD28 ligand or 4-1BB ligand.

In certain embodiments, T cells comprising a CAR or an exogenous TCR, may be manufactured as described in WO2015120096, by a method comprising: enriching a population of lymphocytes obtained from a donor subject; stimulating the population of lymphocytes with one or more T-cell stimulating agents to produce a population of activated T cells, wherein the stimulation is performed in a closed system using serum-free culture medium; transducing the population of activated T cells with a viral vector comprising a nucleic acid molecule which encodes the CAR or TCR, using a single cycle transduction to produce a population of transduced T cells, wherein the transduction is performed in a closed system using serum-free culture medium; and expanding the population of transduced T cells for a predetermined time to produce a population of engineered T cells, wherein the expansion is performed in a closed system using serum-free culture medium. In certain embodiments, T cells comprising a CAR or an exogenous TCR, may be manufactured as described in WO2015120096, by a method comprising: obtaining a population of lymphocytes; stimulating the population of lymphocytes with one or more stimulating agents to produce a population of activated T cells, wherein the stimulation is performed in a closed system using serum-free culture medium; transducing the population of activated T cells with a viral vector comprising a nucleic acid molecule which encodes the CAR or TCR, using at least one cycle transduction to produce a population of transduced T cells, wherein the transduction is performed in a closed system using serum-free culture medium; and expanding the population of transduced T cells to produce a population of engineered T cells, wherein the expansion is performed in a closed system using serum-free culture medium. The predetermined time for expanding the population of transduced T cells may be 3 days. The time from enriching the population of lymphocytes to producing the engineered T cells may be 6 days. The closed system may be a closed bag system. Further provided is population of T cells comprising a CAR or an exogenous TCR obtainable or obtained by said method, and a pharmaceutical composition comprising such cells.

In certain embodiments, T cell maturation or differentiation in vitro may be delayed or inhibited by the method as described in WO2017070395, comprising contacting one or more T cells from a subject in need of a T cell therapy with an AKT inhibitor (such as, e.g., one or a combination of two or more AKT inhibitors disclosed in claim 8 of WO2017070395) and at least one of exogenous Interleukin-7 (IL-7) and exogenous Interleukin-15 (IL-15), wherein the resulting T cells exhibit delayed maturation or differentiation, and/or wherein the resulting T cells exhibit improved T cell function (such as, e.g., increased T cell proliferation; increased cytokine production; and/or increased cytolytic activity) relative to a T cell function of a T cell cultured in the absence of an AKT inhibitor.

In certain embodiments, a patient in need of a T cell therapy may be conditioned by a method as described in WO2016191756 comprising administering to the patient a dose of cyclophosphamide between 200 mg/m2/day and 2000 mg/m2/day and a dose of fludarabine between 20 mg/m2/day and 900 mg/m2/day.

In certain embodiments, a patient in need of adoptive cell transfer may be administered a TLR agonist to enhance anti-tumor immunity (see, e.g., Urban-Wojciuk, et al., The Role of TLRs in Anti-cancer Immunity and Tumor Rejection, Front Immunol. 2019; 10: 2388; and Kaczanowska et al., TLR agonists: our best frenemy in cancer immunotherapy, J Leukoc Biol. 2013 June; 93(6): 847-863). In certain embodiments, TLR agonists are delivered in a nanoparticle system (see, e.g., Buss and Bhatia, Nanoparticle delivery of immunostimulatory oligonucleotides enhances response to checkpoint inhibitor therapeutics, Proc Natl Acad Sci USA. 2020 Jun. 3; 202001569). In certain embodiments, the agonist is a TLR9 agonist. Id.

### Disease Applications

In one example embodiment, the disease is associated with cancer. In particular, the disease is oncogenic. Many oncogenic targets are known and can be regulated by posttranslational modifications. See, e.g. Chen, L., Liu, S. & Tao, Y. Regulating tumor suppressor genes: post-translational modifications. Sig Transduct Target Ther 5, 90 (2020); doi:10.1038/s41392-020-0196-9. Exemplary post-translational modification types of proteins implicated in oncogenesis and their expression pattern are found in Table 1 of Sharma, et al., (2019). Post-Translational Modifications (PTMs), from a Cancer Perspective: An Overview. Oncogen 2(3): 12, specifically incorporated herein by reference. In another embodiment, the chimeric small molecule and/or bifunctional immune cell engager are designed to induce an immune response against an infectious disease., an autoimmune disease, a neurological disease (e.g., Alzheimer's disease, Parkinson's disease), anti-addiction (e.g., nicotine addition, opioid addiction), allergies, cardiovascular disease, or age-related diseases,

### Pharmaceutical Formulations

Also described herein are pharmaceutical formulations that can contain an amount, effective amount, and/or least effective amount, and/or therapeutically effective amount of one or more compounds, molecules, compositions, vectors, vector systems, cells, or a combination thereof (which are also referred to as the primary active agent or ingredient elsewhere herein) described in greater detail elsewhere herein a pharmaceutically acceptable carrier or excipient. As used herein, “pharmaceutical formulation” refers to the combination of an active agent, compound, or ingredient with a pharmaceutically acceptable carrier or excipient, making the composition suitable for diagnostic, therapeutic, or preventive use in vitro, in vivo, or ex vivo. As used herein, “pharmaceutically acceptable carrier or excipient” refers to a carrier or excipient that is useful in preparing a pharmaceutical formulation that is generally safe, non-toxic, and is neither biologically or otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient. When present, the compound can optionally be present in the pharmaceutical formulation as a pharmaceutically acceptable salt. In some embodiments, the pharmaceutical formulation can include, such as an active ingredient, a CRISPR-Cas system or component thereof described in greater detail elsewhere herein. In some embodiments, the pharmaceutical formulation can include, such as an active ingredient, a CRISPR-Cas polynucleotide described in greater detail elsewhere herein. In some embodiments, the pharmaceutical formulation can include, such as an active ingredient one or more modified cells, such as one or more modified cells described in greater detail elsewhere herein.

In some embodiments, the active ingredient is present as a pharmaceutically acceptable salt of the active ingredient. As used herein, “pharmaceutically acceptable salt” refers to any acid or base addition salt whose counter-ions are non-toxic to the subject to which they are administered in pharmaceutical doses of the salts. Suitable salts include, hydrobromide, iodide, nitrate, bisulfate, phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, malonate, mandelate, malate, phthalate, and pamoate.

The pharmaceutical formulations described herein can be administered to a subject in need thereof via any suitable method or route to a subject in need thereof. Suitable administration routes can include, but are not limited to auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratympanic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated and/or the active ingredient(s).

Where appropriate, compounds, molecules, compositions, vectors, vector systems, cells, or a combination thereof described in greater detail elsewhere herein can be provided to a subject in need thereof as an ingredient, such as an active ingredient or agent, in a pharmaceutical formulation. As such, also described are pharmaceutical formulations containing one or more of the compounds and salts thereof, or pharmaceutically acceptable salts thereof described herein. Suitable salts include, hydrobromide, iodide, nitrate, bisulfate, phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, malonate, mandelate, malate, phthalate, and pamoate.

In some embodiments, the subject in need thereof has or is suspected of having a cancer or a symptom thereof. In some embodiments, the subject in need thereof has or is suspected of having, a neurobiological disease or disorder, a psychiatric disease or disorder, a cancer, an autoimmune disease or disorder, a thrombosis disease, a heart disease, a kidney disease, a lung disease, or a blood vessel disease, or a combination thereof. As used herein, “agent” refers to any substance, compound, molecule, and the like, which can be biologically active or otherwise can induce a biological and/or physiological effect on a subject to which it is administered to. As used herein, “active agent” or “active ingredient” refers to a substance, compound, or molecule, which is biologically active or otherwise, induces a biological or physiological effect on a subject to which it is administered to. In other words, “active agent” or “active ingredient” refers to a component or components of a composition to which the whole or part of the effect of the composition is attributed. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed.

### Pharmaceutically Acceptable Carriers and Secondary Ingredients and Agents

The pharmaceutical formulation can include a pharmaceutically acceptable carrier. Suitable pharmaceutically acceptable carriers include, but are not limited to water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid esters, hydroxy methylcellulose, and polyvinyl pyrrolidone, which do not deleteriously react with the active composition.

The pharmaceutical formulations can be sterilized, and if desired, mixed with agents, such as lubricants, preservatives, stabilizers, wetting agents, emulsifiers, salts for influencing osmotic pressure, buffers, coloring, flavoring and/or aromatic substances, and the like which do not deleteriously react with the active compound.

In some embodiments, the pharmaceutical formulation can also include an effective amount of secondary active agents, including but not limited to, biologic agents or molecules including, but not limited to, e.g. polynucleotides, amino acids, peptides, polypeptides, antibodies, aptamers, ribozymes, hormones, immunomodulators, antipyretics, anxiolytics, antipsychotics, analgesics, antispasmodics, anti-inflammatories, anti-histamines, anti-infectives, chemotherapeutics, and combinations thereof.

### Effective Amounts

In some embodiments, the amount of the primary active agent and/or optional secondary agent can be an effective amount, least effective amount, and/or therapeutically effective amount. As used herein, “effective amount” refers to the amount of the primary and/or optional secondary agent included in the pharmaceutical formulation that achieve one or more therapeutic effects or desired effect. As used herein, “least effective” amount refers to the lowest amount of the primary and/or optional secondary agent that achieves the one or more therapeutic or other desired effects. As used herein, “therapeutically effective amount” refers to the amount of the primary and/or optional secondary agent included in the pharmaceutical formulation that achieves one or more therapeutic effects.

The effective amount, least effective amount, and/or therapeutically effective amount of the primary and optional secondary active agent described elsewhere herein contained in the pharmaceutical formulation can range from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pg, ng, g, mg, or g or be any numerical value with any of these ranges.

In some embodiments, the effective amount, least effective amount, and/or therapeutically effective amount can be an effective concentration, least effective concentration, and/or therapeutically effective concentration, which can each range from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pM, nM, M, mM, or M or be any numerical value with any of these ranges.

In other embodiments, the effective amount, least effective amount, and/or therapeutically effective amount of the primary and optional secondary active agent can range from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 IU or be any numerical value with any of these ranges.

In some embodiments, the primary and/or the optional secondary active agent present in the pharmaceutical formulation can range from about 0 to 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.9, to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the pharmaceutical formulation.

In some embodiments where a cell population is present in the pharmaceutical formulation (e.g., as a primary and/or or secondary active agent), the effective amount of cells can range from about 2 cells to 1×10¹/mL, 1×10²⁰/mL or more, such as about 1×10¹/mL, 1×10²/mL, 1×10³/mL, 1×10⁴/mL, 1×10⁵/mL, 1×10⁶/mL, 1×10⁷/mL, 1×10⁸/mL, 1×10⁹/mL, 1×10¹⁰/mL, 1×10¹¹/mL, 1×10¹²/mL, 1×10¹³/mL, 1×10¹⁴/mL, 1×10¹⁵/mL, 1×10¹⁶/mL, 1×10¹⁷/mL, 1×10¹⁸/mL, 1×10¹⁹/mL, to/or about 1×10²⁰/mL.

In some embodiments, the amount or effective amount, particularly where an infective particle is being delivered (e.g. a virus particle having the primary or secondary agent as a cargo), the effective amount of virus particles can be expressed as a titer (plaque forming units per unit of volume) or as a MOI (multiplicity of infection). In some embodiments, the effective amount can be 1×10¹ particles per pL, nL, L, mL, or L to 1×10²⁰/particles per pL, nL, L, mL, or L or more, such as about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, to/or about 1×10²⁰ particles per pL, nL, L, mL, or L. In some embodiments, the effective titer can be about 1×10¹ transforming units per pL, nL, L, mL, or L to 1×10²⁰/transforming units per pL, nL, pL, mL, or L or more, such as about 1×10¹, 1×10², 1×10³, 1×10⁴, 1×10⁵, 1×10⁶, 1×10⁷, 1×10⁸, 1×10⁹, 1×10¹⁰, 1×10¹, 1×10¹², 1×10¹³, 1×10¹⁴, 1×10¹⁵, 1×10¹⁶, 1×10¹⁷, 1×10¹⁸, 1×10¹⁹, to/or about 1×10²⁰ transforming units per pL, nL, L, mL, or L. In some embodiments, the MOI of the pharmaceutical formulation can range from about 0.1 to 10 or more, such as 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10 or more.

In some embodiments, the amount or effective amount of the one or more of the active agent(s) described herein contained in the pharmaceutical formulation can range from about 1 μg/kg to about 10 mg/kg based upon the bodyweight of the subject in need thereof or average bodyweight of the specific patient population to which the pharmaceutical formulation can be administered.

In one example embodiment where there is a secondary agent contained in the pharmaceutical formulation, the effective amount of the secondary active agent will vary depending on the secondary agent, the primary agent, the administration route, subject age, disease, stage of disease, among other things, which will be one of ordinary skill in the art.

When optionally present in the pharmaceutical formulation, the secondary active agent can be included in the pharmaceutical formulation or can exist as a stand-alone compound or pharmaceutical formulation that can be administered contemporaneously or sequentially with the compound, derivative thereof, or pharmaceutical formulation thereof.

In some embodiments, the effective amount of the secondary active agent can range from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the total secondary active agent in the pharmaceutical formulation. In additional embodiments, the effective amount of the secondary active agent can range from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the total pharmaceutical formulation.

### Dosage Forms

In some embodiments, the pharmaceutical formulations described herein can be provided in a dosage form. The dosage form can be administered to a subject in need thereof. The dosage form can be effective generate specific concentration, such as an effective concentration, at a given site in the subject in need thereof. As used herein, “dose,” “unit dose,” or “dosage” can refer to physically discrete units suitable for use in a subject, each unit containing a predetermined quantity of the primary active agent, and optionally present secondary active ingredient, and/or a pharmaceutical formulation thereof calculated to produce the desired response or responses in association with its administration. In some embodiments, the given site is proximal to the administration site. In some embodiments, the given site is distal to the administration site. In some cases, the dosage form contains a greater amount of one or more of the active ingredients present in the pharmaceutical formulation than the final intended amount needed to reach a specific region or location within the subject to account for loss of the active components such as via first and second pass metabolism.

The dosage forms can be adapted for administration by any appropriate route. Appropriate routes include, but are not limited to, oral (including buccal or sublingual), rectal, intraocular, inhaled, intranasal, topical (including buccal, sublingual, or transdermal), vaginal, parenteral, subcutaneous, intramuscular, intravenous, internasal, and intradermal. Other appropriate routes are described elsewhere herein. Such formulations can be prepared by any method known in the art.

Dosage forms adapted for oral administration can discrete dosage units such as capsules, pellets or tablets, powders or granules, solutions, or suspensions in aqueous or non-aqueous liquids; edible foams or whips, or in oil-in-water liquid emulsions or water-in-oil liquid emulsions. In some embodiments, the pharmaceutical formulations adapted for oral administration also include one or more agents which flavor, preserve, color, or help disperse the pharmaceutical formulation. Dosage forms prepared for oral administration can also be in the form of a liquid solution that can be delivered as a foam, spray, or liquid solution. The oral dosage form can be administered to a subject in need thereof. Where appropriate, the dosage forms described herein can be microencapsulated.

The dosage form can also be prepared to prolong or sustain the release of any ingredient. In some embodiments, compounds, molecules, compositions, vectors, vector systems, cells, or a combination thereof described herein can be the ingredient whose release is delayed. In some embodiments the primary active agent is the ingredient whose release is delayed. In some embodiments, an optional secondary agent can be the ingredient whose release is delayed. Suitable methods for delaying the release of an ingredient include, but are not limited to, coating or embedding the ingredients in material in polymers, wax, gels, and the like. Delayed release dosage formulations can be prepared as described in standard references such as “Pharmaceutical dosage form tablets,” eds. Liberman et. al. (New York, Marcel Dekker, Inc., 1989), “Remington—The science and practice of pharmacy”, 20th ed., Lippincott Williams & Wlkins, Baltimore, MD, 2000, and “Pharmaceutical dosage forms and drug delivery systems”, 6th Edition, Ansel et al., (Media, PA: Wlliams and Wlkins, 1995). These references provide information on excipients, materials, equipment, and processes for preparing tablets and capsules and delayed release dosage forms of tablets and pellets, capsules, and granules. The delayed release can be anywhere from about an hour to about 3 months or more.

Examples of suitable coating materials include, but are not limited to, cellulose polymers such as cellulose acetate phthalate, hydroxypropyl cellulose, hydroxypropyl methylcellulose, hydroxypropyl methylcellulose phthalate, and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Coatings may be formed with a different ratio of water-soluble polymer, water insoluble polymers, and/or pH dependent polymers, with or without water insoluble/water soluble non-polymeric excipient, to produce the desired release profile. The coating is either performed on the dosage form (matrix or simple) which includes, but is not limited to, tablets (compressed with or without coated beads), capsules (with or without coated beads), beads, particle compositions, “ingredient as is” formulated as, but not limited to, suspension form or as a sprinkle dosage form.

Where appropriate, the dosage forms described herein can be a liposome. In these embodiments, primary active ingredient(s), and/or optional secondary active ingredient(s), and/or pharmaceutically acceptable salt thereof where appropriate are incorporated into a liposome. In one example embodiment where the dosage form is a liposome, the pharmaceutical formulation is thus a liposomal formulation. The liposomal formulation can be administered to a subject in need thereof. Dosage forms adapted for topical administration can be formulated as ointments, creams, suspensions, lotions, powders, solutions, pastes, gels, sprays, aerosols, or oils. In some embodiments for treatments of the eye or other external tissues, for example the mouth or the skin, the pharmaceutical formulations are applied as a topical ointment or cream. When formulated in an ointment, a primary active ingredient, optional secondary active ingredient, and/or pharmaceutically acceptable salt thereof where appropriate can be formulated with a paraffinic or water-miscible ointment base. In other embodiments, the primary and/or secondary active ingredient can be formulated in a cream with an oil-in-water cream base or a water-in-oil base. Dosage forms adapted for topical administration in the mouth include lozenges, pastilles, and mouth washes.

Dosage forms adapted for nasal or inhalation administration include aerosols, solutions, suspension drops, gels, or dry powders. In some embodiments, a primary active ingredient, optional secondary active ingredient, and/or pharmaceutically acceptable salt thereof where appropriate can be in a dosage form adapted for inhalation is in a particle-size-reduced form that is obtained or obtainable by micronization. In some embodiments, the particle size of the size reduced (e.g. micronized) compound or salt or solvate thereof, is defined by a D₅₀ value of about 0.5 to about 10 microns as measured by an appropriate method known in the art. Dosage forms adapted for administration by inhalation also include particle dusts or mists. Suitable dosage forms wherein the carrier or excipient is a liquid for administration as a nasal spray or drops include aqueous or oil solutions/suspensions of an active (primary and/or secondary) ingredient, which may be generated by various types of metered dose pressurized aerosols, nebulizers, or insufflators. The nasal/inhalation formulations can be administered to a subject in need thereof.

In some embodiments, the dosage forms are aerosol formulations suitable for administration by inhalation. In some of these embodiments, the aerosol formulation contains a solution or fine suspension of a primary active ingredient, secondary active ingredient, and/or pharmaceutically acceptable salt thereof where appropriate and a pharmaceutically acceptable aqueous or non-aqueous solvent. Aerosol formulations can be presented in single or multi-dose quantities in sterile form in a sealed container. For some of these embodiments, the sealed container is a single dose or multi-dose nasal or an aerosol dispenser fitted with a metering valve (e.g. metered dose inhaler), which is intended for disposal once the contents of the container have been exhausted.

Where the aerosol dosage form is contained in an aerosol dispenser, the dispenser contains a suitable propellant under pressure, such as compressed air, carbon dioxide, or an organic propellant, including but not limited to a hydrofluorocarbon. The aerosol formulation dosage forms in other embodiments are contained in a pump-atomizer. The pressurized aerosol formulation can also contain a solution or a suspension of a primary active ingredient, optional secondary active ingredient, and/or pharmaceutically acceptable salt thereof. In further embodiments, the aerosol formulation also contains co-solvents and/or modifiers incorporated to improve, for example, the stability and/or taste and/or fine particle mass characteristics (amount and/or profile) of the formulation. Administration of the aerosol formulation can be once daily or several times daily, for example 2, 3, 4, or 8 times daily, in which 1, 2, 3 or more doses are delivered each time. The aerosol formulations can be administered to a subject in need thereof.

For some dosage forms suitable and/or adapted for inhaled administration, the pharmaceutical formulation is a dry powder inhalable-formulations. In addition to a primary active agent, optional secondary active ingredient, and/or pharmaceutically acceptable salt thereof where appropriate, such a dosage form can contain a powder base such as lactose, glucose, trehalose, manitol, and/or starch. In some of these embodiments, a primary active agent, secondary active ingredient, and/or pharmaceutically acceptable salt thereof where appropriate is in a particle-size reduced form. In further embodiments, a performance modifier, such as L-leucine or another amino acid, cellobiose octaacetate, and/or metals salts of stearic acid, such as magnesium or calcium stearate. In some embodiments, the aerosol formulations are arranged so that each metered dose of aerosol contains a predetermined amount of an active ingredient, such as the one or more of the compositions, compounds, vector(s), molecules, cells, and combinations thereof described herein.

Dosage forms adapted for vaginal administration can be presented as pessaries, tampons, creams, gels, pastes, foams, or spray formulations. Dosage forms adapted for rectal administration include suppositories or enemas. The vaginal formulations can be administered to a subject in need thereof.

Dosage forms adapted for parenteral administration and/or adapted for injection can include aqueous and/or non-aqueous sterile injection solutions, which can contain antioxidants, buffers, bacteriostats, solutes that render the composition isotonic with the blood of the subject, and aqueous and non-aqueous sterile suspensions, which can include suspending agents and thickening agents. The dosage forms adapted for parenteral administration can be presented in a single-unit dose or multi-unit dose containers, including but not limited to sealed ampoules or vials. The doses can be lyophilized and re-suspended in a sterile carrier to reconstitute the dose prior to administration. Extemporaneous injection solutions and suspensions can be prepared in some embodiments, from sterile powders, granules, and tablets. The parenteral formulations can be administered to a subject in need thereof.

For some embodiments, the dosage form contains a predetermined amount of a primary active agent, secondary active ingredient, and/or pharmaceutically acceptable salt thereof where appropriate per unit dose. In an embodiment, the predetermined amount of primary active agent, secondary active ingredient, and/or pharmaceutically acceptable salt thereof where appropriate can be an effective amount, a least effect amount, and/or a therapeutically effective amount. In other embodiments, the predetermined amount of a primary active agent, secondary active agent, and/or pharmaceutically acceptable salt thereof where appropriate, can be an appropriate fraction of the effective amount of the active ingredient.

### Co-Therapies and Combination Therapies

In some embodiments, the pharmaceutical formulation(s) described herein can be part of a combination treatment or combination therapy. The combination treatment can include the pharmaceutical formulation described herein and an additional treatment modality. The additional treatment modality can be a chemotherapeutic, a biological therapeutic, surgery, radiation, diet modulation, environmental modulation, a physical activity modulation, and combinations thereof.

In some embodiments, the co-therapy or combination therapy can additionally include but not limited to, polynucleotides, amino acids, peptides, polypeptides, antibodies, aptamers, ribozymes, hormones, immunomodulators, antipyretics, anxiolytics, antipsychotics, analgesics, antispasmodics, anti-inflammatories, anti-histamines, anti-infectives, chemotherapeutics, and combinations thereof.

### Administration of the Pharmaceutical Formulations

The pharmaceutical formulations or dosage forms thereof described herein can be administered one or more times hourly, daily, monthly, or yearly (e.g. 1, 2, 3, 4, 5, 6, 7, 8, 9, 10, 11, 12, 13, 14, 15, 16, 17, 18, 19, 20, or more times hourly, daily, monthly, or yearly). In some embodiments, the pharmaceutical formulations or dosage forms thereof described herein can be administered continuously over a period of time ranging from minutes to hours to days. Devices and dosages forms are known in the art and described herein that are effective to provide continuous administration of the pharmaceutical formulations described herein. In some embodiments, the first one or a few initial amount(s) administered can be a higher dose than subsequent doses. This is typically referred to in the art as a loading dose or doses and a maintenance dose, respectively. In some embodiments, the pharmaceutical formulations can be administered such that the doses over time are tapered (increased or decreased) overtime so as to wean a subject gradually off of a pharmaceutical formulation or gradually introduce a subject to the pharmaceutical formulation.

As previously discussed, the pharmaceutical formulation can contain a predetermined amount of a primary active agent, secondary active agent, and/or pharmaceutically acceptable salt thereof where appropriate. In some of these embodiments, the predetermined amount can be an appropriate fraction of the effective amount of the active ingredient. Such unit doses may therefore be administered once or more than once a day, month, or year (e.g. 1, 2, 3, 4, 5, 6, or more times per day, month, or year). Such pharmaceutical formulations may be prepared by any of the methods well known in the art.

Where co-therapies or multiple pharmaceutical formulations are to be delivered to a subject, the different therapies or formulations can be administered sequentially or simultaneously. Sequential administration is administration where an appreciable amount of time occurs between administrations, such as more than about 15, 20, 30, 45, 60 minutes or more. The time between administrations in sequential administration can be on the order of hours, days, months, or even years, depending on the active agent present in each administration. Simultaneous administration refers to administration of two or more formulations at the same time or substantially at the same time (e.g. within seconds or just a few minutes apart), where the intent is that the formulations be administered together at the same time.

### Delivery and Administration

Methods for modifying a target of interest comprises administering or delivering or otherwise contacting a cell via one or more methods known in the art, including without limitation, microinjection, electroporation, sonoporation, biolistics, calcium phosphate-mediated transfection, cationic transfection, liposome transfection, dendrimer transfection, heat shock transfection, nucleofection transfection, magnetofection, lipofection, impalefection, optical transfection, proprietary agent-enhanced uptake of nucleic acids, and delivery via liposomes, immunoliposomes, virosomes, or artificial virions. In some methods, the composition is introduced into an embryo by microinjection. The compositions may be microinjected into the nucleus or the cytoplasm of the embryo.

An actively targeting lipid particle or nanoparticle or liposome or lipid bilayer delivery system (generally as to embodiments of the invention, “lipid entity of the invention” delivery systems) are prepared by conjugating targeting moieties, including small molecule ligands, peptides and monoclonal antibodies, on the lipid or liposomal surface; for example, certain receptors, such as folate and transferrin (Tf) receptors (TfR), are overexpressed on many cancer cells and have been used to make liposomes tumor cell specific. Liposomes that accumulate in the tumor microenvironment can be subsequently endocytosed into the cells by interacting with specific cell surface receptors. To efficiently target liposomes to cells, such as cancer cells, it is useful that the targeting moiety have an affinity for a cell surface receptor and to link the targeting moiety in sufficient quantities to have optimum affinity for the cell surface receptors; and determining these aspects are within the ambit of the skilled artisan. In the field of active targeting, there are a number of cell-, e.g., tumor-, specific targeting ligands.

Also as to active targeting, with regard to targeting cell surface receptors such as cancer cell surface receptors, targeting ligands on liposomes can provide attachment of liposomes to cells, e.g., vascular cells, via a noninternalizing epitope; and, this can increase the extracellular concentration of that which is being delivered, thereby increasing the amount delivered to the target cells. A strategy to target cell surface receptors, such as cell surface receptors on cancer cells, such as overexpressed cell surface receptors on cancer cells, is to use receptor-specific ligands or antibodies. Many cancer cell types display upregulation of tumor-specific receptors. For example, TfRs and folate receptors (FRs) are greatly overexpressed by many tumor cell types in response to their increased metabolic demand. Folic acid can be used as a targeting ligand for specialized delivery owing to its ease of conjugation to nanocarriers, its high affinity for FRs and the relatively low frequency of FRs, in normal tissues as compared with their overexpression in activated macrophages and cancer cells, e.g., certain ovarian, breast, lung, colon, kidney and brain tumors. Overexpression of FR on macrophages is an indication of inflammatory diseases, such as psoriasis, Crohn's disease, rheumatoid arthritis and atherosclerosis; accordingly, folate-mediated targeting of the invention can also be used for studying, addressing or treating inflammatory disorders, as well as cancers. Folate-linked lipid particles or nanoparticles or liposomes or lipid bilayers of the invention (“lipid entity of the invention”) deliver their cargo intracellularly through receptor-mediated endocytosis. Intracellular trafficking can be directed to acidic compartments that facilitate cargo release, and, most importantly, release of the cargo can be altered or delayed until it reaches the cytoplasm or vicinity of target organelles. Delivery of cargo using a lipid entity of the invention having a targeting moiety, such as a folate-linked lipid entity of the invention, can be superior to nontargeted lipid entity of the invention. The attachment of folate directly to the lipid head groups may not be favorable for intracellular delivery of folate-conjugated lipid entity of the invention, since they may not bind as efficiently to cells as folate attached to the lipid entity of the invention surface by a spacer, which may can enter cancer cells more efficiently. A lipid entity of the invention coupled to folate can be used for the delivery of complexes of lipid, e.g., liposome, e.g., anionic liposome and virus or capsid or envelope or virus outer protein, such as those herein discussed such as adenovirus or AAV. Tf is a monomeric serum glycoprotein of approximately 80 KDa involved in the transport of iron throughout the body. Tf binds to the TfR and translocates into cells via receptor-mediated endocytosis. The expression of TfR can be higher in certain cells, such as tumor cells (as compared with normal cells and is associated with the increased iron demand in rapidly proliferating cancer cells. Accordingly, the invention comprehends a TfR-targeted lipid entity of the invention, e.g., as to liver cells, liver cancer, breast cells such as breast cancer cells, colon such as colon cancer cells, ovarian cells such as ovarian cancer cells, head, neck and lung cells, such as head, neck and non-small-cell lung cancer cells, cells of the mouth such as oral tumor cells.

Also as to active targeting, a lipid entity of the invention can be multifunctional, i.e., employ more than one targeting moiety such as CPP, along with Tf; a chimeric small system; e.g., a combination of Tf and poly-L-arginine which can provide transport across the endothelium of the blood-brain barrier. EGFR, is a tyrosine kinase receptor belonging to the ErbB family of receptors that mediates cell growth, differentiation and repair in cells, especially non-cancerous cells, but EGF is overexpressed in certain cells such as many solid tumors, including colorectal, non-small-cell lung cancer, squamous cell carcinoma of the ovary, kidney, head, pancreas, neck and prostate, and especially breast cancer. The invention comprehends EGFR-targeted monoclonal antibody(ies) linked to a lipid entity of the invention. HER-2 is often overexpressed in patients with breast cancer, and is also associated with lung, bladder, prostate, brain and stomach cancers. HER-2, encoded by the ERBB2 gene. The invention comprehends a HER-2-targeting lipid entity of the invention, e.g., an anti-HER-2-antibody (or binding fragment thereof)-lipid entity of the invention, a HER-2-targeting-PEGylated lipid entity of the invention (e.g., having an anti-HER-2-antibody or binding fragment thereof), a HER-2-targeting-maleimide-PEG polymer-lipid entity of the invention (e.g., having an anti-HER-2-antibody or binding fragment thereof). Upon cellular association, the receptor-antibody complex can be internalized by formation of an endosome for delivery to the cytoplasm. With respect to receptor-mediated targeting, the skilled artisan takes into consideration ligand/target affinity and the quantity of receptors on the cell surface, and that PEGylation can act as a barrier against interaction with receptors. The use of antibody-lipid entity of the invention targeting can be advantageous. Multivalent presentation of targeting moieties can also increase the uptake and signaling properties of antibody fragments. In practice of the invention, the skilled person takes into account ligand density (e.g., high ligand densities on a lipid entity of the invention may be advantageous for increased binding to target cells). Preventing early by macrophages can be addressed with a sterically stabilized lipid entity of the invention and linking ligands to the terminus of molecules such as PEG, which is anchored in the lipid entity of the invention (e.g., lipid particle or nanoparticle or liposome or lipid bilayer). The microenvironment of a cell mass such as a tumor microenvironment can be targeted; for instance, it may be advantageous to target cell mass vasculature, such as the tumor vasculature microenvironment. Thus, the invention comprehends targeting VEGF. VEGF and its receptors are well-known proangiogenic molecules and are well-characterized targets for antiangiogenic therapy. Many small-molecule inhibitors of receptor tyrosine kinases, such as VEGFRs or basic FGFRs, have been developed as anticancer agents and the invention comprehends coupling any one or more of these peptides n. Matrix metalloproteases (MMPs) belong to the family of zinc-dependent endopeptidases. They are involved in tissue remodeling, tumor invasiveness, resistance to apoptosis and metastasis. There are four MMP inhibitors called TIMP1-4, which determine the balance between tumor growth inhibition and metastasis; a protein involved in the angiogenesis of tumor vessels is MT1-MMP, expressed on newly formed vessels and tumor tissues. The proteolytic activity of MT1-MMP cleaves proteins, such as fibronectin, elastin, collagen and laminin, at the plasma membrane and activates soluble MMPs, such as MMP-2, which degrades the matrix. An antibody or fragment thereof such as a Fab′ fragment can be used in the practice of the invention such as for an antihuman MT1-MMP monoclonal antibody. αβ-integrins or integrins are a group of transmembrane glycoprotein receptors that mediate attachment between a cell and its surrounding tissues or extracellular matrix. Integrins contain two distinct chains (heterodimers) called α- and β-subunits. The tumor tissue-specific expression of integrin receptors can be utilized for targeted delivery in the invention, e.g., whereby the targeting moiety can be an RGD peptide such as a cyclic RGD. Aptamers are ssDNA or RNA oligonucleotides that impart high affinity and specific recognition of the target molecules by electrostatic interactions, hydrogen bonding and hydrophobic interactions as opposed to the Watson-Crick base pairing, which is typical for the bonding interactions of oligonucleotides. Aptamers as a targeting moiety can have advantages over antibodies: aptamers can demonstrate higher target antigen recognition as compared with antibodies; aptamers can be more stable and smaller in size as compared with antibodies; aptamers can be easily synthesized and chemically modified for molecular conjugation; and aptamers can be changed in sequence for improved selectivity and can be developed to recognize poorly immunogenic targets. Such moieties as a sgc8 aptamer can be used as a targeting moiety (e.g., via covalent linking to the lipid entity of the invention, e.g., via a spacer, such as a PEG spacer). The targeting moiety can be stimuli-sensitive, e.g., sensitive to an externally applied stimuli, such as magnetic fields, ultrasound or light; and pH-triggering can also be used, e.g., a labile linkage can be used between a hydrophilic moiety such as PEG and a hydrophobic moiety such as a lipid entity of the invention, which is cleaved only upon exposure to the relatively acidic conditions characteristic of the a particular environment or microenvironment such as an endocytic vacuole or the acidotic tumor mass. pH-sensitive copolymers can also be incorporated in example embodiments of the invention can provide shielding; diortho esters, vinyl esters, cysteine-cleavable lipopolymers, double esters and hydrazones are a few examples of pH-sensitive bonds that are quite stable at pH 7.5, but are hydrolyzed relatively rapidly at pH 6 and below, e.g., a terminally alkylated copolymer of N-isopropylacrylamide and methacrylic acid that copolymer facilitates destabilization of a lipid entity of the invention and release in compartments with decreased pH value; or, the invention comprehends ionic polymers for generation of a pH-responsive lipid entity of the invention (e.g., poly(methacrylic acid), poly(diethylaminoethyl methacrylate), poly(acrylamide) and poly(acrylic acid)). Temperature-triggered delivery is also within the ambit of the invention. Many pathological areas, such as inflamed tissues and tumors, show a distinctive hyperthermia compared with normal tissues. Utilizing this hyperthermia is an attractive strategy in cancer therapy since hyperthermia is associated with increased tumor permeability and enhanced uptake. This technique involves local heating of the site to increase microvascular pore size and blood flow, which, in turn, can result in an increased extravasation of embodiments of the invention. Temperature-sensitive lipid entity of the invention can be prepared from thermosensitive lipids or polymers with a low critical solution temperature. Above the low critical solution temperature (e.g., at site such as tumor site or inflamed tissue site), the polymer precipitates, disrupting the liposomes to release. Lipids with a specific gel-to-liquid phase transition temperature are used to prepare these lipid entities of the invention; and a lipid for a thermosensitive embodiment can be dipalmitoylphosphatidylcholine. Thermosensitive polymers can also facilitate destabilization followed by release, and a useful thermosensitive polymer is poly (N-isopropylacrylamide). Another temperature triggered system can employ lysolipid temperature-sensitive liposomes. The invention also comprehends redox-triggered delivery: The difference in redox potential between normal and inflamed or tumor tissues, and between the intra- and extra-cellular environments has been exploited for delivery; e.g., GSH is a reducing agent abundant in cells, especially in the cytosol, mitochondria and nucleus. The GSH concentrations in blood and extracellular matrix are just one out of 100 to one out of 1000 of the intracellular concentration, respectively. This high redox potential difference caused by GSH, cysteine and other reducing agents can break the reducible bonds, destabilize a lipid entity of the invention and result in release of payload. The disulfide bond can be used as the cleavable/reversible linker in a lipid entity of the invention, because it causes sensitivity to redox owing to the disulfideto-thiol reduction reaction; a lipid entity of the invention can be made reduction sensitive by using two (e.g., two forms of a disulfide-conjugated multifunctional lipid as cleavage of the disulfide bond (e.g., via tris(2-carboxyethyl)phosphine, dithiothreitol, L-cysteine or GSH), can cause removal of the hydrophilic head group of the conjugate and alter the membrane organization leading to release of payload. Calcein release from reduction-sensitive lipid entity of the invention containing a disulfide conjugate can be more useful than a reduction-insensitive embodiment. Enzymes can also be used as a trigger to release payload. Enzymes, including MMPs (e.g. MMP2), phospholipase A2, alkaline phosphatase, transglutaminase or phosphatidylinositol-specific phospholipase C, have been found to be overexpressed in certain tissues, e.g., tumor tissues. In the presence of these enzymes, specially engineered enzyme-sensitive lipid entity of the invention can be disrupted and release the payload. an MMP2-cleavable octapeptide (Gly-Pro-Leu-Gly-Ile-Ala-Gly-Gln) (SEQ ID NO: 1) can be incorporated into a linker, and can have antibody targeting, e.g., antibody 2C5. The invention also comprehends light- or energy-triggered delivery, e.g., the lipid entity of the invention can be light-sensitive, such that light or energy can facilitate structural and conformational changes, which lead to direct interaction of the lipid entity of the invention with the target cells via membrane fusion, photo-isomerism, photofragmentation or photopolymerization; such a moiety therefor can be benzoporphyrin photosensitizer. Ultrasound can be a form of energy to trigger delivery; a lipid entity of the invention with a small quantity of particular gas, including air or perfluorated hydrocarbon can be triggered to release with ultrasound, e.g., low-frequency ultrasound (LFUS). Magnetic delivery: A lipid entity of the invention can be magnetized by incorporation of magnetites, such as Fe3O4 or γ-Fe2O3, e.g., those that are less than 10 nm in size. Targeted delivery can be then by exposure to a magnetic field.

Also as to active targeting, the invention also comprehends intracellular delivery. Since liposomes follow the endocytic pathway, they are entrapped in the endosomes (pH 6.5-6) and subsequently fuse with lysosomes (pH<5), where they undergo degradation that results in a lower therapeutic potential. The low endosomal pH can be taken advantage of to escape degradation. Fusogenic lipids or peptides, which destabilize the endosomal membrane after the conformational transition/activation at a lowered pH. Amines are protonated at an acidic pH and cause endosomal swelling and rupture by a buffer effect Unsaturated dioleoylphosphatidylethanolamine (DOPE) readily adopts an inverted hexagonal shape at a low pH, which causes fusion of liposomes to the endosomal membrane. This process destabilizes a lipid entity containing DOPE and releases the cargo into the cytoplasm; fusogenic lipid GALA (SEQ ID NO: 45), cholesteryl-GALA (SEQ ID NO: 45) and PEG-GALA (SEQ ID NO: 45) may show a highly efficient endosomal release; a pore-forming protein listeriolysin O may provide an endosomal escape mechanism; and, histidine-rich peptides have the ability to fuse with the endosomal membrane, resulting in pore formation, and can buffer the proton pump causing membrane lysis.

Also as to active targeting, cell-penetrating peptides (CPPs) facilitate uptake of macromolecules through cellular membranes and, thus, enhance the delivery of CPP-modified molecules inside the cell. CPPs can be split into two classes: amphipathic helical peptides, such as transportan and MAP, where lysine residues are major contributors to the positive charge; and Arg-rich peptides, such as TATp, Antennapedia or penetratin. TATp is a transcription-activating factor with 86 amino acids that contains a highly basic (two Lys and six Arg among nine residues) protein transduction domain, which brings about nuclear localization and RNA binding. Other CPPs that have been used for the modification of liposomes include the following: the minimal protein transduction domain of Antennapedia, a Drosophila homeoprotein, called penetratin, which is a 16-mer peptide (residues 43-58) present in the third helix of the homeodomain; a 27-amino acid-long chimeric CPP, containing the peptide sequence from the amino terminus of the neuropeptide galanin bound via the Lys residue, multipara, a wasp venom peptide; VP22, a major structural component of HSV-1 facilitating intracellular transport and transportan (18-mer) amphipathic model peptide that translocates plasma membranes of mast cells and endothelial cells by both energy-dependent and -independent mechanisms. The invention comprehends a lipid entity of the invention modified with CPP(s), for intracellular delivery that may proceed via energy dependent micropinocytosis followed by endosomal escape. The invention further comprehends organelle-specific targeting. A lipid entity of the invention surface-functionalized with the triphenyl phosphonium (TPP) moiety or a lipid entity of the invention with a lipophilic cation, rhodamine 123 can be effective in delivery of cargo to mitochondria. DOPE/sphingomyelin/stearyl-octa-arginine can delivers cargos to the mitochondrial interior via membrane fusion. A lipid entity of the invention surface modified with a lysosomotropic ligand, octadecyl rhodamine B can deliver cargo to lysosomes. Ceramides are useful in inducing lysosomal membrane permeabilization; the invention comprehends intracellular delivery of a lipid entity of the invention having a ceramide. The invention further comprehends a lipid entity of the invention targeting the nucleus, e.g., via a DNA-intercalating moiety. The invention also comprehends multifunctional liposomes for targeting, i.e., attaching more than one functional group to the surface of the lipid entity of the invention, for instance to enhances accumulation in a desired site and/or promotes organelle-specific delivery and/or target a particular type of cell and/or respond to the local stimuli such as temperature (e.g., elevated), pH (e.g., decreased), respond to externally applied stimuli such as a magnetic field, light, energy, heat or ultrasound and/or promote intracellular delivery of the cargo. All of these are considered actively targeting moieties.

An embodiment of the system may comprise an actively targeting lipid particle or nanoparticle or liposome or lipid bilayer delivery system; or a lipid particle or nanoparticle or liposome or lipid bilayer comprising a targeting moiety whereby there is active targeting or wherein the targeting moiety is an actively targeting moiety. A targeting moiety can be one or more targeting moieties, and a targeting moiety can be for any desired type of targeting such as, e.g., to target a cell such as any herein-mentioned; or to target an organelle such as any herein-mentioned; or for targeting a response such as to a physical condition such as heat, energy, ultrasound, light, pH, chemical such as enzymatic, or magnetic stimuli; or to target to achieve a particular outcome such as delivery of payload to a particular location, such as by cell penetration.

It should be understood that as to each possible targeting or active targeting moiety herein-discussed, there is one example embodiment of the invention wherein the delivery system comprises such a targeting or active targeting moiety.

Further embodiments are illustrated in the following Examples which are given for illustrative purposes only and are not intended to limit the scope of the invention.

The present application also provides aspects and embodiments as set forth in the following numbered Statements:

Statement 1. An immune cell recruiting chimeric small molecule comprising a target protein binding moiety and an immunogenic display moiety connected via one or more linker molecules, and optionally an electrophilic reactive group. wherein the protein binding moiety facilitates labeling of an amino acid of a protein, via the electrophilic reactive group, with the immunogenic display moiety.

Statement 2. The chimeric small molecule of Statement 1, according to the formula A-L₁-E-B or A-L₁-E-L₂-B or A-E-L₁-B, wherein A is the target protein binding moiety; B is the immunogenic display moiety; L₁ and L₂ are each a linker; and E is an electrophilic reactive group.

Statement 3. The chimeric small molecule of any of the previous Statements wherein the immunogenic display moiety is configured to bind with i) a cell surface of a natural or an engineered immune cell, or (ii) bifunctional bridge molecule comprising a first binding moiety that binds the immunogenic display moiety and a second binding moiety that binds the surface of a natural or engineered immune cell.

Statement 4. The chimeric small molecule of Statement 3, wherein the natural or an engineered immune cell is a CAR T cell, T cell or an NK cell.

Statement 5. The chimeric small molecule of any of the previous Statements, wherein the target protein is a disease-specific protein, optionally an oncogenic-specific protein.

Statement 6. The chimeric small molecule of any of the preceding Statements, wherein the protein binding moiety is a KRAS^{G12C}, EGFR, pan-EGFR, ITK, FGFR4, JAK3, RIP1, MEK1/2, CDK, AKT, TAK, JNK, BMX, LIMK, IRE1, IRE2, ABL1, EphA2 receptor, a human dipeptidyl peptidase IV/CD26, a HER2 receptor, a prostate-specific membrane antigen (PSMA), a folate receptor, or a somatostatin binding moiety.

Statement 7. The chimeric small molecule of any of the preceding Statements, wherein the amino acid is lysine or cysteine.

Statement 8. A bifunctional immune cell engager comprising a first binding moiety capable of binding the immunogenic display moiety of a chimeric small molecule of any one of Statements 1 to 7 and a second binding moiety capable of binding a cell surface receptor of a natural or engineered immune cell.

Statement 9. The bifunctional immune cell engager of Statement 8, wherein the immune cell is a CD8 T cell, a CD4 T cell, a NK cell, a CAR T cell, or an engineered tumor infiltrating lymphocyte (TIL).

Statement 10. The bifunctional immune cell engager of Statements 8 or 9, wherein the cell surface receptor is CD3, CD19, CD20, CD22, CD30, CD33, CD38, CD79B, or SLAMF7.

Statement 11. The bifunctional immune cell engager of any one of Statements 8 to 10, wherein the immunogenic display moiety of the chimeric small molecule and the first binding moiety of the bifunctional immune cell engager together comprise a click chemistry reagent pair.

Statement 12. The bifunctional immune cell engager of Statement 11, wherein a binding domain of the second binding moiety is masked such that the second binding moiety is incapable of binding a cell surface receptor of a natural or engineered immune cell, and wherein the click chemistry reaction of the click chemistry reagent pair unmasks the binding domain of the second binding moiety, such that the second binding moiety is capable of binding the cell surface receptor of the natural or engineered immune cell.

Statement 13. The bifunctional immune cell engager of Statement 11 or 12, wherein the immunogenic display moiety is a tetrazine moiety, or a tetracyclooctene (TCO) moiety, and the first binding moiety is a corresponding TCO moiety, or tetrazine moiety.

Statement 14. The bifunctional immune cell engager of Statements 8 to 10, wherein the immunogenic display moiety is a Halo Tag ligand and the first binding moiety is a Halo Tag protein.

Statement 15. The bifunctional immune cell engager of any one of Statements 8 to 10, wherein the immunogenic display moiety is an E3 ligase ligand, and the first binding moiety is an E3 ligase ligand binding moiety of a CRBN protein, or an antibody or antibody fragment to an E3 ligase ligand.

Statement 16. The bifunctional immune cell engager of any one of Statements 8 to 10, wherein the first binding moiety is an antibody, a scFV fragment, or a nanobody directed against the immunogenic display moiety.

Statement 17. The bifunctional immune cell engager of any one of Statements 8 to 10, wherein the bifunctional immune cell engager is a BiTE, wherein the first binding moiety is a first antibody variable region the binds the immunogenic display moiety and the second binding moiety is a second antibody variable region that binds a cell surface receptor on an immune cell.

Statement 18. A method of inducing immune response, comprising: delivering the immune cell recruiting chimeric small molecule of any one of Statements 1 to 7; labeling one or more target polypeptides of one or more target proteins with the immunogenic display moiety by the immune cell recruiting chimeric small molecule; and displaying the one more target polypeptides labeled with the immunogenic display moiety on the cell surface via a Major Histocompatibility Complex (MHC) molecule.

Statement 19. The method of Statement 18, further comprising eliciting an immune response by binding of the immunogenic display moiety to a natural or an engineered immune cell, thereby activating the immune cell.

Statement 20. The method of Statement 18 or 19, further comprising eliciting an immune response by administering the bifunctional immune cell engager of any one of Statement 8 to 17, wherein the first binding moiety of the bifunctional immune cell engager binds the immunogenic display moiety displayed on the surface of the cell and the second binding moiety of the bifunctional immune cell engager binds a cell surface receptor of a natural or engineered immune cell thereby activating the natural or engineered immune cell.

Statement 21. The method of claim any one of Statements 18 to 20, wherein two or more different target proteins are labeled with the same immunogenic display moiety, whereby each target polypeptide of each different target protein is recognized by the same natural or engineered immune cell.

Statement 22. The method of Statement 21, wherein the chimeric small molecule that labels the two or more different target proteins is the same molecule or different molecules.

Statement 23. A method of labeling cell surface polypeptides, comprising: delivering the chimeric small molecule of any of Statements 1 to 7 to a subject in need thereof, and labeling one or more target cell surface polypeptides with the immunogenic display moiety by the chimeric small molecule.

Statement 24. The method of Statement 23, further comprising eliciting an immune response by binding of the immunogenic display moiety to a natural or an engineered immune cell, thereby activating the natural or engineered immune cell.

Statement 25. The method of Statement 23 or 24, further comprising eliciting an immune response by administering the bifunctional immune cell engager of any one of Statements 8 to 17 to the cell surface, wherein the first binding moiety of the bifunctional immune cell engager binds the immunogenic display moiety and the second binding moiety of the bifunctional immune cell engager binds a cell surface receptor of a natural or engineered immune cell, thereby activating the natural or engineered immune cell.

Statement 26. The method of any one of Statements 23 to 25, wherein two or more different target cell surface polypeptides are labeled with the same immunogenic display moiety, whereby each different target cell surface polypeptide is recognized by the same natural or engineered immune cell.

Statement 27. The method of Statement 26, wherein the chimeric small molecule that labels the two or more different cell surface polypeptides is the same chimeric small molecule or different chimeric small molecules.

Statement 28. The method of any one of Statements 18 to 22, wherein the target protein is a disease-specific protein, optionally an oncogenic-specific protein.

Statement 29. The method of Statement 28, wherein the target protein is KRASG12C, EGFR, pan-EGFR, ITK, FGFR4, JAK3, RIP1, MEK1/2, CDK, AKT, TAK, INK, BMX, LIMK, IRE1, IRE2, ABL1, EphA2 receptor, a human dipeptidyl peptidase IV/CD26, a HER2 receptor, a prostate-specific membrane antigen (PSMA), a folate receptor, or somatostatin.

Statement 30. The method of any one of Statements 23 to 27, wherein the target cell surface polypeptide is a disease-specific polypeptide, optionally an oncogenic-specific polypeptide.

Statement 31. The method of Statement 30, wherein the target cell surface polypeptide is a prostate-specific membrane antigen (PSMA), a folate receptor, a somatostatin receptor, a human dipeptidyl peptidase IV/CD26, a HER2 receptor, or EGFR polypeptide.

### EXAMPLES

### Example 1—MHC Display Mediated Recruitment of T Cells to Cancer Cells

FIG. 1 shows a schematic of an example chimeric small molecule comprising a protein binding moiety (triangle, e.g., a KRAS inhibitor) that binds to a protein (e.g., KRAS^{G12C}) and appends an immunogenic display moiety (sphere) onto the protein (e.g., KRAS). Through HLA display such a molecule can be presented on cells and this molecule can help recruit appropriately engineered T cells.

In one example embodiment, a protein is a gene mutation, a regulator protein, and/or a regulatory enzyme that is specific to or upregulated in cancer cells. In one example embodiment, a protein is an oncoprotein and a protein binding moiety is an inhibitor of said oncoprotein. In one example embodiment, a protein is a kinase, and a protein binding moiety is an inhibitor of said kinase. Using various kinase inhibitors and by targeting amino acids such as lysine or cysteine, Applicants have developed chimeric small molecules for labeling of several kinases. These chimeric small molecules can be adapted to other oncogenic targets.

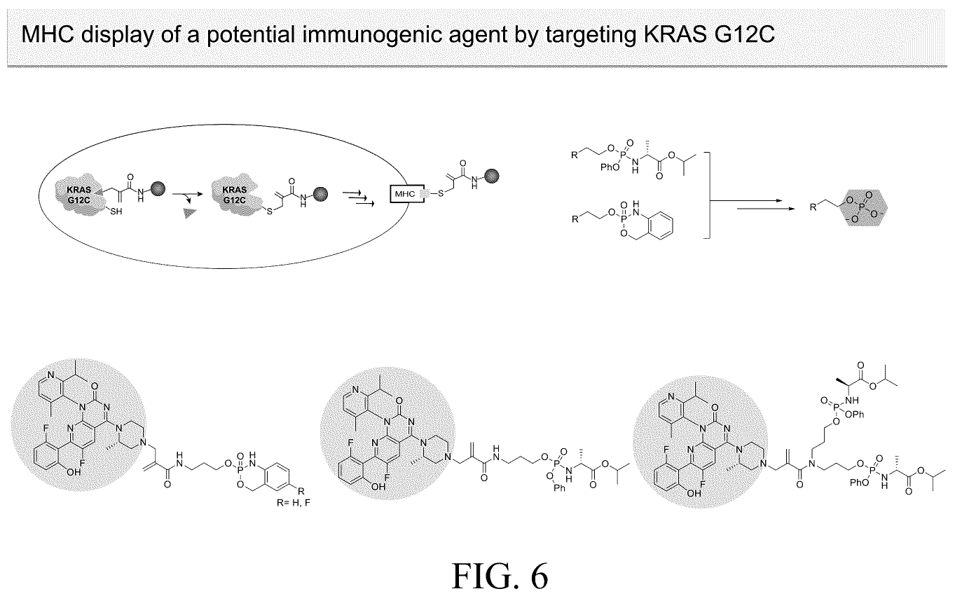

In one example embodiment, an immunogenic display moiety (grey circle of FIGS. 1, 6) is attached to a protein (e.g., KRAS) using an inhibitor of said protein (orange triangle of FIGS. 1, 6). Through major histocompatibility complex (MHC) display (e.g., the human leukocyte antigen (HLA) display in humans), such an immunogenic display moiety can be presented on cells to help recruit appropriately engineered T cells having a receptor that binds to the immunogenic display moiety (e.g., where the immunogenic display moiety is an antigen, e.g., a phospho-antigen, see FIG. 6), or to help recruit an immune cell, such as a T cell, via a ternary complex between the immunogenic display moiety, an immune cell, and a bifunctional protein having a receptor for each of the immunogenic display moiety and the immune cell (FIG. 1). FIG. 2 shows two alternate designs for a bifunctional protein comprising a first antibody capable of binding an immunogenic display moiety (HaloTAG or FKBP^{F36V}) and a second antibody capable of binding an immune cell (scFV for CD3 group).

In an exemplary embodiment, modification of a target substrate, e.g., a protein, may result in immune recruitment to a target substrate, for example, via trigger display of neo-epitomes and T-cell attack on cells displaying epitopes. Modification of key regulator enzymes and proteins implicated in cancer are also within the scope of the methods disclosed herein. FIGS. 3-24 show exemplary chimeric small molecules, and/or portions thereof, suitable for appending proteins with immunogenic moieties, e.g., for MHC display and immune cell recruitment. The following description provides proof of concept of such methods of use.

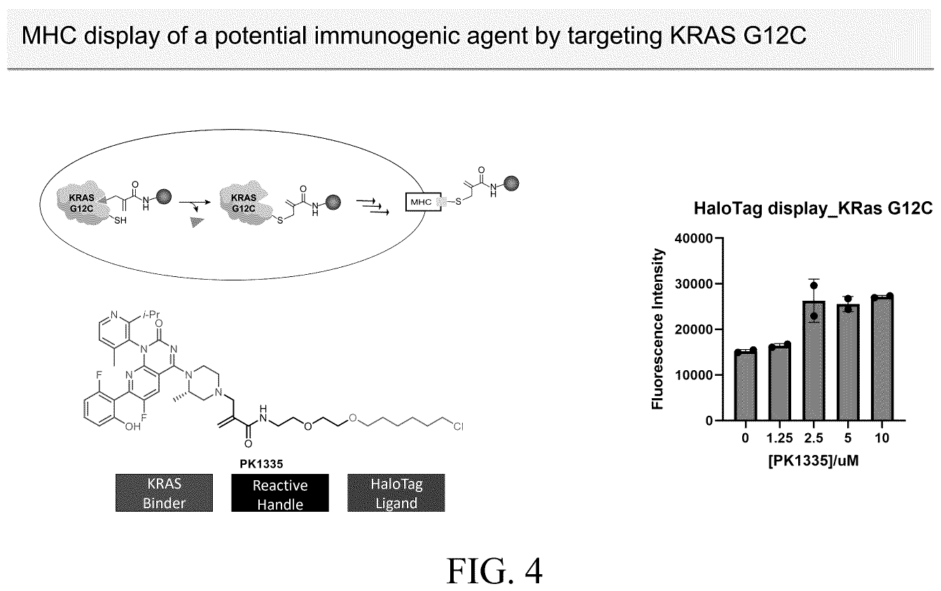

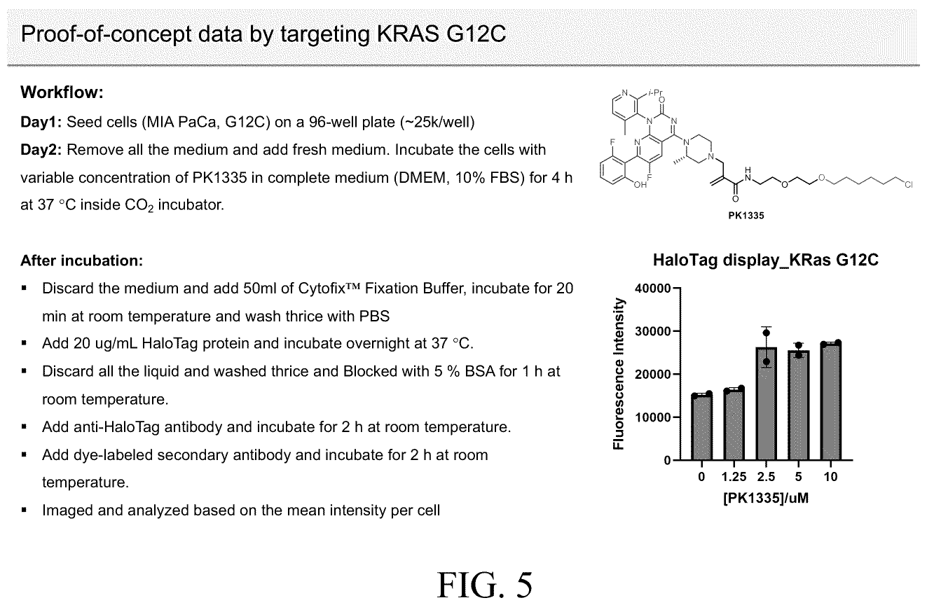

FIG. 3 shows example chimeric small molecules comprising a protein binding group specific for KRAS (left side of the molecule). The KRAS binding group is attached to an electrophilic reactive group, further attached to various immunogenic groups (right side of the molecule, selected from HaloTAG, dinitrophenyl, or FKBP^{F36V} group) via linkers of various lengths. FIGS. 4 and 5 show MHC display of a potential immunogenic agent by targeting KRAS G12C using a molecule comprising a KRAS binder attached to an electrophilic reactive group (a reactive handle, PK1335), further attached to a HaloTag Ligand).

### Example 2—T-Cell Activation Assay

The following protocol was used for obtaining proof-of-concept data, obtained by targeting KRAS G12C as described in FIG. 5: Day1: Seed cells (MIA PaCa, G12C) on a 96-well plate (˜25 k/well). Day2: Remove all the medium and add fresh medium. Incubate the cells with variable concentration of PK 1335 in complete medium (DMEM, 10% FBS) for 4 h at 37° C. inside CO₂ incubator. After incubation. Discard the medium and add 50 ml of Cytofix™ Fixation Buffer, incubate for 20 min at room temperature and wash thrice with PBS. Add 20 μg/mL HaloTag protein and incubate overnight at 37° C. Discard all the liquid and washed thrice and Blocked with 5% BSA for 1 h at room temperature. Add anti-HaloTag antibody and incubate for 2 h at room temperature. Add dye-labeled secondary antibody and incubate for 2 h at room temperature. Imaged and analyzed based on the mean fluorescence intensity per cell.

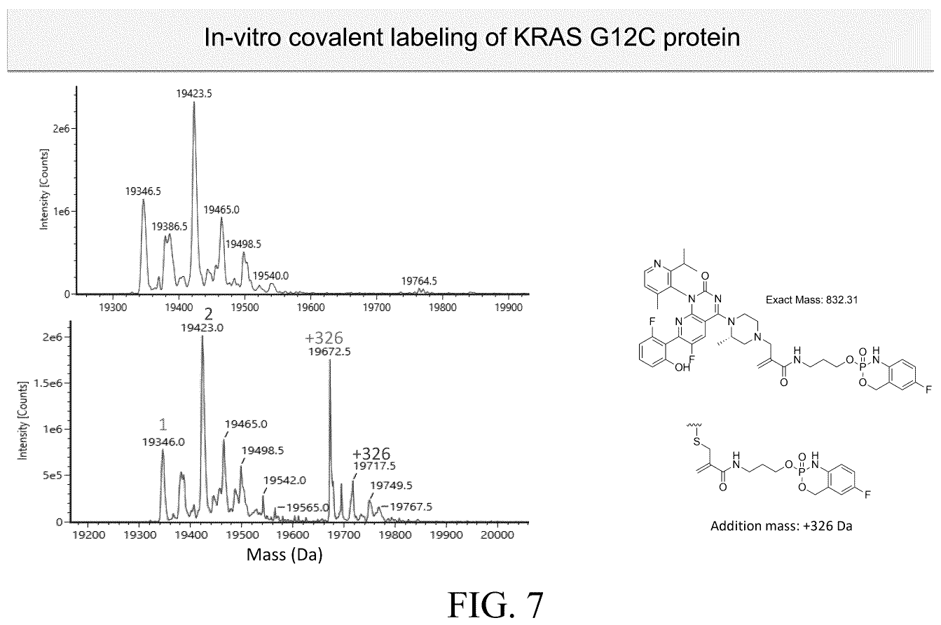

FIGS. 4 and 5 shows data obtained by the fluorescence-based assay. An increase of fluorescence was observed with increasing concentration of the molecule comprising PK1335. FIGS. 6 and 7 shows MHC display of a potential immunogenic agent by targeting KRAS G12C using a molecule comprising a KRAS binder attached to an electrophilic reactive group (a reactive handle), further attached to a phosphor-antigen). FIG. 7 shows mass spectrometry measurement of in-vitro covalent labeling of KRAS G12C protein. Additional mass associated with a cysteine sulfur attached to the reactive handle and phosphor-antigen group was observed.

### Example 3—T-Cell Activation Assay

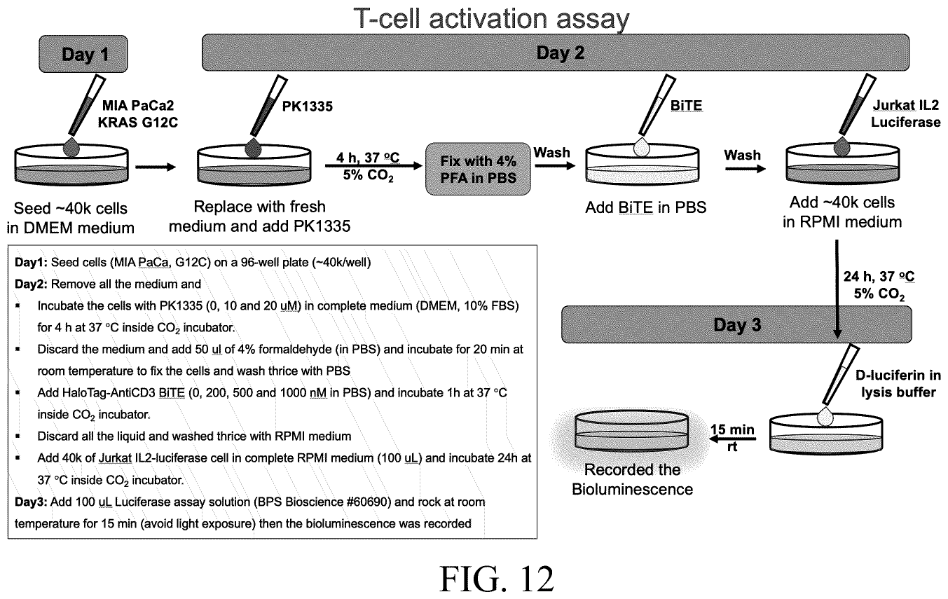

The following protocol was used for obtaining proof-of-concept data, obtained by targeting KRAS G12C as described in FIG. 12: Day 1: Seed cells (MIA PaCa, G12C) on a 96-well plate (˜40 k/well). Day2: Remove all the medium and Incubate the cells with PK1335 (0, 10 and 20 uM) in complete medium (DMEM, 10% FBS) for 4 h at 37° C. inside CO₂ incubator. Discard the medium and add 50 ul of 4% formaldehyde (in PBS) and incubate for 20 min at room temperature to fix the cells and wash three times with PBS. Add HaloTag-AntiCD3 BiTE (0, 200, 500 and 1000 nM in PBS) and incubate 1 h at 37° C. inside CO₂ incubator. Discard all the liquid and washed thrice with RPMI medium. Add 40 k of Jurkat IL2-luciferase cell in complete RPMI medium (100 uL) and incubate 24 h at 37° C. inside CO₂ incubator. Day3: Add 100 uL Luciferase assay solution (BPS Bioscience #60690) and rock at room temperature for 15 min (avoid light exposure) then the bioluminescence was recorded.

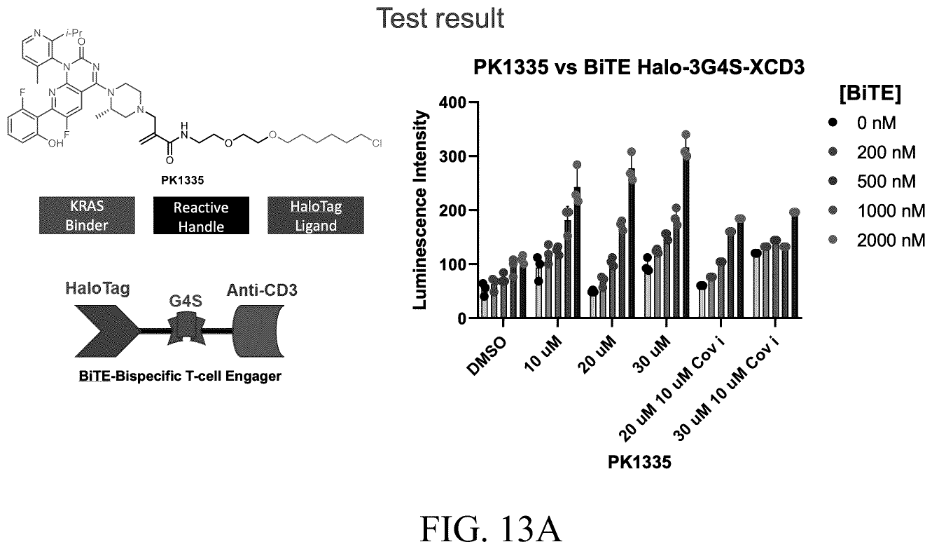

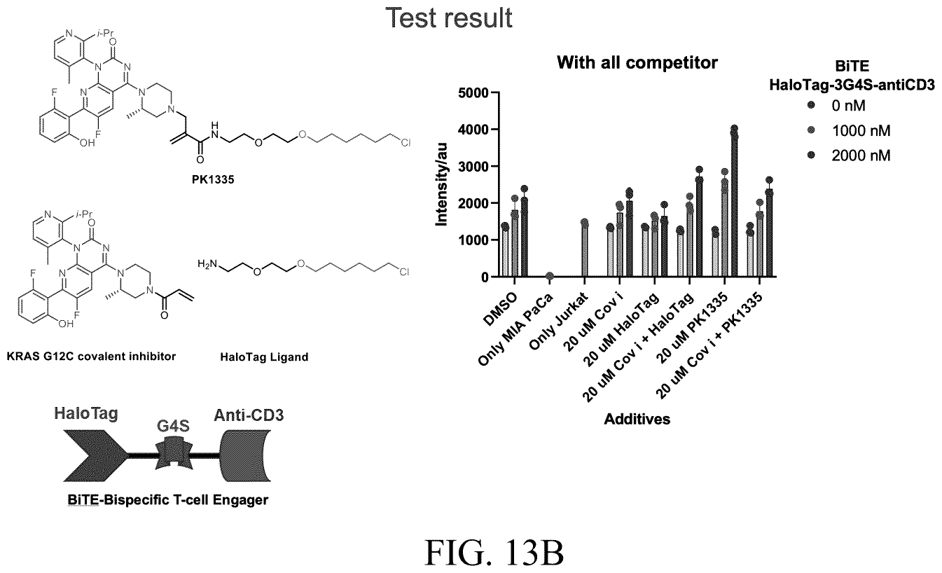

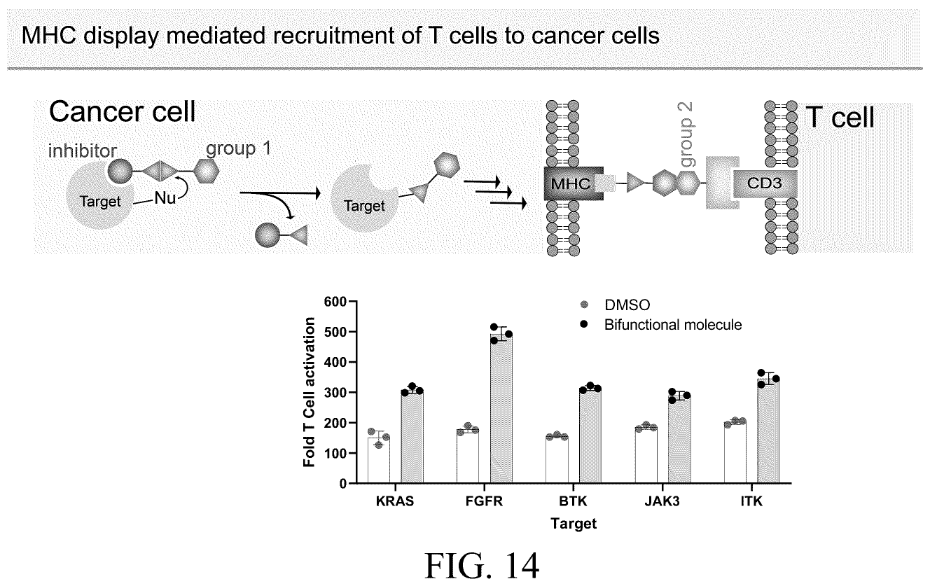

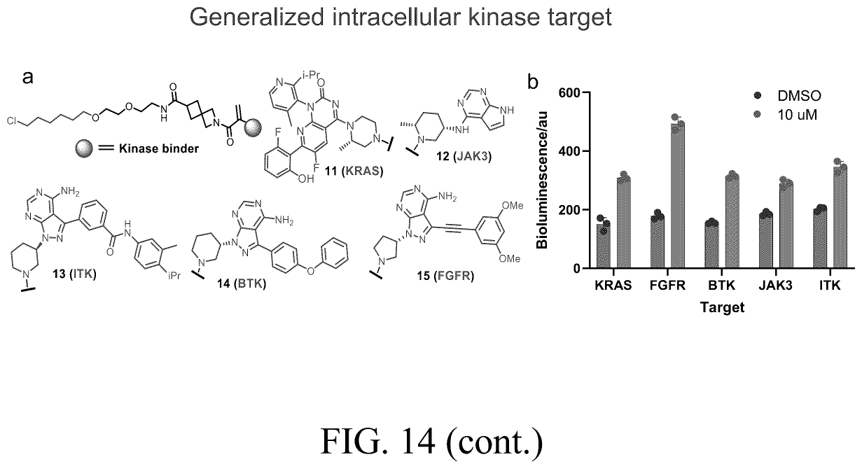

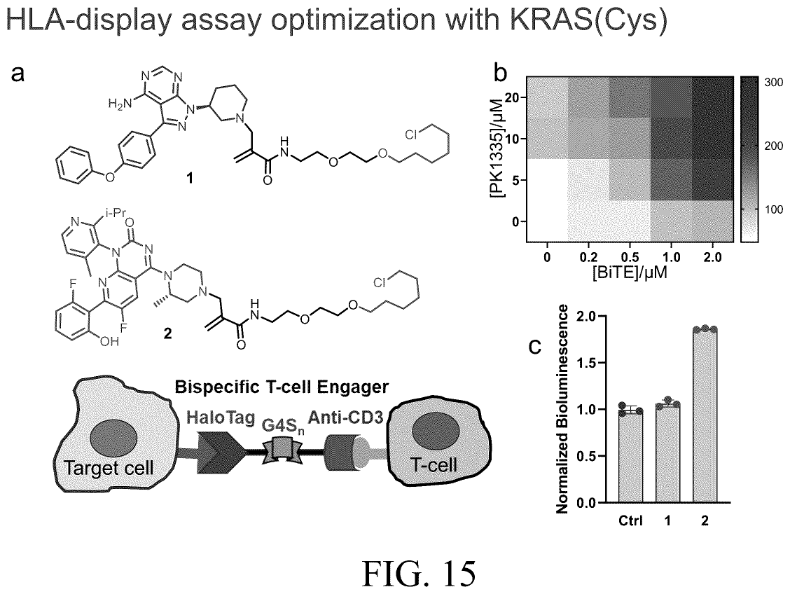

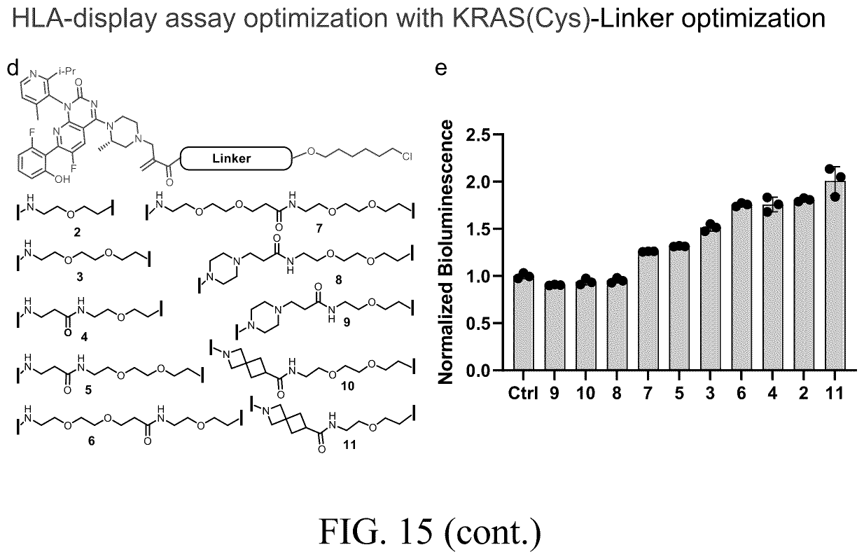

FIG. 13A-13B show the data obtained by the fluorescence-based assay. An increase of fluorescence was observed with increasing concentrations of the molecule comprising PK1335 and/or HaloTag-AntiCD3 BiTE. FIG. 14 shows a schematic of MHC display mediated recruitment of T cells to cancer cells as well as test results for chimeric small molecules targeting various target proteins. An increase of fluorescence was observed with the presence of the molecule compared to the control. FIG. 15 shows test results for chimeric small molecules targeting KRAS (Cys) having different target binding groups or different linker groups. For certain target binding groups, certain linker groups, and/or certain concentrations of the chimeric small molecule and BiTE molecule, an increase of fluorescence was observed compared to the control.

### Example 4—T-Cell Recruiting Chimeras (TRCs) for Extracellular Targets

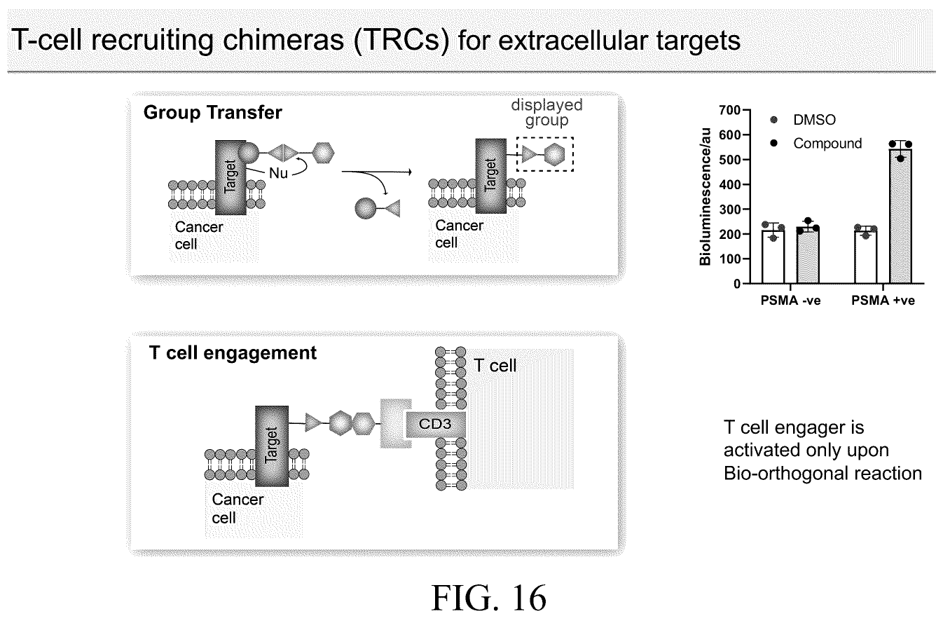

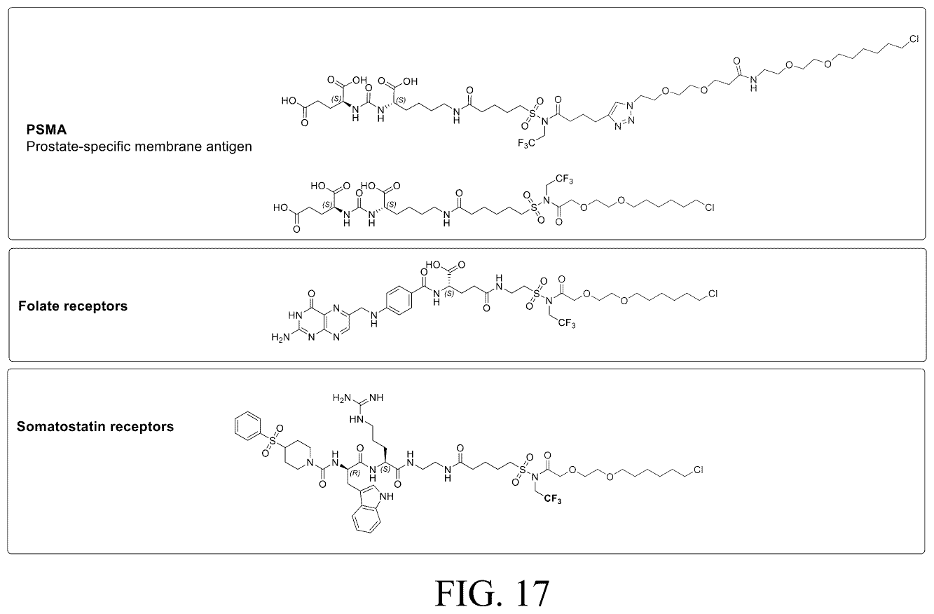

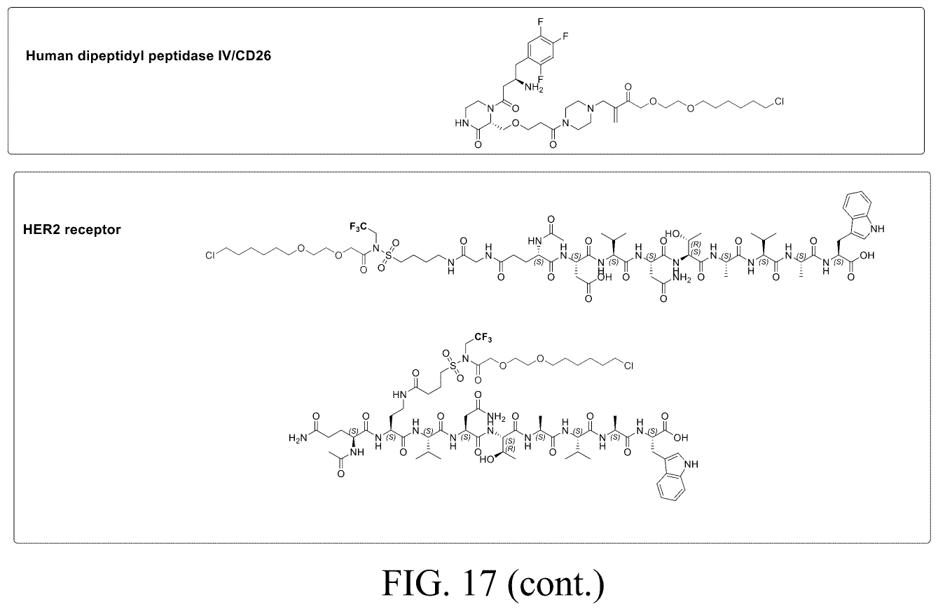

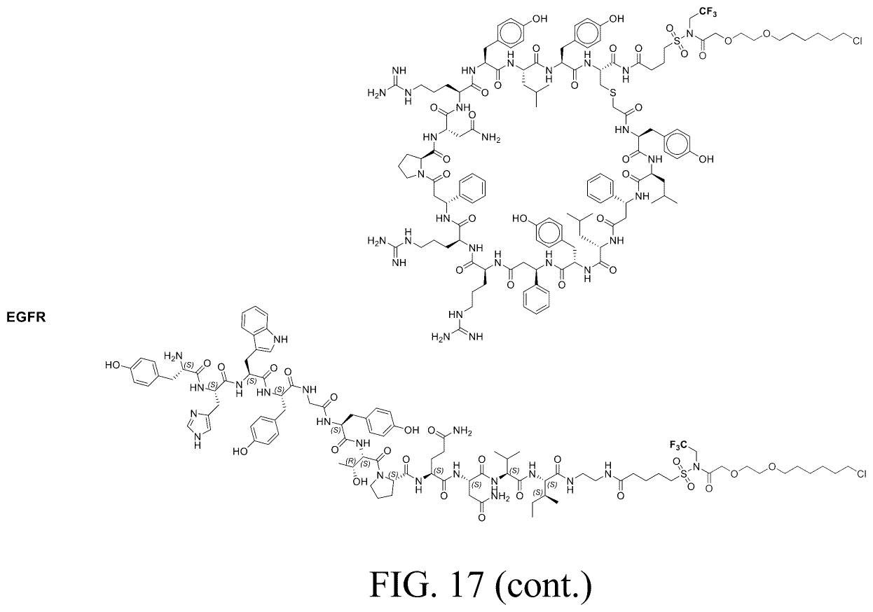

FIGS. 3-24 show exemplary chimeric small molecules, and/or portions thereof, suitable for appending cell surface proteins with immunogenic moieties, e.g., for display and immune cell recruitment. The following description provides proof of concept of such methods of use. FIG. 16 shows a schematic of the use of group transfer chemistry to react T-cell recruiting chimeras (TRCs) onto extracellular targets and the ability of the displayed immunogenic group to engage a T-cell via a BiTE molecule. Proof-of-concept data is also shown using the TRCs to target prostate-specific membrane antigen positive cells. FIG. 17 shows exemplary structures designed to target various extracellular proteins (e.g., oncoproteins) according to this protocol.

### Example 5—Chemical Trigger for Activation of Immune Cell Recruiting

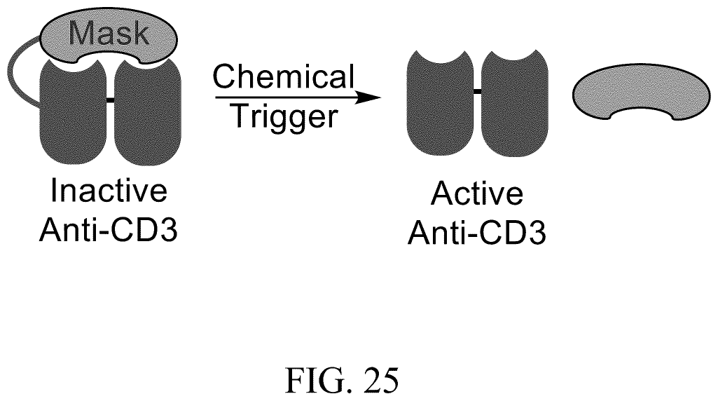

FIGS. 25 and 33 show schematics for preventing activation of an immune cell (e.g., a T-cell) by a bifunctional compound, (e.g., a BiTE compound comprising an Anti-CD3 group) by protecting the bifunctional compound with a masking agent (e.g., a cleavable linker). Only upon reaction of the bifunctional compound with a chimeric small molecule at a surface of a cell will the masking agent be released, activating the immune cell binding group and allowing recruiting of an immune cell to the surface of the cell.

In certain embodiments, click-chemistry occurs between the display functionality (e.g., a HaloTAG ligand) attached to the protein of interest and a masking small molecule attached to the anti-CD3 binding portion of the BiTE. A click-chemistry reaction between the display functionality (e.g., a HaloTAG ligand) and the masking molecule will unmask the anti-CD3 fragment allowing it to recruit a T cell. In certain embodiments, the click chemistry will result in a molecule that unmasks the anti-CD3. In certain embodiments, the BiTE is designed to recognize the display functionality at the surface of a cell. In certain embodiments, the BiTE is designed to recognize the new molecule that results from the click-chemistry reaction between the display functionality and the masking molecule.

In certain embodiments, the display functionality acts as a “chemical protease” for a cleavable linker attached to the bifunctional compound, and upon cleavage of the linker, the bifunctional compound is capable of activating the immune cell binding group and allowing recruiting of an immune cell to the surface of the cell.

### Example 6—Development of T-Cell Recruiting Chimeras for KRAS

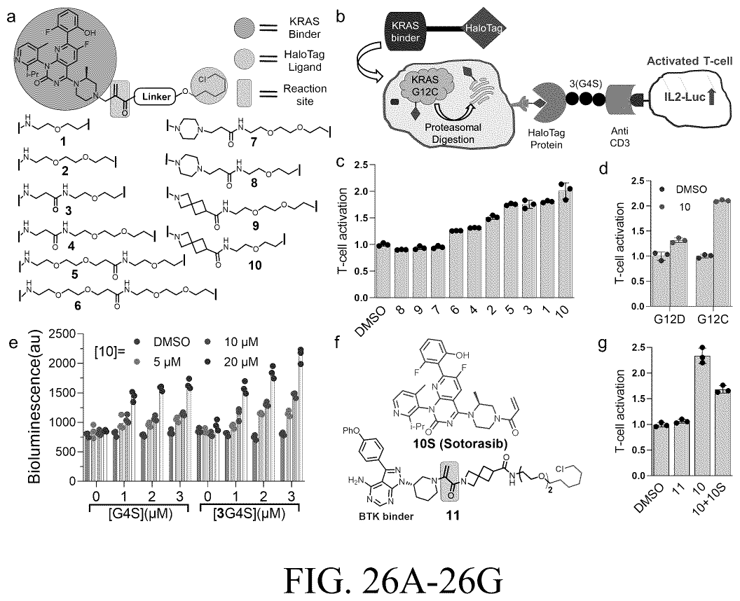

Envisioning the diversity and fidelity of the proximity induced chimeric small molecules which can covalently ligate cysteine on a target protein Applicant thought to extend the space of application. In this regard, Applicant sought to covalently label a cysteine of a target protein inside the cell and display that on the outer cell surface via MHC-I (Human Leukocyte Antigen (HLA)). For a comprehensive demonstration of this modality, Applicant devised a bioluminescence-based T-cell activation assay. Applicant named the assay as T-cell Recruitment via Inside-out Covalent Labelling (TRICL). As a proof of concept, Applicant chose KRAS(G12C) protein as the target because the covalent ligation on that cysteine is observed to be sustained proteasomal digestion and antigen loading and eventually displayed on the cell surface (Ziyang Zhang et al., Cancer Cell, 2022). To understand the MHC display efficiency of the covalently modified allele, first, Applicant synthesized a series of chimeric small molecules keeping same binder (KRAS binder), reaction handle (methacrylamide) and HaloTag ligand (haloalkane) but varying the linker with different chemical constituents, length, and flexibility (FIG. 26A). Now to evaluate the efficiency of synthesized molecules Applicant tested activity using TRICL assay. The TRICL assay works as follows, step-I: the test molecule will covalently label the cysteine residue on the target protein (KRAS^{G12C}) inside the cell via proximity induced chemistry, step-II: KRAS will undergo the proteasomal digestion and a few fragments of the protein typically a few peptide long (including covalently modified) will load on MHC and display on the cell surface, step-III: a bifunctional protein comprise of a HaloTag protein, flexible peptide linker G4S or 3(G4S) and anti-CD3 scFv, will covalently react with displayed haloalkane on MHC and recruit the T-cell via CD3 (on the T-cell surface) recognition with the other end, the T-cell used here is human interleukin-2 luciferase (IL2-Luc) reporter Jurkat line (contains a firefly luciferase gene under the control of a human IL2 promoter stably integrated in the genome), now the activated T-cell will produce luciferase along with IL-2 (FIG. 26B). The produced luciferase can be quantified using bioluminescence. Using TRICL assay (10 mM of small molecule and 2 mM of bifunctional protein), Applicant found that modification with shorter linkers is efficient in general which is further enhanced with the structural rigidity while a tertiary amine (carries a positive due to protonation in the physiological pH) attenuates the same (FIG. 26C). As the production of luciferase depends upon the T-cell activation, Applicant transformed the bioluminescence intensity to T-cell activation. To investigate that the T-cell activation is indeed due the covalent ligation of cysteine on KRAS Applicant compared G12C and G12D mutation containing cells and see that the later shows insignificant activation compared to the former (FIG. 26D). Next, Applicant wanted to test whether the linker of bifunctional protein has effect on the T-cell activation. Applicant used two protein constructs, bifunctional HaloTag/anti-CD3 scFv protein linked with flexible peptides G4S or 3(G4S). Applicant's results suggest that 3(G4S) linker works better (FIG. 26E). To further validate the robustness of the assay Applicant used competition and negative control assay. As negative control Applicant used a bifunctional chimeric small molecule (11) which contains a Bruton's Tyrosine Kinase (BTK) binder in place of KRAS binder and as a competitor Applicant used covalent KRAS(G12C) inhibitor Sotorasib (10S, FIG. 26F). Applicant observed that treatment of MIA PaCa2 cells with 11 (10 mM) fails to activate T-cell and a reduces T-cell activation with 10 (10 mM) in presence of 10 mM 10S (FIG. 26G) which confirms the specificity of the assay.

### Example 7—Generalization of T-Cell Recruiting Chimeras for Other Targets

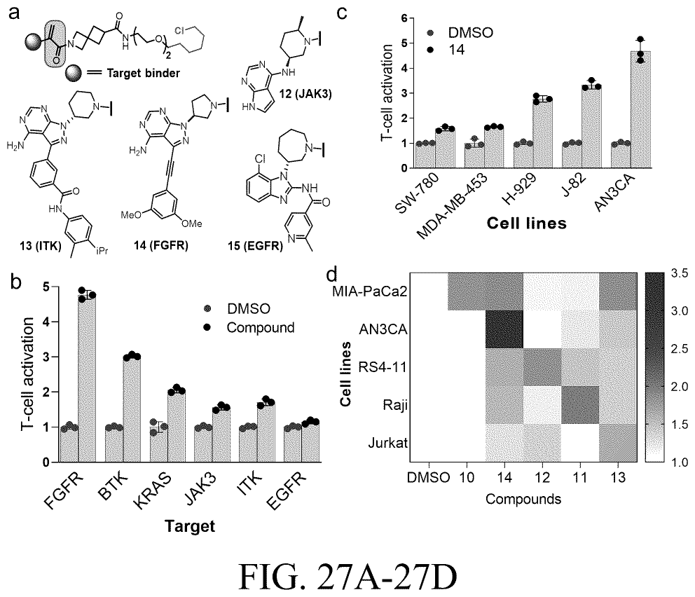

After successfully demonstrating the TRICL assay in studying the neo-HLA display and T-cell activation thereafter, Applicant applied the TRICL assay to other intracellular proteins. Many intracellular kinases are of a great interest in the field of targeted cancer cell killing and other disease condition. Applicant chose Fibroblast growth factor receptor (FGFR), Epidermal growth factor receptor (EGFR), Janus kinase 3 (JAK3), IL2 inducible T cell kinase (ITK) and BTK as the target. First, Applicant designed and synthesized a set of chimeric small molecules equipped with binder of the corresponding target, HaloTag ligand and the best linker identified in previous experiments (FIGS. 26A, 26C, 27A). Next, Applicant perform the TRICL assay by treating cells (known to express the corresponding kinase) with chimeric small molecules (Target:cell-FGFR:AN3CA, BTK:Raji, KRAS:MIA-PaCa2, JAK3:RS4-11, ITK:Jurkat, EGFR:A431) (The Human Protein Atlas database). Different target shows variable T-cell activation which may attribute to a combined effect of target labelling and HLA displaying efficiency (FIG. 27B). Selecting the best performing chimera i.e., FGFR targeting 14 (based on pan-FGFR inhibitor), Applicant tested it in different cell lines which are known to express different level of FGFR to investigate the correlation between the expression vs. HLA display or T-cell activation (FIG. 27C). Result shows that the T-cell activation is indeed positively correlated with the expression level of FGFR. To get a generalized view, Applicant studied all chimeric small molecules with different cell lines and the result exhibits the neo-HLA display-based T-cell activation has a good correlation with the expression of the target proteins (FIG. 27D). Combining Applicant's results show a promising way forward in the field of proximity induced group transfer chemistry and its application in biological application.

### Example 8—Development of Haptenizing Chimeras (HaCs) from Known Drugs for Cancer Immunotherapy

### 1. Background, Significance, and Aims

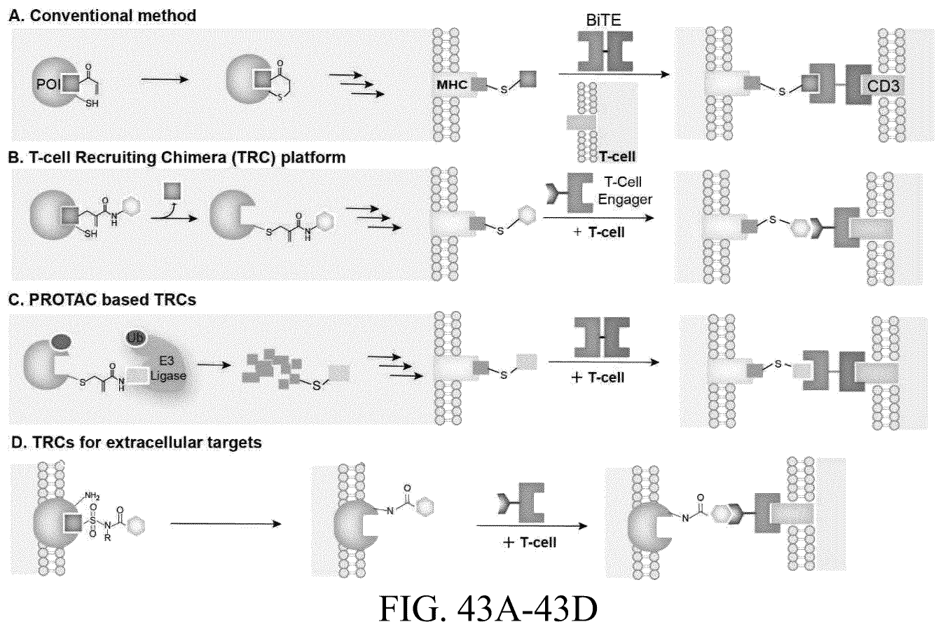

The rapid emergence of acquired resistance is a common failure mode of targeted therapies in cancer. For example, acquired resistance emerged in most patients treated KRAS^{G12C} inhibitors, resulting in poor survival rates and outcomes similar to chemotherapy.(1-4) Cancer cell surfaces display specific antigens that have been targeted. For example, Bispecific T cell engagers (BiTEs) are an emerging therapeutic modality that induces proximity between cancer cells and cytotoxic T cells by simultaneously binding to a cancer-specific antigen and CD3 on T cells.(5) This proximity activates cytotoxic T cells to induce tumor clearance. Recently, some groups have reported a small molecule-based approach for cell surface display of neo-antigen derived from an intracellular oncogene (FIG. 43A).(6,7) Here, the oncogenic KRASG12C labeled with its covalent drug was processed by the immunoproteasome resulting in major histocompatibility complex (MHC) display of a haptenated peptide that bears the covalent drug. Using phage display, antibodies were identified that recognize the covalent drug-MHC peptide complex and this antibody was subsequently used to generate a BiTE that induced proximity between cytotoxic T cells and cancer cells with KRASG12C, triggering cancer cell death. These landmark studies validate the use of covalent inhibitors to generate neo-antigens but suffer from several limitations outlined below, preventing their widespread applications.

Applicant proposes to build a haptenizing chimeras (HaCs) platform consisting of two components: a T cell engager and a chimeric small molecule (termed HaC) that haptenizes an oncogene. HaC is a repurposed covalent drug connected to a bio-orthogonal reactive group (e.g., tetrazine)(8,9) via a group transfer linker. HaC appends the bio-orthogonal reactive group to the oncogene, resulting in the MHC display of that group (FIG. 43B). Subsequently, a click reaction with a T cell engager (i.e., trans-cyclooctene-bearing CD3 binder) assists in inducing proximity between the cancer cell and cytotoxic T cells akin to observed for BiTEs.

The HaC platform may have several advantages over the reported contemporary technologies (FIG. 43A). First, contemporary technologies require the development of a new antibody for every new inhibitor, even for the same target.(6,7) The constancy of the displayed group (e.g., tetrazine) in HaC requires the development of only one T cell engager for multiple inhibitors and targets. This generality across inhibitors/targets enables rapid screening of targets/inhibitors/cancer types, enabling cost-effective target prioritization (see 2.1.1.2 for data). Second, HaC covalently mobilizes T cell engager on cancer cells, resulting in higher residence time of cytotoxic T cells on cancer cells that may enhance cell killing. Furthermore, contemporary BiTEs interact with both MHC-1 and peptide, and since the MHC-1 sequence varies significantly between patients, generating MHC haplotype-independent BiTEs can be challenging.10,11 The covalency afforded by HaCs reduces the need for additional binding interactions between MHC and T cell engager, allowing the HaCs platform to be effective on diverse MHC haplotypes (see 2.1.1.4 for data). Third, displaying a bio-orthogonal reactive group (vs. covalent drug) enables the use of diverse linkers on HaCs, which Applicant's preliminary data (see 2.1.1.1 for data) suggest can impact MHC display, T cell engagement, and efficacy. Finally, tetrazine-TCO click chemistry reduces the size of BiTE by ˜50%, enabling higher tissue penetrance in solid tumors, an issue with current BiTEs. Building on these studies, Applicant will now determine in vivo efficacy of HaCs and build next-generation HaCs through the following aims.

Aim 1. Determine in vivo efficacy of HaCs for KRAS^{G12C} and EGFR^{L858R/790M} drugs.

Using HaloTag and its ligand (i.e., chloroalkane) as bio-orthogonal reactive groups, Applicant validated the HaC platform for several targets (see 2.1.1). However, this HaloTag platform cannot be used clinically for several reasons, including the immunogenicity of the HaloTag protein. Leveraging the recent success of tetrazine-trans-cyclooctene (TCO) click chemistry in clinical trials(12), Applicant will now generate HaCs that display tetrazine-bearing peptides on MHC-1 and demonstrate proximity-induction between cancer and T cells using a T cell engager that bears trans-cyclooctene. By assessing oncogene labeling, MHC labeling, and T cell activation, Applicant will delineate the design principles for generating efficacious HaCs using clinically approved inhibitors for KRAS and EGFR. Finally, Applicant will determine the in vivo efficacy of HaCs in reducing tumor burden.

Aim 2. Develop HaCs from Proteolysis Targeting Chimeras (PROTACs) in Clinical Trials. PROTACs are emergent modalities that also have acquired resistance problems.(13,14) PROTAC-mediated target degradation dramatically increases the number of peptides of the degraded protein on MHC.(15,16) Using Bruton Tyrosine Kinase (BTK) for which Applicant has developed group transfer chemistry(17-22) and for which PROTACs are in clinical trials,(23,24) Applicant will build HaCs from PROTACs that display a cereblon binder (e.g., pomalidomide) on BTK leading to its degradation (FIG. 1C). Addition of T cell engager that can simultaneously bind to pomalidomide and CD3 will induce ternary complex between cancer and T cell, triggering former's death.

### Aim 3. Develop HaCs for Extracellular Targets.

Several cancer cells express selective biomarkers on their cell surface (e.g., prostate-specific membrane antigen (PSMA)) (25). To complement Applicant's approach for intracellular oncogenes in Aims 1 and 2, Applicant will develop a HaC platform for PSMA using the lysine-based group transfer linkers Applicant has developed (FIG. 43D). Overall, Applicant hopes these HaC platforms offer an efficacious approach for repurposing existing covalent drugs and PROTACs to combat resistance development by leveraging the immune system.

### 2. Approach

### Aim 1. Determine In Vivo Efficacy of HaCs for KRASG12C and EGFRL858R/790M Drugs.

### 2.1.1. Preliminary Studies.

### 2.1.1.1. Development of HaCs Platform for KRASG12C Using HaloTag-Based Bio-Orthogonal Chemistry.

To develop HaCs, Applicant choose KRAS^{G12C} as a model target as group transfer-based binders, MHC-I display of haptenated peptide and exploitation of such display for T cell engagement by BiTE has been reported,(6,7) allowing Applicant to develop and benchmark HaCs platform. Applicant synthesized a library of HaCs from Amgen's KRAS drug Sotarasib,(26) a group transfer handle (methacrylamide), and HaloTag ligand (chloroalkane) connected via linkers with various lengths, and flexibility (FIG. 44A). Next, to determine the T-cell activation potential of HaCs, Applicant generated a T cell engager (i.e., HaloTag-bearing anti-CD3 scFv) and used the previously described T cell activation reporter assay.6,7 Here, as a model for cytotoxic T cells, Applicant used a Jurkat cell line with a TL2-promoter driven luciferase (FIG. 44B). Upon formation of complex between the cancer cell and Jurkat cell mediated by the HaloTag-bearing anti-CD3, the signal transduction in Jurkat cells results in TL2-promoter driven expression of the luciferase, whose bioluminescence serves as a proxy for T-cell activation.

Applicant treated KRAS^{G12C} cells (Mia Paca-2) with HaCs (FIG. 44A) followed by washing and incubation with a T cell engager (i.e., HaloTag-bearing anti-CD3 scFv) and Jurkat cells for 4 and 24 hrs, respectively. Subsequently, the cells were lysed, and luciferase activity was quantified. Using this workflow, Applicant found that HaCs with shorter linkers were more efficient at T-cell activation than those with longer linkers for all five series of HaCs (FIG. 44A, 44C)—the spiro-azetidine linker exhibited the most activation (FIG. 44C). With the optimized HaC in hand, Applicant optimized the spacer between the HaloTag and anti-CD3 scFv by testing GGGGS (G4S) (SEQ ID NO: 41) or 3(G4S) (SEQ ID NO: 42) linkers—the 3(G4S) (SEQ ID NO: 42) spacer led to higher T-cell activation (FIG. 44D).

Next, Applicant performed four sets of orthogonal control experiments to confirm that the observed T-cell activation is due to KRAS^{G12C} inhibitor-mediated group transfer and MHC display. First, Applicant compared T-cell activation from different cancer cell lines that lack KRAS^{G12C}. Applicant observed the highest activation only from the MIA-PaCa2 cell line, which expresses KRAS^{G12C}, pointing towards group transfer dependence (FIG. 44E). Then Applicant compared T-cell activation from cells bearing the KRAS^{G12C} or KRAS^{G12D} mutations with the latter lacking the cysteine necessary for group transfer reactivity. As expected, Applicant observed insignificant T-cell activation from KRAS^{G12D} cells compared to KRAS^{G12C} cells (FIG. 44F). Third, Applicant used a negative control compound 7, which contains the same linker and chloroalkane as 5a, but a Bruton's tyrosine kinase (BTK) binder in place of the KRAS^{G12C} binder (FIG. 44G). Treatment of KRAS^{G12C} cells with compound 7 did not result in any significant T-cell activation in the KRAS^{G12C} cells. Finally, Applicant performed a competition experiment wherein the cells were treated with KRAS^{G12C} inhibitor Sotorasib (6, FIG. 44G), which should cap the KRAS^{G12C} cysteine and prevent subsequent group transfer chemistry with HaCs. As expected, the competitor sotorasib 6 reduced T-cell activation by 5a (FIG. 2H). Overall, these four orthogonal control experiments suggest bona fide T-cell activation via proximity induced by T cell engager between Jurkat cell and cancer cells and validates the HaCs platform for KRAS^{G12C} using HaloTag and Amgen's Sotorasib drug.

### 2.1.1.2. Rapid Generalization of HaCs Platform to Multiple Targets.

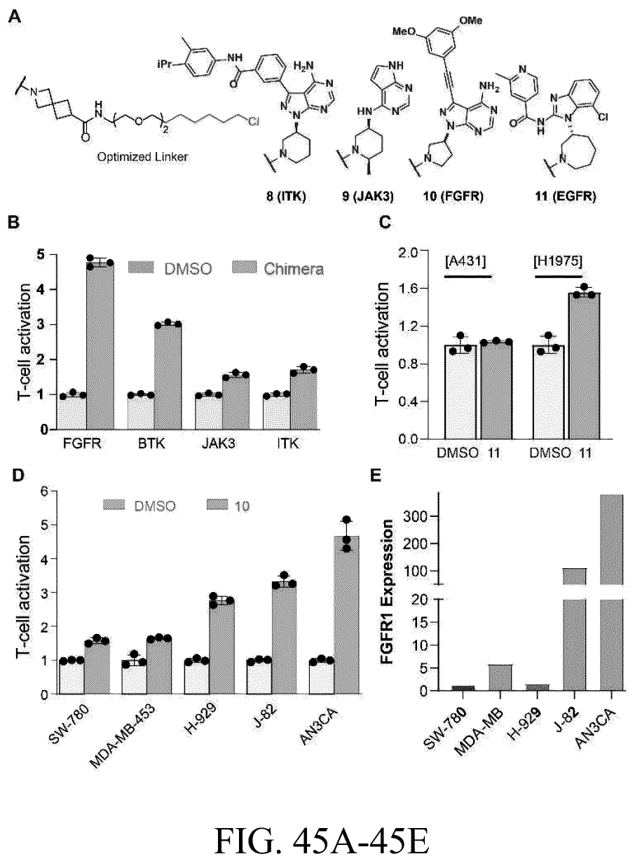

After successfully demonstrating HaCs for KRAS^{G12C}, Applicant demonstrated the generality of the platform by rapidly generating and validating HaCs for five other targets. These studies did not require developing new antibodies for the inhibitor (i.e., no new T cell engager) for the target as each inhibitor displayed chloroalkane (FIG. 45A). Applicant chose high-value cancer targets, including Fibroblast Growth Factor Receptor (FGFR), an oncogenic double mutant of Epidermal Growth Factor Receptor (EGFR^{L858R/790M}) Janus kinase 3 (JAK3), and BTK.

Applicant designed and synthesized the corresponding HaCs from various drugs, including PF-06465469 8 (27) for ITK, PF-06651600 9 (28) for JAK3, Futibatinib 10 (29) for FGFR, Nazartinib 11 (30) for EGFR^{L858R/790M} mutant, and Ibrutinib 7 (31) for BTK. Applicant performed the T cell activation assay by treating cells that express the corresponding targets with the corresponding HaCs (Target: cell line-FGFR: AN3CA; BTK: Raji; JAK3:RS4-11; EGFR: A431). For all targets, Applicant observed T-cell activation, pointing to the generality of the platform (FIG. 45B). For EGFR, Applicant used compound 11, which is selective for EGFR^{T790M/L858R} mutant over wild type. Applicant observed T-cell activation in the mutant line (not wild type) like mutant studies on KRAS^{G12C} (FIG. 45C).

### 2.1.1.3. HaCs Enable the Display of Non-Canonical MHC Peptides.

Per contemporary algorithms that predict the sequence of MHC-1 displayed peptides, the cysteine-bearing peptides for most targets above are not predicted to be displayed. Typically, the MHC-I displayed peptides have anchoring residues to enhance the interaction between the peptide and MHC-1, which restricts the sequence space.(32) However, Applicant hypothesizes that the covalent addition of organic fragments to peptides enhances their binding to MHC-1 via potentially hydrophobic interactions. Furthermore, the covalent modification of the oncogene can alter the nature of peptides processed by the immunoproteasome. Thus, non-canonical peptides can be displayed by chemically modifying the target protein, enabling the generation of neo-antigens for targets not previously predicted to be displayed.

### 2.1.1.4. T-Cell Activation by HaCs is Independent of the HLA Haplotype.

Applicant investigated the correlation between target expression levels, HLA haplotype, and T-cell activation for the HaCs platform. Applicant selected the FGFR targeting HaCs 10 and tested it in different cell lines known to express different levels of FGFR and with different HLA haplotypes. (FIG. 45D, 45E). T-cell activation generally positively correlates with the expression level of FGFR, but not HLA haplotype, indicating that the expression level of the target protein significantly affects the overall level of T-cell activation.

### 2.1.1.5. Development of HaCs Platform for FGFR Using Click Chemistry.

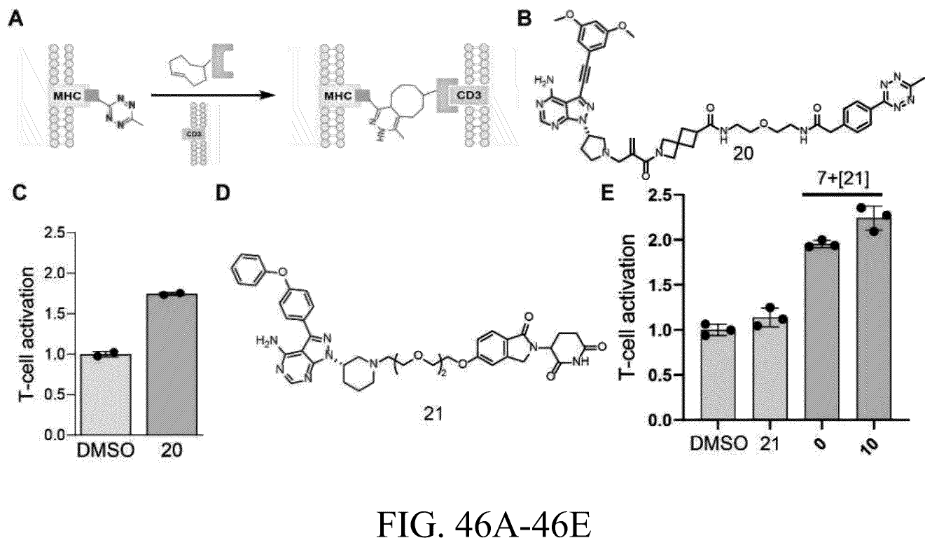

Replacement of HaloTag: chloroalkane bio-orthogonal reactive pair with tetrazine-TCO (33) pair can have several advantages (FIG. 46A). First, the former is a protein: small molecule pair while the latter is completely small molecule-based thereby reducing the size of the T cell engager by ˜50%. Second, HaloTag is a protein of bacterial origin that may be immunogenic and can elicit adverse immune reactions, particularly on being proximal to immune-system machinery. Third, tetrazine-TCO has a reaction rate in humans at 57.7 M⁻¹s⁻¹, which is among the fastest biorthogonal ligations (34). Finally, tetrazine-TCO click chemistry has been optimized and shown to be efficacious in humans, which is relevant from a translational perspective. Given these potential advantages, Applicant synthesized tetrazine-bearing HaCs (20, FIG. 46B) and T-cell engagers conjugated to TCO via N-hydroxysuccinimide (NHS) chemistry. Applicant's results confirm that tetrazine retains the ability to be displayed and activate T-cells via the TCO conjugated T-Cell engager (FIG. 46C). However, the activation levels are lower than the corresponding halo-tag system. Applicant speculates that during NHS conjugation some of the TCO molecules isomerize to the inactive cis isomer (35), reducing the click reaction. Applicant aims to optimize the conjugation conditions to append multiple TCOs on anti-CD3 scFv.

### 2.1.2. Proposed Experimental Plan.

### 2.1.2.1. Synthesis of a Library of HaCs and T Cell Engagers.

Applicant's designed HaCs are composed of 4 parts (FIG. 47A): 1) Target protein binder, 2) Group transfer reactive group, 3) Linkers, and 4) Click chemistry handle (FIG. 47A). For targets, Applicant will focus on KRAS^{G12C} and EGFR^{T790M/L858R} (FIG. 47B), for which resistance is emerging and have a significant unmet clinical need. For reactive groups, Applicant will employ a series of amino-methacrylamides (FIG. 47C), which react with cysteine at different rates, with pyrrolidine substituted as the fastest and piperazine as the slowest, allowing Applicant to fine-tune the degree of target labeling and specificity. (36) For the linker, Applicant has chosen a set of rigid and flexible linkers of varying lengths: short, medium, and long (FIG. 47D). For the click chemistry handle, Applicant has chosen substituted tetrazine with different reactivity and in vivo stability. For example, pyrimidyl-substituted tetrazines react with TCO approximately 100 times faster than the corresponding methyl substitution (37) (FIG. 47E). But enhanced reactivity is also accompanied by instability-pyrimidyl-substituted tetrazines are degraded within 12 h, while insignificant degradation was observed with the methyl-substituted analogs. (38) To generate the T-cell engagers, Applicant aims to attach TCOs to anti-CD3 scFvs via NHS35 or Sortase conjugation. (39) In a similar way to spacer optimization for HaCs, Applicant will optimize the linker of TCO attachment to anti-CD3 by varying length, rigidity, and stoichiometry (FIG. 47F). Applicant has extensive experience with generating chimeras composed of the components described herein (40), thus the rapid assembly of the library should not be challenging.

### 2.1.2.2. Evaluation of the HaCs in Assays for Labeling, MHC Loading, and T Cell Activation.

To develop design principles for HaCs, Applicant will use three sets of previously described assays that report on the following:

### 2.1.2.2.1. Quantification of Target Labeling Studies by HaCs.

Applicant's library of HaCs will have binders of different affinities and reactive groups, which can affect the stoichiometry of labeling of the target oncogene. Applicant will determine the degree of target labeling by intact mass spectroscopy (MS) biochemically and in cells (FIG. 47A). For the intact mass spectroscopy, purified proteins will be incubated with the corresponding HaCs or DMSO and the degree of labeling will be analyzed by intact LC-MS. The HaCs with the best biochemical labeling will be subject to competitive labeling experiments in cells using an Activity-Based Protein Profiling (ABPP) approach that has been used extensively to profile covalent inhibitors. Briefly, cells treated with HaCs or a vehicle will be lysed and labeled with a broadly reactive ABPP-alkyne probe, followed by click chemistry with heavy or light-labeled biotin-azide (41). After enrichment and proteolytic digestions by trypsin, the isotopically labeled peptides will be analyzed by Orbitrap mass spectrometer (Eclipse) to determine their ratio to quantify the extent of covalent modification. Applicant has optimized such workflow.

### 2.1.2.2.2. Quantification of MHC Binding.

Here, Applicant will synthesize peptides for different HLA alleles (e.g., HLA-A*02:01 and HLA-A*03:01) and modify them by Applicant's HaCs biochemically. The affinity of the modified peptides for MHC will be evaluated both biochemically and on cells. For biochemical evaluation, Applicant quantifies MHC binding by measuring the thermal stability of MHC-peptide complexes by differential scanning fluorimetry (DSF, FIG. 48B) following reported procedures.6 For cellular evaluation, Applicant will use T2 cells that are deficient in transporter associated with antigen processing (TAP) protein (FIG. 48C). This results in the generation of empty MHCs, which can form stable complexes only with exogenously supplied cognate peptides (42,43)—the cells bearing such peptides can be detected using FACS sorting with TCO-bearing dye. Finally, Applicant will use the previously reported proximity-ligation assay (PLA) (FIG. 48D), which allows the detection and localization of MHC-1: peptide complexes on cells.

2.1.2.2.2. Quantification of T-cell activation.

To confirm that Applicant's displayed peptides can recruit and activate T-cells, Applicant will use the luciferase reporter assay described above (FIGS. 44B and 48E). Applicant will also validate whether T-cell activation leads to cytotoxicity using peripheral blood mononuclear cells (PBMCs) (FIG. 48F). Here, Applicant will follow a published protocol6 where the target cells bearing a fluorescent marker will be treated with HaCs. In parallel, human PBMCs will be thawed and cultured overnight. The target cells will then be washed gently three times and co-cultured with PBMCs in the presence of T-cell engager. Subsequently, the number of cancer cells will be quantified using the Operetta confocal imager.

### 2.1.2.3. Evaluation of HaCs Platform In Vivo.

Applicant will measure critical physicochemical (e.g., solubility, permeability) and pharmacokinetic (e.g., microsomal stability, plasma binding) properties for downstream development of HaCs. Ideal characteristics include solubility >50 μM in PBS buffer; plasma stability, with >75% parent molecule remaining after 1-hour incubation in human plasma; membrane permeability, as measured by the Caco-2 permeability assay; and liver microsome stability, such that >50% parent molecule remains after 1-hour incubation in human liver microsomes. After identifying the ideal candidate and formulations, Applicant will determine the maximum tolerable dose studies. All assays are available at various CROs.

While budgetary and time constraints may prevent exhaustive in vivo dosing experiments, Applicant wishes to outline a potential in vivo efficacy study should the studies proposed above yield successful candidates early in the optimization cycle. These efficacy studies will follow the reported procedures involving quantifying the tumor burden and other associated metrics. Two groups of tumor-bearing mice (generated by injecting, for example, luciferase-bearing MIA PaCa-2 cells) will receive four cycles of Tetrazine-containing HaCs at a 0.33 mmol kg-1 dose and TCO-conjugated anti-CD3 scFv. Two groups of mice will be injected with four cycles of HaCs at the same dose followed by vehicle and, finally, two more groups of mice will receive four cycles of either the TCO-conjugated anti-CD3 scFv or vehicle. The animals will be randomly grouped, monitored daily by experienced biotechnicians, and removed from the study in case of poor physical condition (e.g., discomfort, reduced motility). Also, the animals will be removed from the study in the cases of excessive body weight loss (>20% with respect to baseline or >15% in two consecutive measurements) or when tumors reach a 1 cm3 size. If these conditions do not occur, the animals will be maintained in the study for up to two months, after which they will be euthanized.

### 2.1.3. Potential Pitfalls and Proposed Solutions.

The low stoichiometry of MHC-1 displayed peptides can reduce the effective concentration of tetrazine and to enhance reactivity increasing the number of TCOs per anti-CD3 conjugate may be required. However, the unmodified TCO is hydrophobic, which might result in poor solubility of the resulting T-cell engagers. For this purpose, Applicant aims to utilize analogs of TCO, such as d-TCO44 and oxo-TCO45, which have better water solubility and enhanced reaction rates. Many cancers evade immune recognition through overexpression of the ligand for programmed cell death-1(PD-L1), which provides an inhibitory signal for T-cell activation. Applicant anticipates the covalent nature of Applicant's T cell engagers to counteract such effects.

Aim 2. Develop HaCs from Proteolysis Targeting Chimeras (PROTACs) in Clinical Trials.

### 2.2.1. Preliminary Studies.

### 2.2.1.1. Target Degradation by PROTACs Enhances T Cell Activation Using HaCs.

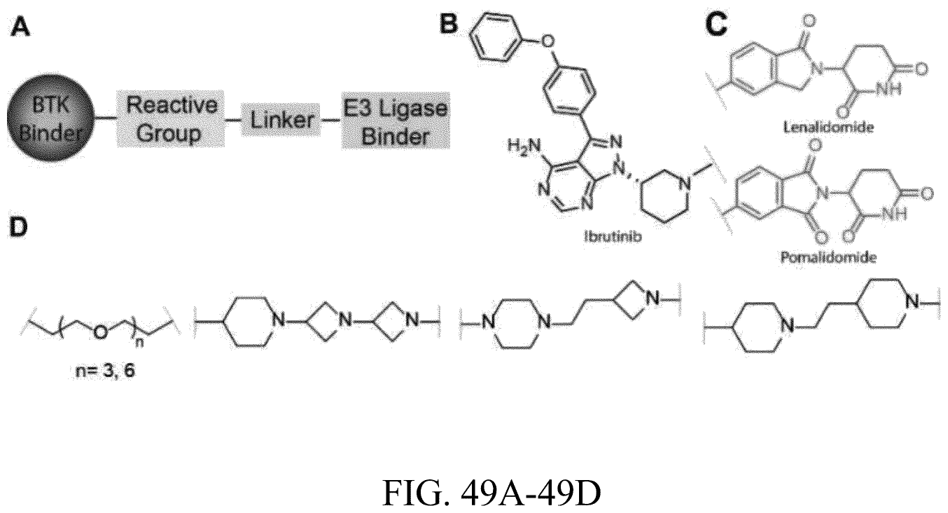

Applicant co-treated cells with the BTK-based HaC 7 and the BTK degrader 21 (FIG. 46D). Applicant noticed a slight increase in T-cell activation (FIG. 46E), which is consistent with previous reports which suggest that PROTACs increase MHC-1 display and the efficacy of BiTEs (16), but further optimization is still required. The co-treatment approach is not ideal because it is a multicomponent system where the molecules might compete. Thus, conversion of the PROTAC molecule to a HaCs, as proposed in Aim 2, will be preferable. To design Applicant's PROTAC-based HaCs (FIG. 49A), Applicant will maintain the same general composition described in 2.1.2.1 using BTK as a model system due to the extensive validation of BTK degraders. (46-49) Thus, Applicant's PROTAC-HaCs will be composed of a BTK binder such as Ibrutinib (FIG. 49B), a reactive group (as described in FIG. 47C), a linker and E3-Ligase binder (e.g., pomalidomide, FIG. 49C). In this case Applicant aim to utilize linkers based on well-validated BTK PROTACs (46-49) (FIG. 49D).

### 2.2.2. Proposed Experimental Plan.

### 2.2.2.1. Synthesis of a Library of HaCs and T Cell Engagers.

For the synthesis of PROTAC-based HaCs, Applicant will maintain a general composition as described in aim 1.1 (FIG. 43C). One significant difference will be that instead of displaying a biorthogonal handle, Applicant will be displaying an E3 ligase binder. Applicant will perform linker optimization of T-cell engager and effective E3 ligase recruitment and subsequent degradation by HaC. Applicant will initially fuse the small molecule binding domain of CRBN proteins with anti-CD3 scFv to generate the corresponding PROTAC-based T-cell engagers (FIG. 44C).

### 2.2.2.2. Evaluation of the HaCs in Assays for Labeling, MHC Loading, and T Cell Activation.

Same as Aim 1.2 and will now include degradation assays.

### 2.2.3. Potential Pitfalls and Proposed Solutions.

In addition to the pitfalls described in 2.1.3., Applicant's PROTAC approach has the potential drawback of reliance on the CRBN binding domain for generating the corresponding T-cell engagers. Herein, Applicant might have to compromise small molecule binding affinity for reduced size. If Applicant's PROTAC T-cell engagers show low binding affinity to the CRBN binders, Applicant can overcome this problem by generating antibodies that will recognize the peptide complex with CRBN binders and fuse them with the anti-CD3. This approach compromises the reduced size of T-cell engagers but retains the mechanistic synergism of PROTACs and HLA display.

### Aim 3. Develop HaCs for Extracellular Targets.

### 2.3.1. Preliminary Studies.

### 2.3.1.1. Development of Lysine-Based Group Transfer Linkers.

Most lysine-targeting group transfer linkers suffer from poor reaction rates, lack of stability/selectivity, and a bulky nature. N-acyl-N-alkyl sulfonamides (NASA) have recently gained interest as covalent warheads for lysine targeting due to their fast reaction rates towards lysine (k≈10⁴ M⁻¹ s⁻¹). (50,51) Despite these promising attributes, NASA is bulky (unfit to be used as a linker for bifunctional molecules that are already large), hydrolyzes rapidly (t^{1/2}≈5 h in PBS), and lacks tunable analogs. Guided by physical organic chemistry, Applicant rationally designed NASA analogs with a smaller footprint, higher stability, and tunable reactivity. The NASA warheads follow a general structural motif as described in FIG. 50A. Traditionally, R₁ is a phenyl ring that, in original reports, served as a convenient handle for attachment to a solid phase resin. (52) Replacement of this ring with short aliphatic groups did not affect its reactivity towards lysine (compare N19 and N20 in FIG. 50B), permitting significant size reduction without loss of reactivity. The electrophilicity of the N-acyl group depends on the electron-withdrawing properties of the N-alkyl group (—R₂), allowing fine-tuning of amine reactivity and hydrolytic stability. Applicant synthesized a series of NASA derivatives (FIG. 50B) and identified the —CH₂CF₃ group as a superior alternative to the typically used —CH₂CN by providing comparable rates towards aminolysis (compare compounds N15 and N20 in FIG. 50B) accompanied by a 5-fold decrease of hydrolysis rates (FIG. 50C). These studies also provided analogs to fine-tune reactivity (FIG. 50B) and effective molarity.

### 2.3.1.2. Development of HaCs Platform for PSMA Using HaloTag for Bio-Orthogonal Chemistry.

Applicant used the optimized NASA group to design a HaC targeting PSMA, which is overexpressed in many prostate cancers by 8-12 over noncancerous prostate cells. (53) Applicant used the PSMA binding motif (DUPA) (FIG. 50D), which has a high affinity for PSMA (Kᵢ=8 nM) (54), and the optimized NASA scaffold (FIG. 50D) to generate the PSMA-based HaCs 19. Applicant's optimized NASA warhead allowed us to capture a deeply buried lysine (Lys537) within the PSMA binding pocket, as identified by LC-MS/MS analysis (FIG. 50E). This compound provided T-cell activation only on PSMA-positive prostate cancer cells (FIG. 50F) and not non-cancerous prostate cells.

### 2.3.2. Proposed Experimental Plan.

### 2.2.2.1. Synthesis of a Library of HaCs and T Cell Engagers.

To synthesize extracellular HaCs, Applicant will use optimized lysine warheads (FIG. 50B) and PSMA binder (55). Through the lysine group chemistry, Applicant will transfer tetrazine handles, which will be recognized by TCO-conjugated T-cell engagers (FIG. 43D), similarly as described in Aim 1.1.

### 2.2.2.2. Evaluation of the HaCs in Assays for Labeling and T Cell Activation.

Same as in 2.1.2.2.

### 2.3.3. Potential Pitfalls and Proposed Solutions.

The anticipated pitfalls for this approach are similar to those described in 2.1.3.

### REFERENCES

(1) Diverse alterations associated with resistance to KRAS(G12C) inhibition. Zhao, Y.; Murciano-Goroff, Y. R.; Xue, J. Y.; Ang, A.; Lucas, J.; Mai, T. T.; Da Cruz Paula, A. F.; Saiki, A. Y.; Mohn, D.; Achanta, P.; Sisk, A. E.; Arora, K. S.; Roy, R. S.; Kim, D.; Li, C.; Lim, L. P.; Li, M.; Bahr, A.; Loomis, B. R.; de Stanchina, E.; Reis-Filho, J. S.; Weigelt, B.; Berger, M.; Riely, G.; Arbour, K. C.; Lipford, J. R.; Li, B. T.; Lito, P. Nature 2021, 599, 679-683.PMC8887821
(2) Mechanisms of Resistance to KRAS(G12C)-Targeted Therapy. Akhave, N. S.; Biter, A. B.; Hong, D. S. Cancer Discov 2021, 11, 1345-1352.PMC8178176
(3) Clinical Acquired Resistance to KRAS(G12C) Inhibition through a Novel KRAS Switch-II Pocket Mutation and Polyclonal Alterations Converging on RAS-MAPK Reactivation. Tanaka, N.; Lin, J. J.; Li, C.; Ryan, M. B.; Zhang, J.; Kiedrowski, L. A.; Michel, A. G.; Syed, M. U.; Fella, K. A.; Sakhi, M.; Baiev, I.; Juric, D.; Gainor, J. F.; Klempner, S. J.; Lennerz, J. K.; Siravegna, G.; Bar-Peled, L.; Hata, A. N.; Heist, R. S.; Corcoran, R. B. Cancer Discov 2021, 11, 1913-1922.PMC8338755
(4) Acquired Resistance to KRAS(G12C) Inhibition in Cancer. Awad, M. M.; Liu, S.; Rybkin, II; Arbour, K. C.; Dilly, J.; Zhu, V. W.; Johnson, M. L.; Heist, R. S.; Patil, T.; Riely, G. J.; Jacobson, J. O.; Yang, X.; Persky, N. S.; Root, D. E.; Lowder, K. E.; Feng, H.; Zhang, S. S.; Haigis, K. M.; Hung, Y. P.; Sholl, L. M.; Wolpin, B. M.; Wiese, J.; Christiansen, J.; Lee, J.; Schrock, A. B.; Lim, L. P.; Garg, K.; Li, M.; Engstrom, L. D.; Waters, L.; Lawson, J. D.; Olson, P.; Lito, P.; Ou, S. I.; Christensen, J. G.; Janne, P. A.; Aguirre, A. J. N Engl J Med 2021, 384, 2382-2393.PMC8864540
(5) T cell-engaging therapies—BiTEs and beyond. Goebeler, M. E.; Bargou, R. C. Nat Rev Clin Oncol 2020, 17, 418-434
(6) A covalent inhibitor of K-Ras(G12C) induces MHC class I presentation of haptenated peptide neoepitopes targetable by immunotherapy. Zhang, Z.; Rohweder, P. J.; Ongpipattanakul, C.; Basu, K.; Bohn, M. F.; Dugan, E. J.; Steri, V.; Hann, B.; Shokat, K. M.; Craik, C. S. Cancer Cell 2022, 40, 1060-1069 e7
(7) Creating MHC-Restricted Neoantigens with Covalent Inhibitors That Can Be Targeted by Immune Therapy. Hattori, T.; Maso, L.; Araki, K. Y.; Koide, A.; Hayman, J.; Akkapeddi, P.; Bang, I.; Neel, B. G.; Koide, S. Cancer Discov 2023, 13, 132-145.PMC9827112
(8) Bioorthogonal chemistry. Scinto, S. L.; Bilodeau, D. A.; Hincapie, R.; Lee, W.; Nguyen, S. S.; Xu, M.; am Ende, C. W.; Finn, M. G.; Lang, K.; Lin, Q.; Pezacki, J. P.; Prescher, J. A.; Robillard, M. S.; Fox, J. M. Nature Reviews Methods Primers 2021, 1, 30
(9) Bioorthogonal Chemistry and Its Applications. Bird, R. E.; Lemmel, S. A.; Yu, X.; Zhou, Q. A. Bioconjugate Chemistry 2021, 32, 2457-2479
(10) Human MHC architecture and evolution: implications for disease association studies. Traherne, J. A. Int J Immunogenet 2008, 35, 179-92.PMC2408657
(11) Human leukocyte antigen super-locus: nexus of genomic supergenes, SNPs, indels, transcripts, and haplotypes. Kulski, J. K.; Suzuki, S.; Shiina, T. Human Genome Variation 2022, 9, 49
(12) ‘Clicked’ drugs: researchers prove the remarkable chemistry in humans. Peplow, M. Nature Biotechnology 2023, 41, 883-885
(13) Proof-of-Concept with PROTACs in Prostate Cancer. Cancer Discovery 2020, 10, 1084-1084
(14) Profiling the Landscape of Drug Resistance Mutations in Neosubstrates to Molecular Glue Degraders. Gosavi, P. M.; Ngan, K. C.; Yeo, M. J. R.; Su, C.; Li, J.; Lue, N. Z.; Hoenig, S. M.; Liau, B. B. ACS Central Science 2022, 8, 417-429
(15) Specific MHC-I Peptides Are Induced Using PROTACs. Jensen, S. M.; Potts, G. K.; Ready, D. B.; Patterson, M. J. Front Immunol 2018, 9, 2697.PMC6262898
(16) Proteolysis-Targeting Chimeras Enhance T Cell Bispecific Antibody-Driven T Cell Activation and Effector Function through Increased MHC Class I Antigen Presentation in Cancer Cells. Massafra, V.; Tundo, S.; Dietzig, A.; Ducret, A.; Jost, C.; Klein, C.; Kontermann, R. E.; Knoetgen, H.; Steegmaier, M.; Romagnani, A.; Nagel, Y. A. J Immunol 2021, 207, 493-504
(17) 6-Substituted-4-(3-bromophenylamino)quinazolines as Putative Irreversible Inhibitors of the Epidermal Growth Factor Receptor (EGFR) and Human Epidermal Growth Factor Receptor (HER-2) Tyrosine Kinases with Enhanced Antitumor Activity. Tsou, H.-R.; Mamuya, N.; Johnson, B. D.; Reich, M. F.; Gruber, B. C.; Ye, F.; Nilakantan, R.; Shen, R.; Discafani, C.; DeBlanc, R.; Davis, R.; Koehn, F. E.; Greenberger, L. M.; Wang, Y.-F.; Wissner, A. Journal of Medicinal Chemistry 2001, 44, 2719-2734
(18) Live-Cell Protein Sulfonylation Based on Proximity-driven N-Sulfonyl Pyridone Chemistry. Matsuo, K.; Nishikawa, Y.; Masuda, M.; Hamachi, I. Angewandte Chemie International Edition 2018, 57, 659-662
(19) Rapid labelling and covalent inhibition of intracellular native proteins using ligand-directed N-acyl-N-alkyl sulfonamide. Tamura, T.; Ueda, T.; Goto, T.; Tsukidate, T.; Shapira, Y.; Nishikawa, Y.; Fujisawa, A.; Hamachi, I. Nature Communications 2018, 9, 1870
(20) A programmable chemical switch based on triggerable Michael acceptors. Zhuang, J.; Zhao, B.; Meng, X.; Schiffman, J. D.; Perry, S. L.; Vachet, R. W.; Thayumanavan, S. Chem Sci 2020, 11, 2103-2111.PMC8150097
(21) Site-Specific Labeling of Endogenous Proteins Using CoLDR Chemistry. Reddi, R. N.; Rogel, A.; Resnick, E.; Gabizon, R.; Prasad, P. K.; Gurwicz, N.; Barr, H.; Shulman, Z.; London, N. Journal of the American Chemical Society 2021, 143, 20095-20108
(22) Development and Applications of Chimera Platforms for Tyrosine Phosphorylation. Pergu, R.; Shoba, V. M.; Chaudhary, S. K.; Munkanatta Godage, D. N. P.; Deb, A.; Singha, S.; Dhawa, U.; Singh, P.; Anokhina, V.; Singh, S.; Siriwardena, S. U.; Choudhary, A. ACS Central Science 2023, 9, 1558-1566
(23) PROTAC targeted protein degraders: the past is prologue. Békés, M.; Langley, D. R.; Crews, C. M. Nature Reviews Drug Discovery 2022, 21, 181-200
(24) PROTACs: past, present and future. Li, K.; Crews, C. M. Chemical Society Reviews 2022, 51, 5214-5236
(25) Using PSMA imaging for prognostication in localized and advanced prostate cancer. Roberts, M. J.; Maurer, T.; Perera, M.; Eiber, M.; Hope, T. A.; Ost, P.; Siva, S.; Hofman, M. S.; Murphy, D. G.; Emmett, L.; Fendler, W. P. Nat Rev Urol 2023, 20, 23-47
(26) The clinical KRAS(G12C) inhibitor AMG 510 drives anti-tumour immunity. Canon, J.; Rex, K.; Saiki, A. Y.; Mohr, C.; Cooke, K.; Bagal, D.; Gaida, K.; Holt, T.; Knutson, C. G.; Koppada, N.; Lanman, B. A.; Werner, J.; Rapaport, A. S.; San Miguel, T.; Ortiz, R.; Osgood, T.; Sun, J.-R.; Zhu, X.; McCarter, J. D.; Volak, L. P.; Houk, B. E.; Fakih, M. G.; O'Neil, B. H.; Price, T. J.; Falchook, G. S.; Desai, J.; Kuo, J.; Govindan, R.; Hong, D. S.; Ouyang, W.; Henary, H.; Arvedson, T.; Cee, V. J.; Lipford, J. R. Nature 2019, 575, 217-223
(27) Covalent inhibitors of interleukin-2 inducible T cell kinase (itk) with nanomolar potency in a whole-blood assay. Zapf, C. W.; Gerstenberger, B. S.; Xing, L.; Limburg, D. C.; Anderson, D. R.; Caspers, N.; Han, S.; Aulabaugh, A.; Kurumbail, R.; Shakya, S.; Li, X.; Spaulding, V.; Czerwinski, R. M.; Seth, N.; Medley, Q. G. J Med Chem 2012, 55, 10047-63
(28) Discovery of a JAK3-Selective Inhibitor: Functional Differentiation of JAK3-Selective Inhibition over pan-JAK or JAK1-Selective Inhibition. Telliez, J. B.; Dowty, M. E.; Wang, L.; Jussif, J.; Lin, T.; Li, L.; Moy, E.; Balbo, P.; Li, W.; Zhao, Y.; Crouse, K.; Dickinson, C.; Symanowicz, P.; Hegen, M.; Banker, M. E.; Vincent, F.; Unwalla, R.; Liang, S.; Gilbert, A. M.; Brown, M. F.; Hayward, M.; Montgomery, J.; Yang, X.; Bauman, J.; Trujillo, J. I.; Casimiro-Garcia, A.; Vajdos, F. F.; Leung, L.; Geoghegan, K. F.; Quazi, A.; Xuan, D.; Jones, L.; Hett, E.; Wright, K.; Clark, J. D.; Thorarensen, A. ACS Chem Biol 2016, 11, 3442-3451
(29) TAS-120 Overcomes Resistance to ATP-Competitive FGFR Inhibitors in Patients with FGFR2 Fusion-Positive Intrahepatic Cholangiocarcinoma. Goyal, L.; Shi, L.; Liu, L. Y.; Fece de la Cruz, F.; Lennerz, J. K.; Raghavan, S.; Leschiner, I.; Elagina, L.; Siravegna, G.; Ng, R. W. S.; Vu, P.; Patra, K. C.; Saha, S. K.; Uppot, R. N.; Arellano, R.; Reyes, S.; Sagara, T.; Otsuki, S.; Nadres, B.; Shahzade, H. A.; Dey-Guha, I.; Fetter, I. J.; Baiev, I.; Van Seventer, E. E.; Murphy, J. E.; Ferrone, C. R.; Tanabe, K. K.; Deshpande, V.; Harding, J. J.; Yaeger, R.; Kelley, R. K.; Bardelli, A.; Iafrate, A. J.; Hahn, W. C.; Benes, C. H.; Ting, D. T.; Hirai, H.; Getz, G.; Juric, D.; Zhu, A. X.; Corcoran, R. B.; Bardeesy, N. Cancer Discov 2019, 9, 1064-1079.PMC6677584
(30) Discovery of (R,E)-N-(7-Chloro-1-(1-[4-(dimethylamino)but-2-enoyl]azepan-3-yl)-1H-benzo[d]imidazol-2-yl)-2-methylisonicotinamide (EGF816), a Novel, Potent, and WT Sparing Covalent Inhibitor of Oncogenic (L858R, ex19del) and Resistant (T790M) EGFR Mutants for the Treatment of EGFR Mutant Non-Small-Cell Lung Cancers. Lelais, G.; Epple, R.; Marsilje, T. H.; Long, Y. O.; McNeill, M.; Chen, B.; Lu, W.; Anumolu, J.; Badiger, S.; Bursulaya, B.; DiDonato, M.; Fong, R.; Juarez, J.; Li, J.; Manuia, M.; Mason, D. E.; Gordon, P.; Groessl, T.; Johnson, K.; Jia, Y.; Kasibhatla, S.; Li, C.; Isbell, J.; Spraggon, G.; Bender, S.; Michellys, P. Y. J Med Chem 2016, 59, 6671-89
(31) The Bruton tyrosine kinase inhibitor PCI-32765 blocks B-cell activation and is efficacious in models of autoimmune disease and B-cell malignancy. Honigberg, L. A.; Smith, A. M.; Sirisawad, M.; Verner, E.; Loury, D.; Chang, B.; Li, S.; Pan, Z.; Thamm, D. H.; Miller, R. A.; Buggy, J. J. Proc Natl Acad Sci USA 2010, 107, 13075-80.PMC2919935
(32) A pattern search method for putative anchor residues in T cell epitopes. Hobohm, U.; Meyerhans, A. Eur J Immunol 1993, 23, 1271-6
(33) Tetrazine ligation: fast bioconjugation based on inverse-electron-demand Diels-Alder reactivity. Blackman, M. L.; Royzen, M.; Fox, J. M. J Am Chem Soc 2008, 130, 13518-9.PMC2653060
(34) Unleashing the Power of Bond Cleavage Chemistry in Living Systems. Wang, J.; Wang, X.; Fan, X.; Chen, P. R. ACS Cent Sci 2021, 7, 929-943.PMC8227596
(35) Enhancing reactivity for bioorthogonal pretargeting by unmasking antibody-conjugated trans-cyclooctenes. Rahim, M. K.; Kota, R.; Haun, J. B. Bioconjug Chem 2015, 26, 352-60.PMC5022565
(36) Systematic Study of the Glutathione Reactivity of N-Phenylacrylamides: 2. Effects of Acrylamide Substitution. Birkholz, A.; Kopecky, D. J.; Volak, L. P.; Bartberger, M. D.; Chen, Y.; Tegley, C. M.; Arvedson, T.; McCarter, J. D.; Fotsch, C.; Cee, V. J. J Med Chem 2020, 63, 11602-11614
(37) Inverse electron demand Diels-Alder reactions in chemical biology. Oliveira, B. L.; Guo, Z.; Bernardes, G. J. L. Chem Soc Rev 2017, 46, 4895-4950
(38) Highly Stable and Selective Tetrazines for the Coordination-Assisted Bioorthogonal Ligation with Vinylboronic Acids. Eising, S.; Engwerda, A. H. J.; Riedijk, X.; Bickelhaupt, F. M.; Bonger, K. M. Bioconjug Chem 2018, 29, 3054-3059.PMC6148442
(39) Sortase-mediated protein ligation: a new method for protein engineering. Mao, H.; Hart, S. A.; Schink, A.; Pollok, B. A. J Am Chem Soc 2004, 126, 2670-1
(40) Development and Applications of Chimera Platforms for Tyrosine Phosphorylation. Pergu, R.; Shoba, V. M.; Chaudhary, S. K.; Munkanatta Godage, D. N. P.; Deb, A.; Singha, S.; Dhawa, U.; Singh, P.; Anokhina, V.; Singh, S.; Siriwardena, S. U.; Choudhary, A. ACS Central Science 2023
(41) Proteome-wide covalent ligand discovery in native biological systems. Backus, K. M.; Correia, B. E.; Lum, K. M.; Forli, S.; Homing, B. D.; Gonzalez-Paez, G. E.; Chatterjee, S.; Lanning, B. R.; Teijaro, J. R.; Olson, A. J.; Wolan, D. W.; Cravatt, B. F. Nature 2016, 534, 570-4.PMC4919207
(42) Assessment of major histocompatibility complex class I interaction with Epstein-Barr virus and human immunodeficiency virus peptides by elevation of membrane H-2 and HLA in peptide loading-deficient cells. Stuber, G.; Modrow, S.; Hoglund, P.; Franksson, L.; Elvin, J.; Wolf, H.; Karre, K.; Klein, G. Eur J Immunol 1992, 22, 2697-703
(43) Identification of wild-type and mutant p53 peptides binding to HLA-A2 assessed by a peptide loading-deficient cell line assay and a novel major histocompatibility complex class I peptide binding assay. Stuber, G.; Leder, G. H.; Storkus, W. T.; Lotze, M. T.; Modrow, S.; Szekely, L.; Wolf, H.; Klein, E.; Karre, K.; Klein, G. Eur J Immunol 1994, 24, 765-8
(44) Conformationally Strained trans-Cyclooctene with Improved Stability and Excellent Reactivity in Tetrazine Ligation. Darko, A.; Wallace, S.; Dmitrenko, O.; Machovina, M. M.; Mehl, R. A.; Chin, J. W.; Fox, J. M. Chem Sci 2014, 5, 3770-3776.PMC4477040
(45) Computationally guided discovery of a reactive, hydrophilic trans-5-oxocene dienophile for bioorthogonal labeling. Lambert, W. D.; Scinto, S. L.; Dmitrenko, O.; Boyd, S. J.; Magboo, R.; Mehl, R. A.; Chin, J. W.; Fox, J. M.; Wallace, S. Org Biomol Chem 2017, 15, 6640-6644.PMC5708333
(46) Delineating the role of cooperativity in the design of potent PROTACs for BTK. Zorba, A.; Nguyen, C.; Xu, Y.; Starr, J.; Borzilleri, K.; Smith, J.; Zhu, H.; Farley, K. A.; Ding, W.; Schiemer, J.; Feng, X.; Chang, J. S.; Uccello, D. P.; Young, J. A.; Garcia-Irrizary, C. N.; Czabaniuk, L.; Schuff, B.; Oliver, R.; Montgomery, J.; Hayward, M. M.; Coe, J.; Chen, J.; Niosi, M.; Luthra, S.; Shah, J. C.; El-Kattan, A.; Qiu, X.; West, G. M.; Noe, M. C.; Shanmugasundaram, V.; Gilbert, A. M.; Brown, M. F.; Calabrese, M. F. Proc Natl Acad Sci USA 2018, 115, E7285-E7292.PMC6077745
(47) Design, synthesis and biological evaluation of Proteolysis Targeting Chimeras (PROTACs) as a BTK degraders with improved pharmacokinetic properties. Jaime-Figueroa, S.; Buhimschi, A. D.; Toure, M.; Hines, J.; Crews, C. M. Bioorg Med Chem Lett 2020, 30, 126877.PMC7318425
(48) Discovery of novel BTK PROTACs with improved metabolic stability via linker rigidification strategy. Chen, S.; Chen, Z.; Lu, L.; Zhao, Y.; Zhou, R.; Xie, Q.; Shu, Y.; Lin, J.; Yu, X.; Wang, Y. Eur J Med Chem 2023, 255, 115403
(49) Discovery of Ibrutinib-based BTK PROTACs with in vivo anti-inflammatory efficacy by inhibiting NF-kappaB activation. Huang, J.; Ma, Z.; Yang, Z.; He, Z.; Bao, J.; Peng, X.; Liu, Y.; Chen, T.; Cai, S.; Chen, J.; Zeng, Z. Eur J Med Chem 2023, 259, 115664
(50) Rapid labelling and covalent inhibition of intracellular native proteins using ligand-directed N-acyl-N-alkyl sulfonamide. Tamura, T.; Ueda, T.; Goto, T.; Tsukidate, T.; Shapira, Y.; Nishikawa, Y.; Fujisawa, A.; Hamachi, I. Nat Commun 2018, 9, 1870.PMC5951806
(51) Enhanced Suppression of a Protein-Protein Interaction in Cells Using Small-Molecule Covalent Inhibitors Based on an N-Acyl-N-alkyl Sulfonamide Warhead. Ueda, T.; Tamura, T.; Kawano, M.; Shiono, K.; Hobor, F.; Wilson, A. J.; Hamachi, I. J Am Chem Soc 2021, 143, 4766-4774
(52) Activation Method to Prepare a Highly Reactive Acylsulfonamide “Safety-Catch” Linker for Solid-Phase Synthesis1. Backes, B. J.; Virgilio, A. A.; Ellman, J. A. Journal of the American Chemical Society 1996, 118, 3055-3056
(53) Comparative analysis of prostate-specific membrane antigen (PSMA) versus a prostate-specific membrane antigen-like gene. O'Keefe, D. S.; Bacich, D. J.; Heston, W. D. Prostate 2004, 58, 200-10
(54) Bispecific small molecule-antibody conjugate targeting prostate cancer. Kim, C. H.; Axup, J. Y.; Lawson, B. R.; Yun, H.; Tardif, V.; Choi, S. H.; Zhou, Q.; Dubrovska, A.; Biroc, S. L.; Marsden, R.; Pinstaff, J.; Smider, V. V.; Schultz, P. G. Proc Natl Acad Sci USA 2013, 110, 17796-801.PMC3816437
(55) PSMA-Targeting Imaging and Theranostic Agents-Current Status and Future Perspective. Debnath, S.; Zhou, N.; McLaughlin, M.; Rice, S.; Pillai, A. K.; Hao, G.; Sun, X. Int J Mol Sci 2022, 23.PMC8835702.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

1. An immune cell recruiting chimeric small molecule comprising a target protein binding moiety and an immunogenic display moiety connected via one or more linker molecules, and optionally an electrophilic reactive group. wherein the protein binding moiety facilitates labeling of an amino acid of a protein, via the electrophilic reactive group, with the immunogenic display moiety.

2. The chimeric small molecule of claim 1, according to the formula 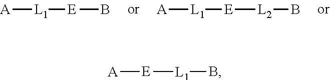
wherein A is the target protein binding moiety;
B is the immunogenic display moiety;
L₁ and L₂ are each a linker; and
E is an electrophilic reactive group.

3. The chimeric small molecule of claim 1, wherein the immunogenic display moiety is configured to bind with i) a cell surface of a natural or an engineered immune cell, or (ii) bifunctional bridge molecule comprising a first binding moiety that binds the immunogenic display moiety and a second binding moiety that binds the surface of a natural or engineered immune cell.

4. The chimeric small molecule of claim 3, wherein the natural or an engineered immune cell is a CAR T cell, T cell or an NK cell.

5. The chimeric small molecule of claim 1, wherein the target protein is a disease-specific protein, optionally an oncogenic-specific protein.

6. The chimeric small molecule of claim 1, wherein the protein binding moiety is a KRAS^{G12C}, EGFR, pan-EGFR, ITK, FGFR4, JAK3, RIP1, MEK1/2, CDK, AKT, TAK, JNK, BMX, LIMK, IRE1, IRE2, ABL1, EphA2 receptor, a human dipeptidyl peptidase IV/CD26, a HER2 receptor, a prostate-specific membrane antigen (PSMA), a folate receptor, or a somatostatin binding moiety.

7. The chimeric small molecule of claim 1, wherein the amino acid is lysine or cysteine.

8. A bifunctional immune cell engager comprising a first binding moiety capable of binding the immunogenic display moiety of the chimeric small molecule of claim 1 and a second binding moiety capable of binding a cell surface receptor of a natural or engineered immune cell.

9. The bifunctional immune cell engager of claim 8, wherein the immune cell is a CD8 T cell, a CD4 T cell, a NK cell, a CAR T cell, or an engineered tumor infiltrating lymphocyte (TIL).

10. The bifunctional immune cell engager of claim 8, wherein the cell surface receptor is CD3, CD19, CD20, CD22, CD30, CD33, CD38, CD79B, or SLAMF7.

11. The bifunctional immune cell engager of claim 8, wherein the immunogenic display moiety of the chimeric small molecule and the first binding moiety of the bifunctional immune cell engager together comprise a click chemistry reagent pair.

12. The bifunctional immune cell engager of claim 11, wherein a binding domain of the second binding moiety is masked such that the second binding moiety is incapable of binding a cell surface receptor of a natural or engineered immune cell, and wherein the click chemistry reaction of the click chemistry reagent pair unmasks the binding domain of the second binding moiety, such that the second binding moiety is capable of binding the cell surface receptor of the natural or engineered immune cell.

13. The bifunctional immune cell engager of claim 11, wherein the immunogenic display moiety is a tetrazine moiety, or a tetracyclooctene (TCO) moiety, and the first binding moiety is a corresponding TCO moiety, or tetrazine moiety.

14. The bifunctional immune cell engager of claim 8, wherein the immunogenic display moiety is a Halo Tag ligand and the first binding moiety is a Halo Tag protein.

15. The bifunctional immune cell engager of claim 8, wherein the immunogenic display moiety is an E3 ligase ligand, and the first binding moiety is an E3 ligase ligand binding moiety of a CRBN protein, or an antibody or antibody fragment to an E3 ligase ligand.

16. The bifunctional immune cell engager of claim 8, wherein the first binding moiety is an antibody, a scFV fragment, or a nanobody directed against the immunogenic display moiety.

17. The bifunctional immune cell engager of claim 8, wherein the bifunctional immune cell engager is a BiTE, wherein the first binding moiety is a first antibody variable region the binds the immunogenic display moiety and the second binding moiety is a second antibody variable region that binds a cell surface receptor on an immune cell.

18. A method of inducing immune response, comprising:
delivering the immune cell recruiting chimeric small molecule of claim 1 to a subject in need thereof;
labeling one or more target polypeptides of one or more target proteins with the immunogenic display moiety by the immune cell recruiting chimeric small molecule; and
displaying the one more target polypeptides labeled with the immunogenic display moiety on the cell surface via a Major Histocompatibility Complex (MHC) molecule.

19. The method of claim 18, further comprising eliciting an immune response by binding of the immunogenic display moiety to a natural or an engineered immune cell, thereby activating the immune cell.

20. The method of claim 18, further comprising eliciting an immune response by administering a bifunctional immune cell engager, wherein the first binding moiety of the bifunctional immune cell engager binds the immunogenic display moiety displayed on the surface of the cell and the second binding moiety of the bifunctional immune cell engager binds a cell surface receptor of a natural or engineered immune cell thereby activating the natural or engineered immune cell.

21. The method of claim 18, wherein two or more different target proteins are labeled with the same immunogenic display moiety, whereby each target polypeptide of each different target protein is recognized by the same natural or engineered immune cell.

22. The method of claim 21, wherein the chimeric small molecule that labels the two or more different target proteins is the same molecule or different molecules.

23. A method of labeling cell surface polypeptides, comprising:
delivering the chimeric small molecule of claim 1 to a cell; and
labeling one or more target cell surface polypeptides with the immunogenic display moiety by the chimeric small molecule.

24. The method of claim 23, further comprising eliciting an immune response by binding of the immunogenic display moiety to a natural or an engineered immune cell, thereby activating the natural or engineered immune cell.

25. The method of claim 23, further comprising eliciting an immune response by administering a bifunctional immune cell engager to the cell surface, wherein the first binding moiety of the bifunctional immune cell engager binds the immunogenic display moiety and the second binding moiety of the bifunctional immune cell engager binds a cell surface receptor of a natural or engineered immune cell, thereby activating the natural or engineered immune cell.

26. The method of claim 23, wherein two or more different target cell surface polypeptides are labeled with the same immunogenic display moiety, whereby each different target cell surface polypeptide is recognized by the same natural or engineered immune cell.

27. The method of claim 26, wherein the chimeric small molecule that labels the two or more different cell surface polypeptides is the same chimeric small molecule or different chimeric small molecules.

28. The method of claim 18, wherein the target protein is a disease-specific protein, optionally an oncogenic-specific protein.

29. The method of claim 28, wherein the target protein is KRASG12C, EGFR, pan-EGFR, ITK, FGFR4, JAK3, RIP1, MEK1/2, CDK, AKT, TAK, INK, BMX, LIMK, IRE1, IRE2, ABL1, EphA2 receptor, a human dipeptidyl peptidase IV/CD26, a HER2 receptor, a prostate-specific membrane antigen (PSMA), a folate receptor, or somatostatin.

30. The method of claim 23, wherein the target cell surface polypeptide is a disease-specific polypeptide, optionally an oncogenic-specific polypeptide.

31. The method of claim 30, wherein the target cell surface polypeptide is a prostate-specific membrane antigen (PSMA), a folate receptor, a somatostatin receptor, a human dipeptidyl peptidase IV/CD26, a HER2 receptor, or EGFR polypeptide.
